(19) United States
(12) Patent Application Publication
(10) Pub. No.: US 2017/0210818 A1
(43) Pub. Date: Jul. 27, 2017

(54) CONSTANT REGION ANTIBODY FUSION PROTEINS AND COMPOSITIONS THEREOF

(71) Applicants: The California Institute for Biomedical Research, La Jolla, CA (US); The Scripps Research Institute, La Jolla, CA (US)

(72) Inventors: Feng WANG, Carlsbad, CA (US); Juanjuan DU, San Diego, CA (US); Travis YOUNG, La Jolla, CA (US); Peter G. SCHULTZ, La Jolla, CA (US)

(21) Appl. No.: 15/315,641

(22) PCT Filed: Jun. 5, 2015
(86) PCT No.: PCT/US15/34541

§ 371 Date: Dec. 1, 2016

Related U.S. Application Data
(60) Provisional application No. 62/064,199, filed on Oct. 15, 2014.
(60) Provisional application No. 62/030,514, filed on Jul. 29, 2014.
(60) Provisional application No. 62/009,054, filed on Jun. 6, 2014.

(51) Int. Cl. C07K 16/32; C07K 14/715; C07K 14/47; C07K 14/705; C07K 14/505; C07K 16/28

(52) U.S. Cl. CPC C07K 16/32; C07K 14/505; C07K 16/2803; C07K 16/2809; C07K 14/47; C07K 14/705; C07K 14/7158; C07K 2317/31; C07K 2317/52; C07K 2317/522; C07K 2317/524; C07K 2317/526; C07K 2317/53; C07K 2317/622; C07K 2319/73; C07K 2319/30; C07K 2319/74; C07K 2317/55

(57) ABSTRACT

Disclosed herein are antibody fusion constructs and uses thereof. The antibody fusion construct may comprise an antibody fusion protein. The antibody fusion protein may comprise a non-antibody peptide inserted into an antibody portion of the antibody fusion protein. Alternatively, the antibody fusion construct may comprise a bispecific antibody. The bispecific antibody may comprise a second antibody or antibody fragment inserted into a first antibody or antibody fragment. Insertion of the non-antibody peptide (or second antibody or antibody fragment) into the antibody portion (or first antibody or antibody fragment) may comprise replacement of one or more amino acids in a constant domain of the antibody portion (or first antibody or antibody fragment). The antibody fusion constructs disclosed herein may be used to treat a disease, such as a cancer, an autoimmune disorder or an infection.

### CROSS-REFERENCE

This application is a U.S. National Stage entry of International Application No. PCT/US15/34541, filed Jun. 5, 2015, which claims the benefit of U.S. provisional application Ser. No. 62/009,054, filed Jun. 6, 2014; U.S. provisional application Ser. No. 62/064,199 filed Oct. 15, 2014; and U.S. provisional application Ser. No. 62/030,514, filed Jul. 29, 2014; which are all incorporated by reference in their entirety.

### SEQUENCE LISTING

The instant application contains a Sequence Listing which has been submitted electronically in ASCII format and is hereby incorporated by reference in its entirety. Said ASCII copy, created on Feb. 23, 2017, is named 41135-729_831_SL.txt and is 198,850 bytes in size.

### BACKGROUND OF THE INVENTION

Antibodies are natural proteins that the vertebrate immune system forms in response to foreign substances (antigens), primarily for defense against infection. For over a century, antibodies have been induced in animals under artificial conditions and harvested for use in therapy or diagnosis of disease conditions, or for biological research. Each individual antibody producing cell produces a single type of antibody with a chemically defined composition, however, antibodies obtained directly from animal serum in response to antigen inoculation actually comprise an ensemble of non-identical molecules (e.g., polyclonal antibodies) made from an ensemble of individual antibody producing cells.

Antibody fusion constructs can be used to improve the delivery of drugs or other agents to target cells, tissues and tumors. Antibody fusion constructs may comprise a chemical linker to attach a drug or other agent to antibody. Exemplary antibody fusion constructs and methods of producing antibody fusion constructs are disclosed in US patent application numbers 20060182751, 20070160617 and U.S. Pat. No. 7,736,652, each of which are incorporated by reference in their entireties.

Disclosed herein are novel constant region antibody fusion proteins and methods of producing such constant region antibodyfusion proteins. Further disclosed herein are uses of the constant region fusion proteins for the treatment of various diseases and conditions.

### SUMMARY OF THE INVENTION

Disclosed herein are antibody fusion proteins. The antibody fusion protein may be a constant region antibody fusion protein. The antibody fusion protein may be a bispecific antibody fusion protein. In some instances, an antibody fusion protein may comprise (a) antibody fusion protein comprising: an antibody region comprising an antibody or antibody fragment, wherein the antibody or antibody fragment comprises a modified constant domain; and a non-antibody polypeptide region comprising 15 or more amino acids, wherein the non-antibody polypeptide region is located within the modified constant domain. The non-antibody polypeptide may be inserted into the modified constant domain by replacing less than about 20 amino acids of the modified constant domain. The the non-antibody polypeptide may be inserted into the modified constant domain without replacing any amino acids of the modified constant domain. The non-antibody polypeptide may be located within a loop of the modified constant domain. The modified constant domain may comprise a heavy chain constant domain or a portion thereof. The heavy chain constant domain may be a CH1 domain. The modified constant domain may comprise a light chain constant domain (CL1) or a portion thereof. The modified constant domain may comprise an antibody hinge region or a portion thereof. The non-antibody polypeptide region may be located between a CH1 or portion thereof of the antibody or antibody fragment and a hinge region or portion thereof of the antibody or antibody fragment. The non-antibody polypeptide region may possess more than about 5 amino acids or more than about 10 amino acids. The non-antibody polypeptide region may possess more than about 15 amino acids, more than about 18 amino acids, more than about 20 amino acids, more than about 22 amino acids, more than about 25 amino acids, more than about 28 amino acids, more than about 30 amino acids, more than about 32 amino acids, more than about 35 amino acids, more than about 40 amino acids, more than about 45 amino acids, or more than about 50 amino acids. The non-antibody polypeptide region may possess more than about 75 amino acids. The non-antibody polypeptide region may possess more than about 100 amino acids. The non-antibody polypeptide region may possess more than about 100 to more than about 150 amino acids. The non-antibody polypeptide region may possess more than about 150 to more than about 200 amino acids. The antibody region may comprise an antibody or antibody fragment selected from an anti-CD19 antibody, an anti-CD20 antibody, an anti-Her2 antibody, UCHT1, palivizumab, and fragments thereof.

The non-antibody peptide may be a non-antigenic peptide. In some instances, the non-antibody peptide is not based on or derived from a T cell epitope. In some instances, the non-antibody peptide is not based on or derived from a B cell epitope. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody.

Disclosed herein are antibody fusion proteins. The antibody fusion protein may be a constant region antibody fusion protein. The antibody fusion protein may be a bispecific antibody fusion protein. In some instances, an antibody fusion protein may comprise (a) an antibody region based on or derived from an antibody or antibody fragment that comprises a modified constant domain; and (b) a non-antibody polypeptide region comprising 15 or more amino acids, wherein the non-antibody polypeptide region is located within the modified constant domain. The non-antibody polypeptide may be inserted into the modified constant domain by replacing less than about 20 amino acids of the modified constant domain. The the non-antibody polypeptide may be inserted into the modified constant domain without replacing any amino acids of the modified constant domain. The non-antibody polypeptide may be located within a loop of the modified constant domain. The non-antibody peptide may be a non-antigenic peptide. In some instances, the non-antibody peptide is not based on or derived from a T cell epitope. In some instances, the non-antibody peptide is not based on or derived from a B cell epitope. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody.

The non-antibody polypeptide region may be inserted into the modified constant domain of the antibody or antibody fragment. The non-antibody polypeptide region may be inserted into a loop region of the antibody or antibody fragment. The non-antibody polypeptide region may be inserted into a loop region of the modified constant domain of the antibody or antibody fragment. The non-antibody polypeptide region may be inserted near a beta strand of the antibody region. The non-antibody polypeptide region may be inserted within 20 amino acids of a beta strand of the antibody region. The non-antibody polypeptide region may be inserted within 15 amino acids of a beta strand of the antibody region. The non-antibody polypeptide region may be inserted within 10 amino acids of a beta strand of the antibody region. The non-antibody polypeptide region may be inserted within 5 amino acids of a beta strand of the antibody region. The less than about 20 amino acid residues to be replaced may be located between two beta strands. The non-antibody polypeptide region may be inserted into the antibody region by replacement of less than about 20 amino acid residues from the modified constant domain of the antibody or antibody fragment with the non-antibody polypeptide region. The less than about 20 amino acid residues to be replaced may be located near a beta strand. The less than about 20 amino acid residues to be replaced may be within 20 amino acids of a beta strand. The less than about 20 amino acid residues to be replaced may be within 15 amino acids of a beta strand. The less than about 20 amino acid residues to be replaced may be within 10 amino acids of a beta strand. The less than about 20 amino acid residues to be replaced may be within 5 amino acids of a beta strand. The less than about 20 amino acid residues to be replaced may be located between two beta strands. The modified constant domain may be from a heavy chain of the antibody or antibody fragment. The modified constant domain may be from a light chain of the antibody or antibody fragment.

The antibody region may comprise a consensus insertion sequence. The consensus insertion sequence may comprise an amino acid sequence that is at least about 50% homologous to an amino acid sequence selected from a group consisting of SEQ ID NO: 89-120. The consensus insertion sequence may comprise an amino acid sequence that is at least about 60% homologous to an amino acid sequence selected from a group consisting of SEQ ID NO: 89-120. The consensus insertion sequence may comprise an amino acid sequence that is at least about 70% homologous to an amino acid sequence selected from a group consisting of SEQ ID NO: 89-120. The consensus insertion sequence may comprise an amino acid sequence that is at least about 80% homologous to an amino acid sequence selected from a group consisting of SEQ ID NO: 89-120. The consensus insertion sequence may comprise an amino acid sequence that is at least about 90% homologous to an amino acid sequence selected from a group consisting of SEQ ID NO: 89-120. The consensus insertion sequence may comprise an amino acid sequence that is at least about 95% homologous to an amino acid sequence selected from a group consisting of SEQ ID NO: 89-120. The consensus insertion sequence may be based on or derived from a constant domain of the antibody or antibody fragment. The consensus insertion sequence may be based on or derived from a loop region of the antibody or antibody fragment. The consensus insertion sequence may be based on or derived from a loop region of a constant domain of the antibody or antibody fragment. The consensus insertion sequence may be based on or derived from a sequence located between two beta strands of the antibody or antibody fragment. The two beta strands may be in a constant domain of the antibody or antibody fragment. The constant domain may be in a heavy chain. The constant domain may be CH1. The constant domain may be CH2. The constant domain may be CH3. The constant domain may be in a light chain. The loop region may be in a heavy chain. The loop region may be in the light chain. The two beta strands may be in a heavy chain. The two beta strands may be in a light chain. The non-antibody polypeptide region may be inserted into the consensus insertion sequence of the antibody region. The non-antibody polypeptide region may be inserted into the antibody region by replacement of less than about 20 amino acids from the consensus insertion sequence of the antibody region. The non-antibody polypeptide region may be inserted into the consensus insertion sequence by replacement of one or more amino acids from the consensus insertion sequence. The non-antibody polypeptide region may be inserted into the consensus insertion sequence by replacement of two or more amino acids from the consensus insertion sequence. The non-antibody polypeptide region may be inserted into the consensus insertion sequence by replacement of three or more amino acids from the consensus insertion sequence. The non-antibody polypeptide region may be inserted into the consensus insertion sequence by replacement of four or more amino acids from the consensus insertion sequence. The non-antibody polypeptide region may be inserted into the consensus insertion sequence by replacement of five or more amino acids from the consensus insertion sequence.

The non-antibody polypeptide region may be inserted into the antibody region by replacement of less than about 20 amino acid residues from a heavy chain of the antibody or antibody fragment with the non-antibody polypeptide region. The non-antibody polypeptide region may be inserted into the antibody region by replacement of less than about 20 amino acid residues from the modified constant domain of the heavy chain of the antibody or antibody fragment with the non-antibody polypeptide region. The modified constant domain of the heavy chain may be CH1. The modified constant domain of the heavy chain may be CH2.The modified constant domain of the heavy chain may be CH3.

The non-antibody polypeptide region may be inserted into the antibody region by replacement of less than about 20 amino acid residues from a light chain of the antibody or antibody fragment with the non-antibody polypeptide region. The non-antibody polypeptide region may be inserted into the antibody region by replacement of less than about 20 amino acid residues from the constant domain of the light chain of the antibody or antibody fragment with the non-antibody polypeptide region.

The replacement of less than about 20 amino acid residues may comprise replacement of at least 1 amino acid residue from the antibody or antibody fragment with the non-antibody polypeptide region. The replacement of less than about 20 amino acid residues may comprise replacement of at least 2 amino acid residues from the antibody or antibody fragment with the non-antibody polypeptide region.

The replacement of less than about 20 amino acid residues may comprise replacement of at least 3 amino acid residues from the antibody or antibody fragment with the non-antibody polypeptide region. The replacement of less than about 20 amino acid residues may comprise replacement of less than 15 amino acid residues from the antibody or antibody fragment with the non-antibody polypeptide region. The replacement of less than about 20 amino acid residues may comprise replacement of less than 10 amino acid residues from the antibody or antibody fragment with the non-antibody polypeptide region. The replacement of less than about 20 amino acid residues may comprise replacement of less than 5 amino acid residues from the antibody or antibody fragment with the non-antibody polypeptide region.

The replacement of less than about 20 amino acid residues may comprise replacement of 5 or fewer amino acid residues from the antibody or antibody fragment with the non-antibody polypeptide region. The replacement of less than about 20 amino acid residues may comprise replacement of 4 or fewer amino acid residues from the antibody or antibody fragment with the non-antibody polypeptide region. The replacement of less than about 20 amino acid residues may comprise replacement of 3 or fewer amino acid residues from the antibody or antibody fragment with the non-antibody polypeptide region.

The replacement of less than about 20 amino acid residues may comprise replacement of 1-15 amino acid residues from the antibody or antibody fragment with the non-antibody polypeptide region. The replacement of less than about 20 amino acid residues may comprise replacement of 1-10 amino acid residues from the antibody or antibody fragment with the non-antibody polypeptide region. The replacement of less than about 20 amino acid residues may comprise replacement of 1-5 amino acid residues from the antibody or antibody fragment with the non-antibody polypeptide region. The replacement of the amino acid residues may comprise replacement of one or more amino acids selected from a group consisting of serine (S), glycine (G), lysine (K), proline (P), threonine (T), glutamine (Q), glutamic acid (E), alanine (A), asparagine (N), and histidine (H). The one or more amino acids may be from a consensus insertion sequence in the antibody region.

The replacement of less than about 20 amino acids may comprise replacement of 5 or fewer amino acid residues from the CH1 domain of the antibody or antibody fragment.The replacement of less than about 20 amino acids may comprise replacement of 4 or fewer amino acid residues from the CH1 domain of the antibody or antibody fragment. The replacement of less than about 20 amino acids may comprise replacement of 3 or fewer amino acid residues from the CH1 domain of the antibody or antibody fragment. The replacement of less than about 20 amino acids may comprise replacement of 2 or fewer amino acid residues from the CH1 domain of the antibody or antibody fragment. The replacement of less than about 20 amino acids may comprise replacement of 1 amino acid residue from the CH1 domain of the antibody or antibody fragment.

The one or more amino acid residues that are replaced may be selected from a group consisting of serine (S), glycine (G), proline (P), threonine (T), and glutamine (Q). The amino acid residues may be from a consensus insertion sequence of the antibody region. The one or more amino acids that are replaced may be in a loop region of the CH1 domain. The replacement of less than about 20 amino acids may comprise replacement of one or more of lysine 136 (K136), serine 137 (S137), threonine 138 (T138) from the Fab heavy chain. The replacement of less than about 20 amino acids may comprise replacement of serine 180 (S180) from the CH1 domain. The replacement of less than about 20 amino acids may comprise replacement of glycine 181 (G181) from the CH1 domain. The replacement of less than about 20 amino acids may comprise replacement of serine 180 (S180) and glycine 181 (G181) from the CH1 domain. The replacement of less than about 20 amino acids may comprise replacement of proline 156 (P156) from the CH1 domain. The replacement of less than about 20 amino acids may comprise replacement of lysine 138 (K138) from the CH1 domain. The replacement of less than about 20 amino acids may comprise replacement of threonine 169 (T169) from the CH1 domain. The replacement of less than about 20 amino acids may comprise replacement of serine 170 (S170) from the CH1 domain. The replacement of less than about 20 amino acids may comprise replacement of threonine 169 (T169) and serine 170 (S170) from the CH1 domain. The replacement of less than about 20 amino acids may comprise replacement of glutamine 201 (Q201) from the CH1 domain. The replacement of less than about 20 amino acids may comprise replacement of proline 211 (P211) from the CH1 domain. The replacement of less than about 20 amino acids may comprise replacement of serine and glycine from the CH1 domain. The serine and glycine may be adjacent to each other. The replacement of less than about 20 amino acids may comprise replacement of threonine and serine from the CH1 domain. The threonine and serine may be adjacent to each other.

The replacement of less than about 20 amino acids may comprise replacement of 5 or fewer amino acid residues from the CH2 domain of the antibody or antibody fragment. The replacement of less than about 20 amino acids may comprise replacement of 4 or fewer amino acid residues from the CH2 domain of the antibody or antibody fragment. The replacement of less than about 20 amino acids may comprise replacement of 3 or fewer amino acid residues from the CH2 domain of the antibody or antibody fragment. The replacement of less than about 20 amino acids may comprise replacement of 2 or fewer amino acid residues from the CH2 domain of the antibody or antibody fragment. The replacement of less than about 20 amino acids may comprise replacement of 1 amino acid residue from the CH2 domain of the antibody or antibody fragment.

The replacement of less than about 20 amino acids may comprise replacement of one or more amino acids from the CH2 domain. The one or more amino acid residues may be selected from a group consisting of glutamic acid (E), alanine (A) and proline (P). The replacement of less than about 20 amino acids may comprise replacement of glutamic acid 274 (E274) from the CH2 domain. The replacement of less than about 20 amino acids may comprise replacement of alanine 302 (A302) from the CH2 domain. The replacement of less than about 20 amino acids may comprise replacement of proline 334 (P334) from the CH2 domain.

The replacement of less than about 20 amino acids may comprise replacement of 5 or fewer amino acid residues from the CH3 domain of the antibody or antibody fragment. The replacement of less than about 20 amino acids may comprise replacement of 4 or fewer amino acid residues from the CH3 domain of the antibody or antibody fragment. The replacement of less than about 20 amino acids may comprise replacement of 3 or fewer amino acid residues from the CH3 domain of the antibody or antibody fragment. The replacement of less than about 20 amino acids may comprise replacement of 2 or fewer amino acid residues from the CH3 domain of the antibody or antibody fragment. The replacement of less than about 20 amino acids may comprise replacement of 1 amino acid residue from the CH3 domain of the antibody or antibody fragment. The replacement of less than about 20 amino acids may comprise replacement of one or more amino acids from the CH3 domain, wherein the one or more amino acid residues may be selected from a group consisting of threonine (T), lysine (K), asparagine (N), and glycine (G).

The replacement of less than about 20 amino acids may comprise replacement of threonine 361 (T361) from the CH3 domain. The replacement of less than about 20 amino acids may comprise replacement of lysine 362 (K362) from the CH3 domain. The replacement of less than about 20 amino acids may comprise replacement of asparagine 363 (N363) from the CH3 domain. The replacement of less than about 20 amino acids may comprise replacement of threonine 361 (T361), lysine 362 (K362), and asparagine 363 (N363) from the CH3 domain. The replacement of less than about 20 amino acids may comprise replacement of asparagine 389 (N389) from the CH3 domain. The replacement of less than about 20 amino acids may comprise replacement of glycine 390 (G390) from the CH3 domain. The replacement of less than about 20 amino acids may comprise replacement of asparagine 389 (N389) and glycine 390 (G390) from the CH3 domain. The replacement of less than about 20 amino acids may comprise replacement of glycine 425 (G425) from the CH3 domain. The replacement of less than about 20 amino acids may comprise replacement of asparagine 426 (N426) from the CH3 domain. The replacement of less than about 20 amino acids may comprise replacement of glycine 425 (G425) and asparagine 363 (N363) from the CH3 domain. The replacement of less than about 20 amino acids may comprise replacement of threonine and asparagine from the CH3 domain. The threonine and asparagine may be adjacent to each other. The the replacement of less than about 20 amino acids may comprise replacement of threonine, lysine, and asparagine from the CH3 domain. The threonine, lysine, and asparagine may be adjacent to each other.

The replacement of less than about 20 amino acids may comprise replacement of 5 or fewer amino acid residues from the constant domain of the light chain of the antibody or antibody fragment. The replacement of less than about 20 amino acids may comprise replacement of 4 or fewer amino acid residues from the constant domain of the light chain of the antibody or antibody fragment. The replacement of less than about 20 amino acids may comprise replacement of 3 or fewer amino acid residues from the constant domain of the light chain of the antibody or antibody fragment. The replacement of less than about 20 amino acids may comprise replacement of 2 or fewer amino acid residues from the constant domain of the light chain of the antibody or antibody fragment. The replacement of less than about 20 amino acids may comprise replacement of 1 amino acid residue from the constant domain of the light chain of the antibody or antibody fragment.

The replacement of less than about 20 amino acids may comprise replacement of one or more amino acids from the constant domain of the light chain; and wherein the one or more amino acid residues may be selected from a group consisting of serine (S), glycine (G), proline (P), lysine (K), asparagine (N) and histidine (H). The replacement of less than about 20 amino acids may comprise replacement of serine 202 (S202) from the constant domain of the light chain. The replacement of less than about 20 amino acids may comprise replacement of glycine 128 (G128) from the constant domain of the light chain. The replacement of less than about 20 amino acids may comprise replacement of lysine 169 (1(169) from the constant domain of the light chain. The replacement of less than about 20 amino acids may comprise replacement of proline 141 (P141) from the constant domain of the light chain. The replacement of less than about 20 amino acids may comprise replacement of asparagine (N152) from the constant domain of the light chain. The replacement of less than about 20 amino acids may comprise replacement of lysine 138 (K138) from the constant domain of the light chain. The replacement of less than about 20 amino acids may comprise replacement of histidine 139 (H139) from the constant domain of the light chain. The replacement of less than about 20 amino acids may comprise replacement of lysine 138 (K138) and histidine (H139) from the constant domain of the light chain. The replacement of less than about 20 amino acids may comprise replacement of lysine and histidine from the constant domain of the light chain. The lysine and histidine may be adjacent to each other.

The non-antibody polypeptide region may be based on or derived from one or more proteins selected from a group consisting of erythropoietin (EPO), a chemokine (CXC Motif) receptor-4 (CXCR4) binding peptide (CXCR4-BP), tumor-homing peptide, integrin αvβ33 binding peptide, and T-cell epitope peptide. The tumor-homing peptide may be NGR. The tumor-homing peptide may be TCP-1. The integrin αvβ33 binding peptide may be Int. The T-cell epitope peptide may be GCN4.

The erythropoietin may comprise an amino acid sequence that is at least 50% homologous to SEQ ID NO: 85. The erythropoietin may comprise an amino acid sequence that is at least 60% homologous to SEQ ID NO: 85. The erythropoietin may comprise an amino acid sequence that is at least 70% homologous to SEQ ID NO: 85. The erythropoietin may comprise an amino acid sequence that is at least 80% homologous to SEQ ID NO: 85. The erythropoietin may comprise an amino acid sequence that is at least 90% homologous to SEQ ID NO: 85.

The CXCR4-BP may comprise an amino acid sequence that is at least 50% homologous to SEQ ID NO: 83. The CXCR4-BP may comprise an amino acid sequence that is at least 60% homologous to SEQ ID NO: 83. The CXCR4-BP may comprise an amino acid sequence that is at least 70% homologous to SEQ ID NO: 83. The CXCR4-BP may comprise an amino acid sequence that is at least 80% homologous to SEQ ID NO: 83. The CXCR4-BP may comprise an amino acid sequence that is at least 90% homologous to SEQ ID NO: 83.

The TCP1 may comprise an amino acid sequence that is at least 50% homologous to SEQ ID NO: 78. The TCP1 may comprise an amino acid sequence that is at least 60% homologous to SEQ ID NO: 78. The TCP1 may comprise an amino acid sequence that is at least 70% homologous to SEQ ID NO: 78. The TCP1 may comprise an amino acid sequence that is at least 80% homologous to SEQ ID NO: 78. The TCP1 may comprise an amino acid sequence that is at least 90% homologous to SEQ ID NO: 78.

The TCP1 may comprise an amino acid sequence that is at least 50% homologous to SEQ ID NO: 79. The TCP1 may comprise an amino acid sequence that is at least 60% homologous to SEQ ID NO: 79. The TCP1 may comprise an amino acid sequence that is at least 70% homologous to SEQ ID NO: 79. The TCP1 may comprise an amino acid sequence that is at least 80% homologous to SEQ ID NO: 79. The TCP1 may comprise an amino acid sequence that is at least 90% homologous to SEQ ID NO: 79.

The NGR may comprise an amino acid sequence that is at least 50% homologous to SEQ ID NO: 80. The NGR may comprise an amino acid sequence that is at least 60% homologous to SEQ ID NO: 80. The NGR may comprise an amino acid sequence that is at least 70% homologous to SEQ ID NO: 80. The NGR may comprise an amino acid sequence that is at least 80% homologous to SEQ ID NO: 80. The NGR may comprise an amino acid sequence that is at least 90% homologous to SEQ ID NO: 80.

The NGR may comprise an amino acid sequence that is at least 50% homologous to SEQ ID NO: 81. The NGR may comprise an amino acid sequence that is at least 60% homologous to SEQ ID NO: 81. The NGR may comprise an amino acid sequence that is at least 70% homologous to SEQ ID NO: 81. The NGR may comprise an amino acid sequence that is at least 80% homologous to SEQ ID NO: 81. The NGR may comprise an amino acid sequence that is at least 90% homologous to SEQ ID NO: 81.

The Int may comprise an amino acid sequence that is at least 50% homologous to SEQ ID NO: 82. The Int may comprise an amino acid sequence that is at least 60% homologous to SEQ ID NO: 82. The Int may comprise an amino acid sequence that is at least 70% homologous to SEQ ID NO: 82. The Int may comprise an amino acid sequence that is at least 80% homologous to SEQ ID NO: 82. The Int may comprise an amino acid sequence that is at least 90% homologous to SEQ ID NO: 82.

The GCN4 may comprise an amino acid sequence that is at least 50% homologous to SEQ ID NO: 84. The GCN4 may comprise an amino acid sequence that is at least 60% homologous to SEQ ID NO: 84. The GCN4 may comprise an amino acid sequence that is at least 70% homologous to SEQ ID NO: 84. The GCN4 may comprise an amino acid sequence that is at least 80% homologous to SEQ ID NO: 84. The GCN4 may comprise an amino acid sequence that is at least 90% homologous to SEQ ID NO: 84.

The antibody fusion protein may comprise an amino acid sequence that is at least 50% homologous homologous to an amino acid sequence selected from a group consisting of SEQ ID NOS: 45-57, 61-66. The antibody fusion protein may comprise an amino acid sequence that is at least 60% homologous to an amino acid sequence selected from a group consisting of SEQ ID NOS: 45-57, 61-66. The antibody fusion protein may comprise an amino acid sequence that is at least 70% homologous to an amino acid sequence selected from a group consisting of SEQ ID NOS: 45-57, 61-66. The antibody fusion protein may comprise an amino acid sequence that is at least 80% homologous to an amino acid sequence selected from a group consisting of SEQ ID NOS: 45-57, 61-66. The antibody fusion protein may comprise an amino acid sequence that is at least 90% homologous to an amino acid sequence selected from a group consisting of SEQ ID NOS: 45-57, 61-66.

The antibody fusion protein may comprise an amino acid sequence that comprises 50 or more consecutive amino acids from any one of SEQ ID NOS: 45-57, 61-66. The antibody fusion protein may comprise an amino acid sequence that comprises 100 or more consecutive amino acids from any one of SEQ ID NOS: 45-57, 61-66. The antibody fusion protein may comprise an amino acid sequence that comprises 150 or more consecutive amino acids from any one of SEQ ID NOS: 45-57, 61-66. The antibody fusion protein may comprise an amino acid sequence that comprises 200 or more consecutive amino acids from any one of SEQ ID NOS: 45-57, 61-66.

The antibody fusion protein may be encoded by a nucleic acid sequence that is at least 50% homologous to a nucleic acid sequence selected from a group consisting of SEQ ID NOS: 11-23, 27 and 28. The antibody fusion protein may be encoded by a nucleic acid sequence that is at least 60% homologous to a nucleic acid sequence selected from a group consisting of SEQ ID NOS: 11-23, 27 and 28. The antibody fusion protein may be encoded by a nucleic acid sequence that is at least 70% homologous to a nucleic acid sequence selected from a group consisting of SEQ ID NOS: 11-23, 27 and 28. The antibody fusion protein may be encoded by a nucleic acid sequence that is at least 80% homologous to a nucleic acid sequence selected from a group consisting of SEQ ID NOS: 11-23, 27 and 28. The antibody fusion protein may be encoded by a nucleic acid sequence that is at least 90% homologous to a nucleic acid sequence selected from a group consisting of SEQ ID NOS: 11-23, 27 and 28.

The antibody fusion protein may further comprise one or more additional antibodies or antibody fragments. The one or more additional antibodies or antibody fragments may comprise an amino acid sequence that is at least 50% homologous to an amino acid sequence selected from a group consisting of SEQ ID NO: 33-44. The one or more additional antibodies or antibody fragments may comprise an amino acid sequence that is at least 60% homologous to an amino acid sequence selected from a group consisting of SEQ ID NO: 33-44. The one or more additional antibodies or antibody fragments may comprise an amino acid sequence that is at least 70% homologous to an amino acid sequence selected from a group consisting of SEQ ID NO: 33-44. The one or more additional antibodies or antibody fragments may comprise an amino acid sequence that is at least 80% homologous to an amino acid sequence selected from a group consisting of SEQ ID NO: 33-44. The one or more additional antibodies or antibody fragments may comprise an amino acid sequence that is at least 90% homologous to an amino acid sequence selected from a group consisting of SEQ ID NO: 33-44.

The one or more additional antibodies or antibody fragments may comprise an amino acid sequence that comprises 50 or more consecutive amino acids from any one of SEQ ID NO: 33-44. The one or more additional antibodies or antibody fragments may comprise an amino acid sequence that comprises 100 or more consecutive amino acids from any one of SEQ ID NO: 33-44. The one or more additional antibodies or antibody fragments may comprise an amino acid sequence that comprises 150 or more consecutive amino acids from any one of SEQ ID NO: 33-44. The one or more additional antibodies or antibody fragments may comprise an amino acid sequence that comprises 200 or more consecutive amino acids from any one of SEQ ID NO: 33-44.

The one or more additional antibodies or antibody fragments may be encoded by a nucleic acid sequence that is at least 50% homologous to a nucleic acid sequence selected from a group consisting of SEQ ID NO: 1-10. The one or more additional antibodies or antibody fragments may be encoded by a nucleic acid sequence that is at least 6% homologous to a nucleic acid sequence selected from a group consisting of SEQ ID NO: 1-10. The one or more additional antibodies or antibody fragments may be encoded by a nucleic acid sequence that is at least 70% homologous to a nucleic acid sequence selected from a group consisting of SEQ ID NO: 1-10. The one or more additional antibodies or antibody fragments may be encoded by a nucleic acid sequence that is at least 80% homologous to a nucleic acid sequence selected from a group consisting of SEQ ID NO: 1-10. The one or more additional antibodies or antibody fragments may be encoded by a nucleic acid sequence that is at least 90% homologous to a nucleic acid sequence selected from a group consisting of SEQ ID NO: 1-10.

The one or more additional antibodies or antibody fragments may be encoded by a nucleic acid sequence that comprises 100 or more consecutive nucleic acids from any one of SEQ ID NO: 1-10. The one or more additional antibodies or antibody fragments may be encoded by a nucleic acid sequence that comprises 200 or more consecutive nucleic acids from any one of SEQ ID NO: 1-10. The one or more additional antibodies or antibody fragments may be encoded by a nucleic acid sequence that comprises 300 or more consecutive nucleic acids from any one of SEQ ID NO: 1-10. The one or more additional antibodies or antibody fragments may be encoded by a nucleic acid sequence that comprises 400 or more consecutive nucleic acids from any one of SEQ ID NO: 1-10.

Further disclosed herein are bispecific antibodies and uses thereof. A bispecific antibody may comprise (a) first antibody or antibody fragment comprising a modified constant; and (b) a second antibody or antibody fragment, wherein the second antibody or antibody fragment may be inserted into the modified constant domain. In some instances, the second antibody or antibody fragment is inserted into the first antibody or antibody fragment by replacement of less than about 20 amino acid residues from the first antibody or antibody fragment with the second antibody or antibody fragment. Alternatively, insertion of the second antibody or antibody fragment does not comprise replacement of one or more amino acid residues from the modified constant domain of the first antibody or antibody fragment. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody.

Further disclosed herein are bispecific antibodies and uses thereof. The bispecific antibody may comprise (a) an antibody region comprising a first antibody or antibody fragment, wherein the first antibody or antibody fragment comprises a modified constant domain; and (b) a second antibody or antibody fragment, wherein the second antibody or antibody fragment may be inserted into the first antibody or antibody fragment by replacement of less than about 20 amino acid residues from the first antibody or antibody fragment with the second antibody or antibody fragment. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody.

The second antibody or antibody fragment may be inserted into the modified constant domain of the antibody or antibody fragment. The second antibody or antibody fragment may be inserted into a loop region of the antibody or antibody fragment. The second antibody or antibody fragment may be inserted into a loop region of the modified constant domain of the antibody or antibody fragment. The second antibody or antibody fragment may be inserted near a beta strand of the first antibody or antibody fragment. The second antibody or antibody fragment may be inserted within 20 amino acids of a beta strand of the first antibody or antibody fragment. The second antibody or antibody fragment may be inserted within 15 amino acids of a beta strand of the first antibody or antibody fragment. The second antibody or antibody fragment may be inserted within 10 amino acids of a beta strand of the first antibody or antibody fragment. The second antibody or antibody fragment may be inserted within 5 amino acids of a beta strand of the first antibody or antibody fragment. The less than about 20 amino acid residues to be replaced may be located between two beta strands. The second antibody or antibody fragment may be inserted into the first antibody or antibody fragment by replacement of less than about 20 amino acid residues from a constant domain of the antibody or antibody fragment with the second antibody or antibody fragment. The less than about 20 amino acid residues to be replaced may be located near a beta strand. The less than about 20 amino acid residues to be replaced may be within 20 amino acids of a beta strand. The less than about 20 amino acid residues to be replaced may be within 15 amino acids of a beta strand. The less than about 20 amino acid residues to be replaced may be within 10 amino acids of a beta strand. The less than about 20 amino acid residues to be replaced may be within 5 amino acids of a beta strand. The less than about 20 amino acid residues to be replaced may be located between two beta strands. The modified constant domain may be from a heavy chain of the antibody or antibody fragment. The modified constant domain may be from a light chain of the antibody or antibody fragment.

The first antibody or antibody fragment may comprise a consensus insertion sequence. The consensus insertion sequence may comprise an amino acid sequence that is at least about 50% homologous to an amino acid sequence selected from a group consisting of SEQ ID NO: 89-120. The consensus insertion sequence may comprise an amino acid sequence that is at least about 60% homologous to an amino acid sequence selected from a group consisting of SEQ ID NO: 89-120. The consensus insertion sequence may comprise an amino acid sequence that is at least about 70% homologous to an amino acid sequence selected from a group consisting of SEQ ID NO: 89-120. The consensus insertion sequence may comprise an amino acid sequence that is at least about 80% homologous to an amino acid sequence selected from a group consisting of SEQ ID NO: 89-120. The consensus insertion sequence may comprise an amino acid sequence that is at least about 90% homologous to an amino acid sequence selected from a group consisting of SEQ ID NO: 89-120. The consensus insertion sequence may comprise an amino acid sequence that is at least about 95% homologous to an amino acid sequence selected from a group consisting of SEQ ID NO: 89-120. The consensus insertion sequence may be based on or derived from a constant domain of the antibody or antibody fragment. The consensus insertion sequence may be based on or derived from a loop region of the antibody or antibody fragment. The consensus insertion sequence may be based on or derived from a loop region of a constant domain of the antibody or antibody fragment. The consensus insertion sequence may be based on or derived from a sequence located between two beta strands of the antibody or antibody fragment. The two beta strands may be in a constant domain of the antibody or antibody fragment. The constant domain may be in a heavy chain. The constant domain may be CH1. The constant domain may be CH2. The constant domain may be CH3. The constant domain may be in a light chain. The loop region may be in a heavy chain. The loop region may be in the light chain. The two beta strands may be in a heavy chain. The two beta strands may be in a light chain. The second antibody or antibody fragment may be inserted into the consensus insertion sequence of the first antibody or antibody fragment. The second antibody or antibody fragment may be inserted into the first antibody or antibody fragment by replacement of less than about 20 amino acids from the consensus insertion sequence of the first antibody or antibody fragment. The second antibody or antibody fragment may be inserted into the consensus insertion sequence by replacement of one or more amino acids from the consensus insertion sequence. The second antibody or antibody fragment may be inserted into the consensus insertion sequence by replacement of two or more amino acids from the consensus insertion sequence. The second antibody or antibody fragment may be inserted into the consensus insertion sequence by replacement of three or more amino acids from the consensus insertion sequence. The second antibody or antibody fragment may be inserted into the consensus insertion sequence by replacement of four or more amino acids from the consensus insertion sequence. The second antibody or antibody fragment may be inserted into the consensus insertion sequence by replacement of five or more amino acids from the consensus insertion sequence.

The second antibody or antibody fragment may be inserted into the antibody region by replacement of less than about 20 amino acid residues from a heavy chain of the first antibody or antibody fragment with the second antibody or antibody fragment. The second antibody or antibody fragment may be inserted into the antibody region by replacement of less than about 20 amino acid residues from a constant domain of the heavy chain of the first antibody or antibody fragment with the second antibody or antibody fragment. The constant domain of the heavy chain may be CH1. The constant domain of the heavy chain may be CH2. The constant domain of the heavy chain may be CH3.

The second antibody or antibody fragment may be inserted into the antibody region by replacement of less than about 20 amino acid residues from a light chain of the first antibody or antibody fragment with the second antibody or antibody fragment. The second antibody or antibody fragment may be inserted into the antibody region by replacement of less than about 20 amino acid residues from constant domain of the light chain of the first antibody or antibody fragment with the second antibody or antibody fragment.

The replacement of less than about 20 amino acid residues may comprise replacement of at least 1 amino acid residue from the first antibody or antibody fragment with the second antibody or antibody fragment.The replacement of less than about 20 amino acid residues may comprise replacement of at least 2 amino acid residues from the first antibody or antibody fragment with the second antibody or antibody fragment. The replacement of less than about 20 amino acid residues may comprise replacement of at least 3 amino acid residues from the first antibody or antibody fragment with the second antibody or antibody fragment.

The replacement of less than about 20 amino acid residues may comprise replacement of less than 15 amino acid residues from the first antibody or antibody fragment with the second antibody or antibody fragment. The replacement of less than about 20 amino acid residues may comprise replacement of less than 10 amino acid residues from the first antibody or antibody fragment with the second antibody or antibody fragment. The replacement of less than about 20 amino acid residues may comprise replacement of less than 5 amino acid residues from the first antibody or antibody fragment with the second antibody or antibody fragment.

The replacement of less than about 20 amino acid residues may comprise replacement of 5 or fewer amino acid residues from the first antibody or antibody fragment with the second antibody or antibody fragment. The replacement of less than about 20 amino acid residues may comprise replacement of 4 or fewer amino acid residues from the first antibody or antibody fragment with the second antibody or antibody fragment. The replacement of less than about 20 amino acid residues may comprise replacement of 3 or fewer amino acid residues from the first antibody or antibody fragment with the second antibody or antibody fragment. The replacement of less than about 20 amino acid residues may comprise replacement of 1-15 amino acid residues from the first antibody or antibody fragment with the second antibody or antibody fragment. The replacement of less than about 20 amino acid residues may comprise replacement of 1-10 amino acid residues from the first antibody or antibody fragment with the second antibody or antibody fragment. The replacement of less than about 20 amino acid residues may comprise replacement of 1-5 amino acid residues from the first antibody or antibody fragment with the second antibody or antibody fragment.

The replacement of the amino acid residues may comprise replacement of one or more amino acids selected from a group consisting of serine (S), glycine (G), lysine (K), proline (P), threonine (T), glutamine (Q), glutamic acid (E), alanine (A), asparagines (N), and histidine (H). The amino acid residues may be in the consensus insertion sequence of the first antibody or antibody fragment. The replacement of less than about 20 amino acids may comprise replacement of 5 or fewer amino acid residues from the CH1 domain of the first antibody or antibody fragment. The replacement of less than about 20 amino acids may comprise replacement of 4 or fewer amino acid residues from the CH1 domain of the first antibody or antibody fragment.

The replacement of less than about 20 amino acids may comprise replacement of 3 or fewer amino acid residues from the CH1 domain of the first antibody or antibody fragment. The replacement of less than about 20 amino acids may comprise replacement of 2 or fewer amino acid residues from the CH1 domain of the first antibody or antibody fragment. The replacement of less than about 20 amino acids may comprise replacement of 1 amino acid residue from the CH1 domain of the first antibody or antibody fragment. The replacement of less than about 20 amino acids may comprise replacement of one or more amino acids from the CH1 domain selected from a group consisting of serine (S), glycine (G), proline (P), threonine (T), and glutamine (Q). The replacement of less than about 20 amino acids may comprise replacement of serine 180 (S180) from the CH1 domain. The replacement of less than about 20 amino acids may comprise replacement of glycine 181 (G181) from the CH1 domain. The replacement of less than about 20 amino acids may comprise replacement of serine 180 (S180) and glycine 181 (G181) from the CH1 domain. The replacement of less than about 20 amino acids may comprise replacement of proline 156 (P156) from the CH1 domain. The replacement of less than about 20 amino acids may comprise replacement of serine and glycine from the CH1 domain. The serine and glycine may be adjacent to each other. The replacement of less than about 20 amino acids may comprise replacement of threonine and serine from the CH1 domain. The threonine and serine may be adjacent to each other.

The replacement of less than about 20 amino acids may comprise replacement of 5 or fewer amino acid residues from the CH2 domain of the first antibody or antibody fragment. The replacement of less than about 20 amino acids may comprise replacement of 4 or fewer amino acid residues from the CH2 domain of the first antibody or antibody fragment. The replacement of less than about 20 amino acids may comprise replacement of 3 or fewer amino acid residues from the CH2 domain of the first antibody or antibody fragment. The replacement of less than about 20 amino acids may comprise replacement of 2 or fewer amino acid residues from the CH2 domain of the first antibody or antibody fragment. The replacement of less than about 20 amino acids may comprise replacement of 1 amino acid residue from the CH2 domain of the first antibody or antibody fragment. The replacement of less than about 20 amino acids may comprise replacement of one or more amino acids from the CH2 domain selected from a group consisting of glutamic acid (E), alanine (A) and proline (P). The replacement of less than about 20 amino acids may comprise replacement of glutamic acid 274 (E274) from the CH2 domain. The replacement of less than about 20 amino acids may comprise replacement of alanine 302 (A302) from the CH2 domain. The replacement of less than about 20 amino acids may comprise replacement of proline 334 (P334) from the CH2 domain.

The replacement of less than about 20 amino acids may comprise replacement of 5 or fewer amino acid residues from the CH3 domain of the first antibody or antibody fragment. The replacement of less than about 20 amino acids may comprise replacement of 4 or fewer amino acid residues from the CH3 domain of the first antibody or antibody fragment. The replacement of less than about 20 amino acids may comprise replacement of 3 or fewer amino acid residues from the CH3 domain of the first antibody or antibody fragment. The replacement of less than about 20 amino acids may comprise replacement of 2 or fewer amino acid residues from the CH3 domain of the first antibody or antibody fragment. The replacement of less than about 20 amino acids may comprise replacement of 1 amino acid residue from the CH3 domain of the first antibody or antibody fragment. The replacement of less than about 20 amino acids may comprise replacement of one or more amino acids from the CH3 domain selected from a group consisting of threonine (T), lysine (K), asparagine (N), and glycine (G). The replacement of less than about 20 amino acids may comprise replacement of threonine 361 (T361) from the CH3 domain. The replacement of less than about 20 amino acids may comprise replacement of lysine 362 (K362) from the CH3 domain. The replacement of less than about 20 amino acids may comprise replacement of asparagine 363 (N363) from the CH3 domain. The replacement of less than about 20 amino acids may comprise replacement of threonine 361 (T361), lysine 362 (K362), and asparagine 363 (N363) from the CH3 domain. The replacement of less than about 20 amino acids may comprise replacement of asparagine 389 (N389) from the CH3 domain. The replacement of less than about 20 amino acids may comprise replacement of glycine 390 (G390) from the CH3 domain. The replacement of less than about 20 amino acids may comprise replacement of asparagine 389 (N389) and glycine 390 (G390) from the CH3 domain. The replacement of less than about 20 amino acids may comprise replacement of glycine 425 (G425) from the CH3 domain. The replacement of less than about 20 amino acids may comprise replacement of asparagine 426 (N426) from the CH3 domain. The replacement of less than about 20 amino acids may comprise replacement of glycine 425 (G425) and asparagine 363 (N363) from the CH3 domain. The replacement of less than about 20 amino acids may comprise replacement of threonine and asparagine from the CH3 domain. The threonine and asparagine may be adjacent to each other. The replacement of less than about 20 amino acids may comprise replacement of threonine, lysine, and asparagine from the CH3 domain. The threonine, lysine, and asparagine may be adjacent to each other.

The replacement of less than about 20 amino acids may comprise replacement of 5 or fewer amino acid residues from the constant domain of the light chain of the first antibody or antibody fragment.The replacement of less than about 20 amino acids may comprise replacement of 4 or fewer amino acid residues from the constant domain of the light chain of the first antibody or antibody fragment. The replacement of less than about 20 amino acids may comprise replacement of 3 or fewer amino acid residues from the constant domain of the light chain of the first antibody or antibody fragment. The replacement of less than about 20 amino acids may comprise replacement of 2 or fewer amino acid residues from the constant domain of the light chain of the first antibody or antibody fragment. The replacement of less than about 20 amino acids may comprise replacement of 1 amino acid residue from the constant domain of the light chain of the first antibody or antibody fragment. The replacement of less than about 20 amino acids may comprise replacement of one or more amino acids from the constant domain of the light chain selected from a group consisting of serine (S), glycine (G), proline (P), lysine (K), asparagine (N) and histidine (H) The replacement of less than about 20 amino acids may comprise replacement of serine 202 (S202) from the constant domain of the light chain. The replacement of less than about 20 amino acids may comprise replacement of glycine 128 (G128) from the constant domain of the light chain. The replacement of less than about 20 amino acids may comprise replacement of lysine 169 (K169) from the constant domain of the light chain. The replacement of less than about 20 amino acids may comprise replacement of proline 141 (P141) from the constant domain of the light chain. The replacement of less than about 20 amino acids may comprise replacement of asparagine (N152) from the constant domain of the light chain. The replacement of less than about 20 amino acids may comprise replacement of lysine 138 (K138) from the constant domain of the light chain. The replacement of less than about 20 amino acids may comprise replacement of histidine 139 (H139) from the constant domain of the light chain. The replacement of less than about 20 amino acids may comprise replacement of lysine 138 (K138) and histidine (H139) from the constant domain of the light chain. The replacement of less than about 20 amino acids may comprise replacement of lysine and histidine from the constant domain of the light chain. The lysine and histidine may be adjacent to each other.

The first antibody or antibody fragment may be based on or derived from a group consisting of UCHT1, anti-CD19, anti-CD20 and Her2. The first antibody or antibody fragment may comprise a fragment antigen binding (Fab), fragment antigen-binding including hinge region (F(ab′)₂), fragment antigen-binding including one hinge region (Fab′), fragment crystallizable (Fc), variable domain (e.g., V_{H} or V_{L}), constant domain (e.g., C_{H1}, C_{H2}, C_{H3}, or C_{L}), single-chain varaible fragment (scFV), di-ScFv, single domain antibody (sdAb), minibody, diabody, tribody, tetrabody, trifunctional antibody. The first antibody or antibody fragment may comprise one or more heavy chains, light chains, or both. The first antibody or antibody fragment may comprise one or more modified constant domains. The first antibody fragment or antibody fragment may comprise one or more variable domains.

The second antibody or antibody fragment may be based on or derived from a group consisting of UCHT1, anti-CD19, anti-CD20, and Her2. The second antibody or antibody fragment may comprise a fragment antigen binding (Fab), fragment antigen-binding including hinge region (F(ab′)₂), fragment antigen-binding including one hinge region (Fab′), fragment crystallizable (Fc), variable domain (e.g., V_{H} or V_{L}), constant domain (e.g., C_{H1}, C_{H2}, C_{H3}, or C_{L}), single-chain varaible fragment (scFV), di-ScFv, single domain antibody (sdAb), minibody, diabody, tribody, tetrabody, trifunctional antibody. The second antibody or antibody fragment may comprise one or more heavy chains, light chains, or both. The second antibody or antibody fragment may comprise one or more constant domains. The second antibody fragment or antibody fragment may comprise one or more variable domains.

The first antibody or antibody fragment may be based on or derived from a UCHT1 antibody or antibody fragment. The second antibody or antibody fragment may be based on or derived from a UCHT1 antibody or antibody fragment. The UCHT1 may be UCHT1scFv. The UCHT1 may be UCHT1 light chain. The UCHT1 may be UCHT1 heavy chain. The UCHT1 may comprise an amino acid sequence that is at least 50% homologous to a sequence selected from a group consisting of SEQ ID NOS: 34, 35, 41, and 88. The UCHT1 may comprise an amino acid sequence that is at least 60% homologous to a sequence selected from a group consisting of SEQ ID NOS: 34, 35, 41, and 88. The UCHT1 may comprise an amino acid sequence that is at least 70% homologous to a sequence selected from a group consisting of SEQ ID NOS: 34, 35, 41, and 88. The UCHT1 may comprise an amino acid sequence that is at least 80% homologous to a sequence selected from a group consisting of SEQ ID NOS: 34, 35, 41, and 88. The UCHT1 may comprise an amino acid sequence that is at least 90% homologous to a sequence selected from a group consisting of SEQ ID NOS: 34, 35, 41, and 88. The UCHT1 may comprise an amino acid sequence that comprises 10 or more consecutive amino acids from a sequence selected from a group consisting of SEQ ID NOS: 34, 35, 41, and 88. The UCHT1 may comprise an amino acid sequence that comprises 50 or more consecutive amino acids from a sequence selected from a group consisting of SEQ ID NOS: 34, 35, 41, and 88. The UCHT1 may comprise an amino acid sequence that comprises 100 or more consecutive amino acids from a sequence selected from a group consisting of SEQ ID NOS: 34, 35, 41, and 88. The UCHT1 may comprise an amino acid sequence that comprises 200 or more consecutive amino acids from a sequence selected from a group consisting of SEQ ID NOS: 34, 35, 41, and 88.

The first antibody or antibody fragment may be based on or derived from an anti-CD19 antibody or antibody fragment. The second antibody or antibody fragment may be based on or derived from an anti-CD19 antibody or antibody fragment. The anti-CD19 may be anti-CD19scFv. The anti-CD19 may be anti-CD19 light chain. The anti-CD19 may be anti-CD19 heavy chain. The anti-CD19 may be anti-CD19 Fab fragment. The anti-CD19 may comprise an amino acid sequence that is at least 50% homologous to SEQ ID NOS: 38, 39, 42, and 87. The anti-CD19 may comprise an amino acid sequence that is at least 60% homologous to SEQ ID NOS: 38, 39, 42, and 87. The anti-CD19 may comprise an amino acid sequence that is at least 70% homologous to SEQ ID NOS: 38, 39, 42, and 87. The anti-CD19 may comprise an amino acid sequence that is at least 80% homologous to SEQ ID NOS: 38, 39, 42, and 87. The anti-CD19 may comprise an amino acid sequence that is at least 90% homologous to SEQ ID NOS: 38, 39, 42, and 87. The anti-CD19 may comprise an amino acid sequence that comprises 10 or more consecutive amino acids from SEQ ID NOS: 38, 39, 42, and 87. The anti-CD19 may comprise an amino acid sequence that comprises 50 or more consecutive amino acids from SEQ ID NOS: 38, 39, 42, and 87. The anti-CD19 may comprise an amino acid sequence that comprises 75 or more consecutive amino acids from SEQ ID NOS: 38, 39, 42, and 87. The anti-CD19 may comprise an amino acid sequence that comprises 100 or more consecutive amino acids from SEQ ID NOS: 38, 39, 42, and 87.

The first antibody or antibody fragment may be based on or derived from a Her2 antibody or antibody fragment. The second antibody or antibody fragment may be based on or derived from a Her2 antibody or antibody fragment. The Her2 may be Her2scFv. The Her2 may be Her2 light chain. The Her2 may be Her2 heavy chain. The Her2 may comprise an amino acid sequence that is at least 50% homologous to a sequence selected from a group consisting of SEQ ID NOS: 33, 40, and 86. The Her2 may comprise an amino acid sequence that is at least 60% homologous to a sequence selected from a group consisting of SEQ ID NOS: 33, 40, and 86. The Her2 may comprise an amino acid sequence that is at least 70% homologous to a sequence selected from a group consisting of SEQ ID NOS: 33, 40, and 86. The Her2 may comprise an amino acid sequence that is at least 80% homologous to a sequence selected from a group consisting of SEQ ID NOS: 33, 40, and 86. The Her2 may comprise an amino acid sequence that is at least 90% homologous to a sequence selected from a group consisting of SEQ ID NOS: 33, 40, and 86. The Her2 may comprise an amino acid sequence that comprises 10 or more consecutive amino acids from a sequence selected from a group consisting of SEQ ID NOS: 33, 40, and 86. The Her2 may comprise an amino acid sequence that comprises 50 or more consecutive amino acids from a sequence selected from a group consisting of SEQ ID NOS: 33, 40, and 86. The Her2 may comprise an amino acid sequence that comprises 100 or more consecutive amino acids from a sequence selected from a group consisting of SEQ ID NOS: 33, 40, and 86. The Her2 may comprise an amino acid sequence that comprises 200 or more consecutive amino acids from a sequence selected from a group consisting of SEQ ID NOS: 33, 40, and 86.

The bispecific antibody may comprise an amino acid sequence that is at least about 50% homologous to an amino acid sequence selected from a group consisting of SEQ ID NOS: 58-60, and 67-70. The bispecific antibody may comprise an amino acid sequence that is at least about 60% homologous to an amino acid sequence selected from a group consisting of SEQ ID NOS: 58-60, and 67-70. The bispecific antibody may comprise an amino acid sequence that is at least about 70% homologous to an amino acid sequence selected from a group consisting of SEQ ID NOS: 58-60, and 67-70. The bispecific antibody may comprise an amino acid sequence that is at least about 80% homologous to an amino acid sequence selected from a group consisting of SEQ ID NOS: 58-60, and 67-70. The bispecific antibody may comprise an amino acid sequence that is at least about 90% homologous to an amino acid sequence selected from a group consisting of SEQ ID NOS: 58-60, and 67-70. The bispecific antibody may comprise an amino acid sequence that comprises 25 or more consecutive amino acids from any one of SEQ ID NOS: 58-60, and 67-70. The bispecific antibody may comprise an amino acid sequence that comprises 50 or more consecutive amino acids from any one of SEQ ID NOS: 58-60, and 67-70. The bispecific antibody may comprise an amino acid sequence that comprises 100 or more consecutive amino acids from any one of SEQ ID NOS: 58-60, and 67-70. The bispecific antibody may comprise an amino acid sequence that comprises 150 or more consecutive amino acids from any one of SEQ ID NOS: 58-60, and 67-70. The bispecific antibody may comprise an amino acid sequence that comprises 200 or more consecutive amino acids from any one of SEQ ID NOS: 58-60, and 67-70. The bispecific antibody may comprise an amino acid sequence that comprises 300 or more consecutive amino acids from any one of SEQ ID NOS: 58-60, and 67-70. The bispecific antibody may comprise an amino acid sequence that comprises 350 or more consecutive amino acids from any one of SEQ ID NOS: 58-60, and 67-70.

The bispecific antibody may further comprise a third antibody or antibody fragment. The third antibody or antibody fragment may comprise an amino acid sequence that is at least 50% homologous to an amino acid sequence selected from a group consisting of SEQ ID NO: 33-44. The third antibody or antibody fragment may comprise an amino acid sequence that is at least 60% homologous to an amino acid sequence selected from a group consisting of SEQ ID NO: 33-44. The third antibody or antibody fragment may comprise an amino acid sequence that is at least 70% homologous to an amino acid sequence selected from a group consisting of SEQ ID NO: 33-44. The the third antibody or antibody fragment may comprise an amino acid sequence that is at least 80% homologous to an amino acid sequence selected from a group consisting of SEQ ID NO: 33-44. The third antibody or antibody fragment may comprise an amino acid sequence that is at least 90% homologous to an amino acid sequence selected from a group consisting of SEQ ID NO: 33-44. The third antibody or antibody fragment may comprise an amino acid sequence that comprises 50 or more consecutive amino acids from any one of SEQ ID NO: 33-44. The third antibody or antibody fragment may comprise an amino acid sequence that comprises 100 or more consecutive amino acids from any one of SEQ ID NO: 33-44. The third antibody or antibody fragment may comprise an amino acid sequence that comprises 150 or more consecutive amino acids from any one of SEQ ID NO: 33-44. The third antibody or antibody fragment may comprise an amino acid sequence that comprises 200 or more consecutive amino acids from any one of SEQ ID NO: 33-44.

### BRIEF DESCRIPTION OF THE DRAWINGS

FIG. 1 shows an SDS gel image of hEPO-coil-Trastuzumab-CL in non-reducing and reducing (with 50 mM DTT) conditions.

FIG. 2 shows Alamar Blue cell proliferation assay of TF-1 cells incubated with different concentration of hEPO-Trastuzumab fusion proteins.

FIG. 3A-D depict the binding affinity of hEPO-coil-Her2-CL, hEPO-coil-Her2-CH1 and wt.trastuzumab against Her2+ SK—BR-3 cells.

FIG. 4 shows SDS gel image of hEPO-coil-Trastuzumab—CH3 in non-reducing and reducing (with 50 mM DTT) conditions.

FIG. 5 shows a SDS gel image of hEPO-G4S-Trastuzumab-CL in non-reducing and reducing (with 50 mM DTT) conditions.

FIG. 6 shows Alamar Blue cell proliferation assay of TF-1 cells incubated with different concentration of hEPO-Trastuzumab fusion proteins.

FIG. 7A-D depict the binding affinity of hEPO-G4S-Her2-CL, hEPO-coil-Her2-CH3 and wt.trastuzumab against Her2+ SK—BR-3 cells.

FIG. 8A-D depict the binding affinity of hEPO-G4S-Her2-CL, hEPO-coil-Her2-CH3 and wt.trastuzumab against Her2+ SK—BR-3 cells.

FIG. 9 shows the binding of various concentrations of wt.Trastuzumab and hEPO-coil-Her2-CH3 against Her2 as determined by ELISA.

FIG. 10 shows a SDS gel image of TCP1-G4S-UCHT1-CL (e.g., TCP1-UCHT1-CL) in non-reducing and reducing (with 50 mM DTT) conditions.

FIG. 11 shows a SDS gel image of NGR-UCHT1-CL in non-reducing and reducing (with 50 mM DTT) conditions.

FIG. 12 shows a SDS gel image of CXCR4-BP-coil-Her2-CH1 fusion proteins in non-reducing and reducing (with 50 mM DTT) conditions.

FIG. 13A-F show graphs of the binding of NGR-G4S-UCHT1-CL against CD13+ positive HT-1080 cells and MDA-MB-435 cells (negative control).

FIG. 14A-F show graphs of the binding of TCP1-G4S-UCHT1-CL against colorectal cancer cells (HT-29) and MDA-MB-435 cells (negative control).

FIG. 15 shows a SDS gel image of Int-coil-UCHT1-CL, CXCR4-BP-coil-CD20-CL(Fab), TCP1-coil-UCHT1-CL, and NGR-coil-UCHT1-CL in non-reducing and reducing (with 50 mM DTT) conditions. Lane 1 represents the protein standard marker, Lane 2 represents Int-coil-UCHT1-CL without DTT treatment and Lane 3 represents Int-coil-UCHT1-CL with DTT treatment, Lane 4 represents CXCR4-BP-coil-CD20-CL(Fab) without DTT treatment and Lane 5 represents CXCR4-BP-coil-CD20-CL(Fab) with DTT treatment, Lane 6 represents TCP1-coil-UCHT1-CL without DTT treatment and Lane 7 represents TCP1-coil-UCHT1-CL with DTT treatment, Lane 8 represents NGR-coil-UCHT1-CL without DTT treatment, and Lane 9 represents NGR-coil-UCHT1-CL with DTT treatment.

FIG. 16 shows a SDS gel image of CXCR4-BP-coil-Her2-CL fusion proteins in non-reducing and reducing (with 50 mM DTT) conditions.

FIG. 17 shows a SDS gel image of CD20 and CXCR4-BP-coil-CD20-CL(IgG) fusion proteins in non-reducing and reducing (with 50 mM DTT) conditions.

FIG. 18A-D show graphs of the binding affinity of CD20Fab, CXCR4-BP-coil-CD20(Fab), and CXCR4-BP-Palivizumab against CD20+/CXCR4-BPdim BJAB cells.

FIG. 19A-D show graphs of the binding affinity of CD20Fab, CXCR4-BP-coil-CD20(Fab), and CXCR4-BP-Palivizumab against CD20dim/CXCR4+ Nalm-6 cells.

FIG. 20A-D show the flow cytometry results for K562 cells incubated with only the secondary antibody.

FIG. 21A-D show graphs of the binding affinity of anti-CD20, CXCR4-BP-coil-CD20(IgG), CXCR4-BP-Her2-CL and CXCR4-BP-Her2-CH1 against CD20+/CXCR4+ Raji cells.

FIG. 22A-D show graphs of the binding affinity of anti-CD20, CXCR4-BP-coil-CD20(IgG), CXCR4-BP-Her2-CL and CXCR4-BP-Her2-CH1 against CD20−/CXCR4+ Nalm-6 cells.

FIG. 23A-D show graphs of the binding affinity of anti-CD20, CXCR4-BP-coil-CD20(IgG), CXCR4-BP-Her2-CL and CXCR4-BP-Her2-CH1 against CD20−/CXCR4+ BJAB cells.

FIG. 24A-D show graphs of the binding affinity of anti-CD20, CXCR4-BP-coil-CD20(IgG), CXCR4-BP-Her2-CL and CXCR4-BP-Her2-CH1 against CD20−/CXCR4− K562 cells.

FIG. 25 shows a SDS gel image of CD19ScFv-UCHT1-CL (Fab) in non-reducing and reducing (with 50 mM DTT) conditions.

FIG. 26A-D show graphs of the binding affinity of CD19ScFv-UCHT1-CL(Fab) against Nalm-6 or K562 cells.

FIG. 27A-B show graphs of the in vitro cytotoxicity of anti-CD19ScFv-UCHT1-CL(Fab) in Nalm-6 and HT-29 cells.

FIG. 28A-B show SDS gel images of GCN4-CD19(IgG) and GCN4-CD19(Fab) in non-reducing and reducing (with 50 mM DTT) conditions.

FIG. 29A shows a non-reducing SDS-PAGE gel of anti-CD19 antibodies or antibody fragments with a GCN4 peptide grafted or fused to various regions or domains of the antibodies or antibody fragments.

FIG. 29B shows a reducing SDS-PAGE gel of anti-CD19 antibodies or antibody fragments with a GCN4 peptide grafted or fused to various regions or domains of the antibodies or antibody fragments.

FIG. 30A shows in vitro cytotoxicity data for Her2ScFv-UCHT1-CL-L2A and Her2ScFv-UCHT1-CL-L2B in Her2-negative MDA-MB-468 cells (L2 indicates a disulfide bond has been engineered relatively upstream in coiled-coil).

FIG. 30B shows in vitro cytotoxicity data for Her2ScFv-UCHT1-CL-L2A and Her2ScFv-UCHT1-CL-L2B in Her2-low MDA-MB-231 cells.

FIG. 30C shows in vitro cytotoxicity data for Her2ScFv-UCHT1-CL-L2A and Her2ScFv-UCHT1-CL-L2B in Her2-high MDA-MB-435 cells.

FIG. 30D shows in vitro cytotoxicity data for Her2ScFv-UCHT1-CL-L3A and Her2ScFv-UCHT1-CL-L3B in Her2-negative MDA-MB-468 cells (L3 indicates a disulfide bond has been engineered relatively upstream in coiled-coil).

FIG. 30E shows in vitro cytotoxicity data for Her2ScFv-UCHT1-CL-L3A and Her2ScFv-UCHT1-CL-L3B in Her2-low MDA-MB-231 cells.

FIG. 30F shows in vitro cytotoxicity data for Her2ScFv-UCHT1-CL-L3A and Her2ScFv-UCHT1-CL-L3B in Her2-high MDA-MB-435 cells.

FIG. 31A shows an SDS gel image of Her2ScFv-UCHT1-CL-L2A and Her2ScFv-UCHT1-CL-L2B.

FIG. 31B shows an SDS gel image of Her2ScFv-UCHT1-CL-L3A and Her2ScFv-UCHT1-CL-L3B.

### DETAILED DESCRIPTION OF THE INVENTION

Disclosed herein are antibody fusion proteins. The antibody fusion protein may be a constant region antibody fusion protein. The antibody fusion protein may be a bispecific antibody fusion protein. In some instances, an antibody fusion protein may comprise an antibody fusion protein comprising: an antibody region comprising an antibody or antibody fragment, wherein the antibody or antibody fragment comprises a modified constant domain; and a non-antibody polypeptide region comprising 15 or more amino acids, wherein the non-antibody polypeptide region is located within the modified constant domain. The non-antibody peptide may be inserted into the modified constant domain of the antibody region by replacement of less than about 20 amino acid residues from the modified constant domain. A limit of repalcing about 20 amino acids of the modified constant domain may be necessary to maintain proper folding of the antibody or antibody fragment. Alternatively, insertion of the non-antibody peptide does not comprise replacement of one or more amino acid residues from the modified constant domain of the antibody region. The non-antibody peptide may be a non-antigenic peptide. In some instances, the non-antibody peptide is not based on or derived from a T cell epitope. In some instances, the non-antibody peptide is not based on or derived from a B cell epitope. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody.

Disclosed herein are antibody fusion proteins. The antibody fusion protein may be a constant region antibody fusion protein. In some instances, the antibody fusion protein may comprise (a) an antibody region based on or derived from an antibody or antibody fragment; and (b) a non-antibody polypeptide region comprising 15 or more amino acids, wherein the non-antibody polypeptide region may be inserted into the antibody region by replacement of less than about 20 amino acid residues from the antibody or antibody fragment with the non-antibody polypeptide region. The non-antibody peptide may be a non-antigenic peptide. In some instances, the non-antibody peptide is not based on or derived from a T cell epitope. In some instances, the non-antibody peptide is not based on or derived from a B cell epitope. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody.

Further disclosed herein are bispecific antibodies and uses thereof. A bispecific antibody may comprise (a) first antibody or antibody fragment; and (b) a second antibody or antibody fragment, wherein the second antibody or antibody fragment may be inserted into a constant domain of the first antibody or antibody fragment. In some instances, the second antibody or antibody fragment is inserted into the first antibody or antibody fragment by replacement of less than about 20 amino acid residues from the first antibody or antibody fragment with the second antibody or antibody fragment. Alternatively, insertion of the second antibody or antibody fragment does not comprise replacement of one or more amino acid residues from the constant domain of the first antibody or antibody fragment. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody.

Further disclosed herein are bispecific antibodies and uses thereof. A bispecific antibody may comprise (a) first antibody or antibody fragment; and (b) a second antibody or antibody fragment, wherein the second antibody or antibody fragment may be inserted into the first antibody or antibody fragment by replacement of less than about 20 amino acid residues from the first antibody or antibody fragment with the second antibody or antibody fragment. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody.

### Antibody Fusion Proteins

Disclosed herein are antibody fusion proteins and uses thereof. An antibody fusion protein may comprise (a) an antibody region based on or derived from an antibody or antibody fragment; and (b) a non-antibody polypeptide region comprising 15 or more amino acids, wherein the non-antibody polypeptide region is inserted into a constant domain of the antibody region. The non-antibody peptide may be inserted into the constant domain of the antibody region by replacement of less than about 20 amino acid residues from the constant domain of the antibody region with the non-antibody polypeptide region. Alternatively, insertion of the non-antibody peptide does not comprise replacement of one or more amino acid residues from the constant domain of the antibody region. The non-antibody peptide may be a non-antigenic peptide. In some instances, the non-antibody peptide is not based on or derived from a T cell epitope. In some instances, the non-antibody peptide is not based on or derived from a B cell epitope. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody. Alternataively, or additionally, an antibody fusion protein may comprise (a) an antibody region based on or derived from an antibody or antibody fragment; and (b) a non-antibody polypeptide region, wherein the non-antibody polypeptide region may be inserted into the antibody region by replacement of less than about 20 amino acid residues from the antibody or antibody fragment with the non-antibody polypeptide region. In some instances, the non-antibody polypeptide is not inserted into a constant domain of the antibody or antibody fragment. The constant domain of the antibody or antibody fragment may be a CH1 domain. The constant domain of the antibody or antibody fragment may be a CL1 domain. The constant domain of the antibody or antibody fragment may be a hinge domain. In some instances, the non-antibody polypeptide is not inserted into a complementarity determining region (CDR) of the antibody or antibody fragment. The CDR may be CDR1. The CDR may be CDR2. The CDR may be CDR3. The non-antibody polypeptide region may comprise 15 or more amino acids. The non-antibody polypeptide region may comprise 16 or more amino acids. The non-antibody polypeptide region may comprise 17 or more amino acids. The non-antibody polypeptide region may comprise 18 or more amino acids. The non-antibody polypeptide region may comprise 19 or more amino acids. The non-antibody polypeptide region may comprise 20 or more amino acids. The non-antibody polypeptide region may comprise 21 or more amino acids. The non-antibody polypeptide region may comprise 22 or more amino acids. The non-antibody polypeptide region may comprise 20, 30, 40, 50, 60, 70, or 80 or more amino acids. The antibody fusion proteins disclosed herein may be used to treat a disease or condition in a subject in need thereof. Further disclosed herein are methods of treating a disease or condition in a subject in need, the method comprising administering to the subject an antibody fusion protein disclosed herein.

The non-antibody polypeptide region may be inserted adjacent to a beta strand secondary structure in constant domain of the antibody or antibody fragment from which the antibody region is based on or derived. The non-antibody polypeptide region may be inserted adjacent to a beta strand secondary structure in the antibody or antibody fragment from which the antibody region is based on or derived. The non-antibody polypeptide region may be inserted between two beta strand secondary structures in constant domain of the antibody or antibody fragment from which the antibody region is based on or derived. The non-antibody polypeptide region may be inserted between two beta strand secondary structures in the antibody or antibody fragment from which the antibody region is based on or derived. The non-antibody polypeptide region may be inserted into a loop region in constant domain of the antibody or antibody fragment from which the antibody region is based on or derived. The non-antibody polypeptide region may be inserted into a loop region in the antibody or antibody fragment from which the antibody region is based on or derived.

The non-antibody polypeptide region may be inserted into a constant domain of the antibody or antibody fragment. The non-antibody polypeptide region may be inserted into a loop region of the antibody or antibody fragment. The non-antibody polypeptide region may be inserted into a loop region of a constant domaino of the antibody or antibody fragment. The non-antibody polypeptide region may be inserted near a beta strand of the antibody region. The non-antibody polypeptide region may be inserted within 20 amino acids of a beta strand of the antibody region. The non-antibody polypeptide region may be inserted within 15 amino acids of a beta strand of the antibody region. The non-antibody polypeptide region may be inserted within 10 amino acids of a beta strand of the antibody region. The non-antibody polypeptide region may be inserted within 5 amino acids of a beta strand of the antibody region. The less than about 20 amino acid residues to be replaced may be located between two beta strands. The non-antibody polypeptide region may be inserted into the antibody region by replacement of less than about 20 amino acid residues from a constant domain of the antibody or antibody fragment with the non-antibody polypeptide region. The less than about 20 amino acid residues to be replaced may be located near a beta strand. The less than about 20 amino acid residues to be replaced may be within 20 amino acids of a beta strand. The less than about 20 amino acid residues to be replaced may be within 15 amino acids of a beta strand. The less than about 20 amino acid residues to be replaced may be within 10 amino acids of a beta strand. The less than about 20 amino acid residues to be replaced may be within 5 amino acids of a beta strand. The less than about 20 amino acid residues to be replaced may be located between two beta strands. The constant domain may be from a heavy chain of the antibody or antibody fragment. The constant domain may be from a light chain of the antibody or antibody fragment.

The antibody region may comprise a consensus insertion sequence. The consensus insertion sequence may comprise an amino acid sequence that is at least about 50% homologous to an amino acid sequence selected from a group consisting of SEQ ID NO: 89-120. The consensus insertion sequence may comprise an amino acid sequence that is at least about 60% homologous to an amino acid sequence selected from a group consisting of SEQ ID NO: 89-120. The consensus insertion sequence may comprise an amino acid sequence that is at least about 70% homologous to an amino acid sequence selected from a group consisting of SEQ ID NO: 89-120. The consensus insertion sequence may comprise an amino acid sequence that is at least about 80% homologous to an amino acid sequence selected from a group consisting of SEQ ID NO: 89-120. The consensus insertion sequence may comprise an amino acid sequence that is at least about 90% homologous to an amino acid sequence selected from a group consisting of SEQ ID NO: 89-120. The consensus insertion sequence may comprise an amino acid sequence that is at least about 95% homologous to an amino acid sequence selected from a group consisting of SEQ ID NO: 89-120. The amino acid sequence may be SEQ ID NO: 89. The amino acid sequence may be SEQ ID NO: 90. The amino acid sequence may be SEQ ID NO: 91. The amino acid sequence may be SEQ ID NO: 92. The amino acid sequence may be SEQ ID NO: 93. The amino acid sequence may be SEQ ID NO: 94. The amino acid sequence may be SEQ ID NO: 95. The amino acid sequence may be SEQ ID NO: 96. The amino acid sequence may be SEQ ID NO: 97. The amino acid sequence may be SEQ ID NO: 98. The amino acid sequence may be SEQ ID NO: 99. The amino acid sequence may be SEQ ID NO: 100. The amino acid sequence may be SEQ ID NO: 101. The amino acid sequence may be SEQ ID NO: 102. The amino acid sequence may be SEQ ID NO: 103. The amino acid sequence may be SEQ ID NO: 104. The amino acid sequence may be SEQ ID NO: 105. The amino acid sequence may be SEQ ID NO: 106. The amino acid sequence may be SEQ ID NO: 107. The amino acid sequence may be SEQ ID NO: 108. The amino acid sequence may be SEQ ID NO: 109. The amino acid sequence may be SEQ ID NO: 110. The amino acid sequence may be SEQ ID NO: 111. The amino acid sequence may be SEQ ID NO: 112. The amino acid sequence may be SEQ ID NO: 113. The amino acid sequence may be SEQ ID NO: 114. The amino acid sequence may be SEQ ID NO: 115. The amino acid sequence may be SEQ ID NO: 116. The amino acid sequence may be SEQ ID NO: 117. The amino acid sequence may be SEQ ID NO: 118. The amino acid sequence may be SEQ ID NO: 119. The amino acid sequence may be SEQ ID NO: 120. The consensus insertion sequence may be based on or derived from a constant domain of the antibody or antibody fragment. The consensus insertion sequence may be based on or derived from a loop region of the antibody or antibody fragment. The consensus insertion sequence may be based on or derived from a loop region of a constant domain of the antibody or antibody fragment. The consensus insertion sequence may be based on or derived from a sequence located between two beta strands of the antibody or antibody fragment. The two beta strands may be in a constant domain of the antibody or antibody fragment. The constant domain may be in a heavy chain. The constant domain may be CH1. The constant domain may be CH2. The constant domain may be CH3. The constant domain may be in a light chain. The loop region may be in a heavy chain. The loop region may be in the light chain. The two beta strands may be in a heavy chain. The two beta strands may be in a light chain. The non-antibody polypeptide region may be inserted into the consensus insertion sequence of the antibody region. The non-antibody polypeptide region may be inserted into the antibody region by replacement of less than about 20 amino acids from the consensus insertion sequence of the antibody region. The non-antibody polypeptide region may be inserted into the consensus insertion sequence by replacement of one or more amino acids from the consensus insertion sequence. The non-antibody polypeptide region may be inserted into the consensus insertion sequence by replacement of two or more amino acids from the consensus insertion sequence. The non-antibody polypeptide region may be inserted into the consensus insertion sequence by replacement of three or more amino acids from the consensus insertion sequence. The non-antibody polypeptide region may be inserted into the consensus insertion sequence by replacement of four or more amino acids from the consensus insertion sequence. The non-antibody polypeptide region may be inserted into the consensus insertion sequence by replacement of five or more amino acids from the consensus insertion sequence.

The non-antibody polypeptide region may be inserted into the antibody region by replacement of less than about 20 amino acid residues from a constant domain of the antibody or antibody fragment with the non-antibody polypeptide region. The constant domain may be from a heavy chain of the antibody or antibody fragment. The constant domain may be from a light chain of the antibody or antibody fragment.

The non-antibody polypeptide region may be inserted into the antibody region by replacement of less than about 20 amino acid residues from a heavy chain of the antibody or antibody fragment with the non-antibody polypeptide region. The non-antibody polypeptide region may be inserted into the antibody region by replacement of less than about 20 amino acid residues from a constant domain of the heavy chain of the antibody or antibody fragment with the non-antibody polypeptide region. The constant domain of the heavy chain may be CH1. The constant domain of the heavy chain may be CH2.The constant domain of the heavy chain may be CH3. In some instances, the constant domain of the heavy chain is not CH2. In some instances, the constant domain of the heavy chain is not CH3. In some instances, the constant domain of the heavy chain is not CH2 or CH3. In some instances, the non-antibody polypeptide region is not inserted into or betweeen CH2 and CH3. In some instances, the antibody fragment is not an Fc fragment. In some embodiments, the antibody fragment is not a human IgG1 Fc. In some embodiments, the non-antibody polypeptide region is not inserted between a Leucine and Threonine of the human IgG1 Fc.

The non-antibody polypeptide region may be inserted into the antibody region by replacement of less than about 20 amino acid residues from a light chain of the antibody or antibody fragment with the non-antibody polypeptide region. The non-antibody polypeptide region may be inserted into the antibody region by replacement of less than about 20 amino acid residues from constant domain of the light chain of the antibody or antibody fragment with the non-antibody polypeptide region.

The replacement of less than about 20 amino acid residues may comprise replacement of at least 1 amino acid residue from the antibody or antibody fragment with the non-antibody polypeptide region. The replacement of less than about 20 amino acid residues may comprise replacement of at least 2 amino acid residues from the antibody or antibody fragment with the non-antibody polypeptide region.

The replacement of less than about 20 amino acid residues may comprise replacement of at least 3 amino acid residues from the antibody or antibody fragment with the non-antibody polypeptide region. The replacement of less than about 20 amino acid residues may comprise replacement of less than 15 amino acid residues from the antibody or antibody fragment with the non-antibody polypeptide region. The replacement of less than about 20 amino acid residues may comprise replacement of less than 10 amino acid residues from the antibody or antibody fragment with the non-antibody polypeptide region. The replacement of less than about 20 amino acid residues may comprise replacement of less than 5 amino acid residues from the antibody or antibody fragment with the non-antibody polypeptide region.

The replacement of less than about 20 amino acid residues may comprise replacement of 5 or fewer amino acid residues from the antibody or antibody fragment with the non-antibody polypeptide region. The replacement of less than about 20 amino acid residues may comprise replacement of 4 or fewer amino acid residues from the antibody or antibody fragment with the non-antibody polypeptide region. The replacement of less than about 20 amino acid residues may comprise replacement of 3 or fewer amino acid residues from the antibody or antibody fragment with the non-antibody polypeptide region.

The replacement of less than about 20 amino acid residues may comprise replacement of 1-15 amino acid residues from the antibody or antibody fragment with the non-antibody polypeptide region. The replacement of less than about 20 amino acid residues may comprise replacement of 1-10 amino acid residues from the antibody or antibody fragment with the non-antibody polypeptide region. The replacement of less than about 20 amino acid residues may comprise replacement of 1-5 amino acid residues from the antibody or antibody fragment with the non-antibody polypeptide region. The replacement of the amino acid residues may comprise replacement of one or more amino acids selected from a group consisting of serine (S), glycine (G), lysine (K), proline (P), threonine (T), glutamine (Q), glutamic acid (E), alanine (A), asparagine (N), and histidine (H).

The replacement of less than about 20 amino acids may comprise replacement of 5 or fewer amino acid residues from the CH1 domain of the antibody or antibody fragment.The replacement of less than about 20 amino acids may comprise replacement of 4 or fewer amino acid residues from the CH1 domain of the antibody or antibody fragment. The replacement of less than about 20 amino acids may comprise replacement of 3 or fewer amino acid residues from the CH1 domain of the antibody or antibody fragment. The replacement of less than about 20 amino acids may comprise replacement of 2 or fewer amino acid residues from the CH1 domain of the antibody or antibody fragment. The replacement of less than about 20 amino acids may comprise replacement of 1 amino acid residue from the CH1 domain of the antibody or antibody fragment.

The one or more amino acid residues that are replaced may be selected from a group consisting of serine (S), glycine (G), proline (P), threonine (T), and glutamine (Q). The replacement of less than about 20 amino acids may comprise replacement of serine 180 (S180) from the CH1 domain. The replacement of less than about 20 amino acids may comprise replacement of glycine 181 (G181) from the CH1 domain. The replacement of less than about 20 amino acids may comprise replacement of serine 180 (S180) and glycine 181 (G181) from the CH1 domain. The replacement of less than about 20 amino acids may comprise replacement of proline 156 (P156) from the CH1 domain. The replacement of less than about 20 amino acids may comprise replacement of serine and glycine from the CH1 domain. The serine and glycine may be adjacent to each other. The replacement of less than about 20 amino acids may comprise replacement of threonine and serine from the CH1 domain. The threonine and serine may be adjacent to each other.

The replacement of less than about 20 amino acids may comprise replacement of 5 or fewer amino acid residues from the CH2 domain of the antibody or antibody fragment. The replacement of less than about 20 amino acids may comprise replacement of 4 or fewer amino acid residues from the CH2 domain of the antibody or antibody fragment. The replacement of less than about 20 amino acids may comprise replacement of 3 or fewer amino acid residues from the CH2 domain of the antibody or antibody fragment. The replacement of less than about 20 amino acids may comprise replacement of 2 or fewer amino acid residues from the CH2 domain of the antibody or antibody fragment. The replacement of less than about 20 amino acids may comprise replacement of 1 amino acid residue from the CH2 domain of the antibody or antibody fragment.

The replacement of less than about 20 amino acids may comprise replacement of one or more amino acids from the CH2 domain; and wherein the one or more amino acid residues may be selected from a group consisting of glutamic acid (E), alanine (A) and proline (P). The replacement of less than about 20 amino acids may comprise replacement of glutamic acid 274 (E274) from the CH2 domain. The replacement of less than about 20 amino acids may comprise replacement of alanine 302 (A302) from the CH2 domain. The replacement of less than about 20 amino acids may comprise replacement of proline 334 (P334) from the CH2 domain.

The replacement of less than about 20 amino acids may comprise replacement of 5 or fewer amino acid residues from the CH3 domain of the antibody or antibody fragment. The replacement of less than about 20 amino acids may comprise replacement of 4 or fewer amino acid residues from the CH3 domain of the antibody or antibody fragment. The replacement of less than about 20 amino acids may comprise replacement of 3 or fewer amino acid residues from the CH3 domain of the antibody or antibody fragment. The replacement of less than about 20 amino acids may comprise replacement of 2 or fewer amino acid residues from the CH3 domain of the antibody or antibody fragment. The replacement of less than about 20 amino acids may comprise replacement of 1 amino acid residue from the CH3 domain of the antibody or antibody fragment. The replacement of less than about 20 amino acids may comprise replacement of one or more amino acids from the CH3 domain, wherein the one or more amino acid residues may be selected from a group consisting of threonine (T), lysine (K), asparagine (N), and glycine (G).

The replacement of less than about 20 amino acids may comprise replacement of threonine 361 (T361) from the CH3 domain. The replacement of less than about 20 amino acids may comprise replacement of lysine 362 (K362) from the CH3 domain. The replacement of less than about 20 amino acids may comprise replacement of asparagine 363 (N363) from the CH3 domain. The replacement of less than about 20 amino acids may comprise replacement of threonine 361 (T361), lysine 362 (K362), and asparagine 363 (N363) from the CH3 domain. The replacement of less than about 20 amino acids may comprise replacement of asparagine 389 (N389) from the CH3 domain. The replacement of less than about 20 amino acids may comprise replacement of glycine 390 (G390) from the CH3 domain. The replacement of less than about 20 amino acids may comprise replacement of asparagine 389 (N389) and glycine 390 (G390) from the CH3 domain. The replacement of less than about 20 amino acids may comprise replacement of glycine 425 (G425) from the CH3 domain. The replacement of less than about 20 amino acids may comprise replacement of asparagine 426 (N426) from the CH3 domain. The replacement of less than about 20 amino acids may comprise replacement of glycine 425 (G425) and asparagine 363 (N363) from the CH3 domain. The replacement of less than about 20 amino acids may comprise replacement of threonine and asparagine from the CH3 domain. The threonine and asparagine may be adjacent to each other. The the replacement of less than about 20 amino acids may comprise replacement of threonine, lysine, and asparagine from the CH3 domain. The threonine, lysine, and asparagine may be adjacent to each other.

The replacement of less than about 20 amino acids may comprise replacement of 5 or fewer amino acid residues from the constant domain of the light chain of the antibody or antibody fragment. The replacement of less than about 20 amino acids may comprise replacement of 4 or fewer amino acid residues from the constant domain of the light chain of the antibody or antibody fragment. The replacement of less than about 20 amino acids may comprise replacement of 3 or fewer amino acid residues from the constant domain of the light chain of the antibody or antibody fragment. The replacement of less than about 20 amino acids may comprise replacement of 2 or fewer amino acid residues from the constant domain of the light chain of the antibody or antibody fragment. The replacement of less than about 20 amino acids may comprise replacement of 1 amino acid residue from the constant domain of the light chain of the antibody or antibody fragment.

The replacement of less than about 20 amino acids may comprise replacement of one or more amino acids from the constant domain of the light chain; and wherein the one or more amino acid residues may be selected from a group consisting of serine (S), glycine (G), proline (P), lysine (K), asparagine (N) and histidine (H). The replacement of less than about 20 amino acids may comprise replacement of serine 202 (S202) from the constant domain of the light chain. The replacement of less than about 20 amino acids may comprise replacement of glycine 128 (G128) from the constant domain of the light chain. The replacement of less than about 20 amino acids may comprise replacement of lysine 169 (K169) from the constant domain of the light chain. The replacement of less than about 20 amino acids may comprise replacement of proline 141 (P141) from the constant domain of the light chain. The replacement of less than about 20 amino acids may comprise replacement of asparagine (N152) from the constant domain of the light chain. The replacement of less than about 20 amino acids may comprise replacement of lysine 138 (K138) from the constant domain of the light chain. The replacement of less than about 20 amino acids may comprise replacement of histidine 139 (H139) from the constant domain of the light chain. The replacement of less than about 20 amino acids may comprise replacement of lysine 138 (K138) and histidine (H139) from the constant domain of the light chain. The replacement of less than about 20 amino acids may comprise replacement of lysine and histidine from the constant domain of the light chain. The lysine and histidine may be adjacent to each other.

The non-antibody polypeptide region may be inserted into the antibody region without replacing any amino acid residues of the antibody or antibody fragment with the non-antibody polypeptide region. The non-antibody polypeptide region may be grafted into the antibody region without replacing any amino acid residues of the antibody or antibody fragment with the non-antibody polypeptide region. The non-antibody polypeptide may comprise a peptide and one or more linkers. The non-antibody polypeptide region may be grafted into a Fab without replacing any amino acid residues of the antibody or antibody fragment. The non-antibody polypeptide region may be grafted into a Fab heavy chain without replacing any amino acid residues of the antibody or antibody fragment. The non-antibody polypeptide region may be grafted into a Fab light chain without replacing any amino acid residues of the antibody or antibody fragment. The non-antibody polypeptide region may be grafted into a constant region without replacing any amino acid residues of the antibody or antibody fragment. The non-antibody polypeptide region may be grafted into a hinge region without replacing any amino acid residues of the antibody or antibody fragment. The non-antibody polypeptide region may be grafted into an antibody region selected from a CH₁ domain, a CH₂ domain, a CH₃ domain, a CL₁ domain, an Fc region, a hinge region, a VH region and a VL region without replacing any amino acid residues of the antibody or antibody fragment. The non-antibody polypeptide may be fused to the C-terminus of the Fab without replacing any amino acid residues of the antibody or antibody fragment. The non-antibody polypeptide may be fused to the C-terminus of the Fab without replacing any amino acid residues of the antibody or antibody fragment via a linker. The non-antibody polypeptide may be fused to the C-terminus of the Fab without replacing any amino acid residues of the antibody or antibody fragment at cysteine 223 (C223). The non-antibody polypeptide may be grafted between the C-terminus of the Fab and the hinge region without replacing any amino acid residues of the antibody or antibody fragment. The non-antibody polypeptide may be grafted between the C-terminus of a Fab heavy chain and the hinge region without replacing any amino acid residues of the antibody or antibody fragment, following cysteine 223 (C223).

The non-antibody polypeptide region may comprise 15 or more amino acids. The non-antibody polypeptide region may comprise 16 or more amino acids. The non-antibody polypeptide region may comprise 17 or more amino acids. The non-antibody polypeptide region may comprise 18 or more amino acids. The non-antibody polypeptide region may comprise 19 or more amino acids. The non-antibody polypeptide region may comprise 20 or more amino acids. The non-antibody polypeptide region may comprise 21 or more amino acids. The non-antibody polypeptide region may comprise 22 or more amino acids. The non-antibody polypeptide region may comprise 20 or more amino acids. The non-antibody polypeptide region may comprise 30 or more amino acids. The non-antibody polypeptide region may comprise 40 or more amino acids. The non-antibody polypeptide region may comprise 50 or more amino acids. The non-antibody polypeptide region may comprise 100 or more amino acids. The non-antibody polypeptide region may comprise 150 or more amino acids.

The non-antibody polypeptide region may comprise a protein-based region. The protein-based region may be based on or derived from one or more proteins selected from a group consisting of erythropoietin (EPO), chemokine (CXC Motif) receptor-4 (CXCR4) binding peptide (CXCR4-BP), tumor-homing peptide, integrin αvβ33 binding peptide, and T-cell epitope peptide. The tumor-homing peptide may be NGR. The tumor-homing peptide may be NGR. The integrin αvβ33 binding peptide may be Int. The T-cell epitope peptide may be GCN4.

The protein-based region of the non-antibody polypeptide region may be based on or derived from erythropoietin. The erythropoietin may comprise an amino acid sequence that is at least 50% homologous to SEQ ID NO: 85. The erythropoietin may comprise an amino acid sequence that is at least 60% homologous to SEQ ID NO: 85. The erythropoietin may comprise an amino acid sequence that is at least 70% homologous to SEQ ID NO: 85. The erythropoietin may comprise an amino acid sequence that is at least 80% homologous to SEQ ID NO: 85. The erythropoietin may comprise an amino acid sequence that is at least 90% homologous to SEQ ID NO: 85.

The protein-based region of the non-antibody peptide may be based on or derived from CXCR4-BP. The CXCR4-BP may comprise an amino acid sequence that is at least 50% homologous to SEQ ID NO: 83. The CXCR4-BP may comprise an amino acid sequence that is at least 60% homologous to SEQ ID NO: 83. The CXCR4-BP may comprise an amino acid sequence that is at least 70% homologous to SEQ ID NO: 83. The CXCR4-BP may comprise an amino acid sequence that is at least 80% homologous to SEQ ID NO: 83. The CXCR4-BP may comprise an amino acid sequence that is at least 90% homologous to SEQ ID NO: 83.

The non-antibody polypeptide region may be based on or derived from TCP1. The TCP1 may comprise an amino acid sequence that is at least 50% homologous to SEQ ID NO: 78. The TCP1 may comprise an amino acid sequence that is at least 60% homologous to SEQ ID NO: 78. The TCP1 may comprise an amino acid sequence that is at least 70% homologous to SEQ ID NO: 78. The TCP1 may comprise an amino acid sequence that is at least 80% homologous to SEQ ID NO: 78. The TCP1 may comprise an amino acid sequence that is at least 90% homologous to SEQ ID NO: 78.

The TCP1 may comprise an amino acid sequence that is at least 50% homologous to SEQ ID NO: 79. The TCP1 may comprise an amino acid sequence that is at least 60% homologous to SEQ ID NO: 79. The TCP1 may comprise an amino acid sequence that is at least 70% homologous to SEQ ID NO: 79. The TCP1 may comprise an amino acid sequence that is at least 80% homologous to SEQ ID NO: 79. The TCP1 may comprise an amino acid sequence that is at least 90% homologous to SEQ ID NO: 79.

The protein-based region of the non-antibody peptide may be based on or derived from NGR. The NGR may comprise an amino acid sequence that is at least 50% homologous to SEQ ID NO: 80. The NGR may comprise an amino acid sequence that is at least 60% homologous to SEQ ID NO: 80. The NGR may comprise an amino acid sequence that is at least 70% homologous to SEQ ID NO: 80. The NGR may comprise an amino acid sequence that is at least 80% homologous to SEQ ID NO: 80. The NGR may comprise an amino acid sequence that is at least 90% homologous to SEQ ID NO: 80.

The NGR may comprise an amino acid sequence that is at least 50% homologous to SEQ ID NO: 81. The NGR may comprise an amino acid sequence that is at least 60% homologous to SEQ ID NO: 81. The NGR may comprise an amino acid sequence that is at least 70% homologous to SEQ ID NO: 81. The NGR may comprise an amino acid sequence that is at least 80% homologous to SEQ ID NO: 81. The NGR may comprise an amino acid sequence that is at least 90% homologous to SEQ ID NO: 81.

The protein-based region of the non-antibody polypeptide region may be based on or derived from Int. The Int may comprise an amino acid sequence that is at least 50% homologous to SEQ ID NO: 82. The Int may comprise an amino acid sequence that is at least 60% homologous to SEQ ID NO: 82. The Int may comprise an amino acid sequence that is at least 70% homologous to SEQ ID NO: 82. The Int may comprise an amino acid sequence that is at least 80% homologous to SEQ ID NO: 82. The Int may comprise an amino acid sequence that is at least 90% homologous to SEQ ID NO: 82.

The protein-based region of the non-antibody polypeptide region may be based on or derived from GCN4. The GCN4 may comprise an amino acid sequence that is at least 50% homologous to SEQ ID NO: 84. The GCN4 may comprise an amino acid sequence that is at least 60% homologous to SEQ ID NO: 84. The GCN4 may comprise an amino acid sequence that is at least 70% homologous to SEQ ID NO: 84. The GCN4 may comprise an amino acid sequence that is at least 80% homologous to SEQ ID NO: 84. The GCN4 may comprise an amino acid sequence that is at least 90% homologous to SEQ ID NO: 84.

The antibody or antibody fragment may be based on or derived from a group consisting of UCHT1, anti-CD19, anti-CD20, and Her2. The antibody or antibody fragment may comprise a fragment antigen binding (Fab), fragment antigen-binding including hinge region (F(ab′)₂), fragment antigen-binding including one hinge region (Fab′), fragment crystallizable (Fc), variable domain (e.g., V_{H} or V_{L}), constant domain (e.g., C_{H1}, C_{H2}, C_{H3}, or C_{L}, single-chain varaible fragment (scFV), di-ScFv, single domain antibody (sdAb), minibody, diabody, tribody, tetrabody, trifunctional antibody. The antibody or antibody fragment may comprise one or more heavy chains, light chains, or both. The antibody or antibody fragment may comprise one or more constant domains.

The antibody or antibody fragment may be based on or derived from a UCHT1 antibody or antibody fragment.The UCHT1 may be UCHT1scFv. The UCHT1 may be UCHT1 Fab fragment. The UCHT1 may be UCHT1 light chain. The UCHT1 may be UCHT1 heavy chain. The UCHT1 may comprise an amino acid sequence that is at least 50% homologous to a sequence selected from SEQ ID NOS: 34, 35, 41, and 88. The UCHT1 may comprise an amino acid sequence that is at least 60% homologous to a sequence selected from SEQ ID NOS: 34, 35, 41, and 88. The UCHT1 may comprise an amino acid sequence that is at least 70% homologous to a sequence selected from SEQ ID NOS: 34, 35, 41, and 88. The UCHT1 may comprise an amino acid sequence that is at least 80% homologous to a sequence selected from SEQ ID NOS: 34, 35, 41, and 88. The UCHT1 may comprise an amino acid sequence that is at least 90% homologous to a sequence selected from SEQ ID NOS: 34, 35, 41, and 88. The UCHT1 may comprise an amino acid sequence that comprises 10 or more consecutive amino acids from a sequence selected from SEQ ID NOS: 34, 35, 41, and 88. The UCHT1 may comprise an amino acid sequence that comprises 50 or more consecutive amino acids from a sequence selected from SEQ ID NOS: 34, 35, 41, and 88. The UCHT1 may comprise an amino acid sequence that comprises 100 or more consecutive amino acids from a sequence selected from SEQ ID NOS: 34, 35, 41, and 88. The UCHT1 may comprise an amino acid sequence that comprises 200 or more consecutive amino acids from a sequence selected from SEQ ID NOS: 34, 35, 41, and 88. The amino acid sequence may be SEQ ID NO: 34. The amino acid sequence may be SEQ ID NO: 35. The amino acid sequence may be SEQ ID NO: 41. The amino acid sequence may be SEQ ID NO: 88.

The antibody or antibody fragment may be based on or derived from an anti-CD19 antibody or antibody fragment.The anti-CD19 may be anti-CD19scFv. The anti-CD19 may be anti-CD19 light chain. The anti-CD19 may be anti-CD19 heavy chain. The anti-CD19 may be anti-CD19 Fab fragment. The anti-CD19 may comprise an amino acid sequence that is at least 50% homologous to a sequence selected from SEQ ID NOS: 38, 39, 42, and 87. The anti-CD19 may comprise an amino acid sequence that is at least 60% homologous to a sequence selected from SEQ ID NOS: 38, 39, 42, and 87. The anti-CD19 may comprise an amino acid sequence that is at least 70% homologous to a sequence selected from SEQ ID NOS: 38, 39, 42, and 87. The anti-CD19 may comprise an amino acid sequence that is at least 80% homologous to a sequence selected from SEQ ID NOS: 38, 39, 42, and 87. The anti-CD19 may comprise an amino acid sequence that is at least 90% homologous to a sequence selected from SEQ ID NOS: 38, 39, 42, and 87. The anti-CD19 may comprise an amino acid sequence that comprises 10 or more consecutive amino acids from a sequence selected from SEQ ID NOS: 38, 39, 42, and 87. The anti-CD19 may comprise an amino acid sequence that comprises 50 or more consecutive amino acids from a sequence selected from SEQ ID NOS: 38, 39, 42, and 87. The anti-CD19 may comprise an amino acid sequence that comprises 75 or more consecutive amino acids from a sequence selected from SEQ ID NOS: 38, 39, 42, and 87. The anti-CD19 may comprise an amino acid sequence that comprises 100 or more consecutive amino acids from a sequence selected from SEQ ID NOS: 38, 39, 42, and 87.

The antibody or antibody fragment may be based on or derived from an anti-CD20 antibody or antibody fragment. The anti-CD20 may be anti-CD20 light chain. The anti-CD20 may comprise an amino acid sequence that is at least 50% homologous to a sequence selected from SEQ ID NOS: 36, 37 and 43. The anti-CD20 may comprise an amino acid sequence that is at least 60% homologous to a sequence selected from SEQ ID NOS: 36, 37 and 43. The anti-CD20 may comprise an amino acid sequence that is at least 70% homologous to a sequence selected from SEQ ID NOS: 36, 37 and 43. The anti-CD20 may comprise an amino acid sequence that is at least 80% homologous to a sequence selected from SEQ ID NOS: 36, 37 and 43. The anti-CD20 may comprise an amino acid sequence that is at least 90% homologous to a sequence selected from SEQ ID NOS: 36, 37 and 43. The anti-CD20 may comprise an amino acid sequence that comprises 50 or more consecutive amino acids from a sequence selected from SEQ ID NOS: 36, 37 and 43. The anti-CD20 may comprise an amino acid sequence that comprises 100 or more consecutive amino acids from a sequence selected from SEQ ID NOS: 36, 37 and 43. The anti-CD20 may comprise an amino acid sequence that comprises 150 or more consecutive amino acids from a sequence selected from SEQ ID NOS: 36, 37 and 43. The anti-CD20 may comprise an amino acid sequence that comprises 200 or more consecutive amino acids from a sequence selected from SEQ ID NOS: 36, 37 and 43. The anti-CD20 may be anti-CD20 heavy chain. The anti-CD20 heavy chain may comprise an amino acid sequence that is at least 50% homologous to an amino acid sequence selected from a group consisting of an amino acid sequence selected from a group consisting of SEQ ID NOS: 36-37. The anti-CD20 heavy chain may comprise an amino acid sequence that is at least 60% homologous to an amino acid sequence selected from a group consisting of SEQ ID NOS: 36-37. The anti-CD20 heavy chain may comprise an amino acid sequence that is at least 70% homologous to an amino acid sequence selected from a group consisting of SEQ ID NOS: 36-37. The anti-CD20 heavy chain may comprise an amino acid sequence that is at least 80% homologous to an amino acid sequence selected from a group consisting of SEQ ID NOS: 36-37. The anti-CD20 may comprise an amino acid sequence that is at least 90% homologous to an amino acid sequence selected from a group consisting of SEQ ID NOS: 36-37. The anti-CD20 heavy chain may comprise an amino acid sequence that comprises 50 or more consecutive amino acids from an amino acid sequence selected from a group consisting of SEQ ID NOS: 36-37. The anti-CD20 heavy chain may comprise an amino acid sequence that comprises 100 or more consecutive amino acids from an amino acid sequence selected from a group consisting of SEQ ID NOS: 36-37. The anti-CD20 heavy chain may comprise an amino acid sequence that comprises 150 or more consecutive amino acids from an amino acid sequence selected from a group consisting of SEQ ID NOS: 36-37. The anti-CD20 heavy chain may comprise an amino acid sequence that comprises 200 or more consecutive amino acids from an amino acid sequence selected from a group consisting of SEQ ID NOS: 36-37. The amino acid sequence may be SEQ ID NO: 36. The amino acid sequence may be SEQ ID NO: 37.

The antibody or antibody fragment may be based on or derived from a Her2 antibody or antibody fragment.The Her2 may be Her2scFv. The Her2 may comprise an amino acid sequence that is at least 50% homologous to an amino acid selected from a group consisting of SEQ ID NOS: 33, 40, and 86. The Her2 may comprise an amino acid sequence that is at least 60% homologous to an amino acid selected from a group consisting of SEQ ID NOS: 33, 40, and 86. The Her2 may comprise an amino acid sequence that is at least 70% homologous to an amino acid selected from a group consisting of SEQ ID NOS: 33, 40, and 86. The Her2 may comprise an amino acid sequence that is at least 80% homologous to an amino acid selected from a group consisting of SEQ ID NOS: 33, 40, and 86. The Her2 may comprise an amino acid sequence that is at least 90% homologous to an amino acid selected from a group consisting of SEQ ID NOS: 33, 40, and 86. The Her2 may comprise an amino acid sequence that comprises 10 or more consecutive amino acids from an amino acid selected from a group consisting of SEQ ID NOS: 33, 40, and 86. The Her2 may comprise an amino acid sequence uence that comprises 50 or more consecutive amino acids from an amino acid selected from a group consisting of SEQ ID NOS: 33, 40, and 86. The Her2 may comprise an amino acid sequence that comprises 100 or more consecutive amino acids from an amino acid selected from a group consisting of SEQ ID NOS: 33, 40, and 86. The Her2 may comprise an amino acid sequence that comprises 200 or more consecutive amino acids from an amino acid selected from a group consisting of SEQ ID NOS: 33, 40, and 86. The Her2 may be Her2 light chain. The Her2 may comprise an amino acid sequence that is at least 50% homologous to SEQ ID NO: 40. The Her2 may comprise an amino acid sequence that is at least 60% homologous to SEQ ID NO: 40. The Her2 may comprise an amino acid sequence that is at least 70% homologous to SEQ ID NO: 40. The Her2 may comprise an amino acid sequence that is at least 80% homologous to SEQ ID NO: 40. The Her2 may comprise an amino acid sequence that is at least 90% homologous to SEQ ID NO: 40. The Her2 may comprise an amino acid sequence that comprises 10 or more consecutive amino acids from SEQ ID NO: 40. The Her2 may comprise an amino acid sequence that comprises 50 or more consecutive amino acids from SEQ ID NO: 40. The Her2 may comprise an amino acid sequence that comprises 100 or more consecutive amino acids from SEQ ID NO: 40. The Her2 may comprise an amino acid sequence that comprises 200 or more consecutive amino acids from SEQ ID NO: 40. The Her2 may be Her2 heavy chain. The Her2 may comprise an amino acid sequence that is at least 50% homologous to SEQ ID NO: 33. The Her2 may comprise an amino acid sequence that is at least 60% homologous to SEQ ID NO: 33. The Her2 may comprise an amino acid sequence that is at least 70% homologous to SEQ ID NO: 33. The Her2 may comprise an amino acid sequence that is at least 80% homologous to SEQ ID NO: 33. The Her2 may comprise an amino acid sequence that is at least 90% homologous to SEQ ID NO: 33. The Her2 may comprise an amino acid sequence that comprises 10 or more consecutive amino acids from SEQ ID NO: 33. The Her2 may comprise an amino acid sequence that comprises 50 or more consecutive amino acids from SEQ ID NO: 33. The Her2 may comprise an amino acid sequence that comprises 100 or more consecutive amino acids from SEQ ID NO: 33. The Her2 may comprise an amino acid sequence that comprises 200 or more consecutive amino acids from SEQ ID NO: 33.

The antibody fusion protein may comprise an amino acid sequence that is at least 50% homologous to an amino acid sequence selected from a group consisting of SEQ ID NOS: 45-57, 61-66. The antibody fusion protein may comprise an amino acid sequence that is at least 60% homologous to an amino acid sequence selected from a group consisting of SEQ ID NOS: 45-57, 61-66. The antibody fusion protein may comprise an amino acid sequence that is at least 70% homologous to an amino acid sequence selected from a group consisting of SEQ ID NOS: 45-57, 61-66. The antibody fusion protein may comprise an amino acid sequence that is at least 80% homologous to an amino acid sequence selected from a group consisting of SEQ ID NOS: 45-57, 61-66. The antibody fusion protein may comprise an amino acid sequence that is at least 90% homologous to an amino acid sequence selected from a group consisting of SEQ ID NOS: 45-57, 61-66. The amino acid sequence may be SEQ ID NO: 45. The amino acid sequence may be SEQ ID NO: 46. The amino acid sequence may be SEQ ID NO: 47. The amino acid sequence may be SEQ ID NO: 48. The amino acid sequence may be SEQ ID NO: 49. The amino acid sequence may be SEQ ID NO: 50. The amino acid sequence may be SEQ ID NO: 51. The amino acid sequence may be SEQ ID NO: 52. The amino acid sequence may be SEQ ID NO: 53. The amino acid sequence may be SEQ ID NO: 54. The amino acid sequence may be SEQ ID NO: 55. The amino acid sequence may be SEQ ID NO: 56. The amino acid sequence may be SEQ ID NO: 57. The amino acid sequence may be SEQ ID NO: 58. The amino acid sequence may be SEQ ID NO: 59. The amino acid sequence may be SEQ ID NO: 60. The amino acid sequence may be SEQ ID NO: 61. The amino acid sequence may be SEQ ID NO: 62. The amino acid sequence may be SEQ ID NO: 63. The amino acid sequence may be SEQ ID NO: 64. The amino acid sequence may be SEQ ID NO: 65. The amino acid sequence may be SEQ ID NO: 66. The amino acid sequence may be SEQ ID NO: 67. The amino acid sequence may be SEQ ID NO: 68. The amino acid sequence may be SEQ ID NO: 69. The amino acid sequence may be SEQ ID NO: 70.

The antibody fusion protein may comprise an amino acid sequence that comprises 50 or more consecutive amino acids from any one of SEQ ID NOS: 45-57, 61-66. The antibody fusion protein may comprise an amino acid sequence that comprises 100 or more consecutive amino acids from any one of SEQ ID NOS: 45-57, 61-66. The antibody fusion protein may comprise an amino acid sequence that comprises 150 or more consecutive amino acids from any one of SEQ ID NOS: 45-57, 61-66. The antibody fusion protein may comprise an amino acid sequence that comprises 200 or more consecutive amino acids from any one of SEQ ID NOS: 45-57, 61-66. The amino acid sequence may be SEQ ID NO: 45. The amino acid sequence may be SEQ ID NO: 46. The amino acid sequence may be SEQ ID NO: 47. The amino acid sequence may be SEQ ID NO: 48. The amino acid sequence may be SEQ ID NO: 49. The amino acid sequence may be SEQ ID NO: 50. The amino acid sequence may be SEQ ID NO: 51. The amino acid sequence may be SEQ ID NO: 2. The amino acid sequence may be SEQ ID NO: 53. The amino acid sequence may be SEQ ID NO: 54. The amino acid sequence may be SEQ ID NO: 55. The amino acid sequence may be SEQ ID NO: 56. The amino acid sequence may be SEQ ID NO: 57. The amino acid sequence may be SEQ ID NO: 58. The amino acid sequence may be SEQ ID NO: 59. The amino acid sequence may be SEQ ID NO: 60. The amino acid sequence may be SEQ ID NO: 61. The amino acid sequence may be SEQ ID NO: 62. The amino acid sequence may be SEQ ID NO: 63. The amino acid sequence may be SEQ ID NO: 64. The amino acid sequence may be SEQ ID NO: 65. The amino acid sequence may be SEQ ID NO: 66. The amino acid sequence may be SEQ ID NO: 67. The amino acid sequence may be SEQ ID NO: 68. The amino acid sequence may be SEQ ID NO: 69. The amino acid sequence may be SEQ ID NO: 70.

The antibody fusion protein may be encoded by a nucleic acid sequence that is at least 50% homologous to a nucleic acid sequence selected from a group consisting of SEQ ID NOS: 11-23, 27 and 28. The antibody fusion protein may be encoded by a nucleic acid sequence that is at least 60% homologous to a nucleic acid sequence selected from a group consisting of SEQ ID NOS: 11-23, 27 and 28. The antibody fusion protein may be encoded by a nucleic acid sequence that is at least 70% homologous to a nucleic acid sequence selected from a group consisting of SEQ ID NOS: 11-23, 27 and 28. The antibody fusion protein may be encoded by a nucleic acid sequence that is at least 80% homologous to a nucleic acid sequence selected from a group consisting of SEQ ID NOS: 11-23, 27 and 28. The antibody fusion protein may be encoded by a nucleic acid sequence that is at least 90% homologous to a nucleic acid sequence selected from a group consisting of SEQ ID NOS: 11-23, 27 and 28. The nucleic acid sequence may be SEQ ID NO: 11. The nucleic acid sequence may be SEQ ID NO: 12. The nucleic acid sequence may be SEQ ID NO: 13. The nucleic acid sequence may be SEQ ID NO: 14. The nucleic acid sequence may be SEQ ID NO: 15. The nucleic acid sequence may be SEQ ID NO: 16. The nucleic acid sequence may be SEQ ID NO: 17. The nucleic acid sequence may be SEQ ID NO: 18. The nucleic acid sequence may be SEQ ID NO: 19. The nucleic acid sequence may be SEQ ID NO: 20. The nucleic acid sequence may be SEQ ID NO: 21. The nucleic acid sequence may be SEQ ID NO: 22. The nucleic acid sequence may be SEQ ID NO: 23. The nucleic acid sequence may be SEQ ID NO: 24. The nucleic acid sequence may be SEQ ID NO: 25. The nucleic acid sequence may be SEQ ID NO: 26. The nucleic acid sequence may be SEQ ID NO: 27. The nucleic acid sequence may be SEQ ID NO: 28. The nucleic acid sequence may be SEQ ID NO: 29. The nucleic acid sequence may be SEQ ID NO: 30. The nucleic acid sequence may be SEQ ID NO: 31. The nucleic acid sequence may be SEQ ID NO: 32.

The antibody fusion protein may further comprise one or more additional antibodies or antibody fragments. The one or more additional antibodies or antibody fragments may be based on or derived from a UCHT1 antibody. The one or more additional antibodies or antibody fragments may be based on or derived from a Her2 antibody. The one or more additional antibodies or antibody fragments may be based on or derived from an anti-CD19 antibody. The one or more additional antibodies or antibody fragments may be based on or derived from an anti-CD20 antibody. The one or more additional antibodies or antibody fragments may comprise a fragment antigen binding (Fab), fragment antigen-binding including hinge region (F(ab′)₂), fragment antigen-binding including one hinge region (Fab′), fragment crystallizable (Fc), variable domain (e.g., V_{H} or V_{L}), constant domain (e.g., C_{H1}, C_{H2}, C_{H3}, or C_{L}), single-chain varaible fragment (scFV), di-ScFv, single domain antibody (sdAb), minibody, diabody, tribody, tetrabody, trifunctional antibody. The one or more additional antibodies or antibody fragments may comprise one or more heavy chains, light chains, or both. The one or more additional antibodies or antibody fragments may comprise one or more constant domains. The one or more additional antibodies or antibody fragments may comprise one or more variable domains. The one or more additional antibodies or antibody fragments may comprise an amino acid sequence that is at least 50% homologous to an amino acid sequence selected from a group consisting of SEQ ID NO: 33-44. The one or more additional antibodies or antibody fragments may comprise an amino acid sequence that is at least 60% homologous to an amino acid sequence selected from a group consisting of SEQ ID NO: 33-44. The one or more additional antibodies or antibody fragments may comprise an amino acid sequence that is at least 70% homologous to an amino acid sequence selected from a group consisting of SEQ ID NO: 33-44. The one or more additional antibodies or antibody fragments may comprise an amino acid sequence that is at least 80% homologous to an amino acid sequence selected from a group consisting of SEQ ID NO: 33-44. The one or more additional antibodies or antibody fragments may comprise an amino acid sequence that is at least 90% homologous to an amino acid sequence selected from a group consisting of SEQ ID NO: 33-44. The amino acid sequence may be SEQ ID NO: 33. The amino acid sequence may be SEQ ID NO: 34. The amino acid sequence may be SEQ ID NO: 35. The amino acid sequence may be SEQ ID NO: 36. The amino acid sequence may be SEQ ID NO: 37. The amino acid sequence may be SEQ ID NO: 38. The amino acid sequence may be SEQ ID NO: 39. The amino acid sequence may be SEQ ID NO: 40. The amino acid sequence may be SEQ ID NO: 41. The amino acid sequence may be SEQ ID NO: 42. The amino acid sequence may be SEQ ID NO: 43. The amino acid sequence may be SEQ ID NO: 44.

The one or more additional antibodies or antibody fragments may comprise an amino acid sequence that comprises 50 or more consecutive amino acids from any one of SEQ ID NO: 33-44. The one or more additional antibodies or antibody fragments may comprise an amino acid sequence that comprises 100 or more consecutive amino acids from any one of SEQ ID NO: 33-44. The one or more additional antibodies or antibody fragments may comprise an amino acid sequence that comprises 150 or more consecutive amino acids from any one of SEQ ID NO: 33-44. The one or more additional antibodies or antibody fragments may comprise an amino acid sequence that comprises 200 or more consecutive amino acids from any one of SEQ ID NO: 33-44. The amino acid sequence may be SEQ ID NO: 33. The amino acid sequence may be SEQ ID NO: 34. The amino acid sequence may be SEQ ID NO: 35. The amino acid sequence may be SEQ ID NO: 36. The amino acid sequence may be SEQ ID NO: 37. The amino acid sequence may be SEQ ID NO: 38. The amino acid sequence may be SEQ ID NO: 39. The amino acid sequence may be SEQ ID NO: 40. The amino acid sequence may be SEQ ID NO: 41. The amino acid sequence may be SEQ ID NO: 42. The amino acid sequence may be SEQ ID NO: 43. The amino acid sequence may be SEQ ID NO: 44.

The one or more additional antibodies or antibody fragments may be encoded by a nucleic acid sequence that is at least 50% homologous to a nucleic acid sequence selected from a group consisting of SEQ ID NO: 1-10. The one or more additional antibodies or antibody fragments may be encoded by a nucleic acid sequence that is at least 60% homologous to a nucleic acid sequence selected from a group consisting of SEQ ID NO: 1-10. The one or more additional antibodies or antibody fragments may be encoded by a nucleic acid sequence that is at least 70% homologous to a nucleic acid sequence selected from a group consisting of SEQ ID NO: 1-10. The one or more additional antibodies or antibody fragments may be encoded by a nucleic acid sequence that is at least 80% homologous to a nucleic acid sequence selected from a group consisting of SEQ ID NO: 1-10. The one or more additional antibodies or antibody fragments may be encoded by a nucleic acid sequence that is at least 90% homologous to a nucleic acid sequence selected from a group consisting of SEQ ID NO: 1-10. The one or more additional antibodies or antibody fragments may be encoded by a nucleic acid sequence that comprises 100 or more consecutive nucleic acids from any one of SEQ ID NO: 1-10. The one or more additional antibodies or antibody fragments may be encoded by a nucleic acid sequence that comprises 200 or more consecutive nucleic acids from any one of SEQ ID NO: 1-10. The one or more additional antibodies or antibody fragments may be encoded by a nucleic acid sequence that comprises 300 or more consecutive nucleic acids from any one of SEQ ID NO: 1-10. The one or more additional antibodies or antibody fragments may be encoded by a nucleic acid sequence that comprises 400 or more consecutive nucleic acids from any one of SEQ ID NO: 1-10. The nucleic acid sequence may be SEQ ID NO: 1. The nucleic acid sequence may be SEQ ID NO: 2. The nucleic acid sequence may be SEQ ID NO: 3. The nucleic acid sequence may be SEQ ID NO: 4. The nucleic acid sequence may be SEQ ID NO: 5. The nucleic acid sequence may be SEQ ID NO: 6. The nucleic acid sequence may be SEQ ID NO: 7. The nucleic acid sequence may be SEQ ID NO: 8. The nucleic acid sequence may be SEQ ID NO: 9. The nucleic acid sequence may be SEQ ID NO: 10.

The non-antibody polypeptide region disclosed herein may further comprise one or more adapter peptides. An adapter peptide may connect the antibody region to the protein-based region of the non-antibody polypeptide region. Alternatively, or additionally, the adapter peptide may be inserted into the protein-based region of the non-antibody polypeptide region. The antibody fusion proteins disclosed herein may comprise 1, 2, 3, 4, 5, 6, 7, 8, 9, 10 or more adapter peptides. The antibody fusion proteins disclosed herein may comprise 1 or more adapter peptides. The antibody fusion proteins disclosed herein may comprise 2 or more adapter peptides. The antibody fusion proteins disclosed herein may comprise 3 or more adapter peptides. The adapter peptide may be a synthetic peptide. In some instances, the adapter peptide is not based on or derived from an antibody or antibody fragment. In some instances, the adapter peptide is not based on or derived from a complementarity determining region (CDR) of an antibody or antibody fragment. The CDR may be CDR1. The CDR may be CDR2. The CDR may be CDR3.

The adapter peptide may comprise 1, 2, 3, 4, 5, 6, 7, 8, 9, 10 or more consecutive amino acids. The adapter peptide may comprise 10, 20, 30, 40, 50, 60, 70, 80, 90, 100 or more consecutive amino acids. The adapter peptide may comprise 1, 2, 3, 4 or more consecutive amino acids based on or derived from an amino acid sequence selected from a group consisting of SEQ ID NO: 71-77. The adapter peptide may comprise 4 or more consecutive amino acids based on or derived from an amino acid sequence selected from a group consisting of SEQ ID NO: 71-77. The adapter peptide may comprise 5, 6, 7, 9, 10, 11, 12, 13, 14, 15 or more consecutive amino acids based on or derived from an amino acid sequence selected from a group consisting of SEQ ID NO: 71-77. The adapter peptide may comprise 15 or more consecutive amino acids based on or derived from an amino acid sequence selected from a group consisting of SEQ ID NO: 71-77. The adapter peptide may comprise 16, 17, 18, 19, 20 or more consecutive amino acids based on or derived from an amino acid sequence selected from a group consisting of SEQ ID NO: 71-77. The adapter peptide may comprise 20 or more consecutive amino acids based on or derived from an amino acid sequence selected from a group consisting of SEQ ID NO: 71-77. The adapter peptide may comprise an amino acid sequence that is at least about 50% homologous to an amino acid sequence selected from a group consisting of SEQ ID NO: 71-77. The adapter peptide may comprise an amino acid sequence that is at least about 60% homologous to an amino acid sequence selected from a group consisting of SEQ ID NO: 71-77. The adapter peptide may comprise an amino acid sequence that is at least about 70% homologous to an amino acid sequence selected from a group consisting of SEQ ID NO: 71-77. The adapter peptide may comprise an amino acid sequence that is at least about 75% homologous to an amino acid sequence selected from a group consisting of SEQ ID NO: 71-77. The adapter peptide may comprise an amino acid sequence that is at least about 80% homologous to an amino acid sequence selected from a group consisting of SEQ ID NO: 71-77. The adapter peptide may comprise an amino acid sequence that is at least about 85% homologous to an amino acid sequence selected from a group consisting of SEQ ID NO: 71-77. The adapter peptide may comprise an amino acid sequence that is at least about 90% homologous to an amino acid sequence selected from a group consisting of SEQ ID NO: 51-57. The adapter peptide may comprise an amino acid sequence that is at least about 95% homologous to an amino acid sequence selected from a group consisting of SEQ ID NO: 71-77. The adapter peptide may comprise an amino acid sequence that is at least about 97% homologous to an amino acid sequence selected from a group consisting of SEQ ID NO: 71-77. The amino acid sequence may be SEQ ID NO: 71. The amino acid sequence may be SEQ ID NO: 72. The amino acid sequence may be SEQ ID NO: 73. The amino acid sequence may be SEQ ID NO: 74. The amino acid sequence may be SEQ ID NO: 75. The amino acid sequence may be SEQ ID NO: 76. The amino acid sequence may be SEQ ID NO: 77.

Further disclosed herein are uses of an antibody fusion protein to treat a disease or condition in a subject. The antibody fusion protein may comprise (a) an antibody region based on or derived from an antibody or antibody fragment; and (b) a non-antibody polypeptide region comprising 15 or more amino acids, wherein the non-antibody polypeptide region is inserted into the antibody region. The non-antibody polypeptide region may be inserted into a constant domain of the antibody region. The non-antibody polypeptide region may be inserted into the antibody region by replacment of less than about 20 amino acid residues from the antibody or antibody fragment. Alternatively, insertion of the non-antibody polypeptide region does not comprise replacement of one or more amino acid residues from the antibody or antibody fragment from which the antibody region is based on or derived. The non-antibody polypeptide region may comprise 15 or more amino acids. The non-antibody polypeptide region may comprise 16 or more amino acids. The non-antibody polypeptide region may comprise 17 or more amino acids. The non-antibody polypeptide region may comprise 18 or more amino acids. The non-antibody polypeptide region may comprise 19 or more amino acids. The non-antibody polypeptide region may comprise 20 or more amino acids. The non-antibody polypeptide region may be a non-antigenic peptide. In some instances, the non-antibody polypeptide region is not based on or derived from a T-cell epitope. In some instances, the non-antibody polypeptide region is not based on or derived from a B-cell epitope.The antibody fusion protein may comprise any of the antibody fusion proteins disclosed herein. The antibody region may comprise any of the antibody regions disclosed herein. In some instances, the antibody region is not based on or derived from an APC-specific antibody. In some instances, the antibody region is not based on or derived from a MHC-specific antibody. In some instances, the antibody region is not based on or derived from a MHC class I-specific antibody. In some instances, the antibody region is not based on or derived from a MHC class II-specific antibody. The non-antibody polypeptide region may comprise any of the non-antibody polypeptide regions disclosed herein. The non-antibody polypeptide region may comprise a protein-based region. The protein-based region may comprise any of the protein-based regions disclosed herein. The non-antibody polypeptide region may comprise one or more adapter peptides. The one or more adapter peptides may comprise any of the adapter peptides disclosed herein. In some instances, the non-antibody region is not inserted into a complementarity determining region (CDR) of the antibody or antibody fragment. The CDR may be CDR1. The CDR may be CDR2. The CDR may be CDR3. The antibody fusion protein may further comprise one or more additional antibodies or antibody fragments. The one or more additional antibodies or antibody fragments may comprise any of the antibodies or antibody fragments disclosed herein.

Further disclosed herein are uses of an antibody fusion protein to treat a disease or condition in a subject. The antibody fusion protein may comprise (a) an antibody region based on or derived from an antibody or antibody fragment; and (b) a non-antibody polypeptide region, wherein the non-antibody polypeptide region is inserted into the antibody region by replacment of less than about 20 amino acid residues from the antibody or antibody fragment. The non-antibody polypeptide region may comprise 15 or more amino acids. The non-antibody polypeptide region may comprise 16 or more amino acids. The non-antibody polypeptide region may comprise 17 or more amino acids. The non-antibody polypeptide region may comprise 18 or more amino acids. The non-antibody polypeptide region may comprise 19 or more amino acids. The non-antibody polypeptide region may comprise 20 or more amino acids. The non-antibody polypeptide region may be a non-antigenic peptide. In some instances, the non-antibody polypeptide region is not based on or derived from a T-cell epitope. In some instances, the non-antibody polypeptide region is not based on or derived from a B-cell epitope.The antibody fusion protein may comprise any of the antibody fusion proteins disclosed herein. The antibody region may comprise any of the antibody regions disclosed herein. In some instances, the antibody region is not based on or derived from an APC-specific antibody. In some instances, the antibody region is not based on or derived from a MHC-specific antibody. In some instances, the antibody region is not based on or derived from a MHC class I-specific antibody. In some instances, the antibody region is not based on or derived from a MHC class II-specific antibody. The non-antibody polypeptide region may comprise any of the non-antibody polypeptide regions disclosed herein. The non-antibody polypeptide region may comprise a protein-based region. The protein-based region may comprise any of the protein-based regions disclosed herein. The non-antibody polypeptide region may comprise one or more adapter peptides. The one or more adapter peptides may comprise any of the adapter peptides disclosed herein. In some instances, the non-antibody region is not inserted into a complementarity determining region (CDR) of the antibody or antibody fragment. The CDR may be CDR1. The CDR may be CDR2. The CDR may be CDR3. The antibody fusion protein may further comprise one or more additional antibodies or antibody fragments. The one or more additional antibodies or antibody fragments may comprise any of the antibodies or antibody fragments disclosed herein.

Further disclosed herein are methods of treating a disease or condition in a subject in need thereof, the method comprising administering to the subject a antibody fusion protein comprising (a) an antibody region based on or derived from an antibody or antibody fragment; and (b) a non-antibody polypeptide region comprising 15 or more amino acids, wherein the non-antibody polypeptide region is inserted into the antibody region. The non-antibody polypeptide region may be inserted into a constant domain of the antibody region. The non-antibody polypeptide region may be inserted into the antibody region by replacment of less than about 20 amino acid residues from the antibody or antibody fragment. Alternatively, insertion of the non-antibody polypeptide region does not comprise replacement of one or more amino acid residues from the antibody or antibody fragment from which the antibody region is based on or derived. The non-antibody polypeptide region may comprise 15 or more amino acids. The non-antibody polypeptide region may comprise 16 or more amino acids. The non-antibody polypeptide region may comprise 17 or more amino acids. The non-antibody polypeptide region may comprise 18 or more amino acids. The non-antibody polypeptide region may comprise 19 or more amino acids. The non-antibody polypeptide region may comprise 20 or more amino acids. The non-antibody polypeptide region may be a non-antigenic peptide. In some instances, the non-antibody polypeptide region is not based on or derived from a T-cell epitope. In some instances, the non-antibody polypeptide region is not based on or derived from a B-cell epitope.The antibody fusion protein may comprise any of the antibody fusion proteins disclosed herein. The antibody region may comprise any of the antibody regions disclosed herein. In some instances, the antibody region is not based on or derived from an APC-specific antibody. In some instances, the antibody region is not based on or derived from a MHC-specific antibody. In some instances, the antibody region is not based on or derived from a MHC class I-specific antibody. In some instances, the antibody region is not based on or derived from a MHC class II-specific antibody. The non-antibody polypeptide region may comprise any of the non-antibody polypeptide regions disclosed herein. The non-antibody polypeptide region may comprise a protein-based region. The protein-based region may comprise any of the protein-based regions disclosed herein. The non-antibody polypeptide region may comprise one or more adapter peptides. The one or more adapter peptides may comprise any of the adapter peptides disclosed herein. In some instances, the non-antibody region is not inserted into a complementarity determining region (CDR) of the antibody or antibody fragment. The CDR may be CDR1. The CDR may be CDR2. The CDR may be CDR3. The antibody fusion protein may further comprise one or more additional antibodies or antibody fragments. The one or more additional antibodies or antibody fragments may comprise any of the antibodies or antibody fragments disclosed herein.

Further disclosed herein are methods of treating a disease or condition in a subject in need thereof, the method comprising administering to the subject a antibody fusion protein comprising (a) an antibody region based on or derived from an antibody or antibody fragment; and (b) a non-antibody polypeptide region, wherein the non-antibody polypeptide region is inserted into the antibody region by replacment of less than about 20 amino acid residues from the antibody or antibody fragment. The non-antibody polypeptide region may comprise 15 or more amino acids. The non-antibody polypeptide region may comprise 16 or more amino acids. The non-antibody polypeptide region may comprise 17 or more amino acids. The non-antibody polypeptide region may comprise 18 or more amino acids. The non-antibody polypeptide region may comprise 19 or more amino acids. The non-antibody polypeptide region may comprise 20 or more amino acids. The non-antibody polypeptide region may be a non-antigenic peptide. In some instances, the non-antibody polypeptide region is not based on or derived from a T-cell epitope. In some instances, the non-antibody polypeptide region is not based on or derived from a B-cell epitope.The antibody fusion protein may comprise any of the antibody fusion proteins disclosed herein. The antibody region may comprise any of the antibody regions disclosed herein. In some instances, the antibody region is not based on or derived from an APC-specific antibody. In some instances, the antibody region is not based on or derived from a MHC-specific antibody. In some instances, the antibody region is not based on or derived from a MHC class I-specific antibody. In some instances, the antibody region is not based on or derived from a MHC class II-specific antibody. The non-antibody polypeptide region may comprise any of the non-antibody polypeptide regions disclosed herein. The non-antibody polypeptide region may comprise a protein-based region. The protein-based region may comprise any of the protein-based regions disclosed herein. The non-antibody polypeptide region may comprise one or more adapter peptides. The one or more adapter peptides may comprise any of the adapter peptides disclosed herein. In some instances, the non-antibody region is not inserted into a complementarity determining region (CDR) of the antibody or antibody fragment. The CDR may be CDR1. The CDR may be CDR2. The CDR may be CDR3. The antibody fusion protein may further comprise one or more additional antibodies or antibody fragments. The one or more additional antibodies or antibody fragments may comprise any of the antibodies or antibody fragments disclosed herein.

The disease or condition may be a cancer. The cancer may be a lymphoma. The cancer may be leukemia. The cancer may be a sarcoma. The cancer may be a carcinoma. The antibody fusion protein may comprise a non-antibody polypeptide region may be based on or derived from Int. The antibody fusion protein may comprise an antibody region based on or derived from UCHT1. The antibody fusion protein may comprise (a) an antibody region based on or derived from UCHT1; and (b) a non-antibody polypeptide region may be based on or derived from Int, wherein the non-antibody polypeptide region is inserted into the antibody region by replacment of less than about 20 amino acid residues from the antibody or antibody fragment. The Int may comprise any of the Int peptides disclosed herein. The UCHT1 may comprise any of the UCHT1 antibodies disclosed herein.

The lymphoma may be a non-Hodgkins lymphoma (NHL). The antibody fusion protein may comprise a non-antibody polypeptide region based on or derived from CXCR4-BP. The antibody fusion protein may comprise an antibody region based on or derived from anti-CD20. The fusion antibody may comprise (a) an antibody region based on or derived from anti-CD20; and (b) a non-antibody polypeptide region based on or derived from CXCR4-BP. The CXCR4-BP may comprise any of the CXCR4-BP peptides disclosed herein. The anti-CD20 may comprise any of the anti-CD20 antibodies disclosed herein.

The lymphoma may comprise a CD19 positive lymphoma. A CD19 positive lymphoma may comprise one or more CD19 positive lymphoma cells. The antibody fusion protein may comprise a non-antibody polypeptide region based on or derived from GCN4. The antibody fusion protein may comprise an antibody region based on or derived from anti-CD19. The fusion antibody may comprise (a) an antibody region based on or derived from anti-CD19; and (b) a non-antibody polypeptide region based on or derived from GCN4. The GCN4 may comprise any of the GCN4 peptides disclosed herein. The anti-CD19 may comprise any of the anti-CD19 antibodies disclosed herein.

The cancer may be a colorectal cancer. The antibody fusion protein may comprise a non-antibody polypeptide region may be based on or derived from TCP1. The antibody fusion protein may comprise an antibody region based on or derived from UCHT1. The antibody fusion protein may comprise (a) an antibody region based on or derived from UCHT1; and (b) a non-antibody polypeptide region may be based on or derived from TCP1, wherein the non-antibody polypeptide region is inserted into the antibody region by replacment of less than about 20 amino acid residues from the antibody or antibody fragment. The TCP1 may comprise any of the TCP1 peptides disclosed herein. The UCHT1 may comprise any of the UCHT1 antibodies disclosed herein.

The cancer may be a colorectal cancer. The antibody fusion protein may comprise a non-antibody polypeptide region may be based on or derived from NGR. The antibody fusion protein may comprise an antibody region based on or derived from UCHT1. The antibody fusion protein may comprise (a) an antibody region based on or derived from UCHT1; and (b) a non-antibody polypeptide region may be based on or derived from NGR, wherein the non-antibody polypeptide region is inserted into the antibody region by replacment of less than about 20 amino acid residues from the antibody or antibody fragment. The NGR may comprise any of the NGR peptides disclosed herein. The UCHT1 may comprise any of the UCHT1 antibodies disclosed herein.

The cancer may be a Her2 positive cancer. The Her2 positive cancer may be breast cancer. The antibody fusion protein may comprise a non-antibody polypeptide region based on or derived from CXCR4-BP. The antibody fusion protein may comprise an antibody region based on or derived from trastuzumab. The fusion antibody may comprise (a) an antibody region based on or derived from trastuzumab; and (b) a non-antibody polypeptide region based on or derived from CXCR4-BP. The CXCR4-BP may comprise any of the CXCR4-BP peptides disclosed herein. The trastuzumab may comprise any of the trastuzumab antibodies disclosed herein.

Further disclosed herein are plasmids comprising a nucleic acid sequence encoding the antibody fusion proteins disclosed herein. The nucleic acid sequence encoding the antibody fusion protein may be at least about 50%, 60%, 70%, 75%, 80%, 85%, 90%, 95%, or 97% or more homologous to a nucleic acid sequence selected from a group consisting of SEQ ID NOS: 11-23, 27 and 28. The nucleic acid sequence encoding the antibody fusion protein may be at least about 60% or more homologous to a nucleic acid sequence selected from a group consisting of SEQ ID NOS: 11-23, 27 and 28. The nucleic acid sequence encoding the antibody fusion protein may be at least about 65% or more homologous to a nucleic acid sequence selected from a group consisting of SEQ ID NOS: 11-23, 27 and 28. The nucleic acid sequence encoding the antibody fusion protein may be at least about 70% or more homologous to a nucleic acid sequence selected from a group consisting of SEQ ID NOS: 11-23, 27 and 28. The nucleic acid sequence encoding the antibody fusion protein may be at least about 75% or more homologous to a nucleic acid sequence selected from a group consisting of SEQ ID NOS: 11-23, 27 and 28. The nucleic acid sequence encoding the antibody fusion protein may be at least about 80% or more homologous to a nucleic acid sequence selected from a group consisting of SEQ ID NOS: 11-23, 27 and 28. The nucleic acid sequence encoding the antibody fusion protein may be at least about 90% or more homologous to a nucleic acid sequence selected from a group consisting of SEQ ID NOS: 11-23, 27 and 28. The nucleic acid sequence encoding the antibody fusion protein may be at least about 95% or more homologous to a nucleic acid sequence selected from a group consisting of SEQ ID NOS: 11-23, 27 and 28. The nucleic acid sequence may be SEQ ID NO: 11. The nucleic acid sequence may be SEQ ID NO: 12. The nucleic acid sequence may be SEQ ID NO: 13. The nucleic acid sequence may be SEQ ID NO: 14. The nucleic acid sequence may be SEQ ID NO: 15. The nucleic acid sequence may be SEQ ID NO: 16. The nucleic acid sequence may be SEQ ID NO: 17. The nucleic acid sequence may be SEQ ID NO: 18. The nucleic acid sequence may be SEQ ID NO: 19. The nucleic acid sequence may be SEQ ID NO: 20. The nucleic acid sequence may be SEQ ID NO: 21. The nucleic acid sequence may be SEQ ID NO: 22. The nucleic acid sequence may be SEQ ID NO: 23. The nucleic acid sequence may be SEQ ID NO: 24. The nucleic acid sequence may be SEQ ID NO: 25. The nucleic acid sequence may be SEQ ID NO: 26. The nucleic acid sequence may be SEQ ID NO: 27. The nucleic acid sequence may be SEQ ID NO: 28. The nucleic acid sequence may be SEQ ID NO: 29. The nucleic acid sequence may be SEQ ID NO: 30. The nucleic acid sequence may be SEQ ID NO: 31. The nucleic acid sequence may be SEQ ID NO: 32.

The antibody fusion protein may comprise an amino acid sequence that is at least about 50%, 60%, 70%, 75%, 80%, 85%, 90%, 95%, or 97% or more homologous to an amino acid sequence selected from a group consisting of SEQ ID NOS: 45-57, 61-66. The antibody fusion protein may comprise an amino acid sequence that is at least about 60% or more homologous to an amino acid sequence selected from a group consisting of SEQ ID NOS: 45-57, 61-66. The antibody fusion protein may comprise an amino acid sequence that is at least about 65% or more homologous to an amino acid sequence selected from a group consisting of SEQ ID NOS: 45-57, 61-66. The antibody fusion protein may comprise an amino acid sequence that is at least about 70% or more homologous to an amino acid sequence selected from a group consisting of SEQ ID NOS: 45-57, 61-66. The antibody fusion protein may comprise an amino acid sequence that is at least about 75% or more homologous to an amino acid sequence selected from a group consisting of SEQ ID NOS: 45-57, 61-66. The antibody fusion protein may comprise an amino acid sequence that is at least about 80% or more homologous to an amino acid sequence selected from a group consisting of SEQ ID NOS: 45-57, 61-66. The antibody fusion protein may comprise an amino acid sequence that is at least about 85% or more homologous to an amino acid sequence selected from a group consisting of SEQ ID NOS: 45-57, 61-66. The antibody fusion protein may comprise an amino acid sequence that is at least about 90% or more homologous to an amino acid sequence selected from a group consisting of SEQ ID NOS: 45-57, 61-66. The antibody fusion protein may comprise an amino acid sequence that is at least about 95% or more homologous to an amino acid sequence selected from a group consisting of SEQ ID NOS: 45-57, 61-66. The amino acid sequence may be SEQ ID NO: 45. The amino acid sequence may be SEQ ID NO: 46. The amino acid sequence may be SEQ ID NO: 47. The amino acid sequence may be SEQ ID NO: 48. The amino acid sequence may be SEQ ID NO: 49. The amino acid sequence may be SEQ ID NO: 50. The amino acid sequence may be SEQ ID NO: 51. The amino acid sequence may be SEQ ID NO: 52. The amino acid sequence may be SEQ ID NO: 53. The amino acid sequence may be SEQ ID NO: 54. The amino acid sequence may be SEQ ID NO: 55. The amino acid sequence may be SEQ ID NO: 56. The amino acid sequence may be SEQ ID NO: 57. The amino acid sequence may be SEQ ID NO: 58. The amino acid sequence may be SEQ ID NO: 59. The amino acid sequence may be SEQ ID NO: 60. The amino acid sequence may be SEQ ID NO: 61. The amino acid sequence may be SEQ ID NO: 62. The amino acid sequence may be SEQ ID NO: 63. The amino acid sequence may be SEQ ID NO: 64. The amino acid sequence may be SEQ ID NO: 65. The amino acid sequence may be SEQ ID NO: 66. The amino acid sequence may be SEQ ID NO: 67. The amino acid sequence may be SEQ ID NO: 68. The amino acid sequence may be SEQ ID NO: 69. The amino acid sequence may be SEQ ID NO: 70.

The antibody fusion protein may comprise an amino acid sequence that comprises 50, 60, 70, 80, 90, 100, 110, 120, 130, 140, 150, 160, 170, 180, 190, 200 or more consecutive amino acids from an amino acid sequence selected from a group consisting of SEQ ID NOS: 45-57, 61-66. The antibody fusion protein comprises an amino acid sequence that comprises 200, 225, 250, 275, 300, 325, 300, 325, 350, 375, 400 or more consecutive amino acids from an amino acid sequence selected from a group consisting of SEQ ID NOs: 45-57, 61-66. The antibody fusion protein may comprise an amino acid sequence that comprises 50 or more amino acids from an amino acid sequence selected from a group consisting of SEQ ID NOs: 45-57, 61-66. The antibody fusion protein may comprise an amino acid sequence that comprises 100 or more amino acids from an amino acid sequence selected from a group consisting of SEQ ID NOs: 45-57, 61-66. The antibody fusion protein may comprise an amino acid sequence that comprises 150 or more amino acids from an amino acid sequence selected from a group consisting of SEQ ID NOs: 45-57, 61-66. The antibody fusion protein may comprise an amino acid sequence that comprises 200 or more amino acids from an amino acid sequence selected from a group consisting of SEQ ID NOs: 45-57, 61-66. The amino acid sequence may be SEQ ID NO: 45. The amino acid sequence may be SEQ ID NO: 46. The amino acid sequence may be SEQ ID NO: 47. The amino acid sequence may be SEQ ID NO: 48. The amino acid sequence may be SEQ ID NO: 49. The amino acid sequence may be SEQ ID NO: 50. The amino acid sequence may be SEQ ID NO: 51. The amino acid sequence may be SEQ ID NO: 52. The amino acid sequence may be SEQ ID NO: 53. The amino acid sequence may be SEQ ID NO: 54. The amino acid sequence may be SEQ ID NO: 55. The amino acid sequence may be SEQ ID NO: 56. The amino acid sequence may be SEQ ID NO: 57. The amino acid sequence may be SEQ ID NO: 58. The amino acid sequence may be SEQ ID NO: 59. The amino acid sequence may be SEQ ID NO: 60. The amino acid sequence may be SEQ ID NO: 61. The amino acid sequence may be SEQ ID NO: 62. The amino acid sequence may be SEQ ID NO: 63. The amino acid sequence may be SEQ ID NO: 64. The amino acid sequence may be SEQ ID NO: 65. The amino acid sequence may be SEQ ID NO: 66. The amino acid sequence may be SEQ ID NO: 67. The amino acid sequence may be SEQ ID NO: 68. The amino acid sequence may be SEQ ID NO: 69. The amino acid sequence may be SEQ ID NO: 70.

Further disclosed herein are one or more cells comprising any of the plasmids disclosed herein. The one or more cells may comprise a plasmid comprising a nucleic acid sequenc encoding a bispecific fusion antibody disclosed herein. The cell may be a eukaryotic cell. The cell may be a prokaryotic cell. The cell may be a mammalian cell. The mammalian cell may be a human cell. The mammalian cell may be HEK 293 T cells. The cell may be a bacterial cell. The bacterial cell may be an E. coli cell. The cell may be an insect cell. The cell may be a yeast cell. The yeast cell may be a sacchromyces cell. The cell may be an immortalized cell.

### Bispecific Antibodies

Further disclosed herein are bispecific antibodies and uses thereof. A bispecific antibody may comprise (a) first antibody or antibody fragment; and (b) a second antibody or antibody fragment, wherein the second antibody or antibody fragment may be inserted into a constant domain of the first antibody or antibody fragment. The second antibody or antibody fragment may be inserted into the constant domain of the first antibody or antibody fragment by replacement of less than about 20 amino acid residues from the constant domain of the first antibody or antibody fragment with the second antibody or antibody fragment. Alternatively, insertion of the second antibody or antibody fragment in to the first antibody or antibody fragment does not comprise replacement of or more amino acids from the constant domain of the first antibody. The second antibody or antibody fragment may be inserted into the constant domain of a heavy chain of the first antibody or antibody fragment. The constant domain of the heavy chain may be CH1. The constant domain of the heavy chain may be CH2. The constant domain of the heavy chain may be CH3.The second antibody or antibody fragment may be inserted into the constant domain of a light chain of the first antibody or antibody fragment.

Further disclosed herein are bispecific antibodies and uses thereof. A bispecific antibody may comprise (a) first antibody or antibody fragment; and (b) a second antibody or antibody fragment, wherein the second antibody or antibody fragment may be inserted into the first antibody or antibody fragment by replacement of less than about 20 amino acid residues from the first antibody or antibody fragment with the second antibody or antibody fragment. In some instances, the second antibody or antibody fragment is not inserted into a complementarity determining region (CDR) of the first antibody or antibody fragment. The CDR may be CDR1. The CDR may be CDR2. The CDR may be CDR3.

The second antibody or antibody fragment may be inserted adjacent to a beta strand secondary structure in constant domain of the first antibody. The second antibody or antibody fragment may be inserted adjacent to a beta strand secondary structure in the first antibody. The second antibody or antibody fragment may be inserted between two beta strand secondary structures in constant domain of the first antibody. The second antibody or antibody fragment may be inserted between two beta strand secondary structures in the first antibody. The second antibody or antibody fragment may be inserted into a loop region in constant domain of the first antibody. The second antibody or antibody fragment may be inserted into a loop region in the first antibody.

The second antibody or antibody fragment may be inserted into a constant domain of the first antibody or antibody fragment. The second antibody or antibody fragment may be inserted into a loop region of the first antibody or antibody fragment. The second antibody or antibody fragment may be inserted into a loop region of a constant domaino of the first antibody or antibody fragment. The second antibody or antibody fragment may be inserted near a beta strand of the antibody region. The second antibody or antibody fragment may be inserted within 20 amino acids of a beta strand of the antibody region. The second antibody or antibody fragment may be inserted within 15 amino acids of a beta strand of the antibody region. The second antibody or antibody fragment may be inserted within 10 amino acids of a beta strand of the antibody region. The second antibody or antibody fragment may be inserted within 5 amino acids of a beta strand of the antibody region. The less than about 20 amino acid residues to be replaced may be located between two beta strands. The second antibody or antibody fragment may be inserted into the antibody region by replacement of less than about 20 amino acid residues from a constant domain of the first antibody or antibody fragment with the second antibody or antibody fragment. The less than about 20 amino acid residues to be replaced may be located near a beta strand. The less than about 20 amino acid residues to be replaced may be within 20 amino acids of a beta strand. The less than about 20 amino acid residues to be replaced may be within 15 amino acids of a beta strand. The less than about 20 amino acid residues to be replaced may be within 10 amino acids of a beta strand. The less than about 20 amino acid residues to be replaced may be within 5 amino acids of a beta strand. The less than about 20 amino acid residues to be replaced may be located between two beta strands. The constant domain may be from a heavy chain of the first antibody or antibody fragment. The constant domain may be from a light chain of the first antibody or antibody fragment.

The first antibody or antibody fragment may comprise a consensus insertion sequence. The consensus insertion sequence may comprise an amino acid sequence that is at least about 50% homologous to an amino acid sequence selected from a group consisting of SEQ ID NO: 89-120. The consensus insertion sequence may comprise an amino acid sequence that is at least about 60% homologous to an amino acid sequence selected from a group consisting of SEQ ID NO: 89-120. The consensus insertion sequence may comprise an amino acid sequence that is at least about 70% homologous to an amino acid sequence selected from a group consisting of SEQ ID NO: 89-120. The consensus insertion sequence may comprise an amino acid sequence that is at least about 80% homologous to an amino acid sequence selected from a group consisting of SEQ ID NO: 89-120. The consensus insertion sequence may comprise an amino acid sequence that is at least about 90% homologous to an amino acid sequence selected from a group consisting of SEQ ID NO: 89-120. The consensus insertion sequence may comprise an amino acid sequence that is at least about 95% homologous to an amino acid sequence selected from a group consisting of SEQ ID NO: 89-120. The amino acid sequence may be SEQ ID NO: 89. The amino acid sequence may be SEQ ID NO: 90. The amino acid sequence may be SEQ ID NO: 91. The amino acid sequence may be SEQ ID NO: 92. The amino acid sequence may be SEQ ID NO: 93. The amino acid sequence may be SEQ ID NO: 94. The amino acid sequence may be SEQ ID NO: 95. The amino acid sequence may be SEQ ID NO: 96. The amino acid sequence may be SEQ ID NO: 97. The amino acid sequence may be SEQ ID NO: 98. The amino acid sequence may be SEQ ID NO: 99. The amino acid sequence may be SEQ ID NO: 100. The amino acid sequence may be SEQ ID NO: 101. The amino acid sequence may be SEQ ID NO: 102. The amino acid sequence may be SEQ ID NO: 103. The amino acid sequence may be SEQ ID NO: 104. The amino acid sequence may be SEQ ID NO: 105. The amino acid sequence may be SEQ ID NO: 106. The amino acid sequence may be SEQ ID NO: 107. The amino acid sequence may be SEQ ID NO: 108. The amino acid sequence may be SEQ ID NO: 109. The amino acid sequence may be SEQ ID NO: 110. The amino acid sequence may be SEQ ID NO: 111. The amino acid sequence may be SEQ ID NO: 112. The amino acid sequence may be SEQ ID NO: 113. The amino acid sequence may be SEQ ID NO: 114. The amino acid sequence may be SEQ ID NO: 115. The amino acid sequence may be SEQ ID NO: 116. The amino acid sequence may be SEQ ID NO: 117. The amino acid sequence may be SEQ ID NO: 118. The amino acid sequence may be SEQ ID NO: 119. The amino acid sequence may be SEQ ID NO: 120. The consensus insertion sequence may be based on or derived from a constant domain of the first antibody or antibody fragment. The consensus insertion sequence may be based on or derived from a loop region of the first antibody or antibody fragment. The consensus insertion sequence may be based on or derived from a loop region of a constant domain of the first antibody or antibody fragment. The consensus insertion sequence may be based on or derived from a sequence located between two beta strands of the first antibody or antibody fragment. The two beta strands may be in a constant domain of the first antibody or antibody fragment. The constant domain may be in a heavy chain. The constant domain may be CH1. The constant domain may be CH2. The constant domain may be CH3. The constant domain may be in a light chain. The loop region may be in a heavy chain. The loop region may be in the light chain. The two beta strands may be in a heavy chain. The two beta strands may be in a light chain. The second antibody or antibody fragment may be inserted into the consensus insertion sequence of the antibody region. The second antibody or antibody fragment may be inserted into the antibody region by replacement of less than about 20 amino acids from the consensus insertion sequence of the antibody region. The second antibody or antibody fragment may be inserted into the consensus insertion sequence by replacement of one or more amino acids from the consensus insertion sequence. The second antibody or antibody fragment may be inserted into the consensus insertion sequence by replacement of two or more amino acids from the consensus insertion sequence. The second antibody or antibody fragment may be inserted into the consensus insertion sequence by replacement of three or more amino acids from the consensus insertion sequence. The second antibody or antibody fragment may be inserted into the consensus insertion sequence by replacement of four or more amino acids from the consensus insertion sequence. The second antibody or antibody fragment may be inserted into the consensus insertion sequence by replacement of five or more amino acids from the consensus insertion sequence.

The second antibody or antibody fragment may be inserted into the antibody region by replacement of less than about 20 amino acid residues from a constant domain of the first antibody or antibody fragment with the second antibody or antibody fragment. The constant domain may be from a heavy chain of the first antibody. The constant domain may be from a light chain of the first antibody.

The second antibody or antibody fragment may be inserted into the antibody region by replacement of less than about 20 amino acid residues from a heavy chain of the first antibody or antibody fragment with the second antibody or antibody fragment. The second antibody or antibody fragment may be inserted into the antibody region by replacement of less than about 20 amino acid residues from a constant domain of the heavy chain of the first antibody or antibody fragment with the second antibody or antibody fragment. The constant domain of the heavy chain may be CH1. The constant domain of the heavy chain may be CH2. The constant domain of the heavy chain may be CH3.

The second antibody or antibody fragment may be inserted into the antibody region by replacement of less than about 20 amino acid residues from a light chain of the first antibody or antibody fragment with the second antibody or antibody fragment. The second antibody or antibody fragment may be inserted into the antibody region by replacement of less than about 20 amino acid residues from constant domain of the light chain of the first antibody or antibody fragment with the second antibody or antibody fragment.

The replacement of less than about 20 amino acid residues may comprise replacement of at least 1 amino acid residue from the first antibody or antibody fragment with the second antibody or antibody fragment.The replacement of less than about 20 amino acid residues may comprise replacement of at least 2 amino acid residues from the first antibody or antibody fragment with the second antibody or antibody fragment. The replacement of less than about 20 amino acid residues may comprise replacement of at least 3 amino acid residues from the first antibody or antibody fragment with the second antibody or antibody fragment.

The replacement of less than about 20 amino acid residues may comprise replacement of less than 15 amino acid residues from the first antibody or antibody fragment with the second antibody or antibody fragment. The replacement of less than about 20 amino acid residues may comprise replacement of less than 10 amino acid residues from the first antibody or antibody fragment with the second antibody or antibody fragment. The replacement of less than about 20 amino acid residues may comprise replacement of less than 5 amino acid residues from the first antibody or antibody fragment with the second antibody or antibody fragment.

The replacement of less than about 20 amino acid residues may comprise replacement of 5 or fewer amino acid residues from the first antibody or antibody fragment with the second antibody or antibody fragment. The replacement of less than about 20 amino acid residues may comprise replacement of 4 or fewer amino acid residues from the first antibody or antibody fragment with the second antibody or antibody fragment. The replacement of less than about 20 amino acid residues may comprise replacement of 3 or fewer amino acid residues from the first antibody or antibody fragment with the second antibody or antibody fragment. The replacement of less than about 20 amino acid residues may comprise replacement of 1-15 amino acid residues from the first antibody or antibody fragment with the second antibody or antibody fragment. The replacement of less than about 20 amino acid residues may comprise replacement of 1-10 amino acid residues from the first antibody or antibody fragment with the second antibody or antibody fragment. The replacement of less than about 20 amino acid residues may comprise replacement of 1-5 amino acid residues from the first antibody or antibody fragment with the second antibody or antibody fragment.

The replacement of the amino acid residues may comprise replacement of one or more amino acids selected from a group consisting of serine (S), glycine (G), lysine (K), proline (P), threonine (T), glutamine (Q), glutamic acid (E), alanine (A), asparagines (N), and histidine (H). The replacement of less than about 20 amino acids may comprise replacement of 5 or fewer amino acid residues from the CH1 domain of the first antibody or antibody fragment. The replacement of less than about 20 amino acids may comprise replacement of 4 or fewer amino acid residues from the CH1 domain of the first antibody or antibody fragment.

The replacement of less than about 20 amino acids may comprise replacement of 3 or fewer amino acid residues from the CH1 domain of the first antibody or antibody fragment. The replacement of less than about 20 amino acids may comprise replacement of 2 or fewer amino acid residues from the CH1 domain of the first antibody or antibody fragment. The replacement of less than about 20 amino acids may comprise replacement of 1 amino acid residue from the CH1 domain of the first antibody or antibody fragment. The replacement of less than about 20 amino acids may comprise replacement of one or more amino acids from the CH1 domain selected from a group consisting of serine (S), glycine (G), proline (P), threonine (T), and glutamine (Q). The replacement of less than about 20 amino acids may comprise replacement of serine 180 (S180) from the CH1 domain. The replacement of less than about 20 amino acids may comprise replacement of glycine 181 (G181) from the CH1 domain. The replacement of less than about 20 amino acids may comprise replacement of serine 180 (S180) and glycine 181 (G181) from the CH1 domain. The replacement of less than about 20 amino acids may comprise replacement of proline 156 (P156) from the CH1 domain. The replacement of less than about 20 amino acids may comprise replacement of serine and glycine from the CH1 domain. The serine and glycine may be adjacent to each other. The replacement of less than about 20 amino acids may comprise replacement of threonine and serine from the CH1 domain. The threonine and serine may be adjacent to each other.

The replacement of less than about 20 amino acids may comprise replacement of 5 or fewer amino acid residues from the CH2 domain of the first antibody or antibody fragment. The replacement of less than about 20 amino acids may comprise replacement of 4 or fewer amino acid residues from the CH2 domain of the first antibody or antibody fragment. The replacement of less than about 20 amino acids may comprise replacement of 3 or fewer amino acid residues from the CH2 domain of the first antibody or antibody fragment. The replacement of less than about 20 amino acids may comprise replacement of 2 or fewer amino acid residues from the CH2 domain of the first antibody or antibody fragment. The replacement of less than about 20 amino acids may comprise replacement of 1 amino acid residue from the CH2 domain of the first antibody or antibody fragment. The replacement of less than about 20 amino acids may comprise replacement of one or more amino acids from the CH2 domain selected from a group consisting of glutamic acid (E), alanine (A) and proline (P). The replacement of less than about 20 amino acids may comprise replacement of glutamic acid 274 (E274) from the CH2 domain. The replacement of less than about 20 amino acids may comprise replacement of alanine 302 (A302) from the CH2 domain. The replacement of less than about 20 amino acids may comprise replacement of proline 334 (P334) from the CH2 domain.

The replacement of less than about 20 amino acids may comprise replacement of 5 or fewer amino acid residues from the CH3 domain of the first antibody or antibody fragment. The replacement of less than about 20 amino acids may comprise replacement of 4 or fewer amino acid residues from the CH3 domain of the first antibody or antibody fragment. The replacement of less than about 20 amino acids may comprise replacement of 3 or fewer amino acid residues from the CH3 domain of the first antibody or antibody fragment. The replacement of less than about 20 amino acids may comprise replacement of 2 or fewer amino acid residues from the CH3 domain of the first antibody or antibody fragment. The replacement of less than about 20 amino acids may comprise replacement of 1 amino acid residue from the CH3 domain of the first antibody or antibody fragment. The replacement of less than about 20 amino acids may comprise replacement of one or more amino acids from the CH3 domain selected from a group consisting of threonine (T), lysine (K), asparagine (N), and glycine (G). The replacement of less than about 20 amino acids may comprise replacement of threonine 361 (T361) from the CH3 domain. The replacement of less than about 20 amino acids may comprise replacement of lysine 362 (K362) from the CH3 domain. The replacement of less than about 20 amino acids may comprise replacement of asparagine 363 (N363) from the CH3 domain. The replacement of less than about 20 amino acids may comprise replacement of threonine 361 (T361), lysine 362 (K362), and asparagine 363 (N363) from the CH3 domain. The replacement of less than about 20 amino acids may comprise replacement of asparagine 389 (N389) from the CH3 domain. The replacement of less than about 20 amino acids may comprise replacement of glycine 390 (G390) from the CH3 domain. The replacement of less than about 20 amino acids may comprise replacement of asparagine 389 (N389) and glycine 390 (G390) from the CH3 domain. The replacement of less than about 20 amino acids may comprise replacement of glycine 425 (G425) from the CH3 domain. The replacement of less than about 20 amino acids may comprise replacement of asparagine 426 (N426) from the CH3 domain. The replacement of less than about 20 amino acids may comprise replacement of glycine 425 (G425) and asparagine 363 (N363) from the CH3 domain. The replacement of less than about 20 amino acids may comprise replacement of threonine and asparagine from the CH3 domain. The threonine and asparagine may be adjacent to each other. The replacement of less than about 20 amino acids may comprise replacement of threonine, lysine, and asparagine from the CH3 domain. The threonine, lysine, and asparagine may be adjacent to each other.

The replacement of less than about 20 amino acids may comprise replacement of 5 or fewer amino acid residues from the constant domain of the light chain of the first antibody or antibody fragment.The replacement of less than about 20 amino acids may comprise replacement of 4 or fewer amino acid residues from the constant domain of the light chain of the first antibody or antibody fragment. The replacement of less than about 20 amino acids may comprise replacement of 3 or fewer amino acid residues from the constant domain of the light chain of the first antibody or antibody fragment. The replacement of less than about 20 amino acids may comprise replacement of 2 or fewer amino acid residues from the constant domain of the light chain of the first antibody or antibody fragment. The replacement of less than about 20 amino acids may comprise replacement of 1 amino acid residue from the constant domain of the light chain of the first antibody or antibody fragment. The replacement of less than about 20 amino acids may comprise replacement of one or more amino acids from the constant domain of the light chain selected from a group consisting of serine (S), glycine (G), proline (P), lysine (K), asparagine (N) and histidine (H) The replacement of less than about 20 amino acids may comprise replacement of serine 202 (S202) from the constant domain of the light chain. The replacement of less than about 20 amino acids may comprise replacement of glycine 128 (G128) from the constant domain of the light chain. The replacement of less than about 20 amino acids may comprise replacement of lysine 169 (K169) from the constant domain of the light chain. The replacement of less than about 20 amino acids may comprise replacement of proline 141 (P141) from the constant domain of the light chain. The replacement of less than about 20 amino acids may comprise replacement of asparagine (N152) from the constant domain of the light chain. The replacement of less than about 20 amino acids may comprise replacement of lysine 138 (K138) from the constant domain of the light chain. The replacement of less than about 20 amino acids may comprise replacement of histidine 139 (H139) from the constant domain of the light chain. The replacement of less than about 20 amino acids may comprise replacement of lysine 138 (K138) and histidine (H139) from the constant domain of the light chain. The replacement of less than about 20 amino acids may comprise replacement of lysine and histidine from the constant domain of the light chain. The lysine and histidine may be adjacent to each other.

The first antibody or antibody fragment may be based on or derived from a group consisting of UCHT1, anti-CD19, anti-CD20 and Her2. The first antibody or antibody fragment may comprise a fragment antigen binding (Fab), fragment antigen-binding including hinge region (F(ab′)₂), fragment antigen-binding including one hinge region (Fab′), fragment crystallizable (Fc), variable domain (e.g., V_{H} or V_{L}), constant domain (e.g., C_{H1}, C_{H2}, C_{H3}, or C_{L}), single-chain varaible fragment (scFV), di-ScFv, single domain antibody (sdAb), minibody, diabody, tribody, tetrabody, trifunctional antibody. The first antibody or antibody fragment may comprise one or more heavy chains, light chains, or both. The first antibody or antibody fragment may comprise one or more constant domains.

The second antibody or antibody fragment may be based on or derived from a group consisting of UCHT1, anti-CD19, anti-CD20, and Her2. The second antibody or antibody fragment may comprise a fragment antigen binding (Fab), fragment antigen-binding including hinge region (F(ab′)₂), fragment antigen-binding including one hinge region (Fab′), fragment crystallizable (Fc), variable domain (e.g., V_{H} or V_{L}), constant domain (e.g., C_{H1}, C_{H2}, C_{H3}, or C_{L}), single-chain varaible fragment (scFV), di-ScFv, single domain antibody (sdAb), minibody, diabody, tribody, tetrabody, trifunctional antibody. The second antibody or antibody fragment may comprise one or more heavy chains, light chains, or both. The second antibody or antibody fragment may comprise one or more constant domains.

The first antibody or antibody fragment may be based on or derived from a UCHT1 antibody or antibody fragment. The second antibody or antibody fragment may be based on or derived from a UCHT1 antibody or antibody fragment.The UCHT1 may be UCHT1scFv. The UCHT1 may be UCHT1 light chain. The UCHT1 may be UCHT1 heavy chain. The UCHT1 may be UCHT1 Fab fragment. The UCHT1 may comprise an amino acid sequence that is at least 50% homologous to a sequence selected from SEQ ID NOS: 34, 35, 41, and 88. The UCHT1 may comprise an amino acid sequence that is at least 60% homologous to a sequence selected from SEQ ID NOS: 34, 35, 41, and 88. The UCHT1 may comprise an amino acid sequence that is at least 70% homologous to a sequence selected from SEQ ID NOS: 34, 35, 41, and 88. The UCHT1 may comprise an amino acid sequence that is at least 80% homologous to a sequence selected from SEQ ID NOS: 34, 35, 41, and 88. The UCHT1 may comprise an amino acid sequence that is at least 90% homologous to a sequence selected from SEQ ID NOS: 34, 35, 41, and 88. The UCHT1 may comprise an amino acid sequence that comprises 10 or more consecutive amino acids from a sequence selected from SEQ ID NOS: 34, 35, 41, and 88. The UCHT1 may comprise an amino acid sequence that comprises 50 or more consecutive amino acids from a sequence selected from SEQ ID NOS: 34, 35, 41, and 88. The UCHT1 may comprise an amino acid sequence that comprises 100 or more consecutive amino acids from a sequence selected from SEQ ID NOS: 34, 35, 41, and 88. The UCHT1 may comprise an amino acid sequence that comprises 200 or more consecutive amino acids from a sequence selected from SEQ ID NOS: 34, 35, 41, and 88. The amino acid may be SEQ ID NO: 34. The amino acid may be SEQ ID NO: 35. The amino acid may be SEQ ID NO: 41. The amino acid may be SEQ ID NO: 88.

The first antibody or antibody fragment may be based on or derived from an anti-CD19 antibody or antibody fragment. The second antibody or antibody fragment may be based on or derived from an anti-CD19 antibody or antibody fragment. The anti-CD19 may be anti-CD19scFv. The anti-CD19 may be anti-CD19 light chain. The anti-CD19 may be anti-CD19 heavy chain. The anti-CD19 may be anti-CD19 Fab fragment. The anti-CD19 may comprise an amino acid sequence that is at least 50% homologous to a sequence selected from SEQ ID NOS: 38, 39, 42, and 87. The anti-CD19 may comprise an amino acid sequence that is at least 60% homologous to a sequence selected from SEQ ID NOS: 38, 39, 42, and 87. The anti-CD19 may comprise an amino acid sequence that is at least 70% homologous to a sequence selected from SEQ ID NOS: 38, 39, 42, and 87. The anti-CD19 may comprise an amino acid sequence that is at least 80% homologous to a sequence selected from SEQ ID NOS: 38, 39, 42, and 87. The anti-CD19 may comprise an amino acid sequence that is at least 90% homologous a sequence selected from SEQ ID NOS: 38, 39, 42, and 87. The anti-CD19 may comprise an amino acid sequence that comprises 10 or more consecutive amino acids from a sequence selected from SEQ ID NOS: 38, 39, 42, and 87. The anti-CD19 may comprise an amino acid sequence that comprises 50 or more consecutive amino acids from a sequence selected from SEQ ID NOS: 38, 39, 42, and 87. The anti-CD19 may comprise an amino acid sequence that comprises 75 or more consecutive amino acids from a sequence selected from SEQ ID NOS: 38, 39, 42, and 87. The anti-CD19 may comprise an amino acid sequence that comprises 100 or more consecutive amino acids from a sequence selected from SEQ ID NOS: 38, 39, 42, and 87.

The first antibody or antibody fragment may be based on or derived from an anti-CD20 antibody or antibody fragment. The second antibody or antibody fragment may be based on or derived from an anti-CD20 antibody or antibody fragment. The anti-CD20 may be anti-CD20 light chain. The anti-CD20 light chain may comprise an amino acid sequence that is at least 50% homologous to SEQ ID NO: 43. The anti-CD20 light chain may comprise an amino acid sequence that is at least 60% homologous to SEQ ID NO: 43. The anti-CD20 light chain may comprise an amino acid sequence that is at least 70% homologous to SEQ ID NO: 43. The anti-CD20 light chain may comprise an amino acid sequence that is at least 80% homologous to SEQ ID NO: 43. The anti-CD20 light chain may comprise an amino acid sequence that is at least 90% homologous to SEQ ID NO: 43. The anti-CD20 light chain may comprise an amino acid sequence that comprises 50 or more consecutive amino acids from SEQ ID NO: 43. The anti-CD20 light chain may comprise an amino acid sequence that comprises 100 or more consecutive amino acids from SEQ ID NO: 43. The anti-CD20 light chain may comprise an amino acid sequence that comprises 150 or more consecutive amino acids from SEQ ID NO: 43. The anti-CD20 light chain may comprise an amino acid sequence that comprises 200 or more consecutive amino acids from SEQ ID NO: 43. The anti-CD20 may be anti-CD20 heavy chain. The anti-CD20 heavy chain may comprise an amino acid sequence that is at least 50% homologous to an amino acid sequence selected from a group consisting of SEQ ID NOS: 36-37. The anti-CD20 heavy chain may comprise an amino acid sequence that is at least 60% homologous to an amino acid sequence selected from a group consisting of SEQ ID NOS: 36-37. The anti-CD20 heavy chain may comprise an amino acid sequence that is at least 70% homologous to an amino acid sequence selected from a group consisting of SEQ ID NOS: 36-37. The anti-CD20 heavy chain may comprise an amino acid sequence that is at least 80% homologous to an amino acid sequence selected from a group consisting of SEQ ID NOS: 36-37. The anti-CD20 heavy chain may comprise an amino acid sequence that is at least 90% homologous to an amino acid sequence selected from a group consisting of SEQ ID NOS: 36-37. The anti-CD20 heavy chain may comprise an amino acid sequence that comprises 50 or more consecutive amino acids from an amino acid sequence selected from a group consisting of SEQ ID NOS: 36-37. The anti-CD20 heavy chain may comprise an amino acid sequence that comprises 100 or more consecutive amino acids from an amino acid sequence selected from a group consisting of SEQ ID NOS: 36-37. The anti-CD20 heavy chain may comprise an amino acid sequence that comprises 150 or more consecutive amino acids from an amino acid sequence selected from a group consisting of SEQ ID NOS: 36-37. The anti-CD20 heavy chain may comprise an amino acid sequence that comprises 200 or more consecutive amino acids from an amino acid sequence selected from a group consisting of SEQ ID NOS: 36-37.

The first antibody or antibody fragment may be based on or derived from a Her2 antibody or antibody fragment. The second antibody or antibody fragment may be based on or derived from a UCHT1 antibody or antibody fragment. The Her2 may be Her2scFv. The Her2 may comprise an amino acid sequence that is at least 50% homologous to an amino acid selected from a group consisting of SEQ ID NOS: 33, 40, and 86. The Her2 may comprise an amino acid sequence that is at least 60% homologous to an amino acid selected from a group consisting of SEQ ID NOS: 33, 40, and 86. The Her2 may comprise an amino acid sequence that is at least 70% homologous to an amino acid selected from a group consisting of SEQ ID NOS: 33, 40, and 86. The Her2 may comprise an amino acid sequence that is at least 80% homologous to an amino acid selected from a group consisting of SEQ ID NOS: 33, 40, and 86. The Her2 may comprise an amino acid sequence that is at least 90% homologous to an amino acid selected from a group consisting of SEQ ID NOS: 33, 40, and 86. The Her2 may comprise an amino acid sequence that comprises 10 or more consecutive amino acids from an amino acid selected from a group consisting of SEQ ID NOS: 33, 40, and 86. The Her2 may comprise an amino acid sequence that comprises 50 or more consecutive amino acids from an amino acid selected from a group consisting of SEQ ID NOS: 33, 40, and 86. The Her2 may comprise an amino acid sequence that comprises 100 or more consecutive amino acids from an amino acid selected from a group consisting of SEQ ID NOS: 33, 40, and 86. The Her2 may comprise an amino acid sequence that comprises 200 or more consecutive amino acids from an amino acid selected from a group consisting of SEQ ID NOS: 33, 40, and 86. The Her2 may be Her2 light chain. The Her2 may comprise an amino acid sequence that is at least 50% homologous to SEQ ID NO: 40. The Her2 light chain may comprise an amino acid sequence that is at least 60% homologous to SEQ ID NO: 40. The Her2 light chain may comprise an amino acid sequence that is at least 70% homologous to SEQ ID NO: 40. The Her2 light chain may comprise an amino acid sequence that is at least 80% homologous to SEQ ID NO: 40. The Her2 light chain may comprise an amino acid sequence that is at least 90% homologous to SEQ ID NO: 40. The Her2 light chain may comprise an amino acid sequence that comprises 10 or more consecutive amino acids from SEQ ID NO: 40. The Her2 light chain may comprise an amino acid sequence that comprises 50 or more consecutive amino acids from SEQ ID NO: 40. The Her2 light chain may comprise an amino acid sequence that comprises 100 or more consecutive amino acids from SEQ ID NO: 40. The Her2 light chain may comprise an amino acid sequence that comprises 200 or more consecutive amino acids from SEQ ID NO: 40. The Her2 may be Her2 heavy chain. The Her2 heavy chain may comprise an amino acid sequence that is at least 50% homologous to SEQ ID NO: 33. The Her2 heavy chain may comprise an amino acid sequence that is at least 60% homologous to SEQ ID NO: 33. The Her2 heavy chain may comprise an amino acid sequence that is at least 70% homologous to SEQ ID NO: 33. The Her2 heavy chain may comprise an amino acid sequence that is at least 80% homologous to SEQ ID NO: 33. The Her2 heavy chain may comprise an amino acid sequence that is at least 90% homologous to SEQ ID NO: 33. The Her2 heavy chain may comprise an amino acid sequence that comprises 10 or more consecutive amino acids from SEQ ID NO: 33. The Her2 heavy chain may comprise an amino acid sequence that comprises 50 or more consecutive amino acids from SEQ ID NO: 33. The Her2 heavy chain may comprise an amino acid sequence that comprises 100 or more consecutive amino acids from SEQ ID NO: 33. The Her2 heavy chain may comprise an amino acid sequence that comprises 200 or more consecutive amino acids from SEQ ID NO: 33.

The bispecific antibody may comprise an amino acid sequence that is at least about 50% homologous to an amino acid sequence selected from a group consisting of SEQ ID NOS: 58-60, and 67-70. The bispecific antibody may comprise an amino acid sequence that is at least about 60% homologous to an amino acid sequence selected from a group consisting of SEQ ID NOS: 58-60, and 67-70. The bispecific antibody may comprise an amino acid sequence that is at least about 70% homologous to an amino acid sequence selected from a group consisting of SEQ ID NOS: 58-60, and 67-70. The bispecific antibody may comprise an amino acid sequence that is at least about 80% homologous to an amino acid sequence selected from a group consisting of SEQ ID NOS: 58-60, and 67-70. The bispecific antibody may comprise an amino acid sequence that is at least about 90% homologous to an amino acid sequence selected from a group consisting of SEQ ID NOS: 58-60, and 67-70. The bispecific antibody may comprise an amino acid sequence that comprises 25 or more consecutive amino acids from any one of SEQ ID NOS: 58-60, and 67-70. The bispecific antibody may comprise an amino acid sequence that comprises 50 or more consecutive amino acids from any one of SEQ ID NOS: 58-60, and 67-70. The bispecific antibody may comprise an amino acid sequence that comprises 100 or more consecutive amino acids from any one of SEQ ID NOS: 58-60, and 67-70. The bispecific antibody may comprise an amino acid sequence that comprises 150 or more consecutive amino acids from any one of SEQ ID NOS: 58-60, and 67-70. The bispecific antibody may comprise an amino acid sequence that comprises 200 or more consecutive amino acids from any one of SEQ ID NOS: 58-60, and 67-70. The bispecific antibody may comprise an amino acid sequence that comprises 300 or more consecutive amino acids from any one of SEQ ID NOS: 58-60, and 67-70. The bispecific antibody may comprise an amino acid sequence that comprises 350 or more consecutive amino acids from any one of SEQ ID NOS: 58-60, and 67-70. The amino acid sequence may be SEQ ID NO: 58. The amino acid sequence may be SEQ ID NO: 59. The amino acid sequence may be SEQ ID NO: 60. The amino acid sequence may be SEQ ID NO: 67. The amino acid sequence may be SEQ ID NO: 68. The amino acid sequence may be SEQ ID NO: 69. The amino acid sequence may be SEQ ID NO: 70.

The bispecific antibody may further comprise a third antibody or antibody fragment. The third antibody or antibody fragment may be based on or derived from a UCHT1 antibody. The third antibody or antibody fragment may be based on or derived from a Her2 antibody. The third antibody or antibody fragment may be based on or derived from an anti-CD19 antibody. The third antibody or antibody fragment may be based on or derived from an anti-CD20 antibody. The third antibody or antibody fragment may comprise a fragment antigen binding (Fab), fragment antigen-binding including hinge region (F(ab′)₂), fragment antigen-binding including one hinge region (Fab′), fragment crystallizable (Fc), variable domain (e.g., V_{H} or V_{L}), constant domain (e.g., C_{H1}, C_{H2}, C_{H3}, or C_{L}), single-chain varaible fragment (scFV), di-ScFv, single domain antibody (sdAb), minibody, diabody, tribody, tetrabody, trifunctional antibody. The third antibody or antibody fragment may comprise one or more heavy chains, light chains, or both. The third antibody or antibody fragment may comprise one or more constant domains. The third antibody fragment or antibody fragment may comprise one or more variable domains. The third antibody or antibody fragment may comprise an amino acid sequence that is at least 50% homologous to an amino acid sequence selected from a group consisting of SEQ ID NO: 33-44. The third antibody or antibody fragment may comprise an amino acid sequence that is at least 60% homologous to an amino acid sequence selected from a group consisting of SEQ ID NO: 33-44. The third antibody or antibody fragment may comprise an amino acid sequence that is at least 70% homologous to an amino acid sequence selected from a group consisting of SEQ ID NO: 33-44. The the third antibody or antibody fragment may comprise an amino acid sequence that is at least 80% homologous to an amino acid sequence selected from a group consisting of SEQ ID NO: 33-44. The third antibody or antibody fragment may comprise an amino acid sequence that is at least 90% homologous to an amino acid sequence selected from a group consisting of SEQ ID NO: 33-44. The third antibody or antibody fragment may comprise an amino acid sequence that comprises 50 or more consecutive amino acids from any one of SEQ ID NO: 33-44. The third antibody or antibody fragment may comprise an amino acid sequence that comprises 100 or more consecutive amino acids from any one of SEQ ID NO: 33-44. The third antibody or antibody fragment may comprise an amino acid sequence that comprises 150 or more consecutive amino acids from any one of SEQ ID NO: 33-44. The third antibody or antibody fragment may comprise an amino acid sequence that comprises 200 or more consecutive amino acids from any one of SEQ ID NO: 33-44. The amino acid sequence may be SEQ ID NO: 33. The amino acid sequence may be SEQ ID NO: 34. The amino acid sequence may be an amino acid sequence selected from a group consisting of SEQ ID NO: 35. The amino acid sequence may be SEQ ID NO: 36. The amino acid sequence may be SEQ ID NO: 37. The amino acid sequence may be SEQ ID NO: 38. The amino acid sequence may be SEQ ID NO: 39. The amino acid sequence may be SEQ ID NO: 40. The amino acid sequence may be SEQ ID NO: 41. The amino acid sequence may be SEQ ID NO: 42. The amino acid sequence may be SEQ ID NO: 43. The amino acid sequence may be SEQ ID NO: 44.

The bispecific antibodies disclosed herein may further comprise one or more adapter peptides. An adapter peptide may connect the antibody region to the non-antibody polypeptide region. Alternatively, or additionally, the adapter peptide may be inserted into the non-antibody polypeptide region. The bispecific antibodies disclosed herein may comprise 1, 2, 3, 4, 5, 6, 7, 8, 9, 10 or more adapter peptides. The bispecific antibodies disclosed herein may comprise 1 or more adapter peptides. The bispecific antibodies disclosed herein may comprise 2 or more adapter peptides. The bispecific antibodies disclosed herein may comprise 3 or more adapter peptides. The adapter peptide may be a synthetic peptide. In some instances, the adapter peptide is not based on or derived from an antibody or antibody fragment. In some instances, the adapter peptide is not based on or derived from a complementarity determining region (CDR) of an antibody or antibody fragment. The CDR may be CDR1. The CDR may be CDR2. The CDR may be CDR3.

The adapter peptide may comprise 1, 2, 3, 4, 5, 6, 7, 8, 9, 10 or more consecutive amino acids. The adapter peptide may comprise 10, 20, 30, 40, 50, 60, 70, 80, 90, 100 or more consecutive amino acids. The adapter peptide may comprise 1, 2, 3, 4 or more consecutive amino acids based on or derived from an amino acid sequence selected from a group consisting of SEQ ID NO: 71-77. The adapter peptide may comprise 4 or more consecutive amino acids based on or derived from an amino acid sequence selected from a group consisting of SEQ ID NO: 71-77. The adapter peptide may comprise 5, 6, 7, 9, 10, 11, 12, 13, 14, 15 or more consecutive amino acids based on or derived from an amino acid sequence selected from a group consisting of SEQ ID NO: 71-77. The adapter peptide may comprise 15 or more consecutive amino acids based on or derived from an amino acid sequence selected from a group consisting of SEQ ID NO: 71-77. The adapter peptide may comprise 16, 17, 18, 19, 20 or more consecutive amino acids based on or derived from an amino acid sequence selected from a group consisting of SEQ ID NO: 71-77. The adapter peptide may comprise 20 or more consecutive amino acids based on or derived from an amino acid sequence selected from a group consisting of SEQ ID NO: 71-77. The adapter peptide may comprise an amino acid sequence that is at least about 50% homologous to an amino acid sequence selected from a group consisting of SEQ ID NO: 71-77. The adapter peptide may comprise an amino acid sequence that is at least about 60% homologous to an amino acid sequence selected from a group consisting of SEQ ID NO: 71-77. The adapter peptide may comprise an amino acid sequence that is at least about 70% homologous to an amino acid sequence selected from a group consisting of SEQ ID NO: 71-77. The adapter peptide may comprise an amino acid sequence that is at least about 75% homologous to an amino acid sequence selected from a group consisting of SEQ ID NO: 71-77. The adapter peptide may comprise an amino acid sequence that is at least about 80% homologous to an amino acid sequence selected from a group consisting of SEQ ID NO: 71-77. The adapter peptide may comprise an amino acid sequence that is at least about 85% homologous to an amino acid sequence selected from a group consisting of SEQ ID NO: 71-77. The adapter peptide may comprise an amino acid sequence that is at least about 90% homologous to an amino acid sequence selected from a group consisting of SEQ ID NO: 71-77. The adapter peptide may comprise an amino acid sequence that is at least about 95% homologous to an amino acid sequence selected from a group consisting of SEQ ID NO: 71-77. The adapter peptide may comprise an amino acid sequence that is at least about 97% homologous to an amino acid sequence selected from a group consisting of SEQ ID NO: 71-77. The adapter peptide may comprise an amino acid sequence that is at least about 100% homologous to an amino acid sequence selected from a group consisting of SEQ ID NO: 71-77. The amino acid sequence may be SEQ ID NO: 71. The amino acid sequence may be SEQ ID NO: 72. The amino acid sequence may be SEQ ID NO: 73. The amino acid sequence may be SEQ ID NO: 74. The amino acid sequence may be SEQ ID NO: 75. The amino acid sequence may be SEQ ID NO: 76. The amino acid sequence may be SEQ ID NO: 77.

Further disclosed herein are uses of a bispecific antibody to treat a disease or condition in a subject. The bispecific antibody may comprise (a) first antibody or antibody fragment; and (b) a second antibody or antibody fragment, wherein the second antibody or antibody fragment may be inserted into the first antibody or antibody fragment by replacement of less than about 20 amino acid residues from the first antibody or antibody fragment with the second antibody or antibody fragment. The bispecific antibody may comprise any of the bispecific antibodies disclosed herein. The first antibody or antibody fragment may comprise any of the first antibody or antibody fragments disclosed herein. The second antibody or antibody fragment may comprise any of the second antibody or antibody fragments disclosed herein. The bispecific antibody may further comprise a third antibody or antibody fragment. The one or more antibody or antibody fragments may comprise any of the antibodies or antibody fragments disclosed herein. The bispecific antibody may further comprise one or more adapter peptides. The one or more adapter peptides may comprise any of the adapter peptides disclosed herein. In some instances, the second antibody or antibody fragment is not inserted into a complementarity determining region (CDR) of the first antibody or antibody fragment. The CDR may be CDR1. The CDR may be CDR2. The CDR may be CDR3.

Further disclosed herein are methods of treating a disease or condition in a subject in need thereof, the method comprising administering to the subject a bispecific antibody comprising (a) first antibody or antibody fragment; and (b) a second antibody or antibody fragment, wherein the second antibody or antibody fragment may be inserted into the first antibody or antibody fragment by replacement of less than about 20 amino acid residues from the first antibody or antibody fragment with the second antibody or antibody fragment. The bispecific antibody may comprise any of the bispecific antibodies disclosed herein. The first antibody or antibody fragment may comprise any of the first antibody or antibody fragments disclosed herein. The second antibody or antibody fragment may comprise any of the second antibody or antibody fragments disclosed herein. The bispecific antibody may further comprise a third antibody or antibody fragment. The one or more antibody or antibody fragments may comprise any of the antibodies or antibody fragments disclosed herein. The bispecific antibody may further comprise one or more adapter peptides. The one or more adapter peptides may comprise any of the adapter peptides disclosed herein. In some instances, the second antibody or antibody fragment is not inserted into a complementarity determining region (CDR) of the first antibody or antibody fragment. The CDR may be CDR1. The CDR may be CDR2. The CDR may be CDR3.

The disease or condition may be a cancer. The cancer may be a lymphoma. The lymphoma may be a non-Hodgkins lymphoma (NHL). The lymphoma may comprise one or more CD19 positive lymphoma cells. The lymphoma may be a B-cell lymphoma. The cancer may be a breast cancer. The first antibody or antibody fragment may be based on or derived from UCHT1. The second antibody or antibody fragment may be based on or derived from trastuzumab. The bispecific antibody may comprise (a) a first antibody or antibody fragment may be based on or derived from UCHT1; and (b) a second antibody or antibody fragment may be based on or derived from trastuzumab, wherein the second antibody or antibody fragment may be inserted into the first antibody or antibody fragment by replacement of less than about 20 amino acid residues from the first antibody or antibody fragment with the second antibody or antibody fragment. The UCHT1 may be any of the UCHT1 antibodies or antibody fragments disclosed herein. The trastuzumab may be any of the trastuzumab antibodies or antibody fragments disclosed herein.

The cancer may be a breast cancer. The first antibody or antibody fragment may be based on or derived from trastuzumab. The second antibody or antibody fragment may be based on or derived from UCHT1. The bispecific antibody may comprise (a) a first antibody or antibody fragment may be based on or derived from trastuzumab; and (b) a second antibody or antibody fragment may be based on or derived from UCHT1, wherein the second antibody or antibody fragment may be inserted into the first antibody or antibody fragment by replacement of less than about 20 amino acid residues from the first antibody or antibody fragment with the second antibody or antibody fragment. The trastuzumab may be any of the trastuzumab antibodies or antibody fragments disclosed herein. The UCHT1 may be any of the UCHT1 antibodies or antibody fragments disclosed herein.

The first antibody or antibody fragment may be based on or derived from UCHT1. The second antibody or antibody fragment may be based on or derived from anti-CD19. The bispecific antibody may comprise (a) a first antibody or antibody fragment may be based on or derived from UCHT1; and (b) a second antibody or antibody fragment may be based on or derived from anti-CD19, wherein the second antibody or antibody fragment may be inserted into the first antibody or antibody fragment by replacement of less than about 20 amino acid residues from the first antibody or antibody fragment with the second antibody or antibody fragment. The UCHT1 may be any of the UCHT1 antibodies or antibody fragments disclosed herein. The anti-CD19 may be any of the anti-CD19 antibodies or antibody fragments disclosed herein.

Further disclosed herein are one or more plasmids comprising a nucleic acid sequence encoding any of the bispecific antibody proteins disclosed herein. The nucleic acid sequence encoding the bispecific antibody may be at least about 50%, 60%, 70%, 75%, 80%, 85%, 90%, 95%, or 97% or more homologous to a nucleic acid sequence selected from a group consisting of SEQ ID NOS: 24-26 and 29-32. The nucleic acid sequence encoding the bispecific antibody may be at least about 60% or more homologous to a nucleic acid sequence selected from a group consisting of SEQ ID NOS: 24-26 and 29-32. The nucleic acid sequence encoding the bispecific antibody may be at least about 65% or more homologous to a nucleic acid sequence selected from a group consisting of SEQ ID NOS: 24-26 and 29-32. The nucleic acid sequence encoding the bispecific antibody may be at least about 70% or more homologous to a nucleic acid sequence selected from a group consisting of SEQ ID NOS: 24-26 and 29-32. The nucleic acid sequence encoding the bispecific antibody may be at least about 75% or more homologous to a nucleic acid sequence selected from a group consisting of SEQ ID NOS: 24-26 and 29-32. The nucleic acid sequence encoding the bispecific antibody may be at least about 80% or more homologous to a nucleic acid sequence selected from a group consisting of SEQ ID NOS: 24-26 and 29-32. The nucleic acid sequence encoding the bispecific antibody may be at least about 85% or more homologous to a nucleic acid sequence selected from a group consisting of SEQ ID NOS: 24-26 and 29-32. The nucleic acid sequence encoding the bispecific antibody may be at least about 90% or more homologous to a nucleic acid sequence selected from a group consisting of SEQ ID NOS: 24-26 and 29-32. The nucleic acid sequence encoding the bispecific antibody may be at least about 95% or more homologous to a nucleic acid sequence selected from a group consisting of SEQ ID NOS: 24-26 and 29-32. The nucleic acid sequence may be SEQ ID NO: 24. The nucleic acid sequence may be SEQ ID NO: 25. The nucleic acid sequence may be SEQ ID NO: 26. The nucleic acid sequence may be SEQ ID NO: 29. The nucleic acid sequence may be SEQ ID NO: 30. The nucleic acid sequence may be SEQ ID NO: 31. The nucleic acid sequence may be SEQ ID NO: 32.

The bispecific antibody may comprise an amino acid sequence that is at least about 50%, 60%, 70%, 75%, 80%, 85%, 90%, 95%, or 97% or more homologous to an amino acid sequence selected from a group consisting of SEQ ID NOS: 58-60, and 67-70. The bispecific antibody may comprise an amino acid sequence that is at least about 60% or more homologous to an amino acid sequence selected from a group consisting of SEQ ID NOS: 58-60, and 67-70. The bispecific antibody may comprise an amino acid sequence that is at least about 65% or more homologous to an amino acid sequence selected from a group consisting of SEQ ID NOS: 58-60, and 67-70. The bispecific antibody may comprise an amino acid sequence that is at least about 70% or more homologous to an amino acid sequence selected from a group consisting of SEQ ID NOS: 58-60, and 67-70. The bispecific antibody may comprise an amino acid sequence that is at least about 75% or more homologous to an amino acid sequence selected from a group consisting of SEQ ID NOS: 58-60, and 67-70. The bispecific antibody may comprise an amino acid sequence that is at least about 80% or more homologous to an amino acid sequence selected from a group consisting of SEQ ID NOS: 58-60, and 67-70. The bispecific antibody may comprise an amino acid sequence that is at least about 85% or more homologous to an amino acid sequence selected from a group consisting of SEQ ID NOS: 58-60, and 67-70. The bispecific antibody may comprise an amino acid sequence that is at least about 90% or more homologous to an amino acid sequence selected from a group consisting of SEQ ID NOS: 58-60, and 67-70. The bispecific antibody may comprise an amino acid sequence that is at least about 95% or more homologous to an amino acid sequence selected from a group consisting of SEQ ID NOS: 58-60, and 67-70. The amino acid sequence may be SEQ ID NO: 58. The amino acid sequence may be SEQ ID NO: 59. The amino acid sequence may be SEQ ID NO: 60. The amino acid sequence may be SEQ ID NO: 67. The amino acid sequence may be SEQ ID NO: 68. The amino acid sequence may be SEQ ID NO: 69. The amino acid sequence may be SEQ ID NO: 70.

The bispecific antibody may comprise an amino acid sequence that comprises 50, 60, 70, 80, 90, 100, 110, 120, 130, 140, 150, 160, 170, 180, 190, 200 or more consecutive amino acids from an amino acid sequence selected from a group consisting of SEQ ID NOS: 58-60, and 67-70. The bispecific antibody comprises an amino acid sequence that comprises 200, 225, 250, 275, 300, 325, 300, 325, 350, 375, 400 or more consecutive amino acids from an amino acid sequence selected from a group consisting of SEQ ID NOS: 58-60, and 67-70. The bispecific antibody comprises an amino acid sequence that comprises 50 or more consecutive amino acids from an amino acid sequence selected from a group consisting of SEQ ID NOS: 58-60, and 67-70. The bispecific antibody comprises an amino acid sequence that comprises 100 or more consecutive amino acids from an amino acid sequence selected from a group consisting of SEQ ID NOS: 58-60, and 67-70. The bispecific antibody comprises an amino acid sequence that comprises 150 or more consecutive amino acids from an amino acid sequence selected from a group consisting of SEQ ID NOS: 58-60, and 67-70. The bispecific antibody comprises an amino acid sequence that comprises 200 or more consecutive amino acids from an amino acid sequence selected from a group consisting of SEQ ID NOS: 58-60, and 67-70. The amino acid sequence may be SEQ ID NO: 58. The amino acid sequence may be SEQ ID NO: 59. The amino acid sequence may be SEQ ID NO: 60. The amino acid sequence may be SEQ ID NO: 67. The amino acid sequence may be SEQ ID NO: 68. The amino acid sequence may be SEQ ID NO: 69. The amino acid sequence may be SEQ ID NO: 70.

Further disclosed herein are one or more cells comprising any of the plasmids disclosed herein. The one or more cells may comprise a plasmid comprising a nucleic acid sequenc encoding a bispecific fusion antibody disclosed herein. The cell may be a eukaryotic cell. The cell may be a prokaryotic cell. The cell may be a mammalian cell. The mammalian cell may be a human cell. The mammalian cell may be HEK 293 T cells.

### Antibody Drug Conjugates

Further disclosed herein are antibody drug conjugates. Generally, an antibody drug conjugate comprises a) an antibody fusion protein disclosed herein; and b) an additional antibody or antibody fragment. The antibody fusion protein may comprise (a) an antibody region based on or derived from an antibody or antibody fragment; and (b) a non-antibody polypeptide region comprising 15 or more amino acids, wherein the non-antibody polypeptide region is inserted into a constant domain of the antibody region. The non-antibody peptide may be inserted into the constant domain of the antibody region by replacement of less than about 20 amino acid residues from the constant domain of the antibody region with the non-antibody polypeptide region. Alternatively, insertion of the non-antibody peptide does not comprise replacement of one or more amino acid residues from the constant domain of the antibody region. The non-antibody peptide may be a non-antigenic peptide. In some instances, the non-antibody peptide is not based on or derived from a T cell epitope. In some instances, the non-antibody peptide is not based on or derived from a B cell epitope. In some instances, the antibody region is not based on or derived from an antigen presenting cell (APC) specific antibody. In some instances, the antibody region is not based on or derived from a major histocompatibilitycomplex (MHC) specific antibody. In some instances, the antibody region is not based on or derived from a major histocompatibilitycomplex class I (MHC class I) specific antibody. In some instances, the antibody region is not based on or derived from a major histocompatibilitycomplex class II (MHC class II) specific antibody. Alternataively, or additionally, the antibody fusion protein may comprise (a) an antibody region based on or derived from an antibody or antibody fragment; and (b) a non-antibody polypeptide region, wherein the non-antibody polypeptide region may be inserted into the antibody region by replacement of less than about 20 amino acid residues from the antibody or antibody fragment with the non-antibody polypeptide region. In some instances, the non-antibody polypeptide is not inserted into a complementarity determining region (CDR) of the antibody or antibody fragment. The CDR may be CDR1. The CDR may be CDR2. The CDR may be CDR3. The non-antibody polypeptide region may comprise 15 or more amino acids. The non-antibody polypeptide region may comprise 16 or more amino acids. The non-antibody polypeptide region may comprise 17 or more amino acids. The non-antibody polypeptide region may comprise 18 or more amino acids. The non-antibody polypeptide region may comprise 19 or more amino acids. The non-antibody polypeptide region may comprise 20 or more amino acids. The non-antibody polypeptide region may comprise 21 or more amino acids. The non-antibody polypeptide region may comprise 22 or more amino acids. The non-antibody polypeptide region may comprise 20, 30, 40, 50, 60, 70, or 80 or more amino acids. The antibody fusion proteins disclosed herein may be used to treat a disease or condition in a subject in need thereof. Further disclosed herein are methods of treating a disease or condition in a subject in need, the method comprising administering to the subject an antibody fusion protein disclosed herein. Alternatively, or additionally, an antibody drug conjugate comprises a) a bispecific antibody disclosed herein; and b) an additional antibody or antibody fragment. The bispecific antibody may comprise any of the bispecific antibodies disclosed herein. The bispecific antibody may comprise (a) first antibody or antibody fragment; and (b) a second antibody or antibody fragment, wherein the second antibody or antibody fragment may be inserted into a constant domain of the first antibody or antibody fragment. The second antibody or antibody fragment may be inserted into the constant domain of the first antibody or antibody fragment by replacement of less than about 20 amino acid residues from the constant domain of the first antibody or antibody fragment with the second antibody or antibody fragment. Alternatively, insertion of the second antibody or antibody fragment in to the first antibody or antibody fragment does not comprise replacement of or more amino acids from the constant domain of the first antibody. The second antibody or antibody fragment may be inserted into the constant domain of a heavy chain of the first antibody or antibody fragment. The constant domain of the heavy chain may be CH1. The constant domain of the heavy chain may be CH2. The constant domain of the heavy chain may be CH3.The second antibody or antibody fragment may be inserted into the constant domain of a light chain of the first antibody or antibody fragment. The bispecific antibody may comprise (a) first antibody or antibody fragment; and (b) a second antibody or antibody fragment, wherein the second antibody or antibody fragment may be inserted into the first antibody or antibody fragment by replacement of less than about 20 amino acid residues from the first antibody or antibody fragment with the second antibody or antibody fragment. In some instances, the second antibody or antibody fragment is not inserted into a complementarity determining region (CDR) of the first antibody or antibody fragment. The CDR may be CDR1. The CDR may be CDR2. The CDR may be CDR3. The first antibody or antibody fragment may comprise any of the first antibodies or antibody fragments disclosed herein. The second antibody or antibody fragment may comprise any of the second antibodies or antibody fragments disclosed herein. The antibody drug conjugate may comprise a CXCR4-BP-Trastuzumab antibody fusion protein. The antibody drug conjugate may comprise a CXCR4-BP-CD20-CL (Fab) antibody fusion protein. The antibody drug conjugate may comprise a CXCR4-BP-CD20-CL (IgG) antibody fusion protein. The antibody drug conjugate may comprise an antibody fusion fusion protein selected from a group consisting of CXCR4-BP-palivizumab, CXCR4-BP-Trastuzumab, CXCR4-BP-CD20-CL (Fab), and CXCR4-BP-CD20-CL (IgG). The additional antibody or antibody region may be selected from a group consisting of trastuzumab light chain and anti-CD20 heavy chain. The anti-CD20 heavy chain may be an anti-CD20 heavy Fab fragment. The anti-Cd20 heavy chain may be a full-length CD-20 heavy chain. The antibody fusion protein may be encoded by a nucleic acid sequence that is at least about 50% homologous to a nucleic acid sequence selected from a group consisting of SEQ ID NOS: 36, 37, and 43. The antibody fusion protein may be encoded by a nucleic acid sequence that is at least about 60% homologous to a nucleic acid sequence selected from a group consisting of SEQ ID NOS: 36, 37, and 43. The antibody fusion protein may be encoded by a nucleic acid sequence that is at least about 70% homologous to a nucleic acid sequence selected from a group consisting of SEQ ID NO: 12, 20, and 21. The antibody fusion protein may be encoded by a nucleic acid sequence that is at least about 80% homologous to a nucleic acid sequence selected from a group consisting of SEQ ID NOS: 36, 37, and 43 The antibody fusion protein may be encoded by a nucleic acid sequence that is at least about 90% homologous to a nucleic acid sequence selected from a group consisting of SEQ ID NOS: 36, 37, and 43. The nucleic acid sequence may be SEQ ID NO: 36. The nucleic acid sequence may be SEQ ID NO: 37. The nucleic acid sequence may be SEQ ID NO: 43. The additional antibody or antibody fragment may be encoded by a nucleic acid sequence that is at least about 50% homologous to a nucleic acid sequence selected from a group consisting of SEQ ID NOS: 1, 4, 5, and 8. The additional antibody or antibody fragment may be encoded by a nucleic acid sequence that is at least about 60% homologous to a nucleic acid sequence selected from a group consisting of SEQ ID NOS: 1, 4, 5, and 8. The additional antibody or antibody fragment may be encoded by a nucleic acid sequence that is at least about 70% homologous to a nucleic acid sequence selected from a group consisting of SEQ ID NOS: 1, 4, 5, and 8. The additional antibody or antibody fragment may be encoded by a nucleic acid sequence that is at least about 80% homologous to a nucleic acid sequence selected from a group consisting of SEQ ID NOS: 1, 4, 5, and 8. The additional antibody or antibody fragment may be encoded by a nucleic acid sequence that is at least about 90% homologous to a nucleic acid sequence selected from a group consisting of SEQ ID NOS: 1, 4, 5, and 8. The nucleic acid sequence may be SEQ ID NO: 1. The nucleic acid sequence may be SEQ ID NO: 4. The nucleic acid sequence may be SEQ ID NO: 5. The nucleic acid sequence may be SEQ ID NO: 8. The antibody fusion protein may comprise an amino acid sequence that is at least about 50% homologous to an amino acid sequence selected from a group consisting of SEQ ID NOS: 47, 55, 56, 65, and 66. The antibody fusion protein may comprise an amino acid sequence that is at least about 60% homologous to an amino acid sequence selected from a group consisting of SEQ ID NOS: 47, 55, 56, 65, and 66. The antibody fusion protein may comprise an amino acid sequence that is at least about 70% homologous to an amino acid sequence selected from a group consisting of SEQ ID NOS: 47, 55, 56, 65, and 66. The antibody fusion protein may comprise an amino acid sequence that is at least about 80% homologous to an amino acid sequence selected from a group consisting of SEQ ID NOS: 47, 55, 56, 65, and 66. The antibody fusion protein may comprise an amino acid sequence that is at least about 90% homologous to an amino acid sequence selected from a group consisting of SEQ ID NOS: 47, 55, 56, 65, and 66. The amino acid sequence may be SEQ ID NO: 47. The amino acid sequence may be an amino acid sequence selected from a group consisting of SEQ ID NO: 55. The amino acid sequence may be SEQ ID NO: 65. The amino acid sequence may be an amino acid sequence selected from a group consisting of SEQ ID NO: 67. The amino acid sequence may be SEQ ID NO: 68. The additional antibody or antibody fragment may comprise an amino acid sequence that is at least about 50% homologous to an amino acid sequence selected from a group consisting of SEQ ID NOS: 47, 55, 56, 65, and 66. The additional antibody or antibody fragment may comprise an amino acid sequence that is at least about 60% homologous to an amino acid sequence selected from a group consisting of SEQ ID NOS: 47, 55, 56, 65, and 66. The additional antibody or antibody fragment may comprise an amino acid sequence that is at least about 70% homologous to an amino acid sequence selected from a group consisting of SEQ ID NOS: 47, 55, 56, 65, and 66. The additional antibody or antibody fragment may comprise an amino acid sequence that is at least about 80% homologous to an amino acid sequence selected from a group consisting of SEQ ID NOS: 47, 55, 56, 65, and 66. The additional antibody or antibody fragment may comprise an amino acid sequence that is at least about 90% homologous to an amino acid sequence selected from a group consisting of SEQ ID NOS: 47, 55, 56, 65, and 66. The amino acid sequence may be SEQ ID NO: 47. The amino acid sequence may be SEQ ID NO: 55. The amino acid sequence may be SEQ ID NO: 56. The amino acid sequence may be SEQ ID NO: 65. The amino acid sequence may be SEQ ID NO: 66.

### EXAMPLES

The following illustrative examples are representative of embodiments of the software applications, systems, and methods described herein and are not meant to be limiting in any way. The activity data provided in the following examples were generally obtained using the immunoglobulin fusion proteins defined in the examples and exemplified by the provided SEQ ID. It is to be understood that the activities of any antibody fusion protein or bispecific antibody disclosed herein may be enhanced or attenuated depending on conditions not relating to antibody fusion protein or bispecific antibody sequence, for example, expression and purification conditions.

### Example 1

### Cloning, Expression and Purification of hEPO-Coil-Trastuzumab-CL

Cloning: Mammalian expression vector of Trastuzumab full-length IgG heavy chain was generated by in-frame ligation of amplified Trastuzumab Fab heavy chain (VH and CHO to pFuse-hIgG1-Fc backbone vector (InvivoGen, CA). A gene encoding antibody Trastuzumab light chain was amplified and cloned into the pFuse vector without hIgG1 Fc fragment. A gene encodinghEPO was synthesized by Genscript (NJ, USA), and amplified by polymerase chain reaction (PCR). Coiled coil stalk was added to both ends of the hEPO insert sequence. The sequence of the ascending adapter peptide with linkers at each end is: H2N-GGSGAKLAALKAKLAALKGGGGS-COOH (SEQ ID NO: 77); the sequence of the descending peptide with linkers at each end is: H2N-GGGGSELAALEAELAALEAGGSG-COOH (SEQ ID NO: 76). Subsequently, hEPO-Her2-CL IgGfusion proteins were created by replacing the K169 in CL region of Trastuzumab light chain with hEPO with coiled-coil stalk. The resulting mammalian expression vectors were confirmed by DNA sequencing.

Expression and Purification: hEPO-coil-Her2-CL IgG full-length IgG was expressed through transient transfection of FreeStyle HEK 293 cells with expression vectors of Trastuzumab heavy chain and hEPO-coil-Her2-CL IgG light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection.hEPO-coil-Her2-CLIgG was purified by Protein G chromatography (Thermo Fisher Scientific, IL). Purified proteins were analyzed by SDS-PAGE gels. FIG. 1 shows an SDS gel image of hEPO-coil-Trastuzumab-CL in non-reducing and reducing (with 50 mM DTT) conditions. As shown in FIG. 1, Lane 1 represents hEPO-coil-Trastuzumab-CL without DTT treatment, Lane 2 represents hEPO-coil-Trastuzumab-CL with DTT treatment and Lane 5 represents the protein standard ladder.

### Example 2

### Cloning, Expression and Purification of hEPO-Coil-Trastuzumab-CH1

Cloning: Mammalian expression vector of Trastuzumab full-length IgG heavy chain was generated by in-frame ligation of amplified Trastuzumab Fab heavy chain (VH and CHO to pFuse-hIgG1-Fc backbone vector (InvivoGen, CA). A gene encoding antibody Trastuzumab light chain was amplified and cloned into the pFuse vector without hIgG1 Fc fragment. A gene encodinghEPO was synthesized by Genscript (NJ, USA), and amplified by polymerase chain reaction (PCR). Coiled coil stalk was added to both ends of the hEPO insert sequence. The sequence of the ascending adapter peptide with linkers at each end is: H2N-GGSGAKLAALKAKLAALKGGGGS-COOH (SEQ ID NO: 77); the sequence of the descending peptide with linkers at each end is: H2N-GGGGSELAALEAELAALEAGGSG-COOH (SEQ ID NO: 76). Subsequently, hEPO-Her2-CH1IgGfusion proteins were created by replacing the S180 and G181 in CH1 region of Trastuzumab heavy chain with hEPO with coiled-coil stalk. The resulting mammalian expression vectors were confirmed by DNA sequencing.

Expression and Purification: hEPO-coil-Her2-CH1IgG full-length IgG was expressed through transient transfection of FreeStyle HEK 293 cells with expression vectors of Trastuzumab light chain and hEPO-coil-Her2-CH1lgGheavy chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection.hEPO-coil-Her2-CH1IgG was purified by Protein G chromatography (Thermo Fisher Scientific, IL). Purified proteins were analyzed by SDS-PAGE gels. FIG. 1 shows an SDS gel image of hEPO-coil-Trastuzumab-CH1 in non-reducing and reducing (with 50 mM DTT) conditions. As shown in FIG. 1, Lane 3 represents hEPO-coil-Trastuzumab-C hEPO-coil-Trastuzumab-CH1L without DTT treatment, Lane 4 represents hEPO-coil-Trastuzumab-CH1 with DTT treatment and Lane 5 represents the protein standard ladder.

### Example 3

### In-Vitro EPO Activity Test of hEPO-Coil-Her2-CL IgG and hEPO-Coil-Her2-CH1 IgG in TF-1 cells

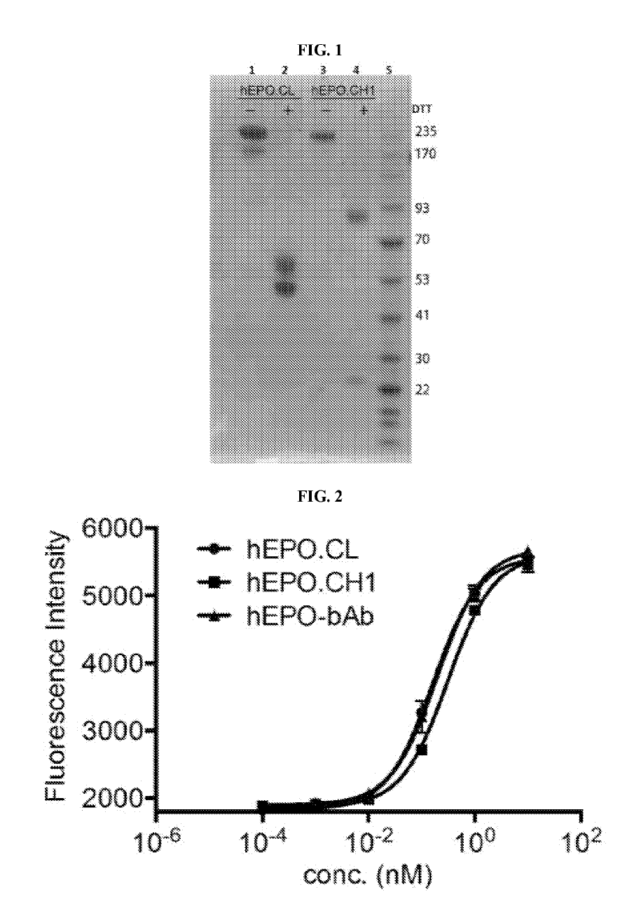

Human TF-1 cells were cultured at 37° C. with 5% CO2 in RPMI-1640 medium containing 10% fetal bovine serum (FBS), penicillin and streptomycin (50 U/mL), and 2 ng/mL human granulocyte macrophage colony stimulating factor (GM-CSF). To test the proliferative activity of Ab-hEPO fusions, cells were washed three times with RPMI-1640 medium plus 10% FBS, resuspended in RPMI-1640 medium with 10% FBS at a density of 1.5×105 cells/ml, plated in 96-well plates (1.5×104 cells per well) with various concentrations of hEPO-coil-Her2-CL (e.g., hEPO.CL), hEPO-coil-Her2-CH1 (e.g., hEPO.CH1), and hEPO-bAb-H3 (positive control, e.g., hEPO-bAb) and then incubated for 72 h at 37° C. with 5% CO2. Cells were then treated with Alamar Blue (Invitrogen) for 4 h at 37° C. Cell viability was quantified using an Alamar Blue (Invitrogen) assay. Fluorescence intensity measured at 595 nm is proportional to cell viability and plotted versus protein concentration. The EC50 values were determined by fitting data into a logistic sigmoidal function: y=A2+(A1−A2)/(1+(x/x0)p), where A1 is the initial value, A2 is the final value, x0 is the inflection point of the curve, and p is the power. FIG. 2 shows a graph of the antibody concentration versus fluorescence intensity. As shown in FIG. 2, Ab-hEPO fusion proteins stimulated proliferation of TF-1 cells in a dose-dependent manner. The EC₅₀ (nM) values of hEPO-coil-Her2-CL, hEPO-coil-Her2-CH1 and hEPO-bAb-H3 were 0.1634, 0.3135 and 0.1973, respectively.

### Example 4

### Binding Affinity of hEPO-Coil-Her2-CL and hEPO-Coil-Her2-CH1 Against Her2+ SK—BR-3 cells

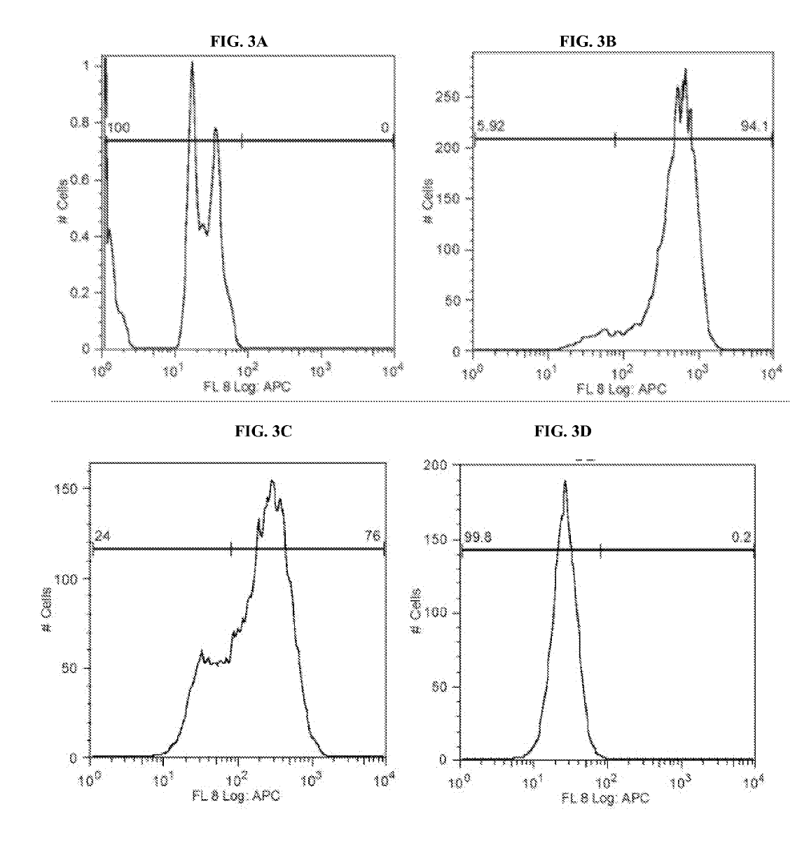

In this example, the binding affinity of hEPO-coil-Her2-CL, hEPO-coil-Her2-CH1 and wild-type trastuzumab (wt.trastuzumab) against Her2+ SK—BR-3 cells was determined by flow cytometry. SKBR3 cells were cultured according to vendor's protocol. Cells were centrifuged and blocked in 1× PBS with 10% FBS at 4° C. for 1 hour. Unconjugated primary antibodies were added to the tubes (approximately 1 μg on unconjugated primary antibody per tube). 10 nM hEPO-coil-Her2-CL, hEPO-coil-Her2-CH1 and wt.trastuzumabwere added to the cell suspensions. The cell suspensions were shaken for 1 hour at 4° C. The cells were washed 3 times with PBS. The cells were then incubated with Fluorescein-anti-human Fc at 4° C. for 1 hour. The cells were washed3 times with PBS and resuspended in PBS. The cellular fluorescence distribution was determined by flow cytometry. FIG. 3A-D depict the binding affinity of hEPO-coil-Her2-CL, hEPO-coil-Her2-CH1 and wt.trastuzumab against Her2+ SK—BR-3 cells. FIG. 3A shows the results for cells incubated with just the secondary antibody (e.g., fluorescein-anti-human). FIG. 3B shows the results for cells incubated with the wt.trastuzumab antibody, followed by the secondary antibody incubation. FIG. 3C shows the results for cells incubated with hEPO-coil-Her2-CH1, followed by the secondary antibody incubation. FIG. 3D shows the results for cells incubated with hEPO-coil-Her2-CL, followed by the secondary antibody incubation.

### Example 5

### Cloning, Expression and Purification of hEPO-Coil-Trastuzumab-CH3

Cloning: Mammalian expression vector of Trastuzumab full-length IgG heavy chain was generated by in-frame ligation of amplified Trastuzumab Fab heavy chain (VH and CHO to pFuse-hIgG1-Fc backbone vector (InvivoGen, CA). A gene encoding antibody Trastuzumab light chain was amplified and cloned into the pFuse vector without hIgG1 Fc fragment. A gene encodinghEPO was synthesized by Genscript (NJ, USA), and amplified by polymerase chain reaction (PCR). Coiled coil stalk was added to both ends of the hEPO insert sequence. The sequence of the ascending adapter peptide with linkers at each end is: H2N-GGSGAKLAALKAKLAALKGGGGS-COOH (SEQ ID NO: 77); the sequence of the descending peptide with linkers at each end is: H2N-GGGGSELAALEAELAALEAGGSG-COOH (SEQ ID NO: 76). Subsequently, hEPO-Her2-CH3IgGfusion proteins were created by replacing the T361, K362 and N363 in CH3 region of Trastuzumab heavy chain with hEPO with coiled-coil stalk. The resulting mammalian expression vectors were confirmed by DNA sequencing.

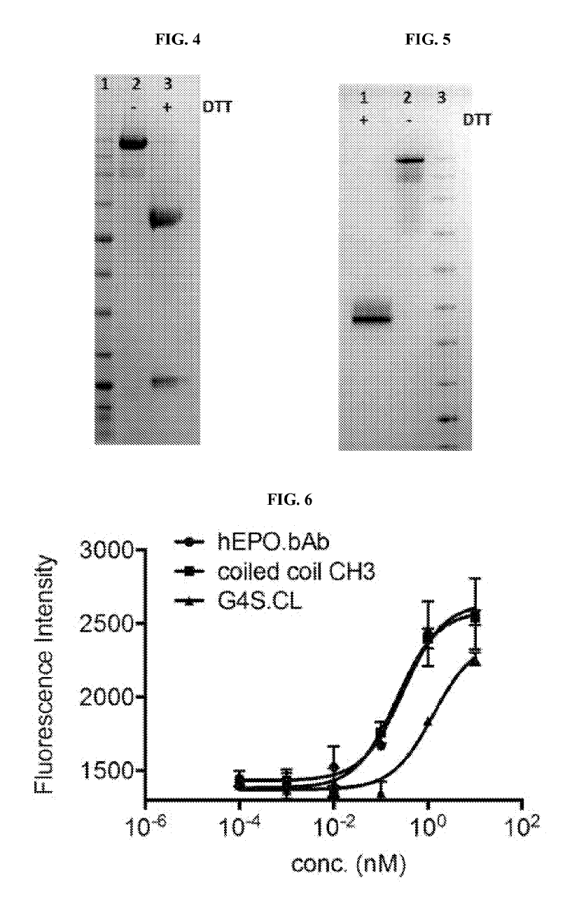

Expression and Purification: hEPO-coil-Her2-CH3IgG full-length IgG was expressed through transient transfection of FreeStyle HEK 293 cells with expression vectors of Trastuzumab light chain and hEPO-coil-Her2-CH3IgGheavy chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection.hEPO-coil-Her2-CH3IgG was purified by Protein G chromatography (Thermo Fisher Scientific, IL). Purified proteins were analyzed by SDS-PAGE gels. FIG. 4 shows SDS gel image of hEPO-coil-Trastuzumab—CH3 in non-reducing and reducing (with 50 mM DTT) conditions. As shown in FIG. 4, Lane 1 represents the protein standard ladder, Lane 2 represents hEPO-coil-Her2-CH3 without DTT treatment and Lane 3 represents hEPO-coil-Her2-CH3 with DTT treatment.

### Example 6

### Cloning, Expression and Purification of hEPO-G4S-Trastuzumab-CL

Cloning: Mammalian expression vector of Trastuzumab full-length IgG heavy chain was generated by in-frame ligation of amplified Trastuzumab Fab heavy chain (VH and CHO to pFuse-hIgG1-Fc backbone vector (InvivoGen, CA). A gene encoding antibody Trastuzumab light chain was amplified and cloned into the pFuse vector without hIgG1 Fc fragment. A gene encodinghEPO was synthesized by Genscript (NJ, USA), and amplified by polymerase chain reaction (PCR). Coiled coil stalk was added to both ends of the hEPO insert sequence. The sequence of the ascending adapter peptide with linkers at each end is: H2N-GGGGS-COOH(SEQ ID NO: 72); the sequence of the descending peptide with linkers at each end is: H2N-GGGGS-COOH(SEQ ID NO: 72). Subsequently, hEPO-G4S-Her2-CL IgGfusion proteins were created by replacing the K169 in CL region of Trastuzumab light chain with hEPO with G4S linker (SEQ ID NO: 72). The resulting mammalian expression vectors were confirmed by DNA sequencing.

Expression and Purification: hEPO-G4S-Her2-CL IgG full-length IgG was expressed through transient transfection of FreeStyle HEK 293 cells with expression vectors of Trastuzumab heavy chain and hEPO-G4S-Her2-CL IgGlight chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection.hEPO-G4S-Her2-CLIgG was purified by Protein G chromatography (Thermo Fisher Scientific, IL). Purified proteins were analyzed by SDS-PAGE gels. FIG. 5 shows a SDS gel image of hEPO-G4S-Trastuzumab-CL in non-reducing and reducing (with 50 mM DTT) conditions. As shown in FIG. 5, Lane 1 represents hEPO-G4S-Trastuzumab-CL without DTT treatment, Lane 2 represents hEPO-G4S-Trastuzumab-CL with DTT treatment and Lane 3 represents the protein standard ladder.

### Example 7

### In-Vitro EPO Activity Test of hEPO-G4S-Her2-CL and hEPO-Coil-Her2-CH3 in TF-1 Cells

Human TF-1 cells were cultured at 37° C. with 5% CO2 in RPMI-1640 medium containing 10% fetal bovine serum (FBS), penicillin and streptomycin (50 U/mL), and 2 ng/mL human granulocyte macrophage colony stimulating factor (GM-CSF). To test the proliferative activity of Ab-hEPO, cells were washed three times with RPMI-1640 medium plus 10% FBS, resuspended in RPMI-1640 medium with 10% FBS at a density of 1.5×10⁵ cells/ml, plated in 96-well plates (1.5×10⁴ cells per well) with various concentrations of hEPO-G4S-Her2-CL (e.g., G4S.CL), hEPO-coil-Her2-CH3 (e.g., coiled coil CH3) and hEPO-bAb-H3 (positive control, e.g., hEPO.baAb) and then incubated for 72 h at 37° C. with 5% CO2. Cells were then treated with Alamar Blue (Invitrogen) for 4 h at 37° C. Cell viability was quantified using an Alamar Blue (Invitrogen) assay. Fluorescence intensity measured at 595 nm is proportional to cell viability and plotted versus protein concentration. The EC50 values were determined by fitting data into a logistic sigmoidal function: y=A2+(A1−A2)/(1+(x/x0)p), where A1 is the initial value, A2 is the final value, x0 is the inflection point of the curve, and p is the power. FIG. 6 shows a graph of antibody concentration versus cell viability. As shown in FIG. 6, Ab-hEPO fusion proteins stimulated proliferation of TF-1 cells in a dose-dependent manner. The EC₅₀ (nM) values for hEPO-G4S-Her2-CL, hEPO-coil-Her2-CH3 and hEPO-bAb-H3 were 1.294, 0.2160, and 0.2976, respectively.

### Example 8

### Binding Affinity of hEPO-G4S-Her2-CL and hEPO-Coil-Her2-CH3 Against Her2+ SK—BR-3 cells

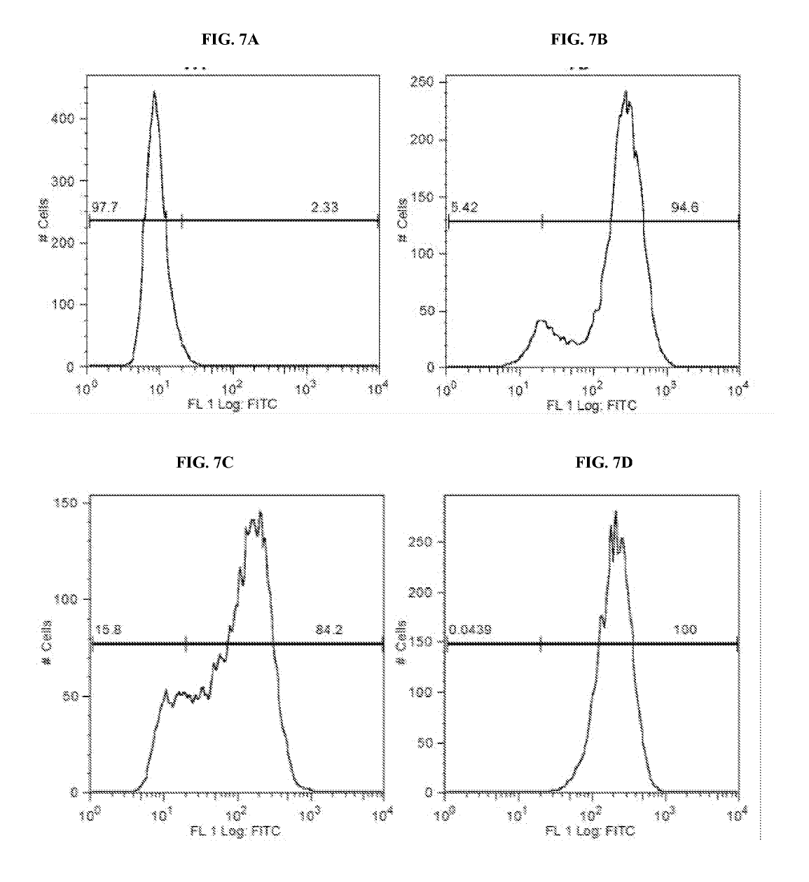

In this example, the binding affinity of of hEPO-G4S-Her2-CL, hEPO-coil-Her2-CH3 and wt.trastuzumab against Her2+ SK—BR-3 cells was determined by flow cytometry. SKBR3 cells were cultured according to vendor's protocol. Cells were centrifuged and blocked in 1× PBS with 10% FBS at 4° C. for 1 hour. 10 nM of hEPO-coil-Her2-CL, hEPO-coil-Her2-CH1 or wt.trastuzumabwas added to the cell suspensions. The cell suspensions were shaken for 1 hour at 4° C.The cells were washed 3 times with PBS. The cells were incubated with secondary antibody (e.g., Fluorescein-anti-human Fc) at 4° C. for 1 hour. The cells were washed 3 times with PBS and resuspended in PBS. The cellular fluorescence distribution was determined by flow cytometry. FIG. 7A-D depict the binding affinity of hEPO-G4S-Her2-CL, hEPO-coil-Her2-CH3 and wt.trastuzumab against Her2+ SK—BR-3 cells. FIG. 7A shows the results for cells incubated with just the secondary antibody (e.g., fluorescein-anti-human). FIG. 7B shows the results for cells incubated with the wt.trastuzumab antibody, followed by the secondary antibody incubation. FIG. 7C shows the results for cells incubated with hEPO-G4S-Her2-CL, followed by the secondary antibody incubation. FIG. 7D shows the results for cells incubated with hEPO-coil-Her2-CH3, followed by the secondary antibody incubation.

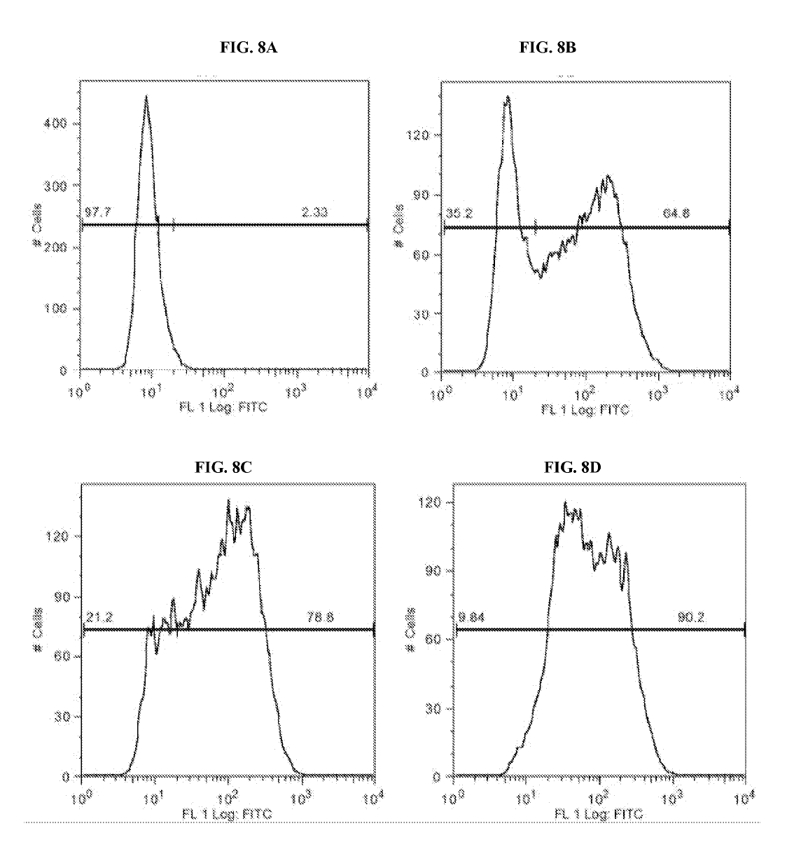

FIG. 8A-D also depicts the binding affinity of hEPO-G4S-Her2-CL, hEPO-coil-Her2-CH3 and wt.trastuzumab against Her2+ SK—BR-3 cells. FIG. 8A shows the results for cells incubated with just the secondary antibody (e.g., fluorescein-anti-human). FIG. 8B shows the results for cells incubated with the wt.trastuzumab antibody, followed by the secondary antibody incubation. FIG. 8C shows the results for cells incubated with hEPO-G4S-Her2-CL, followed by the secondary antibody incubation. FIG. 8D shows the results for cells incubated with hEPO-coil-Her2-CH3, followed by the secondary antibody incubation.

### Example 9

### Binding of wt.Trastuzumab and hEPO-Coil-Her2-CH3 Against Her2 Determined by ELISA

In this example, the binding of wt.Herception and hEPO-coil-Her2-CH3 against Her2 was determined by ELISA. hErbB2-Fc was diluted to a final concentration of 10 μg/ml in PBS. Wells of a PVC microtiter plate were coated with the antigen (e.g., hErbB2-Fc) overnight at 4° C. The coating solution was removed and the plate was washed three times with PBS. The remaining protein-binding sites in the coated wells were blocked by adding 5% serum in PBS. The microtiter plate was incubated at room temperature for 2 hours. The plate was washed twice with PBS. 100 μl of diluted wt.Trastuzumab or hEPO-coil-Her2-CH3 were added to each well. The plate was incubated for 2 h at room temperature. The plate was washed four times with PBS. 100 μl of HRP-anti-kappa was added to each well. The plate was covered with an adhesive plastic and incubated for 1-2 hrs at room temperature. The plate was washed four times with PBS. 100 μL of QuantaBlu WS was added to each well and incubated for 1.5-90 minutes at RT. Fluorescence intensity was determined with fluorescence plate reader with λex=325 nm and λem=420 nm. FIG. 9 shows the binding of various concentrations of wt.Trastuzumab and hEPO-coil-Her2-CH3 against Her2 as determined by ELISA. As shown in FIG. 9, the concentration of the antibody or antibody fusions was plotted against the relative luciferase units. For each concentration, the first bar represents hEPO-coil-Her2-CH3 and the second bar represents wt.Trastuzumab. As shown in FIG. 9, wt.Trastuzumab and the trastuzumab fusion proteins had similar binding affinity to Her2.

### Example 10

### Cloning, Expression and Purification of Anti-CD19ScFv-UCHT1-CL(Fab)

Cloning: Mammalian expression vector of UCHT1Fab heavy chain was generated by ligation of amplified UCHT1 Fab heavy chain (VH and CH1) to pFuse-hIgG1-Fc backbone vector (InvivoGen, CA) without Fc fragment. A gene encoding antibody UCHT1 light chain was amplified and cloned into the pFuse vector without hIgG1 Fc fragment. A gene encodinganti-CD19ScFv (with (GGGGS)3 (SEQ ID NO: 73) as a linker between heavy and light chain of anti-CD19) was synthesized by Genscript (NJ, USA), and amplified by polymerase chain reaction (PCR). A floppy linker was added to each end of the anti-CD19ScFv insert. The sequence of the ascending adapter peptide with linkers at each end is: H2N-GGGGSGGGGSGGGGS-COOH (SEQ ID NO: 73); the sequence of the descending peptide with linkers at each end is: H2N-GGGGS-COOH (SEQ ID NO: 72). Subsequently, anti-CD19ScFv-UCHT1-CL fusion proteins were created by replacing the K169 in CL region of UCHT1 light chain with anti-CD19ScFv with linker sequences at both ends. The resulting mammalian expression vectors were confirmed by DNA sequencing.

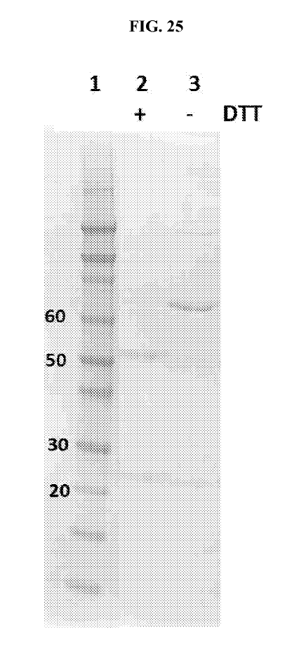

Expression and Purification: anti-CD19ScFv-UCHT1-CL(Fab) was expressed through transient transfection of FreeStyle HEK 293 cells with expression vectors of UCHT1-Fab heavy chain and anti-CD19ScFv-UCHT1-CLlight chain, according to the manufacturer's protocol. Briefly, 28 mL FreeStyle HEK 293 cells containing 3×107 cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection.anti-CD19ScFv-UCHT1-CL(Fab) was purified by Protein G chromatography (Thermo Fisher Scientific, IL). Purified proteins were analyzed by SDS-PAGE gels. FIG. 25 shows a SDS gel image of CD19ScFv-UCHT1-CL (Fab) in non-reducing and reducing (with 50 mM DTT) conditions. As shown in FIG. 25, Lane 1 represents the protein standard ladder, Lane 2 represents CD19ScFv-UCHT1-CL(Fab) with DTT treatment and Lane 3 represents CD19ScFv-UCHT1-CL(Fab) without DTT treatment.

### Example 11

### Binding Affinity of CD19ScFv-UCHT1-CL(Fab) Against Nalm-6 and K562 Cells

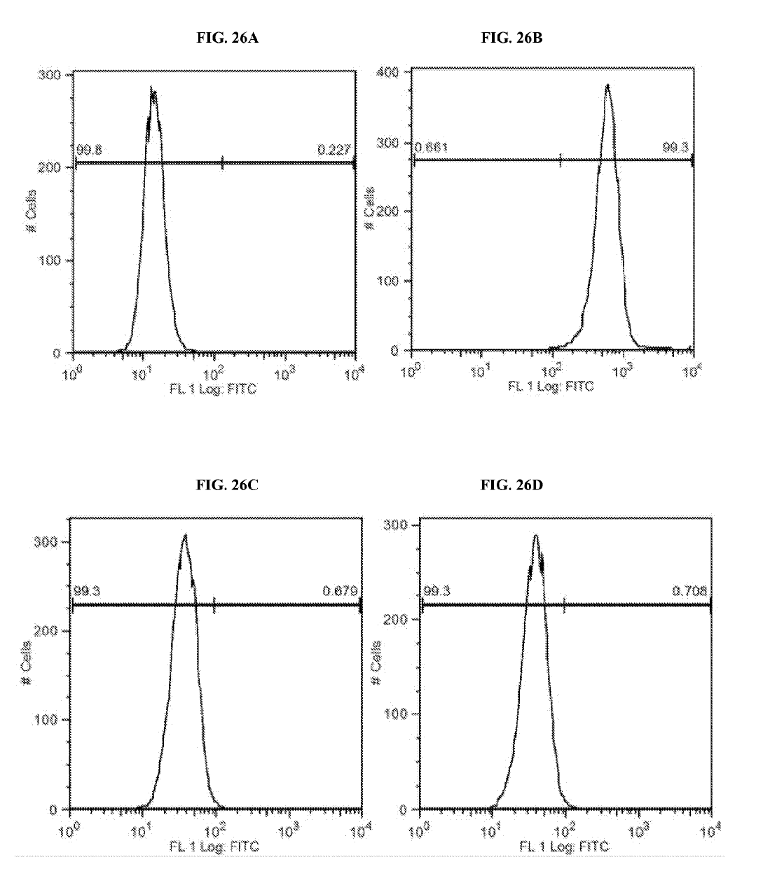

Nalm-6 and K562 cells were cultured according to vendor's protocol. Cells were centrifuged and blocked in 1× PBS with 10% FBS at 4° C. for 1 hour. 10 nM of CD19ScFv-UCHT1-CL(Fab) was added to the cell suspensions. The cell suspensions were shaken for 1 hour at 4° C. The cells were washed 3 times with PBS. The cells were incubated with a secondary antibody (e.g., Fluorescein-anti-human IgG or A488-anti-hIgG) at 4° C. for 1 hour. The cells were washed 3 times with PBS and resuspended in PBS. The cellular fluorescence distribution was determined by flow cytometry. FIG. 26A-D show graphs of the binding affinity of CD19ScFv-UCHT1-CL(Fab) against Nalm-6 or K562 cells. FIG. 26A shows the flow cytometry results for Nalm-6 cells incubated with only the secondary antibody. FIG. 26B shows the flow cytometry results for Nalm-6 cells incubated with CD19ScFv-UCHT1-CL(Fab) and the secondary antibody. FIG. 26C shows the flow cytometry results for K562 cells incubated with only the secondary antibody. FIG. 26D shows the flow cytometry results for K562 cells incubated with CD19ScFv-UCHT1-CL(Fab) and the secondary antibody. As shown in FIG. 26B, CD19ScFv-UCHT1-CL(Fab) binds to the Nalm-6 cells, which are CD19 positive cells. However, as shown in FIG. 26D, CD19ScFv-UCHT1-CL(Fab) does not bind to K562 cells, which are CD19 negative cells.

### Example 12

### Cloning, Expression and Purification of TCP1-Coil-UCHT1-CL (Fab)

Cloning: Mammalian expression vector of UCHT1 Fab heavy chain was generated by ligation of amplified UCHT1 Fab heavy chain (VH and CH1) to pFuse-hIgG1-Fc backbone vector (InvivoGen, CA) without Fc fragment. A gene encoding antibody UCHT1 light chain was amplified and cloned into the pFuse vector without hIgG1 Fc fragment. A gene encoding TCP1 (TPSPFSH=SEQ ID NO: 78) with an ascending adapter peptide of H2N-GGSGAKLAALKAKLAALKAKL-COOH (SEQ ID NO: 75) and a descending peptide of H2N-LEAELAALEAELAALEAGGSG-COOH (SEQ ID NO: 74) was synthesized by IDT gBlock gene synthesis. Subsequently, TCP1-UCHT1-CL fusion proteins were created by replacing the K169 in CL region of UCHT1 light chain with TCP1 with linker sequences at both ends. The resulting mammalian expression vectors were confirmed by DNA sequencing.

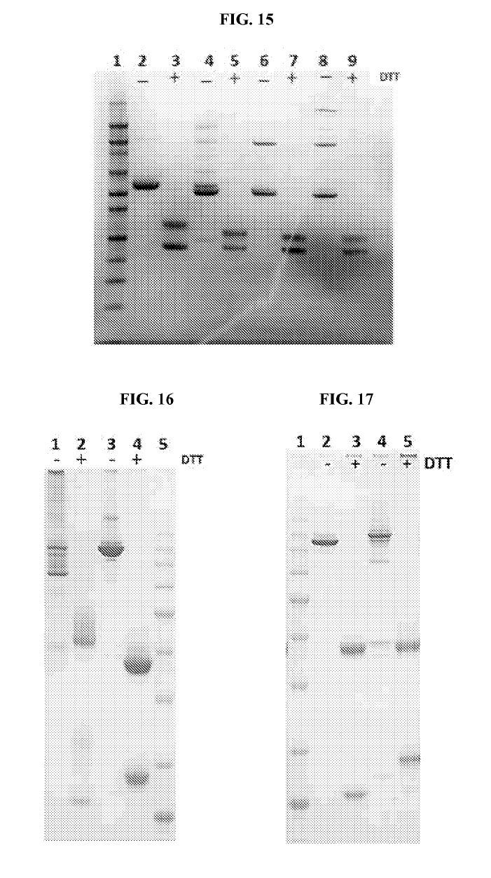

Expression and Purification: TCP1-coil-UCHT1-CLwas expressed through transient transfection of FreeStyle HEK 293 cells with expression vectors of UCHT1-Fab heavy chain and TCP1-coil-UCHT1-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. TCP1-coil-UCHT1-CL was purified by Protein G chromatography (Thermo Fisher Scientific, IL). Purified proteins were analyzed by SDS-PAGE gels. FIG. 15 shows a SDS gel image of TCP1-coil-UCHT1-CL. As shown in FIG. 15, Lane 1 represents the protein standard marker, Lane 6 represents TCP1-coil-UCHT1-CL without DTT treatment and Lane 7 represents TCP1-coil-UCHT1-CL with DTT treatment.

### Example 13

### Cloning, Expression and Purification of TCP1-Coil-UCHT1-CL (IgG)

Cloning: Mammalian expression vector of UCHT1 IgG heavy chain was generated by in-frame ligation of amplified UCHT1 Fab heavy chain (VH and CH1) to pFuse-hIgG1-Fc backbone vector (InvivoGen, CA). A gene encoding antibody UCHT1 light chain was amplified and cloned into the pFuse vector without hIgG1 Fc fragment. A gene encoding TCP1 (TPSPFSH=SEQ ID NO: 78) with an ascending adapter peptide of H2N-GGSGAKLAALKAKLAALKAKL-COOH (SEQ ID NO: 75) and a descending peptide of H2N-LEAELAALEAELAALEAGGSG-COOH (SEQ ID NO: 74) was synthesized by IDT gBlock gene synthesis. Subsequently, TCP1-UCHT1-CL fusion proteins were created by replacing the K169 in CL region of UCHT1 light chain with TCP1 with linker sequences at both ends. The resulting mammalian expression vectors were confirmed by DNA sequencing.

Expression and Purification: TCP1-coil-UCHT1-CL was expressed through transient transfection of FreeStyle HEK 293 cells with expression vectors of UCHT1-IgG heavy chain and TCP1-coil-UCHT1-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. TCP1-coil-UCHT1-CL was purified by Protein G chromatography (Thermo Fisher Scientific, IL). Purified proteins were analyzed by SDS-PAGE gels.

### Example 14

### Cloning, Expression and Purification of TCP1-UCHT1-CL

Cloning: Mammalian expression vector of UCHT1 IgG heavy chain was generated by in-frame ligation of amplified UCHT1 Fab heavy chain (VH and CH1) to pFuse-hIgG1-Fc backbone vector (InvivoGen, CA). A gene encoding antibody UCHT1 light chain was amplified and cloned into the pFuse vector without hIgG1 Fc fragment. A gene encoding TCP1 (CTPSPFSHC=SEQ ID NO: 79) with GGGGS (SEQ ID NO: 72) linker at both ends was synthesized as oligonucleotides. Subsequently, TCP1-UCHT1-CL fusion proteins were created by replacing the K169 in CL region of UCHT1 light chain with TCP1 with linker sequences at both ends. The resulting mammalian expression vectors were confirmed by DNA sequencing.

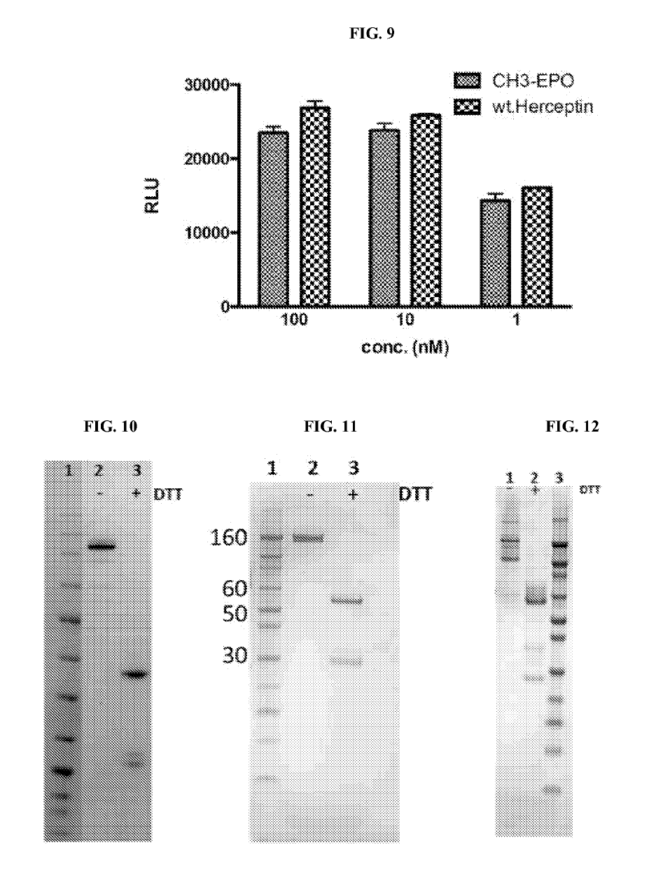

Expression and Purification: TCP1-UCHT1-CL was expressed through transient transfection of FreeStyle HEK 293 cells with expression vectors of UCHT1-IgG heavy chain and TCP1-UCHT1-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. TCP1-UCHT1-CL was purified by Protein G chromatography (Thermo Fisher Scientific, IL). Purified proteins were analyzed by SDS-PAGE gels. FIG. 10 shows a SDS gel image of TCP1-G4S-UCHT1-CL (e.g., TCP1-UCHT1-CL) in non-reducing and reducing (with 50 mM DTT) conditions. As shown in FIG. 10, Lane 1 represents the protein standard ladder, Lane 2 represents TCP1-G4S-UCHT1-CL without DTT treatment and Lane 3 represents TCP1-G4S-UCHT1-CL with DTT treatment.

### Example 15

### Cloning, Expression and Purification of NGR-Coil-UCHT1-CL

Cloning: Mammalian expression vector of UCHT1 Fab heavy chain was generated by ligation of amplified UCHT1 Fab heavy chain (VH and CH1) to pFuse-hIgG1-Fc backbone vector (InvivoGen, CA) without Fc fragment. A gene encoding antibody UCHT1 light chain was amplified and cloned into the pFuse vector without hIgG1 Fc fragment. A gene encoding NGR (TYNGRT=SEQ ID NO: 80) with an ascending adapter peptide of H2N-GGSGAKLAALKAKLAALKAKL-COOH (SEQ ID NO: 75) and a descending peptide of H2N-LEAELAALEAELAALEAGGSG-COOH (SEQ ID NO: 74) was synthesized by IDT gBlock gene synthesis. Subsequently, NGR-UCHT1-CL fusion proteins were created by replacing the K169 in CL region of UCHT1 light chain with NGR with linker sequences at both ends. The resulting mammalian expression vectors were confirmed by DNA sequencing.

Expression and Purification: NGR-coil-UCHT1-CL was expressed through transient transfection of FreeStyle HEK 293 cells with expression vectors of UCHT1-Fab heavy chain and NGR-coil-UCHT1-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. NGR-coil-UCHT1-CL was purified by Protein G chromatography (Thermo Fisher Scientific, IL). Purified proteins were analyzed by SDS-PAGE gels. As shown in FIG. 15, Lane 1 represents the protein standard marker, Lane 8 represents NGR-coil-UCHT1-CL without DTT treatment and Lane 9 represents NGR-coil-UCHT1-CL with DTT treatment.

### Example 16

### Cloning, Expression and Purification of NGR-UCHT1-CL (e.g., NGR-G4S-UCHT1-CL)

Cloning: Mammalian expression vector of UCHT1 IgG heavy chain was generated by in-frame ligation of amplified UCHT1 Fab heavy chain (VH and CH1) to pFuse-hIgG1-Fc backbone vector (InvivoGen, CA). A gene encoding antibody UCHT1 light chain was amplified and cloned into the pFuse vector without hIgG1 Fc fragment. A gene encoding NGR (CNGRCVSGCAGRC=SEQ ID NO: 81) with GGGGS (SEQ ID NO: 72) linker at both ends was synthesized as oligonucleotides. Subsequently, NGR-UCHT1-CL fusion proteins were created by replacing the K169 in CL region of UCHT1 light chain with NGR with linker sequences at both ends. The resulting mammalian expression vectors were confirmed by DNA sequencing.

Expression and Purification: NGR-UCHT1-CL was expressed through transient transfection of FreeStyle HEK 293 cells with expression vectors of UCHT1-IgG heavy chain and NGR-UCHT1-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. NGR-UCHT1-CL was purified by Protein G chromatography (Thermo Fisher Scientific, IL). Purified proteins were analyzed by SDS-PAGE gels. FIG. 11 shows a SDS gel image of the recombinant protein expression in 30 ml 293 free cells system. UCHT1 heavy chain is paired with NGR-UCHT11 light chain. As shown in FIG. 11, Lane 1 represents the protein standard ladder, Lane 2 represents NGR-UCHT1-CL without DTT treatment and Lane 3 represents NGR-UCHT1-CL with DTT treatment. The yield of UCTH1/NGR-UCTH1 was 1.59 mg/L

### Example 17

### Binding of NGR-G4S-UCHT1-CL Against CD13+ Positive HT-1080 Cells and MDA-MB-435 Cells (Negative Control

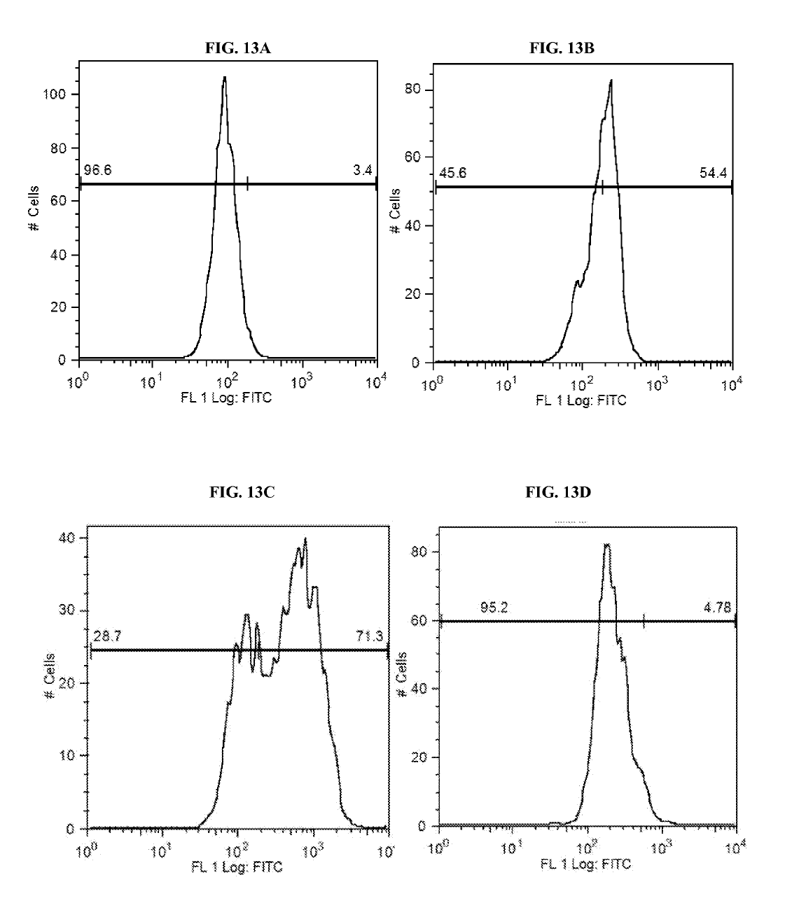

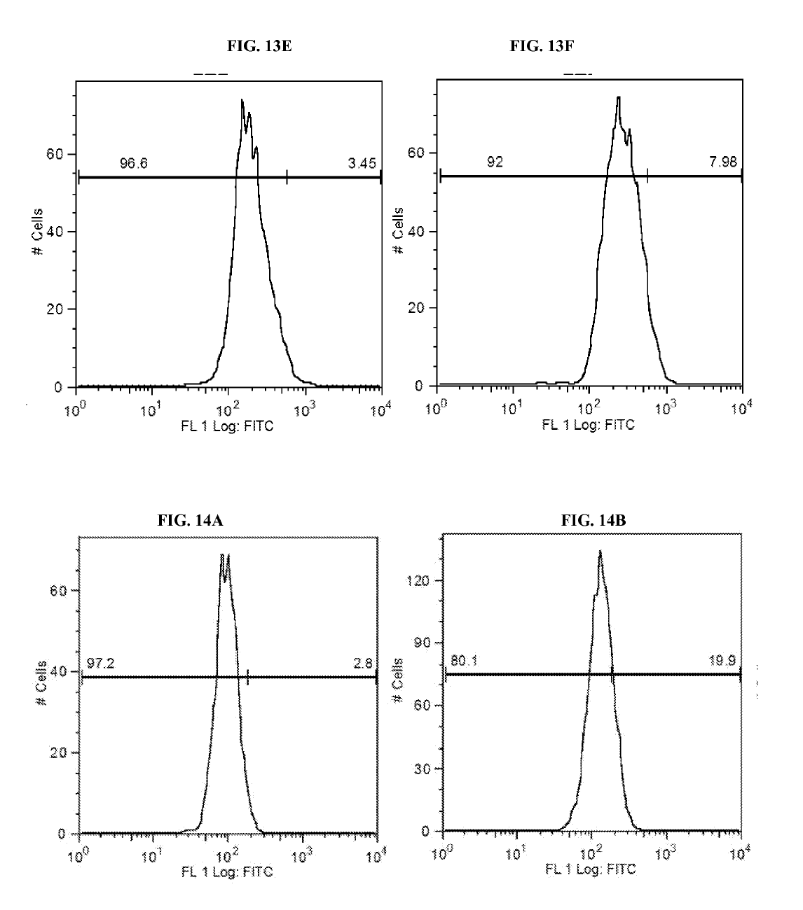

HT-1080 and MDA-MB-435 cells were cultured according to vendor's protocol. Cells were centrifuged and blocked in 1× PBS with 10% FBS at 4° C. for 1 hour. 0 nM, 10 nM, or 100 nM of NGR-G4S-UCHT1-CL (e.g., NGR-UCHT1-CL) was added to the cell suspensions. The cell suspensions were shaken for 1 hour at 4° C.The cells were washed 3 times with PBS. The cells were incubated with a secondary antibody (e.g., Fluorescein-anti-human Fc) at 4° C. for 1 hour. The cells were washed 3 times with PBS and resuspended in PBS. The cellular fluorescence distribution was determined by flow cytometry. FIG. 13A-F shows graphs of the binding of NGR-G4S-UCHT1-CL against CD13+ positive HT-1080 cells and MDA-MB-435 cells (negative control). FIG. 13A-C shows the binding of NGR-G4S-UCHT1 against HT-1080 cells with 0 nM, 10 nM or 100 nM of NGR-G4S-UCHT1-CL, respectively. FIG. 13D-F shows the binding of NGR-G4S-UCHT1 against MDA-MD-435 cells with 0 nM, 10 nM or 100 nM of NGR-G4S-UCHT1-CL, respectively.

### Example 18

### Binding of TCP1-G4S-UCHT1-CL Against Colorectal Cancer Cells (HT-29) and MDA-MB-435 Cells (Negative Control)

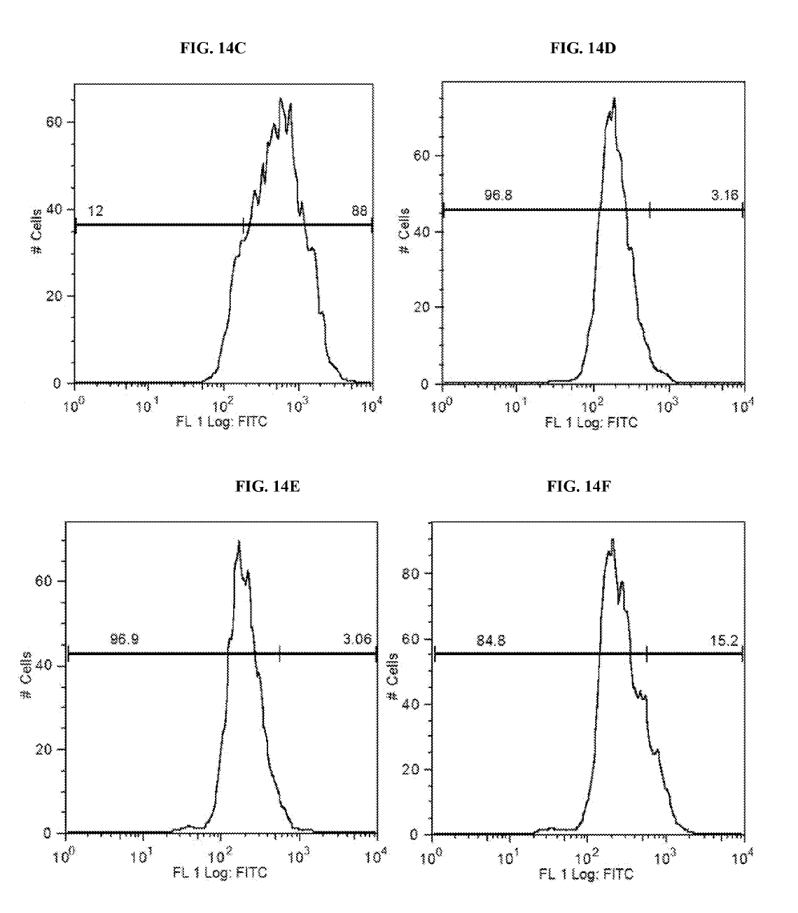

HT-29 and MDA-MB-435 cells were cultured according to vendor's protocol. Cells were centrifuged and blocked in 1+ PBS with 10% FBS at 4° C. for 1 hour. 0 nM, 10 nM, or 100 nM of TCP1-G4S-UCHT1-CL (e.g., TCP1-UCHT1-CL) was added to the cell suspensions. The cell suspensions were shaken for 1 hour at 4° C. The cells were washed 3 times with PBS. The cells were incubated with a secondary antibody (e.g., Fluorescein-anti-human Fc) at 4° C. for 1 hour. The cells were washed 3 times with PBS and resuspended in PBS. The cellular fluorescence distribution was determined by flow cytometry. FIG. 14A-F shows graphs of the binding of TCP1-G4S-UCHT1-CL against colorectal cancer cells (HT-29) and MDA-MB-435 cells (negative control). FIG. 14A-C shows the binding of TCP1-G4S-UCHT1-CL against HT-29 cells with 0 nM, 10 nM or 100 nM of TCP1-G4S-UCHT1-CL, respectively. FIG. 14D-F shows the binding of TCP1-G4S-UCHT1-CL against MDA-MD-435 cells with 0 nM, 10 nM or 100 nM of TCP1-G4S-UCHT1-CL, respectively.

### Example 19

### Cloning, Expression and Purification of Integrin-UCHT1-CL (Fab) (e.g., Int-Coil-UCHT1-CL)

Cloning: Mammalian expression vector of UCHT1 Fab heavy chain was generated by ligation of amplified UCHT1 Fab heavy chain (VH and CH1) to pFuse-hIgG1-Fc backbone vector (InvivoGen, CA) without Fc fragment. A gene encoding antibody UCHT1 light chain was amplified and cloned into the pFuse vector without hIgG1 Fc fragment. A gene encoding Int (GCPQGRGDWAPTSCKQDSDCRAGCVCGPNGFCG=SEQ ID NO: 82) with an ascending adapter peptide of H2N-GGSGAKLAALKAKLAALKGGGGS-COOH (SEQ ID NO: 77) and a descending peptide of H2N-GGGGSELAALEAELAALEAGGSG-COOH (SEQ ID NO: 76) was synthesized by IDT gBlock gene synthesis. Subsequently, Int-UCHT1-CL fusion proteins were created by replacing the K169 in CL region of UCHT1 light chain with Int with linker sequences at both ends. The resulting mammalian expression vectors were confirmed by DNA sequencing.

Expression and Purification: Int-coil-UCHT1-CLwas expressed through transient transfection of FreeStyle HEK 293 cells with expression vectors of UCHT1-Fab heavy chain and Int-coil-UCHT1-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. Int-coil-UCHT1-CL was purified by Protein G chromatography (Thermo Fisher Scientific, IL). Purified proteins were analyzed by SDS-PAGE gels. FIG. 15 shows a SDS gel image of Int-coil-UCHT1-CL. As shown in FIG. 15, Lane 1 represents the protein standard marker, Lane 2 represents Int-coil-UCHT1-CL without DTT treatment and Lane 3 represents Int-coil-UCHT1-CL with DTT treatment.

### Example 20

### Cloning, Expression and Purification of CXCR4-BP-Coil-CD20-CL (Fab)

Cloning: A mammalian expression vector of CD20 Fab heavy chain was generated by ligation of amplified CD20 Fab heavy chain (VH and CH1) to pFuse-hIgG1-Fc backbone vector (InvivoGen, CA) without Fc fragment. A gene encoding antibody CD20 light chain were amplified and cloned into the pFuse vector without hIgG1 Fc fragment. A gene encoding CXCR4-BP (YRKCRGGRRWCYQK=SEQ ID NO: 83) with an ascending adapter peptide of H2N-GGSGAKLAALKAKLAALKAKL-COOH (SEQ ID NO: 75) and a descending peptide of H2N-LEAELAALEAELAALEAGGSG-COOH (SEQ ID NO: 74) was synthesized by IDT gBlock gene synthesis. Subsequently, CXCR4-BP-CD20-CL fusion proteins were created by replacing the K169 in CL region of CD20 light chain with CXCR4-BP with linker sequences at both ends. The resulting mammalian expression vectors were confirmed by DNA sequencing.

Expression and Purification: CXCR4-BP-coil-CD20-CL was expressed through transient transfection of FreeStyle HEK 293 cells with expression vectors of CD20-Fab heavy chain and CXCR4-BP-coil-CD20-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. CXCR4-BP-coil-CD20-CL was purified by Protein G chromatography (Thermo Fisher Scientific, IL). Purified proteins were analyzed by SDS-PAGE gels. FIG. 15 shows a SDS gel image of CXCR4-BP-coil-CD20-CL(Fab). As shown in FIG. 15, Lane 1 represents the protein standard marker, Lane 4 represents CXCR4-BP-coil-CD20-CL(Fab) without DTT treatment and Lane 5 represents CXCR4-BP-coil-CD20-CL(Fab) with DTT treatment.

### Example 21

### Cloning, Expression and Purification of CXCR4-BP-Coil-CD20-CL (IgG)

Cloning: Mammalian expression vector of CD20 IgG heavy chain was generated by in-frame ligation of amplified CD20 Fab heavy chain (VH and CH1) to pFuse-hIgG1-Fc backbone vector (InvivoGen, CA). A gene encoding antibody CD20 light chain was amplified and cloned into the pFuse vector without hIgG1 Fc fragment. A gene encoding CXCR4-BP (YRKCRGGRRWCYQK=SEQ ID NO: 83) with an ascending adapter peptide (H2N-GGSGAKLAALKAKLAALKAKL-COOH=SEQ ID NO: 75) and a descending peptide (H2N-LEAELAALEAELAALEAGGSG-COOH=SEQ ID NO: 74) was synthesized by IDT gBlock gene synthesis. Subsequently, CXCR4-BP-CD20-CL fusion proteins were created by replacing the K169 in CL region of CD20 light chain with CXCR4-BP with linker sequences at both ends. The resulting mammalian expression vectors were confirmed by DNA sequencing.

Expression and Purification: CXCR4-BP-coil-CD20-CL was expressed through transient transfection of FreeStyle HEK 293 cells with expression vectors of CD20-IgG heavy chain and CXCR4-BP-coil-CD20-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. CXCR4-BP-coil-CD20-CL was purified by Protein G chromatography (Thermo Fisher Scientific, IL). Purified proteins were analyzed by SDS-PAGE gels. FIG. 17 shows a SDS gel image of CD20 and CXCR4-BP-coil-CD20-CL(IgG) fusion proteins. As shown in FIG. 17, Lane 1 represents the protein standard ladder, Lane 2 represents CD20 without DTT treatment, Lane 3 represents CD20 with DTT treatment, Lane 4 represents CXCR4-BP-coil-CD20-CL(IgG) without DTT treatment and Lane 5 represents CXCR4-BP-coil-CD20-CL(IgG) with DTT treatment.

### Example 22

### Binding Affinity of of CD20Fab, CXCR4-BP-Coil-CD20(Fab), and CXCR4-BP-Palivizumab Against CD20+/CXCR4dim BJAB Cells

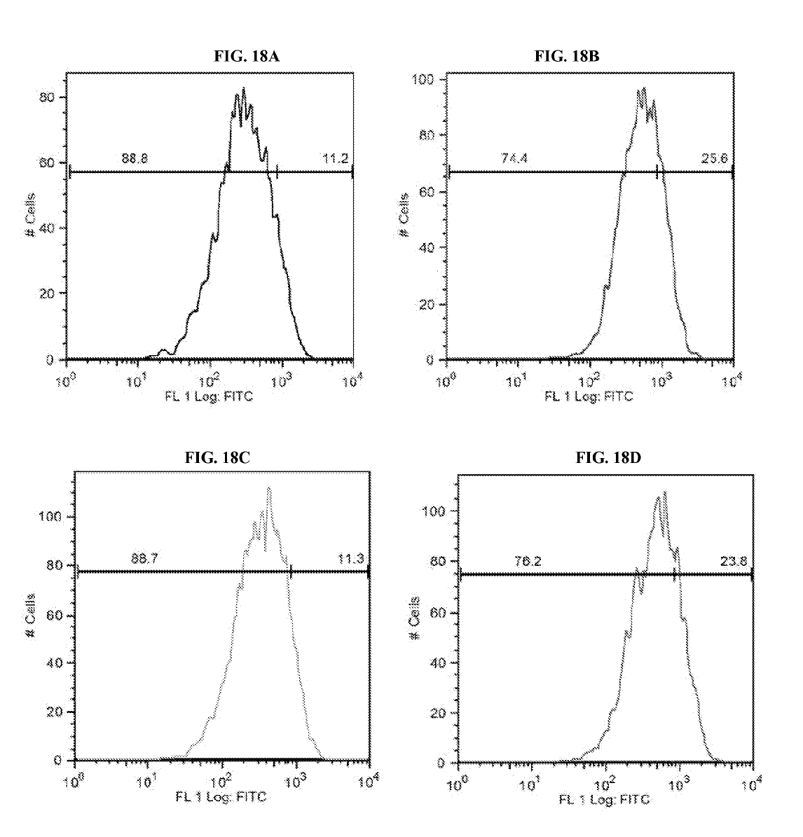

BJAB cells were cultured according to vendor's protocol. Cells were centrifuged and blocked in 1× PBS with 10% FBS at 4° C. for 1 hour. 50 nM of CD20Fab, CXCR4-BP-coil-CD20(Fab), or CXCR4-BP-Palivizumab were added to the cell suspensions. The cell suspensions were shaken for 1 hour at 4° C.The cells were washed 3 times with PBS. The cells were incubated with a secondary antibody (e.g., A488-anti-hIgG) at 4° C. for 1 hour. The cells were washed 3 times with PBS and resuspended in PBS. The cellular fluorescence distribution was determined by flow cytometry. FIG. 18A-D show graphs of the binding affinity of CD20Fab, CXCR4-BP-coil-CD20(Fab), and CXCR4-BP-Palivizumab against CD20+/CXCR4dim BJAB cells. FIG. 18A shows the flow cytometry results for BJAB cells incubated with only the secondary antibody. FIG. 18B shows the flow cytometry results for BJAB cells incubated with CD20Fab and the secondary antibody. FIG. 18C shows the flow cytometry results for BJAB cells incubated with CXCR4-BP-coil-CD20Fab and the secondary antibody. FIG. 18D shows the flow cytometry results for BJAB cells incubated with CXCR4-BP-Palivizumab and the secondary antibody.

### Example 23

### Binding Affinity of CD20Fab, CXCR4-BP-Coil-CD20(Fab), and CXCR4-BP-Palivizumab Against CD20dim/CXCR4+ Nalm-6 Cells

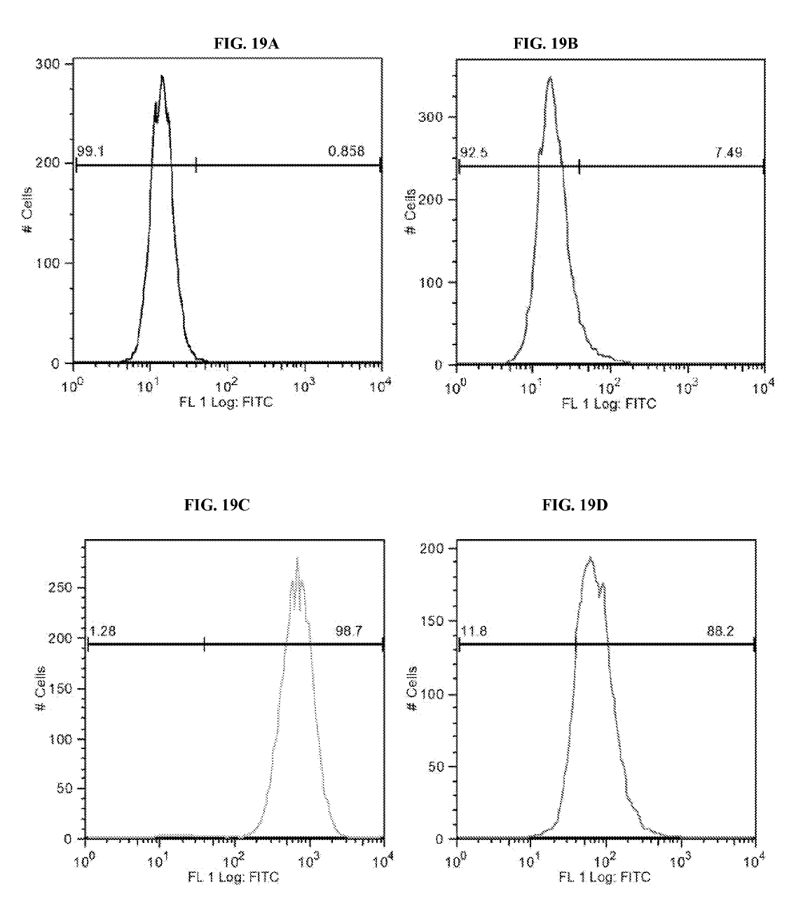

Nalm-6 cells were cultured according to vendor's protocol. Cells were centrifuged and blocked in 1× PBS with 10% FBS at 4° C. for 1 hour. 50 nM of CD20Fab, CXCR4-BP-coil-CD20(Fab), or CXCR4-BP-Palivizumab were added to the cell suspensions. The cell suspensions were shaken for 1 hour at 4° C.The cells were washed 3 times with PBS. The cells were incubated with a secondary antibody (e.g., A488-anti-hIgG) at 4° C. for 1 hour. The cells were washed 3 times with PBS and resuspended in PBS. The cellular fluorescence distribution was determined by flow cytometry. FIG. 19A-D show graphs of the binding affinity of CD20Fab, CXCR4-BP-coil-CD20(Fab), and CXCR4-BP-Palivizumab against CD20dim/CXCR4+ Nalm-6 cells. FIG. 19A shows the flow cytometry results for Nalm-6 cells incubated with only the secondary antibody. FIG. 19B shows the flow cytometry results for Nalm-6 cells incubated with CD20Fab and the secondary antibody. FIG. 19C shows the flow cytometry results for Nalm-6 cells incubated with CXCR4-BP-coil-CD20Fab and the secondary antibody. FIG. 19D shows the flow cytometry results for Nalm-6 cells incubated with CXCR4-BP-Palivizumab and the secondary antibody.

### Example 24

### Binding Affinity of CD20Fab, CXCR4-BP-Coil-CD20(Fab), and CXCR4-BP-Palivizumab Against CD20−/CXCR4dim K562 Cells

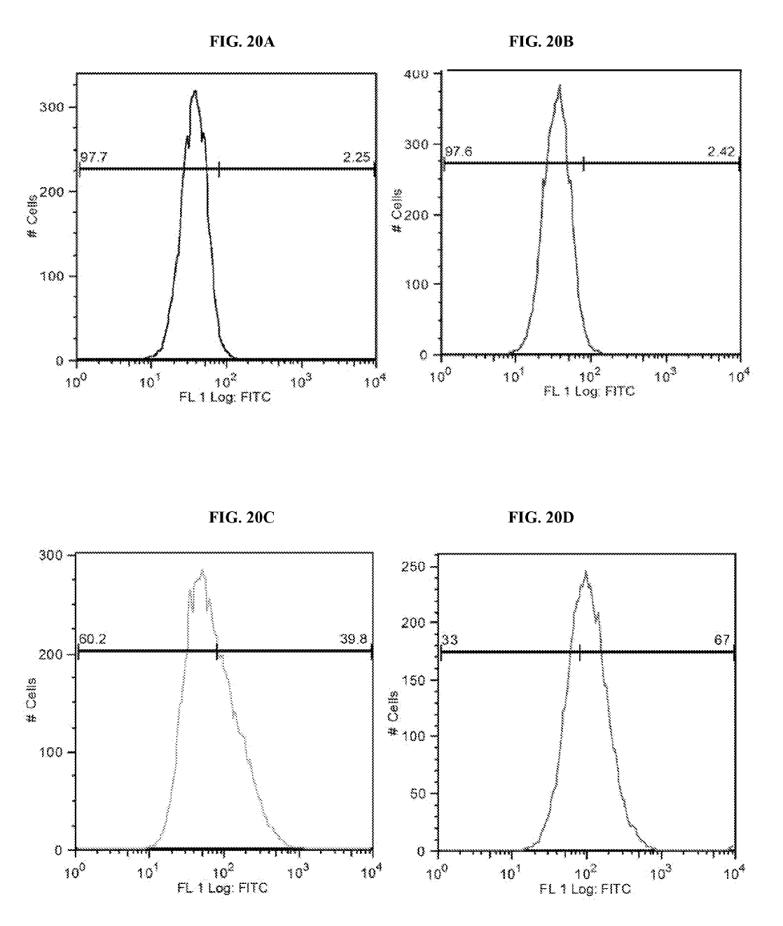

K562 cells were cultured according to vendor's protocol. Cells were centrifuged and blocked in 1× PBS with 10% FBS at 4° C. for 1 hour. 50 nM of CD20Fab, CXCR4-BP-coil-CD20(Fab), or CXCR4-BP-Palivizumab were added to the cell suspensions. The cell suspensions were shaken for 1 hour at 4° C.The cells were washed 3 times with PBS. The cells were incubated with a secondary antibody (e.g., A488-anti-hIgG) at 4° C. for 1 hour. The cells were washed 3 times with PBS and resuspended in PBS. The cellular fluorescence distribution was determined by flow cytometry. FIG. 20A-D show graphs of the binding affinity of CD20Fab, CXCR4-BP-coil-CD20(Fab), and CXCR4-BP-Palivizumab against CD20−/CXCR4dim K562 cells. FIG. 20A show the flow cytometry results for K562 cells incubated with only the secondary antibody. FIG. 20B shows the flow cytometry results for K562cells incubated with CD20Fab and the secondary antibody. FIG. 20C shows the flow cytometry results for K562 cells incubated with CXCR4-BP-coil-CD20Fab and the secondary antibody. FIG. 20D shows the flow cytometry results for K562 cells incubated with CXCR4-BP-Palivizumab and the secondary antibody.

### Example 25

### Binding Affinity of Anti-CD20, CXCR4-BP-Coil-CD20(IgG), CXCR4-BP-Her2-CL and CXCR4-BP-Her2-CH1 Against CD20+/CXCR4+ Raji Cells

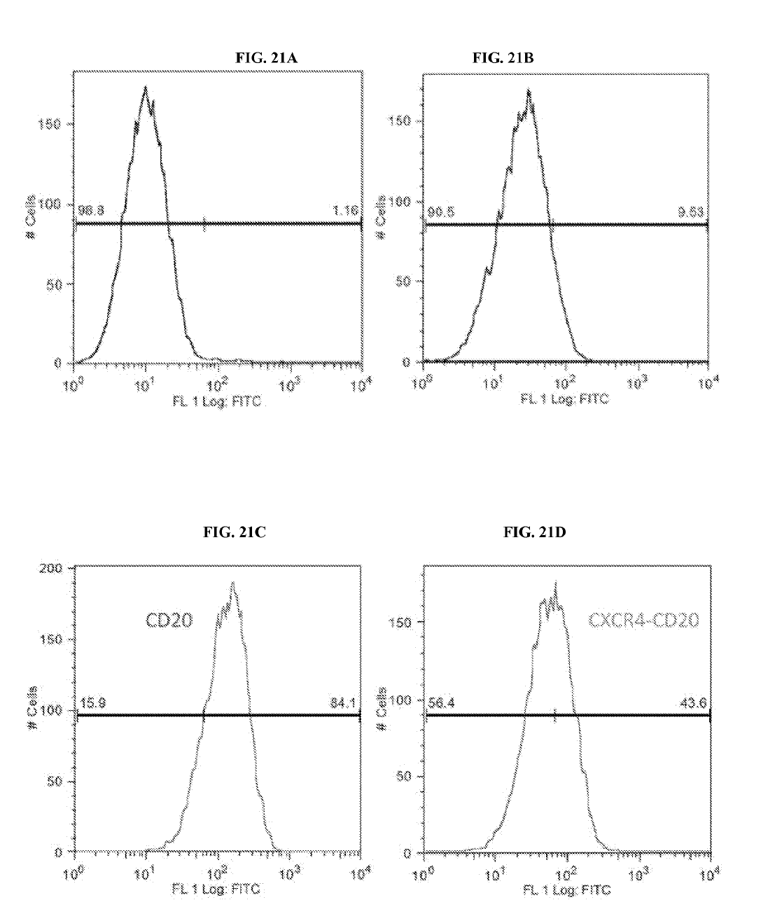

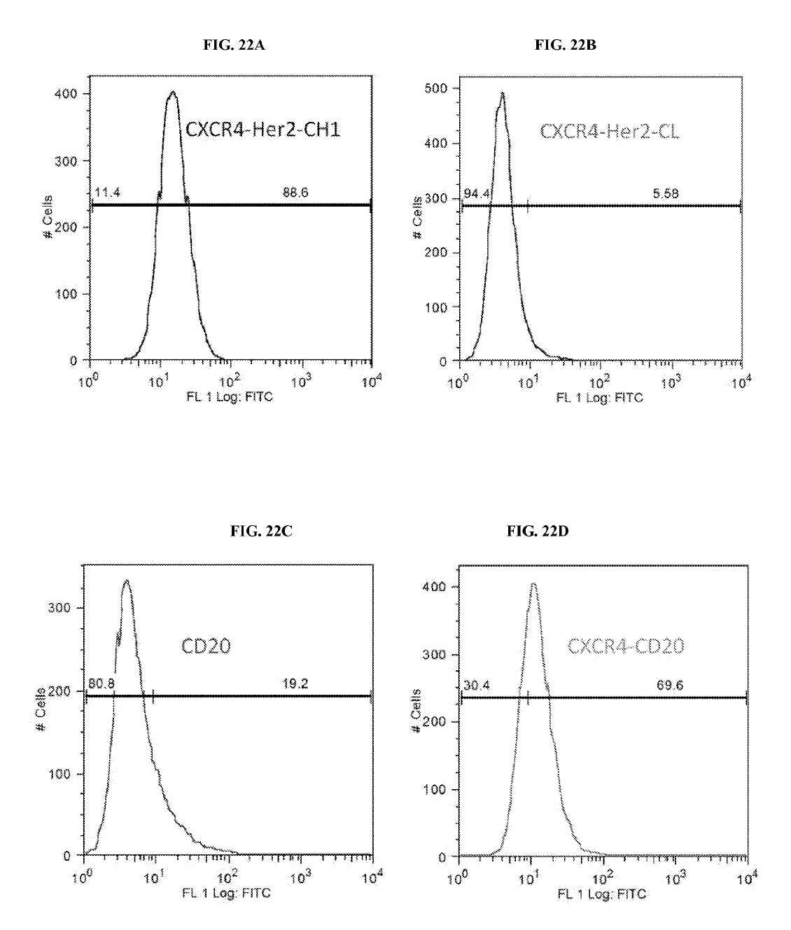

Raji cells were cultured according to vendor's protocol. Cells were centrifuged and blocked in 1× PBS with 10% FBS at 4° C. for 1 hour. 50 nM of anti-CD20, CXCR4-BP-coil-CD20(IgG), CXCR4-BP-Her2-CL and CXCR4-BP-Her2-CH1 were added to the cell suspensions. The cell suspensions were shaken for 1 hour at 4° C.The cells were washed 3 times with PBS. The cells were incubated with a secondary antibody (e.g., fluorescein-anti-hFc) at 4° C. for 1 hour. The cells were washed 3 times with PBS and resuspended in PBS. The cellular fluorescence distribution was determined by flow cytometry. FIG. 21A-D show graphs of the binding affinity of anti-CD20, CXCR4-BP-coil-CD20(IgG), CXCR4-BP-Her2-CL and CXCR4-BP-Her2-CH1 against CD20+/CXCR4+ Raji cells. FIG. 21A shows the flow cytometry results for Raji cells incubated with CXCR4-BP-Her2-CH1. FIG.21B shows the flow cytometry results for Raji cells incubated with CXCR4-BP-Her2-CL. FIG. 21C shows the flow cytometry results for Raji cells incubated with anti-CD20. FIG. 21D shows the flow cytometry results for Raji cells incubated with CXCR4-BP-coil-CD20(IgG).

### Example 26

### Binding Affinity of Anti-CD20, CXCR4-BP-Coil-CD20(IgG), CXCR4-BP-Her2-CL and CXCR4-BP-Her2-CH1 Against CD20−/CXCR4+ Nalm-6 Cells

Nalm-6 cells were cultured according to vendor's protocol. Cells were centrifuged and blocked in 1× PBS with 10% FBS at 4° C. for 1 hour. 50 nM of anti-CD20, CXCR4-BP-coil-CD20(IgG), CXCR4-BP-Her2-CL and CXCR4-BP-Her2-CH1 were added to the cell suspensions. The cell suspensions were shaken for 1 hour at 4° C. The cells were washed 3 times with PBS. The cells were incubated with a secondary antibody (e.g., fluorescein-anti-hFc) at 4° C. for 1 hour. The cells were washed 3 times with PBS and resuspended in PBS. The cellular fluorescence distribution was determined by flow cytometry. FIG. 22A-D show graphs of the binding affinity of anti-CD20, CXCR4-BP-coil-CD20(IgG), CXCR4-BP-Her2-CL and CXCR4-BP-Her2-CH1 against CD20−/CXCR4+ Nalm-6 cells. FIG. 22A shows the flow cytometry results for Nalm-6 cells incubated with CXCR4-BP-Her2-CH1. FIG. 22B shows the flow cytometry results for Nalm-6 cells incubated with CXCR4-BP-Her2-CL. FIG. 22C shows the flow cytometry results for Nalm-6 cells incubated with anti-CD20. FIG. 22D shows the flow cytometry results for Nalm-6 cells incubated with CXCR4-BP-coil-CD20(IgG).

### Example 27

### Binding Affinity of Anti-CD20, CXCR4-BP-Coil-CD20(IgG), CXCR4-BP-Her2-CL and CXCR4-BP-Her2-CH1 Against CD20+/CXCR4dim BJAB Cells

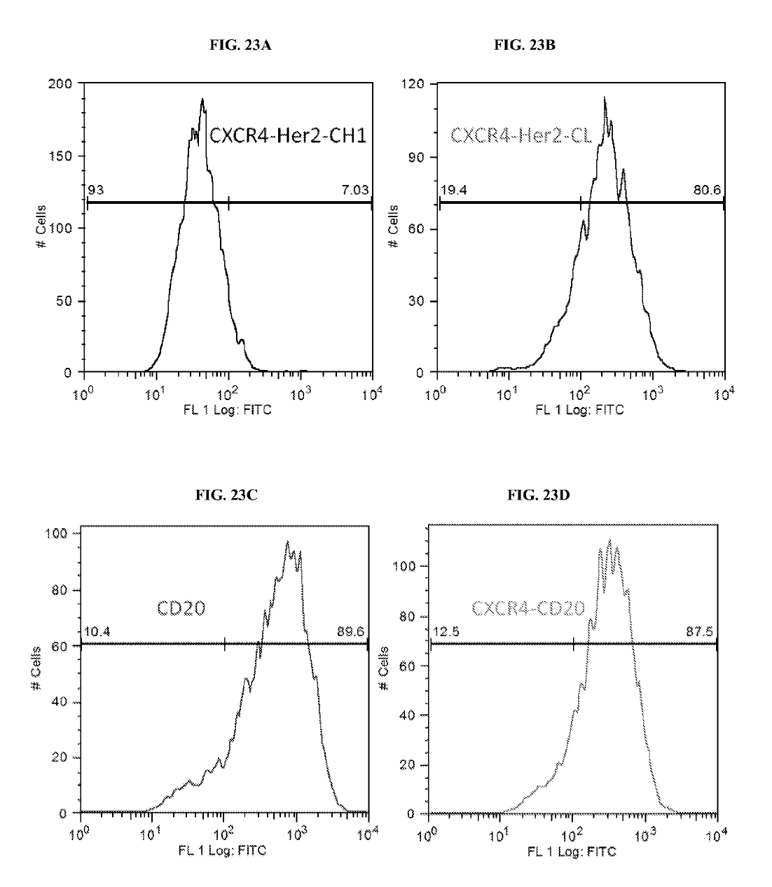

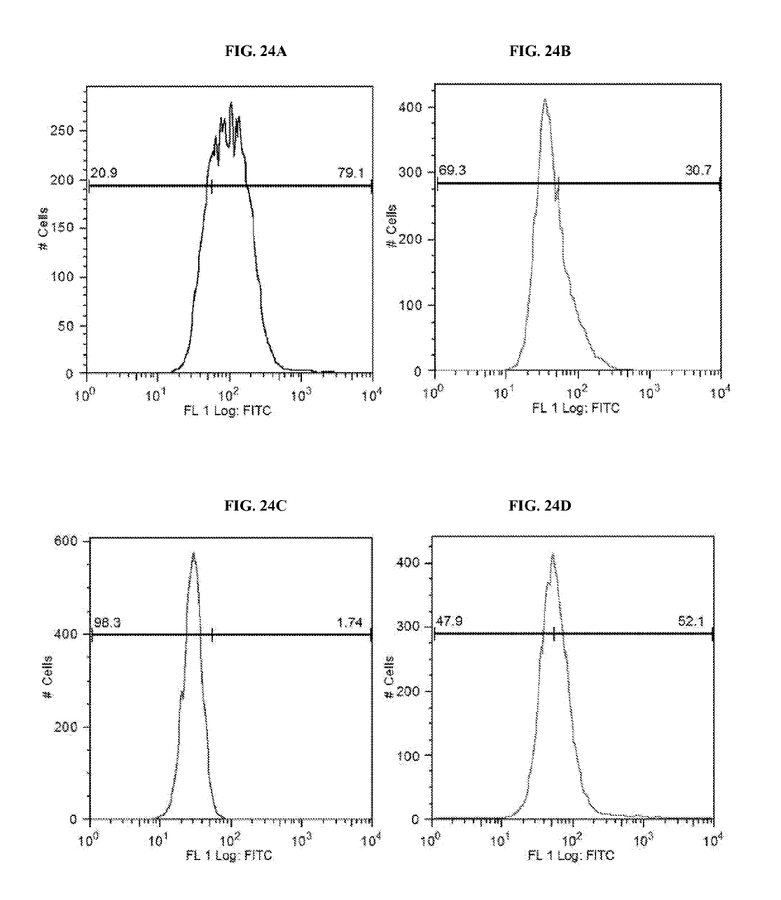

BJAB cells were cultured according to vendor's protocol. Cells were centrifuged and blocked in 1× PBS with 10% FBS at 4° C. for 1 hour. 50 nM of anti-CD20, CXCR4-BP-coil-CD20(IgG), CXCR4-BP-Her2-CL and CXCR4-BP-Her2-CH1 were added to the cell suspensions. The cell suspensions were shaken for 1 hour at 4° C. The cells were washed 3 times with PBS. The cells were incubated with a secondary antibody (e.g., fluorescein-anti-hFc) at 4° C. for 1 hour. The cells were washed 3 times with PBS and resuspended in PBS. The cellular fluorescence distribution was determined by flow cytometry. FIG. 23A-D show graphs of the binding affinity of anti-CD20, CXCR4-BP-coil-CD20(IgG), CXCR4-BP-Her2-CL and CXCR4-BP-Her2-CH1 against CD20−/CXCR4+ BJAB cells. FIG. 23A shows the flow cytometry results for BJAB cells incubated with CXCR4-BP-Her2-CH1. FIG. 23B shows the flow cytometry results for BJAB cells incubated with CXCR4-BP-Her2-CL. FIG. 23C shows the flow cytometry results for BJAB cells incubated with anti-CD20. FIG. 23D shows the flow cytometry results for BJAB cells incubated with CXCR4-BP-coil-CD20(IgG).

### Example 28

### Binding Affinity of Anti-CD20, CXCR4-BP-Coil-CD20(IgG), CXCR4-BP-Her2-CL and CXCR4-BP-Her2-CH1 Against CD20−/CXCR4− K562 Cells

K562 cells were cultured according to vendor's protocol. Cells were centrifuged and blocked in 1× PBS with 10% FBS at 4° C. for 1 hour. 50 nM of anti-CD20, CXCR4-BP-coil-CD20(IgG), CXCR4-BP-Her2-CL and CXCR4-BP-Her2-CH1 were added to the cell suspensions. The cell suspensions were shaken for 1 hour at 4° C. The cells were washed 3 times with PBS. The cells were incubated with a secondary antibody (e.g., fluorescein-anti-hFc) at 4° C. for 1 hour. The cells were washed 3 times with PBS and resuspended in PBS. The cellular fluorescence distribution was determined by flow cytometry. FIG. 24A-D show graphs of the binding affinity of anti-CD20, CXCR4-BP-coil-CD20(IgG), CXCR4-BP-Her2-CL and CXCR4-BP-Her2-CH1 against CD20−/CXCR4− K562 cells. FIG. 24A shows the flow cytometry results for K562 cells incubated with CXCR4-BP-Her2-CH1. FIG. 24B shows the flow cytometry results for K562 cells incubated with CXCR4-BP-Her2-CL. FIG. 24C shows the flow cytometry results for K562 cells incubated with anti-CD20. FIG. 24D shows the flow cytometry results for K562 cells incubated with CXCR4-BP-coil-CD20(IgG).

### Example 29

### Cloning, Expression and Purification of CXCR4-BP-Coil-Her2-CH1

Cloning: Mammalian expression vector of HER2 IgG heavy chain was generated by in-frame ligation of amplified HER2 Fab heavy chain (VH and CH1) to pFuse-hIgG1-Fc backbone vector (InvivoGen, CA). A gene encoding antibody HER2 light chain was amplified and cloned into the pFuse vector without hIgG1 Fc fragment. A gene encoding CXCR4-BP (YRKCRGGRRWCYQK=SEQ ID NO: 83) with an ascending adapter peptide (H2N-GGSGAKLAALKAKLAALKAKL-COOH=SEQ ID NO: 75) and a descending peptide (H2N-LEAELAALEAELAALEAGGSG-COOH=SEQ ID NO: 74) was synthesized by IDT gBlock gene synthesis. Subsequently, CXCR4-BP-HER2-CL fusion proteins were created by replacing the S180 and G181 in CH1 region of Trastuzumab heavy chain with CXCR4-BP with linker sequences at both ends. The resulting mammalian expression vectors were confirmed by DNA sequencing.

Expression and Purification: CXCR4-BP-coil-HER2-CH1 was expressed through transient transfection of FreeStyle HEK 293 cells with expression vectors of Trastuzumab light chain and CXCR4-BP-coil-HER2-CH1 heavy chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. CXCR4-BP-coil-HER2-CH1 was purified by Protein G chromatography (Thermo Fisher Scientific, IL). Purified proteins were analyzed by SDS-PAGE gels. FIG. 12 shows a SDS gel image of CXCR4-BP-coil-Her2-CH1 fusion proteins. As shown in FIG. 12, Lane 1 represents CXCR4-BP-coil-Her2-CH1 without DTT treatment, Lane 2 represents CXCR4-BP-coil-Her2-CH1 with DTT treatment and Lane 3 represents the protein standard marker. FIG. 16 also shows a SDS gel image of CXCR4-BP-coil-Her2-CH1 fusion proteins. As shown in FIG. 16, Lane 1 represents CXCR4-BP-coil-Her2-CH1 without DTT treatment, Lane 2 represents CXCR4-BP-coil-Her2-CH1 with DTT treatment and Lane 5 represents the protein standard ladder.

### Example 30

### Cloning, Expression and Purification of CXCR4-BP-Coil-Her2-CL

Cloning: Mammalian expression vector of HER2 IgG heavy chain was generated by in-frame ligation of amplified HER2 Fab heavy chain (VH and CH1) to pFuse-hIgG1-Fc backbone vector (InvivoGen, CA). A gene encoding antibody HER2 light chain was amplified and cloned into the pFuse vector without hIgG1 Fc fragment. A gene encoding CXCR4-BP (YRKCRGGRRWCYQK=SEQ ID NO: 83) with an ascending adapter peptide (H2N-GGSGAKLAALKAKLAALKAKL-COOH=SEQ ID NO: 75) and a descending peptide (H2N-LEAELAALEAELAALEAGGSG-COOH=SEQ ID NO: 74) was synthesized by IDT gBlock gene synthesis. Subsequently, CXCR4-BP-HER2-CL fusion proteins were created by replacing the K169 in CL region of HER2 light chain with CXCR4-BP with linker sequences at both ends. The resulting mammalian expression vectors were confirmed by DNA sequencing.

Expression and Purification: CXCR4-BP-coil-HER2-CL was expressed through transient transfection of FreeStyle HEK 293 cells with expression vectors of HER2-IgG heavy chain and CXCR4-BP-coil-HER2-CL light chain, according to the manufacturer's protocol. Briefly, 28 mL FreeStyle HEK 293 cells containing 3×10⁷ cells were seeded in a 125 mL shaking flask. 15 μl light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. CXCR4-BP-coil-HER2-CL was purified by Protein G chromatography (Thermo Fisher Scientific, IL). Purified proteins were analyzed by SDS-PAGE gels. FIG. 16 shows a SDS gel image of CXCR4-BP-coil-Her2-CL fusion proteins. As shown in FIG. 16, Lane 3 represents CXCR4-BP-coil-Her2-CL without DTT treatment, Lane 4 represents CXCR4-BP-coil-Her2-CL with DTT treatment and Lane 5 represents the protein standard ladder.

### Example 31

### Cloning, Expression and Purification of GCN4-CD19-Fab

Cloning: Mammalian expression vector of CD19 Fab heavy chain was generated by ligation of amplified CD19 Fab heavy chain (VH and CH1) to pFuse-hIgG1-Fc backbone vector (InvivoGen, CA) without Fc fragment. A gene encoding antibody CD light chain was amplified and cloned into the pFuse vector without hIgG1 Fc fragment. A gene encoding GCN4 (NYHLENEVARLKKL=SEQ ID NO: 84) with GGGGS (SEQ ID NO: 72) linker at both ends was synthesized as oligonucleotides. Subsequently, GCN4-CD19-CL fusion proteins were created by replacing the K169 in CL region of CD light chain with GCN4 with linker sequences at both ends. The resulting mammalian expression vectors were confirmed by DNA sequencing.

Expression and Purification: GCN4-CD19-CL was expressed through transient transfection of FreeStyle HEK 293 cells with expression vectors of CD19-Fab heavy chain and GCN4-CD19-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. GCN4-CD19-CL was purified by Protein G chromatography (Thermo Fisher Scientific, IL). Purified proteins were analyzed by SDS-PAGE gels. FIG. 28B shows a SDS gel image of GCN4-CD19(Fab) in non-reducing and reducing (with 50 mM DTT) conditions. As shown in FIG. 28B, Lane 1 represents the protein standard ladder, Lane 2 represents GCN4-CD19(Fab) without DTT treatment and Lane 3 represents GCN4-CD19(Fab) with DTT treatment.

### Example 32

### Cloning, Expression and Purification of GCN4-CD19-IgG

Cloning: Mammalian expression vector of CD19 IgG heavy chain was generated by in-frame ligation of amplified CD19 Fab heavy chain (VH and CH1) to pFuse-hIgG1-Fc backbone vector (InvivoGen, CA). A gene encoding antibody CD19 light chain was amplified and cloned into the pFuse vector without hIgG1 Fc fragment. A gene encoding GCN4 (NYHLENEVARLKKL=SEQ ID NO: 84) with GGGGS (SEQ ID NO: 72) linker at both ends was synthesized as oligonucleotides. Subsequently, GCN4-CD19-CL fusion proteins were created by replacing the K169 in CL region of CD light chain with GCN4 with linker sequences at both ends. The resulting mammalian expression vectors were confirmed by DNA sequencing.

Expression and Purification: GCN4-CD19-CL was expressed through transient transfection of FreeStyle HEK 293 cells with expression vectors of CD19-IgG heavy chain and GCN4-CD19-CL light chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. GCN4-CD19-CL was purified by Protein G chromatography (Thermo Fisher Scientific, IL). Purified proteins were analyzed by SDS-PAGE gels. FIG. 28A shows a SDS gel image of GCN4-CD19(IgG) in non-reducing and reducing (with 50 mM DTT) conditions. As shown in FIG. 28A, Lane 1 represents GCN4-CD19(IgG) without DTT treatment, Lane 2 represents GCN4-CD19(IgG) with DTT treatment and Lane 3 represents the protein standard ladder.

### Example 33

### Cloning, Expression and Purification of Her2ScFv-UCHT1-CL

Cloning: Mammalian expression vector of UCHT1 IgG heavy chain was generated by in-frame ligation of amplified UCHT1 Fab heavy chain (VH and CH1) to pFuse-hIgG1-Fc backbone vector (InvivoGen, CA). A gene encoding antibody UCHT1 light chain was amplified and cloned into the pFuse vector without hIgG1 Fc fragment. A gene encoding Her2ScFv (with (GGGGS)3 (SEQ ID NO: 73) as a linker between heavy and light chain of Her2) was synthesized by Genscript (NJ, USA), and amplified by polymerase chain reaction (PCR). A floppy linker was added to each end of the Her2ScFv insert. The sequence of the ascending adapter peptide with linkers at each end is: H2N-GGGGSGGGGSGGGGS-COOH (SEQ ID NO: 73); the sequence of the descending peptide with linkers at each end is: H2N-GGGGS-COOH (SEQ ID NO: 72). Subsequently, Her2ScFv-UCHT1-CL fusion proteins were created by replacing the K169 in CL region of UCHT1 light chain with Her2ScFv with linker sequences at both ends. The resulting mammalian expression vectors were confirmed by DNA sequencing.

Expression and Purification: Her2ScFv-UCHT1-CL was expressed through transient transfection of FreeStyle HEK 293 cells with expression vectors of UCHT1-IgG heavy chain and Her2ScFv-UCHT1-CL light chain, according to the manufacturer's protocol. Briefly, 28 mL FreeStyle HEK 293 cells containing 3×107 cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. Her2ScFv-UCHT1-CL was purified by Protein G chromatography (Thermo Fisher Scientific, IL). Purified proteins were analyzed by SDS-PAGE gels.

### Example 34

### Cloning, Expression and Purification of UCHT1ScFv-Her2-CH1

Cloning: Mammalian expression vector of HER2 IgG heavy chain was generated by in-frame ligation of amplified HER2 Fab heavy chain (VH and CH1) to pFuse-hIgG1-Fc backbone vector (InvivoGen, CA). A gene encoding antibody HER2 light chain was amplified and cloned into the pFuse vector without hIgG1 Fc fragment. A gene encoding UCHT1ScFv (with (GGGGS)3 (SEQ ID NO: 73) as a linker between heavy and light chain of UCHT1) was synthesized by Genscript (NJ, USA), and amplified by polymerase chain reaction (PCR). A floppy linker was added to each end of the UCHT1ScFv insert. The sequence of the ascending adapter peptide with linkers at each end is: H2N-GGGGSGGGGSGGGGS-COOH (SEQ ID NO: 73); the sequence of the descending peptide with linkers at each end is: H2N-GGGGS-COOH (SEQ ID NO: 72). Subsequently, UCHT1ScFv-HER2-CL fusion proteins were created by replacing the S180 and G181 in CH1 region of HER2 light chain with UCHT1ScFv with linker sequences at both ends. The resulting mammalian expression vectors were confirmed by DNA sequencing.

Expression and Purification: UCHT1ScFv-HER2-CH1 was expressed through transient transfection of FreeStyle HEK 293 cells with expression vectors of HER2-IgG light chain and UCHT1ScFv-HER2-CH1 heavy chain, according to the manufacturer's protocol. Briefly, 28 mL FreeStyle HEK 293 cells containing 3×107 cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. UCHT1ScFv-HER2-CH1 was purified by Protein G chromatography (Thermo Fisher Scientific, IL). Purified proteins were analyzed by SDS-PAGE gels.

### Example 35

### In Vitro Cytotoxicity of Anti-CD19ScFv-UCHT1-CL(Fab) by LDH Assay

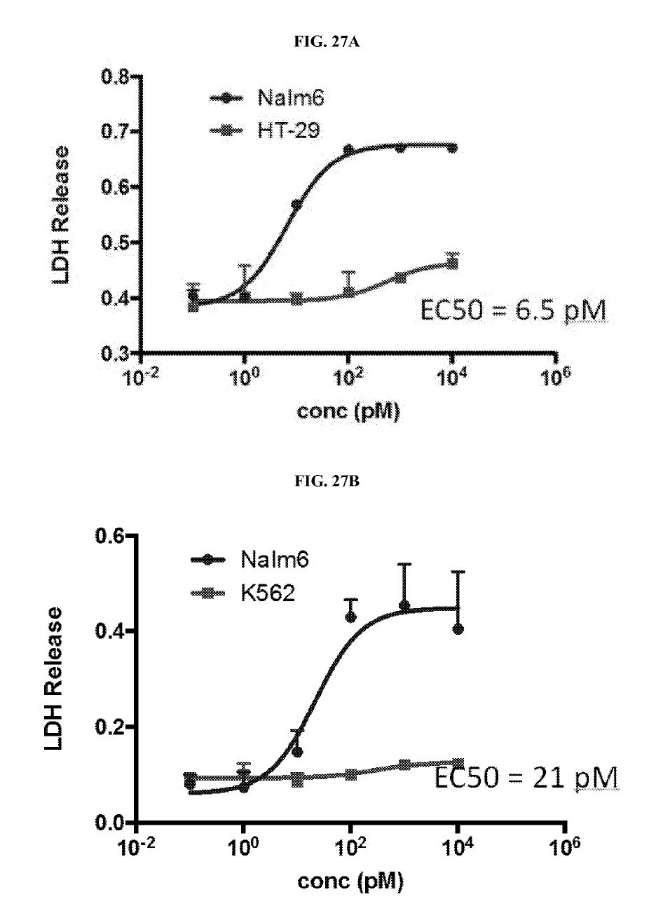

For in vitro cytotoxicity assays, PBMCs were purified from fresh healthy human donor blood (from The Scripps Research Institute normal blood donor service) by conventional Ficoll-Hypaque gradient centrifugation (GE Healthcare). Purified PBMCs were washed and incubated in flasks in RPMI with 10% (vol/vol) FBS and were incubated with target cells and different concentrations of anti-CD19ScFv-UCHT1-CL(Fab) fusion proteins (10 μL in medium) for 24 h at 37° C. Cytotoxicity of each well was measured for LDH levels in supernatant using the Cytotox-96 nonradioactive cytotoxicity assay kit (Promega). Lysis solution provided in the same kit (10 μL) was added to wells containing only target cells to achieve the maximum killing, and spontaneous killing was measured in wells with effector and target cells treated with vehicle (10 μL PBS). The absorbance at 490 nm was recorded using a SpectraMax 250 plate reader (Molecular Devices Corp.). FIG. 27A-B show graphs of the in vitro cytotoxicity of anti-CD19ScFv-UCHT1-CL(Fab) in Nalm-6 and HT-29 cells. For FIG. 27A, LDH Release=LDH readout in sample—LDH readout in medium only. For FIG. 27B, LDH Release=LDH readout in sample—LDH readout in PBMC only. The EC50 values were 6.5 pM and 21 pM for FIG. 27A-B, respectively.

### Example 36

### Cloning, Expression and Purification of GCN4-CD19-HC1 Fab

Cloning: Mammalian expression vector of CD19 Fab heavy chain was generated by ligation of amplified CD19 Fab heavy chain (VH and CH1) to pFuse-hIgG1-Fc backbone vector (InvivoGen, CA) without Fc fragment. A gene encoding antibody CD light chain was amplified and cloned into the pFuse vector without hIgG1 Fc fragment. A gene encoding GCN4 (NYHLENEVARLKKL=SEQ ID NO: 84) with was synthesized as oligonucleotides. Subsequently, GCN4-CD19-HC1 fusion proteins were created by grafting GCN4 into the mature heavy chain of the CD19 Fab following S135 of the CD19 Fab heavy chain. The resulting mammalian expression vectors were confirmed by DNA sequencing.

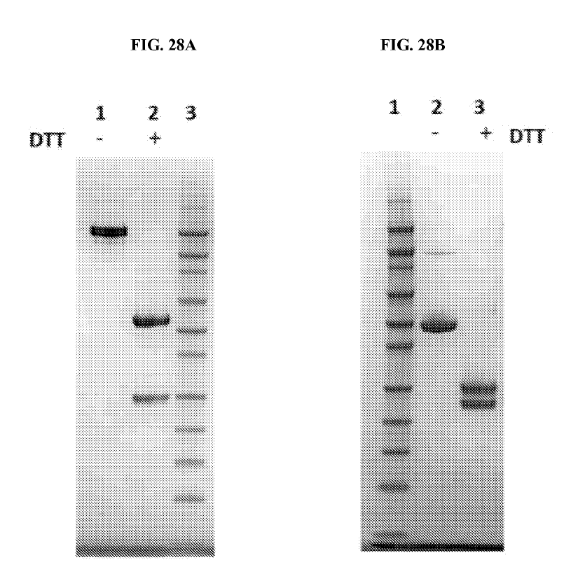

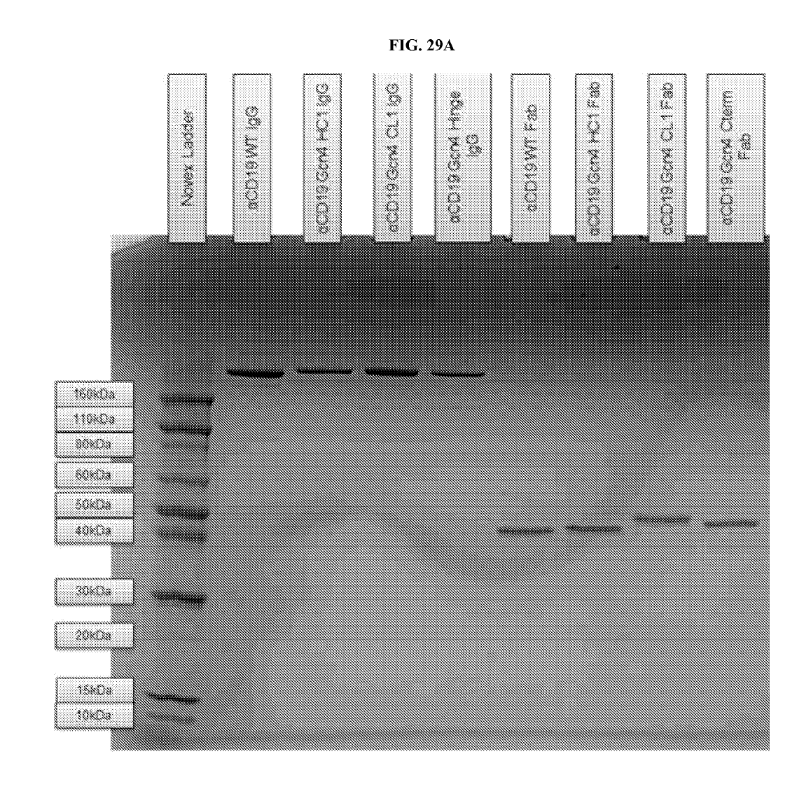

Expression and Purification: GCN4-CD19-HC1 Fab was expressed through transient transfection of FreeStyle HEK 293 cells with expression vectors of CD19-Fab light chain and GCN4-CD19-HC1, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. GCN4-CD19-CH1 Fab was purified by Protein G chromatography (Thermo Fisher Scientific, IL). Purified proteins were analyzed by SDS-PAGE gels. FIGS. 29A and 29B show SDS gel images of GCN4-CD19-HC1 Fab (Lane 7) in non-reducing and reducing (with 50 mM DTT) conditions.

### Example 37

### Cloning, Expression and Purification of GCN4-CD19-HC1 IgG

Cloning: Mammalian expression vector of CD19 IgG heavy chain was generated by in-frame ligation of amplified CD19 Fab heavy chain (VH and CH1) to pFuse-hIgG1-Fc backbone vector (InvivoGen, CA). A gene encoding antibody CD19 light chain was amplified and cloned into the pFuse vector without hIgG1 Fc fragment. A gene encoding GCN4 (NYHLENEVARLKKL=SEQ ID NO: 84) was synthesized as oligonucleotides. Subsequently, GCN4-CD19-HC1 IgG fusion proteins were created by inserting GCN4 following S135 of the mature heavy chain of the CD19 IgG. The resulting mammalian expression vectors were confirmed by DNA sequencing.

Expression and Purification: GCN4-CD19-HC1 IgG was expressed through transient transfection of FreeStyle HEK 293 cells with expression vectors of CD19-IgG light chain and GCN4-CD19 heavy chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. GCN4-CD19 heavy chain was purified by Protein G chromatography (Thermo Fisher Scientific, IL). Purified proteins were analyzed by SDS-PAGE gels. FIGS. 29A & 29B show SDS gel images of GCN4-CD19 IgG (Lane 3) in non-reducing and reducing (with 50 mM DTT) conditions.

### Example 38

### Cloning, Expression and Purification of GCN4-CD19-C-Term Fab

Cloning: Mammalian expression vector of CD19 Fab heavy chain was generated by ligation of amplified CD19 Fab heavy chain (VH and CH1) to pFuse-hIgG1-Fc backbone vector (InvivoGen, CA) without Fc fragment. A gene encoding antibody CD light chain was amplified and cloned into the pFuse vector without hIgG1 Fc fragment. A gene encoding GCN4 (NYHLENEVARLKKL=SEQ ID NO: 84) with GGGGS (SEQ ID NO: 72) linker at N-terminal end of GCN4 with was synthesized as oligonucleotides. Subsequently, GCN4-CD19-C-term Fab fusion proteins were created by fusing the linker-GCN4 to the C terminus of the Fab heavy chain at C223. The resulting mammalian expression vectors were confirmed by DNA sequencing.

Expression and Purification: GCN4-CD19-C-term Fab was expressed through transient transfection of FreeStyle HEK 293 cells with expression vectors of CD19-Fab light chain and GCN4-CD19-C-term, according to the manufacturer's protocol. Briefly, 28 mL FreeStyle HEK 293 cells containing 3×10⁷ cells were seeded in a 125 mL shaking flask. 15 μg light chain plasmid and 15 ρl heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. GCN4-CD19-C-term Fab was purified by Protein G chromatography (Thermo Fisher Scientific, IL). Purified proteins were analyzed by SDS-PAGE gels. FIGS. 29A and 29B show SDS gel images of GCN4-CD19-HC1 Fab (Lane 9) in non-reducing and reducing (with 50 mM DTT) conditions.

### Example 39

### Cloning, Expression and Purification of GCN4-CD19-Hinge IgG

Cloning: Mammalian expression vector of CD19 IgG heavy chain was generated by in-frame ligation of amplified CD19 Fab heavy chain (VH and CH1) to pFuse-hIgG1-Fc backbone vector (InvivoGen, CA). A gene encoding antibody CD19 light chain was amplified and cloned into the pFuse vector without hIgG1 Fc fragment. A gene encoding GCN4 (NYHLENEVARLKKL=SEQ ID NO: 84) with GGGGS (SEQ ID NO: 72) linker at N-terminal end of GCN4 and GGS at C-terminal of GCN4 (“linker-GCN4-linker”) was synthesized as oligonucleotides. Subsequently, GCN4-CD19-hinge IgG fusion proteins were created by grafting the linker-GCN4-linker between the C terminus of the Fab heavy chain at C223 and the hinge region. Thus, the linker-GCN4-linker extends the hinge region of the IgG, mimicking an IgG3 structure with an elongated hinge region. The resulting mammalian expression vectors were confirmed by DNA sequencing.

Expression and Purification: GCN4-CD19-HC1 IgG was expressed through transient transfection of FreeStyle HEK 293 cells with expression vectors of CD19-IgG light chain and GCN4-CD19 heavy chain, according to the manufacturer's protocol. Briefly, 28 mL FreeStyle HEK 293 cells containing 3×10⁷ cells were seeded in a 125 mL shaking flask. 15 μg light chain plasmid and 15 μg heavy chain plasmid diluted in 1 mL Opti-MEM medium were added in 1 mL Opti-MEM containing 60 μL 293fectin (Invitrogen, Inc). After the plasmids were incubated with 293fectin for 30 min, the lipoplex mixture was added to the cell suspension. Cells were then shaken at 125 rpm in a 5% CO2 environment at 37° C. Culture medium containing secreted proteins was harvested at 48 and 96 hours after transfection. GCN4-CD19 hinge IgG was purified by Protein G chromatography (Thermo Fisher Scientific, IL). Purified proteins were analyzed by SDS-PAGE gels. FIGS. 29A & 29B show SDS gel images of GCN4-CD19 hinge IgG (Lane 5) in non-reducing and reducing (with 50 mM DTT) conditions.

### Example 40

### T-Cell Mediated Cytotoxicity of GCN4-CD19 (IgG) and GCN4-CD19 (Fab) on CD19+ Cells RS4.11 and CD19− cells K562 or RPMI8226

The cytotoxic activities of various anti-CD19-GCN4 CAR-EC switches grafted/fused to different regions of anti-CD19 FMC63 antibodies or antibody fragments were assessed with the human PBMCs transduced with LV-EF1a-GCN4(52SR4) to create CAR-T-GCN4 at E:T ratios of 10:1 and 24 hour incubation. Switches tested were anti-CD19 FabCL1-GCN4 (“CL1 Fab), anti-CD19-GCN4 FabC-term (”C-term Fab), anti-CD19 IgGHC1-GCN4 (“HC1 IgG”), anti-CD19 IgGCL1-GCN4 (“CL1 IgG”), anti-CD19 IgGHinge-GCN4 (“Hinge IgG”), anti-CD19 IgGWT -GCN4 (“Wt IgG”), and anti-CD19 FabHC1-GCN4 (“HC1 Fab”). GCN4-CAR T cells were produced by transduction of human T cells with lentiviral anti-GCN4ScFv-CAR plasmids. Target cells, 104 RS4;11, K562 or RPMI8226 were mixed with 15 GCN4-CAR T cells. To the cell mixture, different amount of GCN4-CD19 fusion proteins were added. The cells were then incubated for 24 hours and the cytotoxicity was determined by LDH release assay (Table 1).

TABLE 1
Cytotoxicity of anti-CD19-GCN4 switches

| Switch | | | | | | | |
|---|---|---|---|---|---|---|---|
| Conc (nM) | CL1 Fab | C-term Fab | HC1 IgG | CL1 IgG | Hinge IgG | WT IgG | HC1 Fab |
| 10 | 70.10483 | 63.81551 | 47.46331 | 54.02444 | 67.4252 | 1.785714 | 41.07143 |
| 1 | 58.28092 | 59.53878 | 39.91614 | 59.58702 | 52.76022 | 2.040816 | 43.87755 |
| 0.1 | 60.54507 | 55.26205 | 39.16142 | 58.3228 | 40.62368 | 3.061224 | 44.38776 |
| 0.01 | 46.96017 | 33.37526 | 28.09225 | 56.80573 | 35.0611 | 2.55102 | 20.66327 |
| 0.001 | 4.444445 | −2.09644 | 1.174004 | 24.18879 | 2.697009 | 2.55102 | −0.2551 |
| 0.0001 | 2.180294 | −4.61216 | −2.09644 | 1.685631 | −5.14117 | 2.040817 | −0.5102 |
| 0.00001 | 1.425577 | −3.60587 | −1.09015 | 0.927097 | −6.65823 | 1.785714 | −0.7653 |
| 1E−07 | 0.922432 | −1.34172 | 0.419288 | 0.674253 | −1.60135 | 1.27551 | −1.27551 |

### Example 41

### In Vitro Cytotoxicity of Her2ScFv-UCHT1 CL bispecific antibodies by LDH assay.

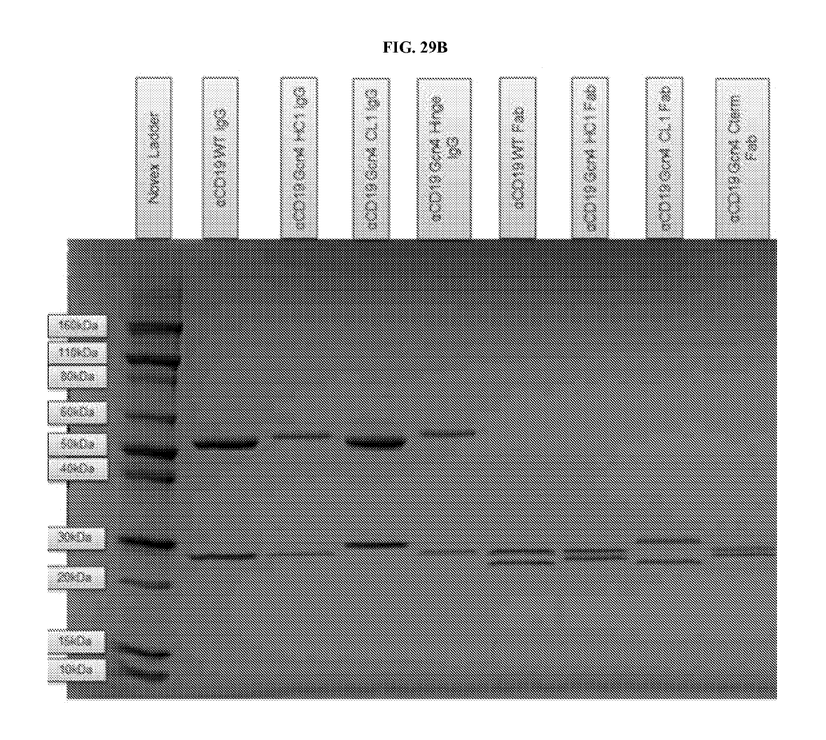

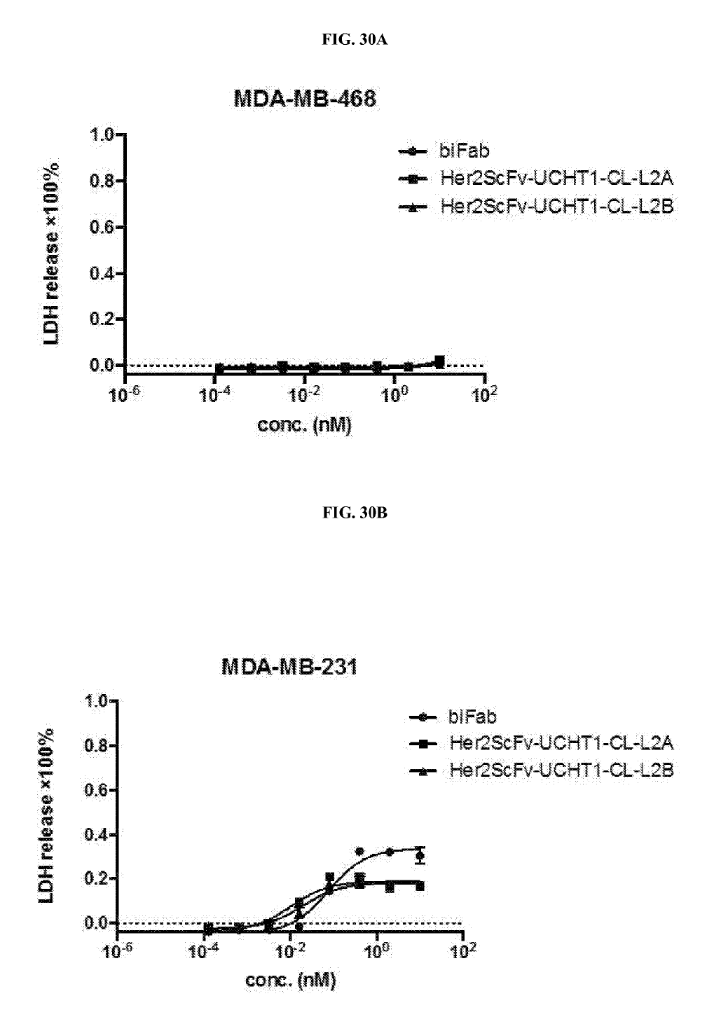

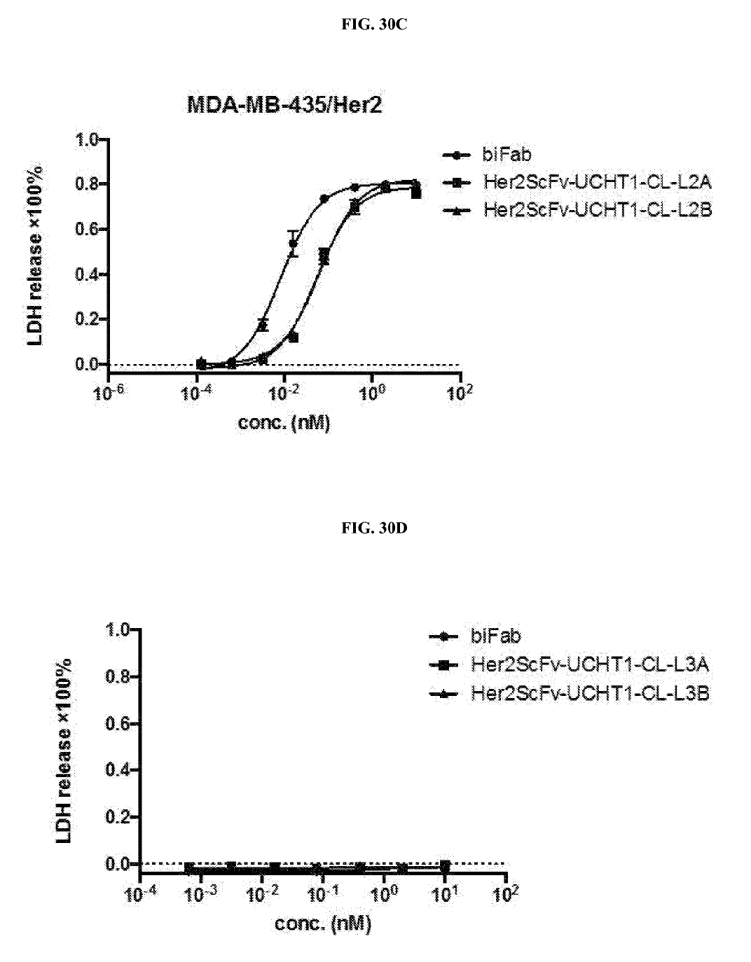

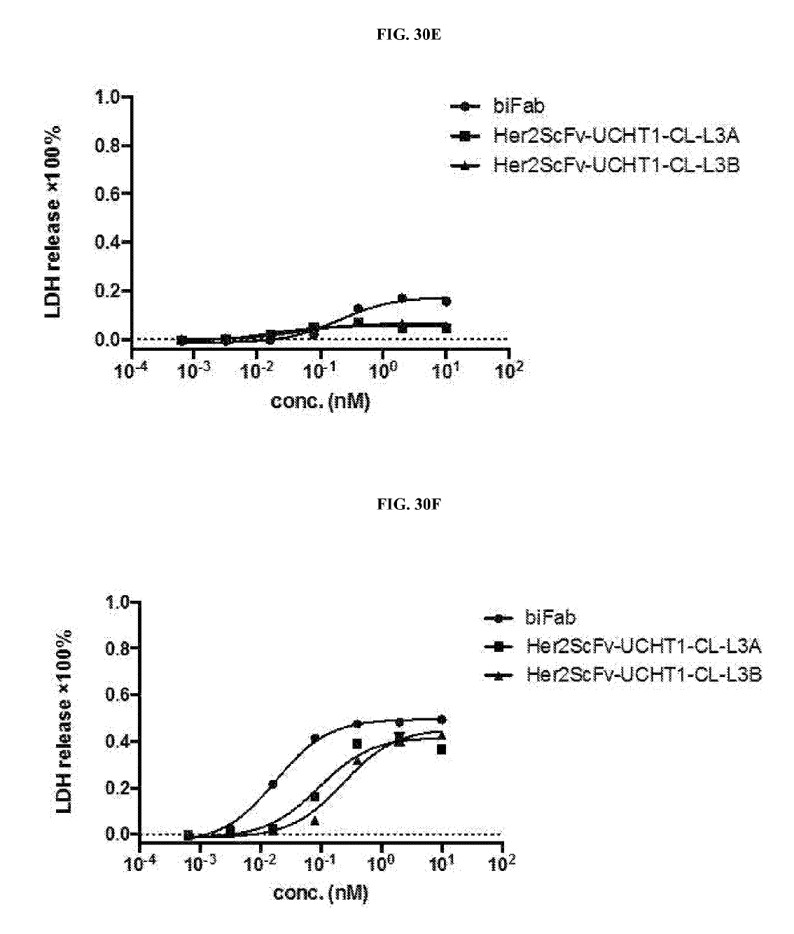

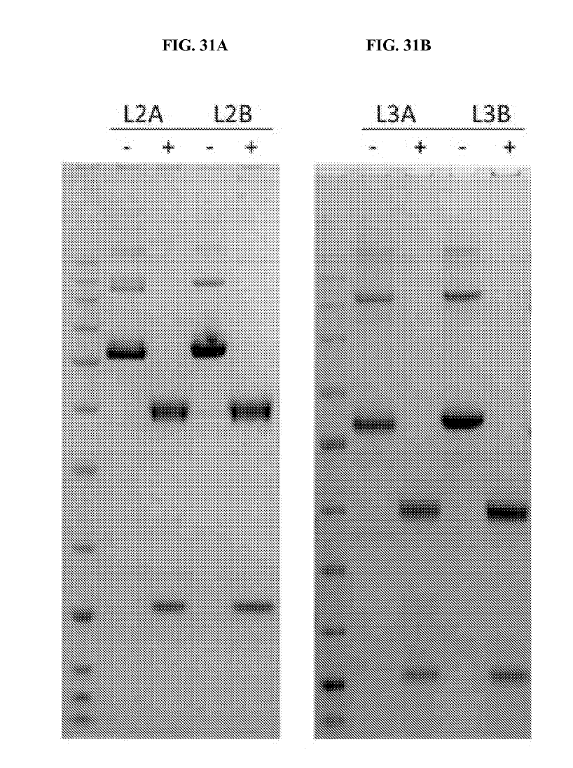

For in vitro cytotoxicity assays, PBMCs were purified from fresh healthy human donor blood (from The Scripps Research Institute normal blood donor service) by conventional Ficoll-Hypaque gradient centrifugation (GE Healthcare). Purified PBMCs were washed and incubated in flasks in RPMI with 10% (vol/vol) FBS and were incubated with target cells and different concentrations of bispecific fusion proteins (10 μL in medium) for 24 h at 37° C. Cytotoxicity of each well was measured for LDH levels in supernatant using the Cytotox-96 nonradioactive cytotoxicity assay kit (Promega). Lysis solution provided in the same kit (10 pL) was added to wells containing only target cells to achieve the maximum killing, and spontaneous killing was measured in wells with effector and target cells treated with vehicle (10 μL PBS). The absorbance at 490 nm was recorded using a SpectraMax 250 plate reader (Molecular Devices Corp.). Percent cytotoxicity was calculated by: % cytotoxicity=(absorbance experimental−absorbance spontaneous average)/(absorbance maximum killing average−absorbance spontaneous average). See FIGS. 30A-C for results of cytotoxicity assay and FIGS. 31A-B for SDS-PAGE gel images of Her2ScFv-UCHT1 CL bispecific antibodies.

TABLE 2
Antibody or Antibody-fusion proteins-Nucleotide Sequence

| SEQ | | |
|---|---|---|
| ID NO: | Description | Sequence |
| 1. | Trastuzumab | GAAGTGCAGCTGGTGGAGTCTGGAGGAGGCTTGGTCCAGCCTGGGGGGTCCCTGAG |
| | Heavy | ACTCTCCTGTGCAGCCTCTGGGTTCAATATTAAGGACACTTACATCCACTGGGTCCG |
| | Chain | CCAGGCTCCAGGGAAGGGGCTGGAGTGGGTCGCACGTATTTATCCTACCAATGGTT |
| | | ACACACGCTACGCAGACTCCGTGAAGGGCCGATTCACCATCTCCGCAGACACTTCC |
| | | AAGAACACGGCGTATCTTCAAATGAACAGCCTGAGAGCCGAGGACACGGCCGTGTA |
| | | TTACTGTTCGAGATGGGGCGGTGACGGCTTCTATGCCATGGACTACTGGGGCCAAG |
| | | GAACCCTGGTCACCGTCTCCTCAGCCTCCACCAAGGGCCCATCGGTCTTCCCCCTGG |
| | | CACCCTCCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGACCAGCGG |
| | | CGTGCACACCTTCCCGGCTGTCCTACAGTCCTCAGGACTCTACTCCCTCAGCAGCGT |
| | | GGTGACTGTGCCCTCTAGCAGCTTGGGCACCCAGACCTACATCTGCAACGTGAATCA |
| | | CAAGCCCAGCAACACCAAGGTGGACAAGAAAGTTGAACCCAAATCTTGCGACAAA |
| | | ACTCACACATGCCCACCGTGCCCAGCACCTCCAGTCGCCGGACCGTCAGTCTTCCTC |
| | | TTCCCTCCAAAACCCAAGGACACCCTCATGATCTCCCGGACCCCTGAGGTCACATGC |
| | | GTGGTGGTGGACGTGAGCCACGAAGACCCTGAGGTCAAGTTCAACTGGTACGTGGA |
| | | CGGCGTGGAGGTGCATAATGCCAAGACAAAGCCGCGGGAGGAGCAGTACAACAGC |
| | | ACGTACCGTGTGGTCAGCGTCCTCACCGTCCTGCACCAGGACTGGCTGAATGGCAA |
| | | GGAGTACAAGTGCAAGGTCTCCAACAAAGGCCTCCCAAGCTCCATCGAGAAAACCA |
| | | TCTCCAAAGCCAAAGGGCAGCCCCGAGAACCACAGGTGTACACCCTGCCTCCATCC |
| | | CGGGATGAGCTGACCAAGAACCAGGTCAGCCTGACCTGCCTGGTCAAAGGCTTCTA |
| | | TCCCAGCGACATCGCCGTGGAGTGGGAGAGCAATGGGCAGCCGGAGAACAACTAC |
| | | AAGACCACGCCTCCCGTGCTGGACTCCGACGGCTCCTTCTTCCTCTACAGCAAGCTC |
| | | ACCGTGGACAAGAGCAGGTGGCAGCAGGGGAACGTCTTCTCATGCTCCGTGATGCA |
| | | TGAGGCTCTGCACAACCACTACACGCAGAAGAGCCTCTCCCTGTCTCCGGGTAAAT |
| | | GATAA |
| 2. | UCHT1 | GAGGTCCAGCTGCAGCAGAGTGGTCCTGAACTGGTTAAGCCTGGGGCATCAATGAA |
| | Heavy | AATCTCCTGTAAAGCAAGTGGTTATTCCTTCACCGGCTATACAATGAACTGGGTGAA |
| | Chain | GCAGTCTCACGGAAAAAACCTGGAATGGATGGGGCTGATTAATCCGTATAAGGGTG |
| | IgG | TTAGCACCTACAACCAGAAATTCAAAGATAAGGCAACACTGACTGTCGACAAAAGC |
| | | TCCTCTACCGCTTATATGGAACTGCTGAGCCTGACATCCGAGGATTCTGCCGTTTAT |
| | | TACTGCGCGCGCAGCGGTTATTACGGGGATTCCGACTGGTACTTTGACGTGTGGGGC |
| | | CAGGGTACCACACTGACCGTTTTCAGCGCTAGCACCAAGGGCCCATCGGTCTTCCCC |
| | | CTGGCACCCTCCTCCAAGAGCACCTCTGGGGGCACAGCGGCCCTGGGCTGCCTGGT |
| | | CAAGGACTACTTCCCCGAACCGGTGACGGTGTCGTGGAACTCAGGCGCCCTGACCA |
| | | GCGGCGTGCACACCTTCCCGGCTGTCCTACAGTCCTCAGGACTCTACTCCCTCAGCA |
| | | GCGTGGTGACTGTGCCCTCTAGCAGCTTGGGCACCCAGACCTACATCTGCAACGTGA |
| | | ATCACAAGCCCAGCAACACCAAGGTGGACAAGAAAGTTGAGCCCAAATCTTGTGAC |
| | | AAAACTCACACATGCCCACCGTGCCCAGCACCTGAACTCCTGGGGGGACCGTCAGT |
| | | CTTCCTCTTCCCCCCAAAACCCAAGGACACCCTCATGATCTCCCGGACCCCTGAGGT |
| | | CACATGCGTGGTGGTGGACGTGAGCCACGAAGACCCTGAGGTCAAGTTCAACTGGT |
| | | ACGTGGACGGCGTGGAGGTGCATAATGCCAAGACAAAGCCGCGGGAGGAGCAGTA |
| | | CGCCAGCACGTACCGTGTGGTCAGCGTCCTCACCGTCCTGCACCAGGACTGGCTGA |
| | | ATGGCAAGGAGTACAAGTGCAAGGTCTCCAACAAAGCCCTCCCAGCCCCCATCGAG |
| | | AAAACCATCTCCAAAGCCAAAGGGCAGCCCCGAGAACCACAGGTGTACACCCTGCC |
| | | CCCATCCCGGGATGAGCTGACCAAGAACCAGGTCAGCCTGACCTGCCTGGTCAAAG |
| | | GCTTCTATCCCAGCGACATCGCCGTGGAGTGGGAGAGCAATGGGCAGCCGGAGAAC |
| | | AACTACAAGACCACGCCTCCCGTGCTGGACTCCGACGGCTCCTTCTTCCTCTACAGC |
| | | AAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCTCCGG |
| | | GTAAATGATAA |
| 3. | UCHT1 | GAGGTCCAGCTGCAGCAGAGTGGTCCTGAACTGGTTAAGCCTGGGGCATCAATGAA |
| | Heavy | AATCTCCTGTAAAGCAAGTGGTTATTCCTTCACCGGCTATACAATGAACTGGGTGAA |
| | Chain | GCAGTCTCACGGAAAAAACCTGGAATGGATGGGGCTGATTAATCCGTATAAGGGTG |
| | Fab | TTAGCACCTACAACCAGAAATTCAAAGATAAGGCAACACTGACTGTCGACAAAAGC |
| | | TCCTCTACCGCTTATATGGAACTGCTGAGCCTGACATCCGAGGATTCTGCCGTTTAT |
| | | TACTGCGCGCGCAGCGGTTATTACGGGGATTCCGACTGGTACTTTGACGTGTGGGGC |
| | | CAGGGTACCACACTGACCGTTTTCAGCGCTAGCACCAAGGGCCCATCGGTCTTCCCC |
| | | CTGGCACCCTCCTCCAAGAGCACCTCTGGGGGCACAGCGGCCCTGGGCTGCCTGGT |
| | | CAAGGACTACTTCCCCGAACCGGTGACGGTGTCGTGGAACTCAGGCGCCCTGACCA |
| | | GCGGCGTGCACACCTTCCCGGCTGTCCTACAGTCCTCAGGACTCTACTCCCTCAGCA |
| | | GCGTGGTGACTGTGCCCTCTAGCAGCTTGGGCACCCAGACCTACATCTGCAACGTGA |
| | | ATCACAAGCCCAGCAACACCAAGGTGGACAAGAAAGTTGAGCCCAAATCTTGT |
| 4. | Anti- | CAGGTGCAGCTCCAGCAGCCGGGAGCAGAATTGGTTAAGCCTGGGGCCTCAGTGAA |
| | CD20 | AATGAGCTGTAAGGCCAGCGGCTACACCTTCACCTCCTATAACATGCATTGGGTAA |
| | Heavy | AACAGACCCCCGGCAGAGGTCTCGAGTGGATCGGAGCGATTTATCCGGGCAATGGA |
| | Chain | GACACTTCCTATAATCAGAAATTTAAGGGCAAGGCCACTCTCACAGCCGACAAGTC |
| | IgG | TTCATCCACCGCTTATATGCAGCTGAGCTCCTTGACCTCTGAGGACAGCGCCGTTTA |
| | | CTATTGCGCACGAAGCACGTACTACGGGGGAGATTGGTACTTTAACGTGTGGGGGG |
| | | CCGGAACCACCGTGACTGTGTCTGCTGCCTCCACCAAGGGCCCATCGGTCTTCCCCC |
| | | TGGCACCCTCCTCCAAGAGCACCTCTGGGGGCACAGCGGCCCTGGGCTGCCTGGTC |
| | | AAGGACTACTTCCCCGAACCGGTGACGGTGTCGTGGAACTCAGGCGCCCTGACCAG |
| | | CGGCGTGCACACCTTCCCGGCTGTCCTACAGTCCTCAGGACTCTACTCCCTCAGCAG |
| | | CGTGGTGACTGTGCCCTCTAGCAGCTTGGGCACCCAGACCTACATCTGCAACGTGAA |
| | | TCACAAGCCCAGCAACACCAAGGTGGACAAGAAAGTTGAACCCAAATCTTGCGACA |
| | | AAACTCACACATGCCCACCGTGCCCAGCACCTCCAGTCGCCGGACCGTCAGTCTTCC |
| | | TCTTCCCTCCAAAACCCAAGGACACCCTCATGATCTCCCGGACCCCTGAGGTCACAT |
| | | GCGTGGTGGTGGACGTGAGCCACGAAGACCCTGAGGTCAAGTTCAACTGGTACGTG |
| | | GACGGCGTGGAGGTGCATAATGCCAAGACAAAGCCGCGGGAGGAGCAGTACAACA |
| | | GCACGTACCGTGTGGTCAGCGTCCTCACCGTCCTGCACCAGGACTGGCTGAATGGC |
| | | AAGGAGTACAAGTGCAAGGTCTCCAACAAAGGCCTCCCAAGCTCCATCGAGAAAAC |
| | | CATCTCCAAAGCCAAAGGGCAGCCCCGAGAACCACAGGTGTACACCCTGCCTCCAT |
| | | CCCGGGATGAGCTGACCAAGAACCAGGTCAGCCTGACCTGCCTGGTCAAAGGCTTC |
| | | TATCCCAGCGACATCGCCGTGGAGTGGGAGAGCAATGGGCAGCCGGAGAACAACT |
| | | ACAAGACCACGCCTCCCGTGCTGGACTCCGACGGCTCCTTCTTCCTCTACAGCAAGC |
| | | TCACCGTGGACAAGAGCAGGTGGCAGCAGGGGAACGTCTTCTCATGCTCCGTGATG |
| | | CATGAGGCTCTGCACAACCACTACACGCAGAAGAGCCTCTCCCTGTCTCCGGGTAA |
| | | ATGATAA |
| 5. | Anti- | CAGGTGCAGCTCCAGCAGCCGGGAGCAGAATTGGTTAAGCCTGGGGCCTCAGTGAA |
| | CD20 | AATGAGCTGTAAGGCCAGCGGCTACACCTTCACCTCCTATAACATGCATTGGGTAA |
| | Heavy | AACAGACCCCCGGCAGAGGTCTCGAGTGGATCGGAGCGATTTATCCGGGCAATGGA |
| | Chain | GACACTTCCTATAATCAGAAATTTAAGGGCAAGGCCACTCTCACAGCCGACAAGTC |
| | Fab | TTCATCCACCGCTTATATGCAGCTGAGCTCCTTGACCTCTGAGGACAGCGCCGTTTA |
| | | CTATTGCGCACGAAGCACGTACTACGGGGGAGATTGGTACTTTAACGTGTGGGGGG |
| | | CCGGAACCACCGTGACTGTGTCTGCTGCTAGCACCAAGGGCCCATCGGTCTTCCCCC |
| | | TGGCACCCTCCTCCAAGAGCACCTCTGGGGGCACAGCGGCCCTGGGCTGCCTGGTC |
| | | AAGGACTACTTCCCCGAACCGGTGACGGTGTCGTGGAACTCAGGCGCCCTGACCAG |
| | | CGGCGTGCACACCTTCCCGGCTGTCCTACAGTCCTCAGGACTCTACTCCCTCAGCAG |
| | | CGTGGTGACTGTGCCCTCTAGCAGCTTGGGCACCCAGACCTACATCTGCAACGTGAA |
| | | TCACAAGCCCAGCAACACCAAGGTGGACAAGAAAGTTGAGCCCAAATCTTGT |
| 6. | Anti- | GAGGTGAAACTGCAGGAGTCAGGACCTGGCCTGGTGGCGCCCTCACAGAGCCTGTC |
| | CD19 | CGTCACATGCACTGTCTCAGGGGTCTCATTACCCGACTATGGTGTAAGCTGGATTCG |
| | Heavy | CCAGCCTCCACGAAAGGGTCTGGAGTGGCTGGGAGTAATATGGGGTAGTGAAACCA |
| | Chain | CATACTATAATTCAGCTCTCAAATCCAGACTGACCATCATCAAGGACAACTCCAAG |
| | IgG | AGCCAAGTTTTCTTAAAAATGAACAGTCTGCAAACTGATGACACAGCCATTTACTAC |
| | | TGTGCCAAACATTATTACTACGGTGGTAGCTATGCTATGGACTACTGGGGCCAAGG |
| | | AACCTCAGTCACCGTCTCCTCAGCCTCCACCAAGGGCCCATCGGTCTTCCCCCTGGC |
| | | ACCCTCCTCCAAGAGCACCTCTGGGGGCACAGCGGCCCTGGGCTGCCTGGTCAAGG |
| | | ACTACTTCCCCGAACCGGTGACGGTGTCGTGGAACTCAGGCGCCCTGACCAGCGGC |
| | | GTGCACACCTTCCCGGCTGTCCTACAGTCCTCAGGACTCTACTCCCTCAGCAGCGTG |
| | | GTGACTGTGCCCTCTAGCAGCTTGGGCACCCAGACCTACATCTGCAACGTGAATCAC |
| | | AAGCCCAGCAACACCAAGGTGGACAAGAAAGTTGAGCCCAAATCTTGTGACAAAA |
| | | CTCACACATGCCCACCGTGCCCAGCACCTCCAGTCGCCGGACCGTCAGTCTTCCTCT |
| | | TCCCTCCAAAACCCAAGGACACCCTCATGATCTCCCGGACCCCTGAGGTCACATGCG |
| | | TGGTGGTGGACGTGAGCCACGAAGACCCTGAGGTCAAGTTCAACTGGTACGTGGAC |
| | | GGCGTGGAGGTGCATAATGCCAAGACAAAGCCGCGGGAGGAGCAGTACAACAGCA |
| | | CGTACCGTGTGGTCAGCGTCCTCACCGTCCTGCACCAGGACTGGCTGAATGGCAAG |
| | | GAGTACAAGTGCAAGGTCTCCAACAAAGGCCTCCCAAGCTCCATCGAGAAAACCAT |
| | | CTCCAAAGCCAAAGGGCAGCCCCGAGAACCACAGGTGTACACCCTGCCTCCATCCC |
| | | GGGATGAGCTGACCAAGAACCAGGTCAGCCTGACCTGCCTGGTCAAAGGCTTCTAT |
| | | CCCAGCGACATCGCCGTGGAGTGGGAGAGCAATGGGCAGCCGGAGAACAACTACA |
| | | AGACCACGCCTCCCGTGCTGGACTCCGACGGCTCCTTCTTCCTCTACAGCAAGCTCA |
| | | CCGTGGACAAGAGCAGGTGGCAGCAGGGGAACGTCTTCTCATGCTCCGTGATGCAT |
| | | GAGGCTCTGCACAACCACTACACGCAGAAGAGCCTCTCCCTGTCTCCGGGTAAA |
| 7. | Anti- | GAGGTGAAACTGCAGGAGTCAGGACCTGGCCTGGTGGCGCCCTCACAGAGCCTGTC |
| | CD19 | CGTCACATGCACTGTCTCAGGGGTCTCATTACCCGACTATGGTGTAAGCTGGATTCG |
| | Heavy | CCAGCCTCCACGAAAGGGTCTGGAGTGGCTGGGAGTAATATGGGGTAGTGAAACCA |
| | Chain | CATACTATAATTCAGCTCTCAAATCCAGACTGACCATCATCAAGGACAACTCCAAG |
| | Fab | AGCCAAGTTTTCTTAAAAATGAACAGTCTGCAAACTGATGACACAGCCATTTACTAC |
| | | TGTGCCAAACATTATTACTACGGTGGTAGCTATGCTATGGACTACTGGGGCCAAGG |
| | | AACCTCAGTCACCGTCTCCTCAGCCTCCACCAAGGGCCCATCGGTCTTCCCCCTGGC |
| | | ACCCTCCTCCAAGAGCACCTCTGGGGGCACAGCGGCCCTGGGCTGCCTGGTCAAGG |
| | | ACTACTTCCCCGAACCGGTGACGGTGTCGTGGAACTCAGGCGCCCTGACCAGCGGC |
| | | GTGCACACCTTCCCGGCTGTCCTACAGTCCTCAGGACTCTACTCCCTCAGCAGCGTG |
| | | GTGACTGTGCCCTCTAGCAGCTTGGGCACCCAGACCTACATCTGCAACGTGAATCAC |
| | | AAGCCCAGCAACACCAAGGTGGACAAGAAAGTTGAGCCCAAATCTTGT |
| 8. | Trastuzumab | GACATCCAGATGACCCAGTCTCCATCCTCCCTGTCTGCATCTGTAGGAGACAGAGTC |
| | Light | ACCATCACTTGCCGGGCAAGTCAGGATGTGAATACCGCGGTCGCATGGTATCAGCA |
| | Chain | GAAACCAGGGAAAGCCCCTAAGCTCCTGATCTATTCTGCATCCTTCTTGTATAGTGG |
| | | GGTCCCATCAAGGTTCAGTGGCAGTAGATCTGGGACAGATTTCACTCTCACCATCAG |
| | | CAGTCTGCAACCTGAAGATTTTGCAACTTACTACTGTCAACAGCATTACACTACCCC |
| | | TCCGACGTTCGGCCAAGGTACCAAGCTTGAGATCAAACGAACTGTGGCTGCACCAT |
| | | CTGTCTTCATCTTCCCGCCATCTGATGAGCAGTTGAAATCTGGAACTGCCTCTGTCGT |
| | | GTGCCTGCTGAATAACTTCTATCCCAGAGAGGCCAAAGTACAGTGGAAGGTGGATA |
| | | ACGCCCTCCAATCGGGTAACTCCCAGGAGAGTGTCACAGAGCAGGACAGCAAGGAC |
| | | AGCACCTACAGCCTCAGCAGCACCCTGACGCTGAGCAAAGCAGACTACGAGAAACA |
| | | CAAAGTCTACGCCTGCGAAGTCACCCATCAGGGCCTGTCCTCGCCCGTCACAAAGA |
| | | GCTTCAACAGGGGAGAGTGT |
| 9. | UCHT1 | GATATTCAGATGACTCAGACTACCAGTTCACTGAGCGCCTCCCTGGGCGATCGCGTG |
| | Light | ACAATTAGTTGTCGTGCGTCACAGGACATCCGGAACTATCTGAATTGGTACCAGCA |
| | Chain | GAAGCCGGACGGCACAGTCAAACTGCTGATCTATTACACTAGCCGTCTGCATTCCG |
| | | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGCGTACGGTGGCTGCACC |
| | | ATCTGTCTTCATCTTCCCGCCATCTGATGAGCAGTTGAAATCTGGAACTGCCTCTGTT |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCAAAG |
| | | ACAGCACCTACAGCCTCAGCAGCACCCTGACGCTGAGCAAAGCAGACTACGAGAAA |
| | | CACAAAGTCTACGCCTGCGAAGTCACCCATCAGGGCCTGAGCTCGCCCGTCACAAA |
| | | GAGCTTCAACAGGGGAGAGTGT |
| 10. | Palivizumab | CAGGTGACCCTGCGCGAGTCCGGCCCtGCaCTGGTGAAGCCCACCCAGACCCTGACC |
| | Heavy | CTGACCTGCACCTTCTCCGGCTTCTCCCTGTCCACCTCCGGCATGTCCGTGGGCTGG |
| | Chain | ATCCGgCAGCCtCCCGGCAAGGCCCTGGAGTGGCTGGCtGACATCTGGTGGGACGAC |
| | | AAGAAGGACTACAACCCCTCCCTGAAGTCCCGCCTGACCATCTCCAAGGACACCTC |
| | | CAAGAACCAGGTGGTGCTGAAGGTGACCAACATGGACCCCGCCGACACCGCCACCT |
| | | ACTACTGCGCCCGCTCAATGATTACCAACTGGTACTTCGACGTGTGGGGaGCCGGtA |
| | | CCACCGTGACCGTGTCtTCCgcctccaccaagggcccatcggtottccccctggcaccct |
| | | cctccaagagcacctctgggggcacagcggccctgggctgcctggtcaaggactacttcc |
| | | ccgaaccggtgacggtgtcgtggaactcaggcgccctgaccagcggcgtgcacaccttcc |
| | | cggctgtcctacagtcctcaggactctactccctcagcagcgtggtgactgtgccctcta |
| | | gcagcttgggcacccagacctacatctgcaacgtgaatcacaagcccagcaacaccaagg |
| | | tggacaagaaagttgaacccaaatcttgcgacaaaactcacacatgcccaccgtgcccag |
| | | cacctCCaGtcGCcggaccgtcagtottcctcttcccTccaaaacccaaggacaccctca |
| | | tgatctcccggacccctgaggtcacatgcgtggtggtggacgtgagccacgaagaccctg |
| | | aggtcaagttcaactggtacgtggacggcgtggaggtgcataatgccaagacaaagccgc |
| | | gggaggagcagtacaacagcacgtaccgtgtggtcagcgtcctcaccgtcctgcaccagg |
| | | actggctgaatggcaaggagtacaagtgcaaggtctccaacaaagGcctcccaAGcTcca |
| | | tcgagaaaaccatctccaaagccaaagggcagccccgagaaccacaggtgtacaccctgc |
| | | cTccatcccgggatgagctgaccaagaaccaggtcagcctgacctgcctggtcaaaggct |
| | | tctatcccagcgacatcgccgtggagtgggagagcaatgggcagccggagaacaactaca |
| | | agaccacgcctcccgtgctggactccgacggctccttcttcctctacagcaagctcaccg |
| | | tggacaagagcaggtggcagcaggggaacgtcttctcatgctccgtgatgcatgaggctc |
| | | tgcacaaccactacacgcagaagagcctctccctgtctccgggtaaa |
| 11. | hEPO- | GAAGTGCAGCTGGTGGAGTCTGGAGGAGGCTTGGTCCAGCCTGGGGGGTCCCTGAG |
| | coil- | ACTCTCCTGTGCAGCCTCTGGGTTCAATATTAAGGACACTTACATCCACTGGGTCCG |
| | Her2- | CCAGGCTCCAGGGAAGGGGCTGGAGTGGGTCGCACGTATTTATCCTACCAATGGTT |
| | CH1 | ACACACGCTACGCAGACTCCGTGAAGGGCCGATTCACCATCTCCGCAGACACTTCC |
| | | AAGAACACGGCGTATCTTCAAATGAACAGCCTGAGAGCCGAGGACACGGCCGTGTA |
| | | TTACTGTTCGAGATGGGGCGGTGACGGCTTCTATGCCATGGACTACTGGGGCCAAG |
| | | GAACCCTGGTCACCGTCTCCTCAGCCTCCACCAAGGGCCCATCGGTCTTCCCCCTGG |
| | | CACCCTCCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGACCAGCGG |
| | | CGTGCACACCTTCCCGGCTGTCCTACAGTCCGGCGGAAGCGGAGCAAAGCTCGCCG |
| | | CACTGAAAGCCAAGCTGGCCGCTCTGAAGGGGGGTGGCGGAAGCGCCCCACCACGC |
| | | CTCATCTGTGACAGCCGAGTCCTGGAGAGGTACCTCTTGGAGGCCAAGGAGGCCGA |
| | | GAATATCACGACGGGCTGTGCTGAACACTGCAGCTTGAATGAGAATATCACTGTCC |
| | | CAGACACCAAAGTTAATTTCTATGCCTGGAAGAGGATGGAGGTCGGGCAGCAGGCC |
| | | GTAGAAGTCTGGCAGGGCCTGGCCCTGCTGTCGGAAGCTGTCCTGCGGGGCCAGGC |
| | | CCTGTTGGTCAACTCTTCCCAGCCGTGGGAGCCCCTGCAGCTGCATGTGGATAAAGC |
| | | CGTCAGTGGCCTTCGCAGCCTCACCACTCTGCTTCGGGCTCTGGGAGCCCAGAAGGA |
| | | AGCCATCTCCCCTCCAGATGCGGCCTCAGCTGCTCCACTCCGAACAATCACTGCTGA |
| | | CACTTTCCGCAAACTCTTCCGAGTCTACTCCAATTTCCTCCGGGGAAAGCTGAAGCT |
| | | GTACACAGGGGAGGCCTGCAGGACAGGGGACAGAGGCGGAGGTGGGAGTGAACTG |
| | | GCCGCACTGGAAGCTGAGCTGGCTGCCCTCGAAGCTGGAGGCTCTGGACTCTACTC |
| | | CCTCAGCAGCGTGGTGACTGTGCCCTCTAGCAGCTTGGGCACCCAGACCTACATCTG |
| | | CAACGTGAATCACAAGCCCAGCAACACCAAGGTGGACAAGAAAGTTGAACCCAAA |
| | | TCTTGCGACAAAACTCACACATGCCCACCGTGCCCAGCACCTCCAGTCGCCGGACC |
| | | GTCAGTCTTCCTCTTCCCTCCAAAACCCAAGGACACCCTCATGATCTCCCGGACCCC |
| | | TGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGA |
| | | GCAGTACAACAGCACGTACCGTGTGGTCAGCGTCCTCACCGTCCTGCACCAGGACT |
| | | GGCTGAATGGCAAGGAGTACAAGTGCAAGGTCTCCAACAAAGGCCTCCCAAGCTCC |
| | | ATCGAGAAAACCATCTCCAAAGCCAAAGGGCAGCCCCGAGAACCACAGGTGTACA |
| | | CCCTGCCTCCATCCCGGGATGAGCTGACCAAGAACCAGGTCAGCCTGACCTGCCTG |
| | | GTCAAAGGCTTCTATCCCAGCGACATCGCCGTGGAGTGGGAGAGCAATGGGCAGCC |
| | | GGAGAACAACTACAAGACCACGCCTCCCGTGCTGGACTCCGACGGCTCCTTCTTCCT |
| | | CTACAGCAAGCTCACCGTGGACAAGAGCAGGTGGCAGCAGGGGAACGTCTTCTCAT |
| | | GCTCCGTGATGCATGAGGCTCTGCACAACCACTACACGCAGAAGAGCCTCTCCCTGT |
| | | CTCCGGGTAAATGATAA |
| 12. | hEPO- | GAAGTGCAGCTGGTGGAGTCTGGAGGAGGCTTGGTCCAGCCTGGGGGGTCCCTGAG |
| | coil- | ACTCTCCTGTGCAGCCTCTGGGTTCAATATTAAGGACACTTACATCCACTGGGTCCG |
| | Her2- | CCAGGCTCCAGGGAAGGGGCTGGAGTGGGTCGCACGTATTTATCCTACCAATGGTT |
| | CH3 | ACACACGCTACGCAGACTCCGTGAAGGGCCGATTCACCATCTCCGCAGACACTTCC |
| | | AAGAACACGGCGTATCTTCAAATGAACAGCCTGAGAGCCGAGGACACGGCCGTGTA |
| | | TTACTGTTCGAGATGGGGCGGTGACGGCTTCTATGCCATGGACTACTGGGGCCAAG |
| | | GAACCCTGGTCACCGTCTCCTCAGCCTCCACCAAGGGCCCATCGGTCTTCCCCCTGG |
| | | CACCCTCCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGACCAGCGG |
| | | CGTGCACACCTTCCCGGCTGTCCTACAGTCCTCAGGACTCTACTCCCTCAGCAGCGT |
| | | GGTGACTGTGCCCTCTAGCAGCTTGGGCACCCAGACCTACATCTGCAACGTGAATCA |
| | | CAAGCCCAGCAACACCAAGGTGGACAAGAAAGTTGAACCCAAATCTTGCGACAAA |
| | | ACTCACACATGCCCACCGTGCCCAGCACCTCCAGTCGCCGGACCGTCAGTCTTCCTC |
| | | TTCCCTCCAAAACCCAAGGACACCCTCATGATCTCCCGGACCCCTGAGGTCACATGC |
| | | GTGGTGGTGGACGTGAGCCACGAAGACCCTGAGGTCAAGTTCAACTGGTACGTGGA |
| | | CGGCGTGGAGGTGCATAATGCCAAGACAAAGCCGCGGGAGGAGCAGTACAACAGC |
| | | ACGTACCGTGTGGTCAGCGTCCTCACCGTCCTGCACCAGGACTGGCTGAATGGCAA |
| | | GGAGTACAAGTGCAAGGTCTCCAACAAAGGCCTCCCAAGCTCCATCGAGAAAACCA |
| | | TCTCCAAAGCCAAAGGGCAGCCCCGAGAACCACAGGTGTACACCCTGCCTCCATCC |
| | | CGGGATGAGCTGGGCGGAAGCGGAGCAAAGCTCGCCGCACTGAAAGCCAAGCTGG |
| | | CCGCTCTGAAGGGGGGTGGCGGAAGCGCCCCACCACGCCTCATCTGTGACAGCCGA |
| | | GTCCTGGAGAGGTACCTCTTGGAGGCCAAGGAGGCCGAGAATATCACGACGGGCTG |
| | | TGCTGAACACTGCAGCTTGAATGAGAATATCACTGTCCCAGACACCAAAGTTAATTT |
| | | CTATGCCTGGAAGAGGATGGAGGTCGGGCAGCAGGCCGTAGAAGTCTGGCAGGGC |
| | | CTGGCCCTGCTGTCGGAAGCTGTCCTGCGGGGCCAGGCCCTGTTGGTCAACTCTTCC |
| | | CAGCCGTGGGAGCCCCTGCAGCTGCATGTGGATAAAGCCGTCAGTGGCCTTCGCAG |
| | | CCTCACCACTCTGCTTCGGGCTCTGGGAGCCCAGAAGGAAGCCATCTCCCCTCCAGA |
| | | TGCGGCCTCAGCTGCTCCACTCCGAACAATCACTGCTGACACTTTCCGCAAACTCTT |
| | | CCGAGTCTACTCCAATTTCCTCCGGGGAAAGCTGAAGCTGTACACAGGGGAGGCCT |
| | | GCAGGACAGGGGACAGAGGCGGAGGTGGGAGTGAACTGGCCGCACTGGAAGCTGA |
| | | GCTGGCTGCCCTCGAAGCTGGAGGCTCTGGACAGGTCAGCCTGACCTGCCTGGTCA |
| | | AAGGCTTCTATCCCAGCGACATCGCCGTGGAGTGGGAGAGCAATGGGCAGCCGGAG |
| | | AACAACTACAAGACCACGCCTCCCGTGCTGGACTCCGACGGCTCCTTCTTCCTCTAC |
| | | AGCAAGCTCACCGTGGACAAGAGCAGGTGGCAGCAGGGGAACGTCTTCTCATGCTC |
| | | CGTGATGCATGAGGCTCTGCACAACCACTACACGCAGAAGAGCCTCTCCCTGTCTCC |
| | | GGGTAAATGATAA |
| 13. | CXCR4- | GAAGTGCAGCTGGTGGAGTCTGGAGGAGGCTTGGTCCAGCCTGGGGGGTCCCTGAG |
| | BP- | ACTCTCCTGTGCAGCCTCTGGGTTCAATATTAAGGACACTTACATCCACTGGGTCCG |
| | coil- | CCAGGCTCCAGGGAAGGGGCTGGAGTGGGTCGCACGTATTTATCCTACCAATGGTT |
| | Her2- | ACACACGCTACGCAGACTCCGTGAAGGGCCGATTCACCATCTCCGCAGACACTTCC |
| | CH1 | AAGAACACGGCGTATCTTCAAATGAACAGCCTGAGAGCCGAGGACACGGCCGTGTA |
| | | TTACTGTTCGAGATGGGGCGGTGACGGCTTCTATGCCATGGACTACTGGGGCCAAG |
| | | GAACCCTGGTCACCGTCTCCTCAGCCTCCACCAAGGGCCCATCGGTCTTCCCCCTGG |
| | | CACCCTCCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGACCAGCGG |
| | | CGTGCACACCTTCCCGGCTGTCCTACAGTCCGGCGGAAGCGGAGCAAAGCTCGCCG |
| | | CACTGAAAGCCAAGCTGGCCGCTCTGAAGGCTAAGTTGTATCGCAAATGTAGAGGA |
| | | GGCCGAAGGTGGTGCTACCAAAAGCTTGAGGCTGAACTGGCCGCACTGGAAGCTGA |
| | | GCTGGCTGCCCTCGAAGCTGGAGGCTCTGGACTCTACTCCCTCAGCAGCGTGGTGAC |
| | | TGTGCCCTCTAGCAGCTTGGGCACCCAGACCTACATCTGCAACGTGAATCACAAGCC |
| | | CAGCAACACCAAGGTGGACAAGAAAGTTGAACCCAAATCTTGCGACAAAACTCACA |
| | | CATGCCCACCGTGCCCAGCACCTGAACTCCTGGGGGGACCGTCAGTCTTCCTCTTCC |
| | | CCCCAAAACCCAAGGACACCCTCATGATCTCCCGGACCCCTGAGGTCACATGCGTG |
| | | GTGGTGGACGTGAGCCACGAAGACCCTGAGGTCAAGTTCAACTGGTACGTGGACGG |
| | | CGTGGAGGTGCATAATGCCAAGACAAAGCCGCGGGAGGAGCAGTACAACAGCACG |
| | | TACCGTGTGGTCAGCGTCCTCACCGTCCTGCACCAGGACTGGCTGAATGGCAAGGA |
| | | GTACAAGTGCAAGGTCTCCAACAAAGCCCTCCCAGCCCCCATCGAGAAAACCATCT |
| | | CCAAAGCCAAAGGGCAGCCCCGAGAACCACAGGTGTACACCCTGCCCCCATCCCGG |
| | | GATGAGCTGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAG |
| | | ACCACGCCTCCCGTGCTGGACTCCGACGGCTCCTTCTTCCTCTACAGCAAGCTCACC |
| | | GTGGACAAGAGCAGGTGGCAGCAGGGGAACGTCTTCTCATGCTCCGTGATGCATGA |
| | | GGCTCTGCACAACCACTACACGCAGAAGAGCCTCTCCCTGTCTCCGGGTAAATGAT |
| | | AA |
| 14. | hEPO- | GACATCCAGATGACCCAGTCTCCATCCTCCCTGTCTGCATCTGTAGGAGACAGAGTC |
| | coil- | ACCATCACTTGCCGGGCAAGTCAGGATGTGAATACCGCGGTCGCATGGTATCAGCA |
| | Her2- | GAAACCAGGGAAAGCCCCTAAGCTCCTGATCTATTCTGCATCCTTCTTGTATAGTGG |
| | CL | GGTCCCATCAAGGTTCAGTGGCAGTAGATCTGGGACAGATTTCACTCTCACCATCAG |
| | | CAGTCTGCAACCTGAAGATTTTGCAACTTACTACTGTCAACAGCATTACACTACCCC |
| | | TCCGACGTTCGGCCAAGGTACCAAGCTTGAGATCAAACGAACTGTGGCTGCACCAT |
| | | CTGTCTTCATCTTCCCGCCATCTGATGAGCAGTTGAAATCTGGAACTGCCTCTGTCGT |
| | | GTGCCTGCTGAATAACTTCTATCCCAGAGAGGCCAAAGTACAGTGGAAGGTGGATA |
| | | ACGCCCTCCAATCGGGTAACTCCCAGGAGAGTGTCACAGAGCAGGACAGCGGCGGA |
| | | AGCGGAGCAAAGCTCGCCGCACTGAAAGCCAAGCTGGCCGCTCTGAAGGGGGGTG |
| | | GCGGAAGCGCCCCACCACGCCTCATCTGTGACAGCCGAGTCCTGGAGAGGTACCTC |
| | | TTGGAGGCCAAGGAGGCCGAGAATATCACGACGGGCTGTGCTGAACACTGCAGCTT |
| | | GAATGAGAATATCACTGTCCCAGACACCAAAGTTAATTTCTATGCCTGGAAGAGGA |
| | | TGGAGGTCGGGCAGCAGGCCGTAGAAGTCTGGCAGGGCCTGGCCCTGCTGTCGGAA |
| | | GCTGTCCTGCGGGGCCAGGCCCTGTTGGTCAACTCTTCCCAGCCGTGGGAGCCCCTG |
| | | CAGCTGCATGTGGATAAAGCCGTCAGTGGCCTTCGCAGCCTCACCACTCTGCTTCGG |
| | | GCTCTGGGAGCCCAGAAGGAAGCCATCTCCCCTCCAGATGCGGCCTCAGCTGCTCC |
| | | ACTCCGAACAATCACTGCTGACACTTTCCGCAAACTCTTCCGAGTCTACTCCAATTT |
| | | CCTCCGGGGAAAGCTGAAGCTGTACACAGGGGAGGCCTGCAGGACAGGGGACAGA |
| | | GGCGGAGGTGGGAGTGAACTGGCCGCACTGGAAGCTGAGCTGGCTGCCCTCGAAGC |
| | | TGGAGGCTCTGGAGACAGCACCTACAGCCTCAGCAGCACCCTGACGCTGAGCAAAG |
| | | CAGACTACGAGAAACACAAAGTCTACGCCTGCGAAGTCACCCATCAGGGCCTGTCC |
| | | TCGCCCGTCACAAAGAGCTTCAACAGGGGAGAGTGT |
| 15. | hEPO- | GACATCCAGATGACCCAGTCTCCATCCTCCCTGTCTGCATCTGTAGGAGACAGAGTC |
| | G4S- | ACCATCACTTGCCGGGCAAGTCAGGATGTGAATACCGCGGTCGCATGGTATCAGCA |
| | Trastuzumab- | GAAACCAGGGAAAGCCCCTAAGCTCCTGATCTATTCTGCATCCTTCTTGTATAGTGG |
| | CL | GGTCCCATCAAGGTTCAGTGGCAGTAGATCTGGGACAGATTTCACTCTCACCATCAG |
| | | CAGTCTGCAACCTGAAGATTTTGCAACTTACTACTGTCAACAGCATTACACTACCCC |
| | | TCCGACGTTCGGCCAAGGTACCAAGCTTGAGATCAAACGAACTGTGGCTGCACCAT |
| | | CTGTCTTCATCTTCCCGCCATCTGATGAGCAGTTGAAATCTGGAACTGCCTCTGTCGT |
| | | GTGCCTGCTGAATAACTTCTATCCCAGAGAGGCCAAAGTACAGTGGAAGGTGGATA |
| | | ACGCCCTCCAATCGGGTAACTCCCAGGAGAGTGTCACAGAGCAGGACAGCGGGGGT |
| | | GGCGGAAGCGCCCCACCACGCCTCATCTGTGACAGCCGAGTCCTGGAGAGGTACCT |
| | | CTTGGAGGCCAAGGAGGCCGAGAATATCACGACGGGCTGTGCTGAACACTGCAGCT |
| | | TGAATGAGAATATCACTGTCCCAGACACCAAAGTTAATTTCTATGCCTGGAAGAGG |
| | | ATGGAGGTCGGGCAGCAGGCCGTAGAAGTCTGGCAGGGCCTGGCCCTGCTGTCGGA |
| | | AGCTGTCCTGCGGGGCCAGGCCCTGTTGGTCAACTCTTCCCAGCCGTGGGAGCCCCT |
| | | GCAGCTGCATGTGGATAAAGCCGTCAGTGGCCTTCGCAGCCTCACCACTCTGCTTCG |
| | | GGCTCTGGGAGCCCAGAAGGAAGCCATCTCCCCTCCAGATGCGGCCTCAGCTGCTC |
| | | CACTCCGAACAATCACTGCTGACACTTTCCGCAAACTCTTCCGAGTCTACTCCAATT |
| | | TCCTCCGGGGAAAGCTGAAGCTGTACACAGGGGAGGCCTGCAGGACAGGGGACAG |
| | | AGGCGGAGGTGGGAGTGACAGCACCTACAGCCTCAGCAGCACCCTGACGCTGAGCA |
| | | AAGCAGACTACGAGAAACACAAAGTCTACGCCTGCGAAGTCACCCATCAGGGCCTG |
| | | TCCTCGCCCGTCACAAAGAGCTTCAACAGGGGAGAGTGT |
| 16. | TCP1- | GATATTCAGATGACTCAGACTACCAGTTCACTGAGCGCCTCCCTGGGCGATCGCGTG |
| | coil- | ACAATTAGTTGTCGTGCGTCACAGGACATCCGGAACTATCTGAATTGGTACCAGCA |
| | UCHT1- | GAAGCCGGACGGCACAGTCAAACTGCTGATCTATTACACTAGCCGTCTGCATTCCG |
| | CL | GTGTGCCCTCTAAGTTTTCTGGGAGTGGATCAGGCACTGATTATAGTCTGACCATTT |
| | (IgG) | CAAACCTGGAACAGGAAGATATCGCCACCTACTTCTGTCAGCAGGGGAATACTCTG |
| | | CCGTGGACTTTCGCCGGAGGAACCAAACTGGAGATTAAGCGAACTGTGGCTGCACC |
| | | ATCTGTCTTCATCTTCCCGCCATCTGATGAGCAGTTGAAATCTGGAACTGCCTCTGTC |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCGGCG |
| | | GAAGCGGAGCAAAGCTCGCCGCACTGAAAGCCAAGCTGGCCGCTCTGAAGGCCAA |
| | | GCTGACTCCCAGCCCTTTCTCACACCTGGAAGCTGAACTGGCCGCACTGGAAGCTGA |
| | | GCTGGCTGCCCTCGAAGCTGGAGGCTCTGGAGACAGCACCTACAGCCTCAGCAGCA |
| | | CCCTGACGCTGAGCAAAGCAGACTACGAGAAACACAAAGTCTACGCCTGCGAAGTC |
| | | ACCCATCAGGGCCTGTCCTCGCCCGTCACAAAGAGCTTCAACAGGGGAGAGTGT |
| 17. | TCP1- | GATATTCAGATGACTCAGACTACCAGTTCACTGAGCGCCTCCCTGGGCGATCGCGTG |
| | UCHT1- | ACAATTAGTTGTCGTGCGTCACAGGACATCCGGAACTATCTGAATTGGTACCAGCA |
| | CL | GAAGCCGGACGGCACAGTCAAACTGCTGATCTATTACACTAGCCGTCTGCATTCCG |
| | | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGCGTACGGTGGCTGCACC |
| | | ATCTGTCTTCATCTTCCCGCCATCTGATGAGCAGTTGAAATCTGGAACTGCCTCTGTT |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCGGCG |
| | | GAGGCGGGAGCTGTACTCCCAGCCCTTTCTCACACTGTGGTGGCGGAGGCAGCGAC |
| | | AGCACCTACAGCCTCAGCAGCACCCTGACGCTGAGCAAAGCAGACTACGAGAAACA |
| | | CAAAGTCTACGCCTGCGAAGTCACCCATCAGGGCCTGAGCTCGCCCGTCACAAAGA |
| | | GCTTCAACAGGGGAGAGTGT |
| 18. | NGR- | GATATTCAGATGACTCAGACTACCAGTTCACTGAGCGCCTCCCTGGGCGATCGCGTG |
| | coil- | ACAATTAGTTGTCGTGCGTCACAGGACATCCGGAACTATCTGAATTGGTACCAGCA |
| | UCHT1- | GAAGCCGGACGGCACAGTCAAACTGCTGATCTATTACACTAGCCGTCTGCATTCCG |
| | CL | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGCGTACGGTGGCTGCACC |
| | | ATCTGTCTTCATCTTCCCGCCATCTGATGAGCAGTTGAAATCTGGAACTGCCTCTGTC |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCGGCG |
| | | GAAGCGGAGCAAAGCTCGCCGCACTGAAAGCCAAGCTGGCCGCTCTGAAGGCTAA |
| | | GTTGACATATAATGGGAGGACACTTGAGGCTGAACTGGCCGCACTGGAAGCTGAGC |
| | | TGGCTGCCCTCGAAGCTGGAGGCTCTGGAGACAGCACCTACAGCCTCAGCAGCACC |
| | | CTGACGCTGAGCAAAGCAGACTACGAGAAACACAAAGTCTACGCCTGCGAAGTCAC |
| | | CCATCAGGGCCTGTCCTCGCCCGTCACAAAGAGCTTCAACAGGGGAGAGTGT |
| 19. | NGR- | GATATTCAGATGACTCAGACTACCAGTTCACTGAGCGCCTCCCTGGGCGATCGCGTG |
| | UCHT1- | ACAATTAGTTGTCGTGCGTCACAGGACATCCGGAACTATCTGAATTGGTACCAGCA |
| | CL | GAAGCCGGACGGCACAGTCAAACTGCTGATCTATTACACTAGCCGTCTGCATTCCG |
| | | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGCGTACGGTGGCTGCACC |
| | | ATCTGTCTTCATCTTCCCGCCATCTGATGAGCAGTTGAAATCTGGAACTGCCTCTGTT |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCGGCG |
| | | GAGGCGGGAGCTGTAACGGAAGATGTGTGTCCGGTTGCGCTGGCCGCTGTGGTGGC |
| | | GGAGGCAGCGACAGCACCTACAGCCTCAGCAGCACCCTGACGCTGAGCAAAGCAG |
| | | ACTACGAGAAACACAAAGTCTACGCCTGCGAAGTCACCCATCAGGGCCTGAGCTCG |
| | | CCCGTCACAAAGAGCTTCAACAGGGGAGAGTGT |
| 20. | Int- | GATATTCAGATGACTCAGACTACCAGTTCACTGAGCGCCTCCCTGGGCGATCGCGTG |
| | coil- | ACAATTAGTTGTCGTGCGTCACAGGACATCCGGAACTATCTGAATTGGTACCAGCA |
| | UCHT1- | GAAGCCGGACGGCACAGTCAAACTGCTGATCTATTACACTAGCCGTCTGCATTCCG |
| | CL | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGCGAACTGTGGCTGCACC |
| | | ATCTGTCTTCATCTTCCCGCCATCTGATGAGCAGTTGAAATCTGGAACTGCCTCTGTC |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCGGCG |
| | | GAAGCGGAGCAAAGCTCGCCGCACTGAAAGCCAAGCTGGCCGCTCTGAAGGGGGG |
| | | TGGCGGAAGCGGTTGCCCTCAAGGGCGCGGGGATTGGGCACCCACCTCCTGTAAGC |
| | | AAGACTCTGACTGCCGCGCTGGCTGCGTGTGCGGTCCCAATGGTTTTTGCGGGGGAG |
| | | GCGGTGGGAGCGAACTGGCCGCACTGGAAGCTGAGCTGGCTGCCCTCGAAGCTGGA |
| | | GGCTCTGGAGACAGCACCTACAGCCTCAGCAGCACCCTGACGCTGAGCAAAGCAGA |
| | | CTACGAGAAACACAAAGTCTACGCCTGCGAAGTCACCCATCAGGGCCTGTCCTCGC |
| | | CCGTCACAAAGAGCTTCAACAGGGGAGAGTGT |
| 21. | CXCR4- | CAGATTGTGTTGTCTCAGTCCCCCGCAATTCTCAGTGCGTCCCCCGGCGAAAAGGTG |
| | BP- | ACCATGACCTGCCGCGCTTCCTCCTCAGTGAGTTATATCCACTGGTTCCAGCAGAAG |
| | coil- | CCAGGATCAAGCCCGAAGCCGTGGATCTACGCCACCAGCAACCTGGCCAGCGGAGT |
| | CD20- | GCCTGTGAGGTTCTCTGGTTCTGGCAGCGGGACCAGTTACTCACTCACCATTTCCCG |
| | CL | GGTTGAGGCCGAAGATGCCGCTACTTATTATTGCCAACAGTGGACCTCCAATCCGCC |
| | (IgG) | AACATTTGGGGGAGGGACTAAACTGGAGATTAAACGAACTGTGGCTGCACCATCTG |
| | | TCTTCATCTTCCCGCCATCTGATGAGCAGTTGAAATCTGGAACTGCCTCTGTCGTGT |
| | | GCCTGCTGAATAACTTCTATCCCAGAGAGGCCAAAGTACAGTGGAAGGTGGATAAC |
| | | GCCCTCCAATCGGGTAACTCCCAGGAGAGTGTCACAGAGCAGGACAGCGGCGGAA |
| | | GCGGAGCAAAGCTCGCCGCACTGAAAGCCAAGCTGGCCGCTCTGAAGGCTAAGTTG |
| | | TATCGCAAATGTAGAGGAGGCCGAAGGTGGTGCTACCAAAAGCTTGAGGCTGAACT |
| | | GGCCGCACTGGAAGCTGAGCTGGCTGCCCTCGAAGCTGGAGGCTCTGGAGACAGCA |
| | | CCTACAGCCTCAGCAGCACCCTGACGCTGAGCAAAGCAGACTACGAGAAACACAAA |
| | | GTCTACGCCTGCGAAGTCACCCATCAGGGCCTGTCCTCGCCCGTCACAAAGAGCTTC |
| | | AACAGGGGAGAGTGT |
| 22. | CXCR4- | GACATCCAGATGACCCAGTCTCCATCCTCCCTGTCTGCATCTGTAGGAGACAGAGTC |
| | BP- | ACCATCACTTGCCGGGCAAGTCAGGATGTGAATACCGCGGTCGCATGGTATCAGCA |
| | coil- | GAAACCAGGGAAAGCCCCTAAGCTCCTGATCTATTCTGCATCCTTCTTGTATAGTGG |
| | Her2- | GGTCCCATCAAGGTTCAGTGGCAGTAGATCTGGGACAGATTTCACTCTCACCATCAG |
| | CL | CAGTCTGCAACCTGAAGATTTTGCAACTTACTACTGTCAACAGCATTACACTACCCC |
| | | TCCGACGTTCGGCCAAGGTACCAAGCTTGAGATCAAACGAACTGTGGCTGCACCAT |
| | | CTGTCTTCATCTTCCCGCCATCTGATGAGCAGTTGAAATCTGGAACTGCCTCTGTCGT |
| | | GTGCCTGCTGAATAACTTCTATCCCAGAGAGGCCAAAGTACAGTGGAAGGTGGATA |
| | | ACGCCCTCCAATCGGGTAACTCCCAGGAGAGTGTCACAGAGCAGGACAGCGGCGGA |
| | | AGCGGAGCAAAGCTCGCCGCACTGAAAGCCAAGCTGGCCGCTCTGAAGGCTAAGTT |
| | | GTATCGCAAATGTAGAGGAGGCCGAAGGTGGTGCTACCAAAAGCTTGAGGCTGAAC |
| | | TGGCCGCACTGGAAGCTGAGCTGGCTGCCCTCGAAGCTGGAGGCTCTGGAGACAGC |
| | | ACCTACAGCCTCAGCAGCACCCTGACGCTGAGCAAAGCAGACTACGAGAAACACAA |
| | | AGTCTACGCCTGCGAAGTCACCCATCAGGGCCTGTCCTCGCCCGTCACAAAGAGCTT |
| | | CAACAGGGGAGAGTGT |
| 23. | GCN4- | GACATCCAGATGACACAGACTACATCCTCCCTGTCTGCCTCTCTGGGAGACAGAGTC |
| | CD19- | ACCATCAGTTGCAGGGCAAGTCAGGACATTAGTAAATATTTAAATTGGTATCAGCA |
| | CL | GAAACCAGATGGAACTGTTAAACTCCTGATCTACCATACATCAAGATTACACTCAG |
| | | GAGTCCCATCAAGGTTCAGTGGCAGTGGGTCTGGAACAGATTATTCTCTCACCATTA |
| | | GCAACCTGGAGCAAGAAGATATTGCCACTTACTTTTGCCAACAGGGTAATACGCTTC |
| | | CGTACACGTTCGGAGGGGGGACCAAGCTTGAGATCAAACGAACTGTGGCTGCACCA |
| | | TCTGTCTTCATCTTCCCGCCATCTGATGAGCAGTTGAAATCTGGAACTGCCTCTGTCG |
| | | TGTGCCTGCTGAATAACTTCTATCCCAGAGAGGCCAAAGTACAGTGGAAGGTGGAT |
| | | AACGCCCTCCAATCGGGTAACTCCCAGGAGAGTGTCACAGAGCAGGACAGCGGCGG |
| | | AGGCGGGAGCAATTATCATCTTGAAAATGAGGTCGCTCGTCTCAAGAAACTCGGTG |
| | | GCGGAGGCAGCGACAGCACCTACAGCCTCAGCAGCACCCTGACGCTGAGCAAAGC |
| | | AGACTACGAGAAACACAAAGTCTACGCCTGCGAAGTCACCCATCAGGGCCTGTCCT |
| | | CGCCCGTCACAAAGAGCTTCAACAGGGGAGAGTGT |
| 24. | Her2ScFv- | GATATTCAGATGACTCAGACTACCAGTTCACTGAGCGCCTCCCTGGGCGATCGCGTG |
| | UCHT1- | ACAATTAGTTGTCGTGCGTCACAGGACATCCGGAACTATCTGAATTGGTACCAGCA |
| | CL | GAAGCCGGACGGCACAGTCAAACTGCTGATCTATTACACTAGCCGTCTGCATTCCG |
| | | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGCGTACGGTGGCTGCACC |
| | | ATCTGTCTTCATCTTCCCGCCATCTGATGAGCAGTTGAAATCTGGAACTGCCTCTGTT |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCGGGG |
| | | GCGGCGGATCTGGCGGAGGCGGGTCTGGCGGGGGTGGATCTGATATTCAGATGACC |
| | | CAGAGCCCTAGCTCTCTTAGCGCATCCGTTGGTGACCGCGTAACTATTACTTGCAGA |
| | | GCCAGTCAGGATGTGAATACGGCTGTGGCCTGGTATCAGCAGAAACCTGGGAAAGC |
| | | CCCCAAGCTGCTGATCTACTCCGCCAGCTTCCTGTATTCTGGTGTGCCGAGCAGATT |
| | | TAGCGGGTCCAGAAGCGGCACCGACTTTACCCTTACTATTTCATCCCTGCAGCCGGA |
| | | GGATTTCGCCACATATTATTGTCAGCAGCACTACACCACACCTCCCACATTCGGCCA |
| | | GGGCACTAAGGTGGAGATCAAACGCACAGGGTCAACTTCAGGTTCCGGCAAGCCCG |
| | | GTTCTGGAGAGGGGAGCGAAGTGCAGCTCGTCGAGTCCGGCGGTGGTCTGGTCCAG |
| | | CCGGGAGGAAGCCTGCGACTGAGCTGTGCAGCGTCTGGATTCAACATCAAGGACAC |
| | | CTACATCCACTGGGTGCGCCAGGCACCCGGCAAAGGCCTTGAGTGGGTGGCACGGA |
| | | TCTACCCAACTAACGGGTATACCAGATACGCCGATAGCGTGAAGGGACGGTTCACA |
| | | ATAAGCGCAGATACTTCTAAGAACACTGCCTATCTGCAGATGAACTCACTGCGGGC |
| | | TGAGGACACTGCCGTGTATTATTGTAGCAGATGGGGTGGCGATGGGTTCTACGCCAT |
| | | GGATGTCTGGGGTCAGGGTACTTTGGTGACCGTGTCTTCAGGGGGCGGCGGCAGTG |
| | | ACAGCACCTACAGCCTCAGCAGCACCCTGACGCTGAGCAAAGCAGACTACGAGAAA |
| | | CACAAAGTCTACGCCTGCGAAGTCACCCATCAGGGCCTGAGCTCGCCCGTCACAAA |
| | | GAGCTTCAACAGGGGAGAGTGT |
| 25. | anti- | GATATTCAGATGACTCAGACTACCAGTTCACTGAGCGCCTCCCTGGGCGATCGCGTG |
| | CD19 | ACAATTAGTTGTCGTGCGTCACAGGACATCCGGAACTATCTGAATTGGTACCAGCA |
| | ScFv- | GAAGCCGGACGGCACAGTCAAACTGCTGATCTATTACACTAGCCGTCTGCATTCCG |
| | UCHT1- | GTGTGCCCTCTAAGTTTTCTGGGAGTGGATCAGGCACTGATTATAGTCTGACCATTT |
| | CL(Fab) | CAAACCTGGAACAGGAAGATATCGCCACCTACTTCTGTCAGCAGGGGAATACTCTG |
| | | CCGTGGACTTTCGCCGGAGGAACCAAACTGGAGATTAAGCGTACGGTGGCTGCACC |
| | | ATCTGTCTTCATCTTCCCGCCATCTGATGAGCAGTTGAAATCTGGAACTGCCTCTGTT |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCGGGG |
| | | GTGGCGGAAGTGGGGGCGGAGGCAGTGGGGGAGGCGGTAGTGAGGTGAAACTGCA |
| | | GGAGTCAGGACCTGGCCTGGTGGCGCCCTCACAGAGCCTGTCCGTCACATGCACTG |
| | | TCTCAGGGGTCTCATTACCCGACTATGGTGTAAGCTGGATTCGCCAGCCTCCACGAA |
| | | GCTCTCAAATCCAGACTGACCATCATCAAGGACAACTCCAAGAGCCAAGTTTTCTTA |
| | | AAAATGAACAGTCTGCAAACTGATGACACAGCCATTTACTACTGTGCCAAACATTA |
| | | TTACTACGGTGGTAGCTATGCTATGGACTACTGGGGCCAAGGAACCTCAGTCACCGT |
| | | CTCCTCAGGAGGCGGAGGATCCGGAGGCGGTGGCAGCGGCGGCGGAGGTTCTGAC |
| | | ATCCAGATGACACAGACTACATCCTCCCTGTCTGCCTCTCTGGGAGACAGAGTCACC |
| | | ATCAGTTGCAGGGCAAGTCAGGACATTAGTAAATATTTAAATTGGTATCAGCAGAA |
| | | ACCAGATGGAACTGTTAAACTCCTGATCTACCATACATCAAGATTACACTCAGGAGT |
| | | CCCATCAAGGTTCAGTGGCAGTGGGTCTGGAACAGATTATTCTCTCACCATTAGCAA |
| | | CCTGGAGCAAGAAGATATTGCCACTTACTTTTGCCAACAGGGTAATACGCTTCCGTA |
| | | CACGTTCGGAGGGGGGACCAAGCTTGAGATCGGTGGCGGTGGGTCTGACAGCACCT |
| | | ACAGCCTCAGCAGCACCCTGACGCTGAGCAAAGCAGACTACGAGAAACACAAAGT |
| | | CTACGCCTGCGAAGTCACCCATCAGGGCCTGAGCTCGCCCGTCACAAAGAGCTTCA |
| | | ACAGGGGAGAGTGT |
| 26 | UCHT1 | GAAGTGCAGCTGGTGGAGTCTGGAGGAGGCTTGGTCCAGCCTGGGGGGTCCCTGAG |
| | ScFv- | ACTCTCCTGTGCAGCCTCTGGGTTCAATATTAAGGACACTTACATCCACTGGGTCCG |
| | Her2- | CCAGGCTCCAGGGAAGGGGCTGGAGTGGGTCGCACGTATTTATCCTACCAATGGTT |
| | CH1 | ACACACGCTACGCAGACTCCGTGAAGGGCCGATTCACCATCTCCGCAGACACTTCC |
| | | AAGAACACGGCGTATCTTCAAATGAACAGCCTGAGAGCCGAGGACACGGCCGTGTA |
| | | TTACTGTTCGAGATGGGGCGGTGACGGCTTCTATGCCATGGACTACTGGGGCCAAG |
| | | GAACCCTGGTCACCGTCTCCTCAGCCTCCACCAAGGGCCCATCGGTCTTCCCCCTGG |
| | | CACCCTCCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGACCAGCGG |
| | | CGTGCACACCTTCCCGGCTGTCCTACAGTCCGGAGGGGGAGGAAGTGGTGGCGGGG |
| | | GGAGCGGCGGAGGAGGCTCCGACATTCAGATGACCCAGACCACCAGCTCTCTGAGT |
| | | GCCAGCCTTGGGGATCGGGTGACAATTTCCTGCCGGGCCTCTCAGGATATACGCAA |
| | | CTACCTGAACTGGTACCAGCAGAAGCCTGATGGCACAGTGAAACTGCTGATTTACT |
| | | ATACGTCCAGACTGCACTCAGGGGTTCCCAGTAAATTCAGCGGCTCCGGCTCCGGA |
| | | ACGGACTACTCACTGACCATCTCAAACTTGGAGCAGGAGGACATTGCCACTTATTTC |
| | | TGCCAACAGGGGAACACCCTCCCCTGGACTTTCGCTGGAGGAACTAAGCTCGAAAT |
| | | AAAGGGATCAACTTCAGGGTCAGGGAAGCCTGGTAGCGGTGAGGGGTCCACGAAG |
| | | GGTGAAGTGCAGCTGCAGCAGTCTGGACCCGAGCTGGTGAAGCCGGGTGCATCTAT |
| | | GAAAATTTCCTGCAAAGCAAGCGGGTATTCCTTTACCGGGTACACTATGAATTGGGT |
| | | GAAGCAGAGCCACGGGAAGAATCTGGAATGGATGGGACTGATAAATCCTTACAAG |
| | | GGCGTCAGCACATACAATCAGAAATTCAAGGATAAGGCTACACTTACAGTAGACAA |
| | | AAGTTCCTCCACTGCATATATGGAGCTGCTTTCACTCACCTCAGAAGACTCCGCCGT |
| | | GTATTATTGTGCTAGATCAGGGTACTATGGCGACTCAGACTGGTACTTCGATGTATG |
| | | GGGACAGGGTACCACACTGACCGTGTTCAGCGGAGGAGGCGGCAGCCTCTACTCCC |
| | | TCAGCAGCGTGGTGACTGTGCCCTCTAGCAGCTTGGGCACCCAGACCTACATCTGCA |
| | | ACGTGAATCACAAGCCCAGCAACACCAAGGTGGACAAGAAAGTTGAACCCAAATCT |
| | | TGCGACAAAACTCACACATGCCCACCGTGCCCAGCACCTCCAGTCGCCGGACCGTC |
| | | AGTCTTCCTCTTCCCTCCAAAACCCAAGGACACCCTCATGATCTCCCGGACCCCTGA |
| | | GGTCACATGCGTGGTGGTGGACGTGAGCCACGAAGACCCTGAGGTCAAGTTCAACT |
| | | GGTACGTGGACGGCGTGGAGGTGCATAATGCCAAGACAAAGCCGCGGGAGGAGCA |
| | | GTACAACAGCACGTACCGTGTGGTCAGCGTCCTCACCGTCCTGCACCAGGACTGGCT |
| | | GAATGGCAAGGAGTACAAGTGCAAGGTCTCCAACAAAGGCCTCCCAAGCTCCATCG |
| | | AGAAAACCATCTCCAAAGCCAAAGGGCAGCCCCGAGAACCACAGGTGTACACCCTG |
| | | CCTCCATCCCGGGATGAGCTGACCAAGAACCAGGTCAGCCTGACCTGCCTGGTCAA |
| | | AGGCTTCTATCCCAGCGACATCGCCGTGGAGTGGGAGAGCAATGGGCAGCCGGAGA |
| | | ACAACTACAAGACCACGCCTCCCGTGCTGGACTCCGACGGCTCCTTCTTCCTCTACA |
| | | GCAAGCTCACCGTGGACAAGAGCAGGTGGCAGCAGGGGAACGTCTTCTCATGCTCC |
| | | GTGATGCATGAGGCTCTGCACAACCACTACACGCAGAAGAGCCTCTCCCTGTCTCCG |
| | | GGTAAATGATAA |
| 27. | CXCR4- | CAGATTGTGTTGTCTCAGTCCCCCGCAATTCTCAGTGCGTCCCCCGGCGAAAAGGTG |
| | BP- | ACCATGACCTGCCGCGCTTCCTCCTCAGTGAGTTATATCCACTGGTTCCAGCAGAAG |
| | coil- | CCAGGATCAAGCCCGAAGCCGTGGATCTACGCCACCAGCAACCTGGCCAGCGGAGT |
| | CD20- | GCCTGTGAGGTTCTCTGGTTCTGGCAGCGGGACCAGTTACTCACTCACCATTTCCCG |
| | CL | GGTTGAGGCCGAAGATGCCGCTACTTATTATTGCCAACAGTGGACCTCCAATCCGCC |
| | (IgG) | AACATTTGGGGGAGGGACTAAACTGGAGATTaaacgaactgtggctgcaccatctgtct |
| | | tcatcttcccgccatctgatgagcagttgaaatctggaactgcctctgtcgtgtgcctg |
| | | ctgaataacttctatcccagagaggccaaagtacagtggaaggtggataacgccctccaa |
| | | tcgggtaactcccaggagagtgtcacagagcaggacagcGGCGGAAGCGGAGCAAAGCTC |
| | | GCCGCACTGAAAGCCAAGCTGGCCGCTCTGAAGGGGGGTGGCGGAAGCTGCTATCGCAA |
| | | ATGTAGAGGAGGCCGAAGGTGGTGCTACCAAAAGTGTGGCGGAGGTGGGAGTGAACTGG |
| | | CCGCACTGGAAGCTGAGCTGGCTGCCCTCGAAGCTGGAGGCTCTGGAgacagcaccta |
| | | cagcctcagcagcaccctgacgctgagcaaagcagactacgagaaacacaaagtctacg |
| | | cctgcgaagtcacccatcagggcctgtcctcgcccgtcacaaagagcttcaacagggga |
| | | gagtgt |
| 28. | CXCR4- | GACATCCAGATGACCCAGTCCCCCTCCACCCTGTCCGCCTCCGTGGGCGACCGCGTG |
| | BP- | ACCATCACCTGCAAGTGCCAGCTGTCCGTGGGCTACATGCACTGGTACCAGCAGAA |
| | coil- | GCCCGGCAAGGCCCCCAAGCTGCTGATCTACGACACCTCCAAGCTGGCCTCCGGCG |
| | Syn- | TGCCCTCCCGCTTCTCCGGCTCCGGCTCCGGCACCGAGTTCACCCTGACCATCTCCTC |
| | CL | CCTGCAGCCCGACGACTTCGCCACCTACTACTGCTTCCAGGGCTCCGGCTACCCCTT |
| | | CACCTTCGGCGGCGGCACCAAGCTGGAGATCaaacgaactgtggctgcaccatctgtctt |
| | | catcttcccgccatctgatgagcagttgaaatctggaactgcctctgtcgtgtgcctgct |
| | | gaataacttctatcccagagaggccaaagtacagtggaaggtggataacgccctccaatcg |
| | | ggtaactcccaggagagtgtcacagagcaggacagcGGCGGAAGCGGAGCAAAGCTCGCCG |
| | | CACTGAAAGCCAAGCTGGCCGCTCTGAAGGGGGGTGGCGGAAGCTGCTATCGCAAATGTAG |
| | | AGGAGGCCGAAGGTGGTGCTACCAAAAGTGTGGCGGAGGTGGGAGTGAACTGGCCGCACT |
| | | GGAAGCTGAGCTGGCTGCCCTCGAAGCTGGAGGCTCTGGAgacagcacctacagcctcagc |
| | | agcaccctgacgctgagcaaagcagactacgagaaacacaaagtctacgcctgcgaagtc |
| | | acccatcagggcctgtcctcgcccgtcacaaagagcttcaacaggggagagtgt |
| 29. | Her2S | GATATTCAGATGACTCAGACTACCAGTTCACTGAGCGCCTCCCTGGGCGATCGCGTG |
| | cFv- | ACAATTAGTTGTCGTGCGTCACAGGACATCCGGAACTATCTGAATTGGTACCAGCA |
| | UCHT1- | GAAGCCGGACGGCACAGTCAAACTGCTGATCTATTACACTAGCCGTCTGCATTCCG |
| | CL- | GTGTGCCCTCTAAGTTTTCTGGGAGTGGATCAGGCACTGATTATAGTCTGACCATTT |
| | L2A | CAAACCTGGAACAGGAAGATATCGCCACCTACTTCTGTCAGCAGGGGAATACTCTG |
| | | CCGTGGACTTTCGCCGGAGGAACCAAACTGGAGATTAAGcgtacggtggctgcaccatc |
| | | tgtcttcatcttcccgccatctgatgagcagttgaaatctggaactgcctctgtcgtgt |
| | | gcctgctgaataacttctatcccagagaggccaaagtacagtggaaggtggataacgccc |
| | | tccaatcgggtaactcccaggagagtgtcacagagcaggacagcGGCGGAAGCGGAGCAAA |
| | | GCTCGCCGCACTGAAAGCCAAGCTGGCCGCTCTGAAGTGTGGAGGAGGAGGAAGTGGGGGA |
| | | GGCGGCAGCGGGGGAGGTGGATCCGACATTCAAATGACGCAGTCACCCTCTTCCCTGTCC |
| | | GCCAGCGTGGGGGATCGCGTCACAATCACATGTCGCGCCTCTCAGGATGTGAACACCGCG |
| | | GTGGCTTGGTATCAACAGAAGCCAGGCAAAGCACCTAAGCTCCTGATCTACTCTGCCAGC |
| | | TTTTTGTACAGCGGCGTGCCAAGTAGGTTTTCAGGCTCTAGAAGCGGCACAGACTTTACAC |
| | | TGACTATCTCATCCCTGCAGCCTGAGGACTTTGCTACATATTATTGTCAACAACATTATA |
| | | CTACTCCACCCACTTTCGGACAGGGCACCAAAGTGGAGATCAAACGCACCGGCTCCACCA |
| | | GTGGAAGCGGTAAGCCTGGCTCTGGCGAAGGCTCAGAAGTGCAACTTGTGGAGTCTGGAG |
| | | GGGGGCTCGTCCAGCCCGGCGGTAGTCTGAGGCTCAGCTGCGCCGCATCTGGCTTTAATA |
| | | TCAAGGACACATATATCCACTGGGTACGGCAAGCACCAGGTAAGGGACTGGAGTGGGTCG |
| | | CCAGAATCTACCCCACAAACGGGTACACTCGCTATGCCGACTCAGTCAAGGGACGCTTTA |
| | | CAATAAGCGCAGACACAAGCAAGAACACCGCTTATCTGCAGATGAATAGCTTGCGGGCGG |
| | | AGGATACAGCTGTGTACTACTGCAGCAGATGGGGGGGCGACGGCTTTTACGCTATGGATG |
| | | TGTGGGGCCAGGGTACTCTGGTGACCGTCTCCTCCGGAGGCGGTGGGAGCTGTGAACTGGC |
| | | CGCACTGGAAGCTGAGCTGGCTGCCCTCGAAGCTGGAGGCTCTGGAgacagcacctacag |
| | | cctcagcagcaccctgacgctgagcaaagcagactacgagaaacacaaagtctacgcctg |
| | | cgaagtcacccatcagggcctgtcctcgcccgtcacaaagagcttcaacaggggagagtgt |
| 30. | Her2ScFv- | GATATTCAGATGACTCAGACTACCAGTTCACTGAGCGCCTCCCTGGGCGATCGCGTG |
| | UCHT1- | ACAATTAGTTGTCGTGCGTCACAGGACATCCGGAACTATCTGAATTGGTACCAGCA |
| | CL- | GAAGCCGGACGGCACAGTCAAACTGCTGATCTATTACACTAGCCGTCTGCATTCCG |
| | L2B | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGcgtacggtggctgcaccatct |
| | | gtcttcatcttcccgccatctgatgagcagttgaaatctggaactgcctctgtcgtgtg |
| | | cctgctgaataacttctatcccagagaggccaaagtacagtggaaggtggataacgccct |
| | | ccaatcgggtaactcccaggagagtgtcacagagcaggacagcTGTGGCGGAAGCGGAGC |
| | | AAAGCTCGCCGCACTGAAAGCCAAGCTGGCCGCTCTGAAGGGAGGAGGAGGAAGT |
| | | GGGGGAGGCGGCAGCGGGGGAGGTGGATCCGACATTCAAATGACGCAGTCACCCT |
| | | CTTCCCTGTCCGCCAGCGTGGGGGATCGCGTCACAATCACATGTCGCGCCTCTCAGG |
| | | ATGTGAACACCGCGGTGGCTTGGTATCAACAGAAGCCAGGCAAAGCACCTAAGCTC |
| | | CTGATCTACTCTGCCAGCTTTTTGTACAGCGGCGTGCCAAGTAGGTTTTCAGGCTCT |
| | | AGAAGCGGCACAGACTTTACACTGACTATCTCATCCCTGCAGCCTGAGGACTTTGCT |
| | | ACATATTATTGTCAACAACATTATACTACTCCACCCACTTTCGGACAGGGCACCAAA |
| | | GTGGAGATCAAACGCACCGGCTCCACCAGTGGAAGCGGTAAGCCTGGCTCTGGCGA |
| | | AGGCTCAGAAGTGCAACTTGTGGAGTCTGGAGGGGGGCTCGTCCAGCCCGGCGGTA |
| | | GTCTGAGGCTCAGCTGCGCCGCATCTGGCTTTAATATCAAGGACACATATATCCACT |
| | | GGGTACGGCAAGCACCAGGTAAGGGACTGGAGTGGGTCGCCAGAATCTACCCCACA |
| | | AACGGGTACACTCGCTATGCCGACTCAGTCAAGGGACGCTTTACAATAAGCGCAGA |
| | | CACAAGCAAGAACACCGCTTATCTGCAGATGAATAGCTTGCGGGCGGAGGATACAG |
| | | CTGTGTACTACTGCAGCAGATGGGGGGGCGACGGCTTTTACGCTATGGATGTGTGG |
| | | GGCCAGGGTACTCTGGTGACCGTCTCCTCCGGAGGCGGTGGGAGCGAACTGGCCGC |
| | | ACTGGAAGCTGAGCTGGCTGCCCTCGAAGCTGGAGGCTCTGGATGTgacagcacctaca |
| | | gcctcagcagcaccctgacgctgagcaaagcagactacgagaaacacaaagtctacgc |
| | | ctgcgaagtcacccatcagggcctgtcctcgcccgtcacaaagagcttcaacaggggag |
| | | agtgt |
| 31. | Her2ScFv- | GATATTCAGATGACTCAGACTACCAGTTCACTGAGCGCCTCCCTGGGCGATCGCGTG |
| | UCHT1- | ACAATTAGTTGTCGTGCGTCACAGGACATCCGGAACTATCTGAATTGGTACCAGCA |
| | CL- | GAAGCCGGACGGCACAGTCAAACTGCTGATCTATTACACTAGCCGTCTGCATTCCG |
| | L3A | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGcgtacggtggctgcaccatc |
| | | tgtcttcatcttcccgccatctgatgagcagttgaaatctggaactgcctctgttgtgt |
| | | gcctgctgaataacttctatcccagagaggccaaagtacagtggaaggtggataacgcc |
| | | ctccaatcgggtaactcccaggagagtgtcacagagcaggacagcaaggacagcacctac |
| | | agcctcagcagcaccctgacgctgagcaaagcagactacgagaaacacaaagtctacgc |
| | | ctgcgaagtcacccatcagggcctgGGCGGAAGCGG |
| | | AGCAAAGCTCGCCGCACTGAAAGCCAAGCTGGCCGCTCTGAAGTGTGGAGGAGGA |
| | | GGAAGTGGGGGAGGCGGCAGCGGGGGAGGTGGATCCGACATTCAAATGACGCAGT |
| | | CACCCTCTTCCCTGTCCGCCAGCGTGGGGGATCGCGTCACAATCACATGTCGCGCCT |
| | | CTCAGGATGTGAACACCGCGGTGGCTTGGTATCAACAGAAGCCAGGCAAAGCACCT |
| | | AAGCTCCTGATCTACTCTGCCAGCTTTTTGTACAGCGGCGTGCCAAGTAGGTTTTCA |
| | | GGCTCTAGAAGCGGCACAGACTTTACACTGACTATCTCATCCCTGCAGCCTGAGGAC |
| | | TTTGCTACATATTATTGTCAACAACATTATACTACTCCACCCACTTTCGGACAGGGC |
| | | ACCAAAGTGGAGATCAAACGCACCGGCTCCACCAGTGGAAGCGGTAAGCCTGGCTC |
| | | TGGCGAAGGCTCAGAAGTGCAACTTGTGGAGTCTGGAGGGGGGCTCGTCCAGCCCG |
| | | GCGGTAGTCTGAGGCTCAGCTGCGCCGCATCTGGCTTTAATATCAAGGACACATATA |
| | | TCCACTGGGTACGGCAAGCACCAGGTAAGGGACTGGAGTGGGTCGCCAGAATCTAC |
| | | CCCACAAACGGGTACACTCGCTATGCCGACTCAGTCAAGGGACGCTTTACAATAAG |
| | | CGCAGACACAAGCAAGAACACCGCTTATCTGCAGATGAATAGCTTGCGGGCGGAGG |
| | | ATACAGCTGTGTACTACTGCAGCAGATGGGGGGGCGACGGCTTTTACGCTATGGAT |
| | | GTGTGGGGCCAGGGTACTCTGGTGACCGTCTCCTCCGGAGGCGGTGGGAGCTGTGA |
| | | ACTGGCCGCACTGGAAGCTGAGCTGGCTGCCCTCGAAGCTGGAGGCTCTGGAtcgcccg |
| | | tcacaaagagcttcaacaggggagagtgt |
| 32. | Her2ScFv- | GATATTCAGATGACTCAGACTACCAGTTCACTGAGCGCCTCCCTGGGCGATCGCGTG |
| | UCHT1- | ACAATTAGTTGTCGTGCGTCACAGGACATCCGGAACTATCTGAATTGGTACCAGCA |
| | CL- | GAAGCCGGACGGCACAGTCAAACTGCTGATCTATTACACTAGCCGTCTGCATTCCG |
| | L3B | GTGTGCCCTCTAAGTTTTCTGGGAGTGGATCAGGCACTGATTATAGTCTGACCATTT |
| | | CAAACCTGGAACAGGAAGATATCGCCACCTACTTCTGTCAGCAGGGGAATACTCTG |
| | | CCGTGGACTTTCGCCGGAGGAACCAAACTGGAGATTAAGcgtacggtggctgcaccatct |
| | | gtcttcatcttcccgccatctgatgagcagttgaaatctggaactgcctctgttgtgtg |
| | | cctgctgaataacttctatcccagagaggccaaagtacagtggaaggtggataacgccct |
| | | ccaatcgggtaactcccaggagagtgtcacagagcaggacagcaaggacagcacctaca |
| | | gcctcagcagcaccctgacgctgagcaaagcagactacgagaaacacaaagtctacgc |
| | | ctgcgaagtcacccatcagggcctgTGTGGCGGAAG |
| | | CGGAGCAAAGCTCGCCGCACTGAAAGCCAAGCTGGCCGCTCTGAAGGGAGGAGGA |
| | | GGAAGTGGGGGAGGCGGCAGCGGGGGAGGTGGATCCGACATTCAAATGACGCAGT |
| | | CACCCTCTTCCCTGTCCGCCAGCGTGGGGGATCGCGTCACAATCACATGTCGCGCCT |
| | | CTCAGGATGTGAACACCGCGGTGGCTTGGTATCAACAGAAGCCAGGCAAAGCACCT |
| | | AAGCTCCTGATCTACTCTGCCAGCTTTTTGTACAGCGGCGTGCCAAGTAGGTTTTCA |
| | | GGCTCTAGAAGCGGCACAGACTTTACACTGACTATCTCATCCCTGCAGCCTGAGGAC |
| | | TTTGCTACATATTATTGTCAACAACATTATACTACTCCACCCACTTTCGGACAGGGC |
| | | ACCAAAGTGGAGATCAAACGCACCGGCTCCACCAGTGGAAGCGGTAAGCCTGGCTC |
| | | TGGCGAAGGCTCAGAAGTGCAACTTGTGGAGTCTGGAGGGGGGCTCGTCCAGCCCG |
| | | GCGGTAGTCTGAGGCTCAGCTGCGCCGCATCTGGCTTTAATATCAAGGACACATATA |
| | | TCCACTGGGTACGGCAAGCACCAGGTAAGGGACTGGAGTGGGTCGCCAGAATCTAC |
| | | CCCACAAACGGGTACACTCGCTATGCCGACTCAGTCAAGGGACGCTTTACAATAAG |
| | | CGCAGACACAAGCAAGAACACCGCTTATCTGCAGATGAATAGCTTGCGGGCGGAGG |
| | | ATACAGCTGTGTACTACTGCAGCAGATGGGGGGGCGACGGCTTTTACGCTATGGAT |
| | | GTGTGGGGCCAGGGTACTCTGGTGACCGTCTCCTCCGGAGGCGGTGGGAGCGAACT |
| | | GGCCGCACTGGAAGCTGAGCTGGCTGCCCTCGAAGCTGGAGGCTCTGGATGTtcgcccg |
| | | tcacaaagagcttcaacaggggagagtgt |

TABLE 3
Antibody or Antibody-fusion proteins-Amino Acid Sequence

| SEQ | | |
|---|---|---|
| ID NO: | Description | Sequence |
| 33. | Trastuzumab | EVQLVESGGGLVQPGGSLRLSCAASGFNIKDTYIHWVRQAPGKGLEWVARTYPTNG |
| | Heavy | YTRYADSVKGRFTISADTSKNTAYLQMNSLRAEDTAVYYCSRWGGDGFYAMDYW |
| | Chain | GQGTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCDK |
| | | THTCPPCPAPPVAGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKGLPSSIEKTI |
| | | SKAKGQPREPQVYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQKSLSLSPGK. |
| 34. | UCHT1 | EVQLQQSGPELVKPGASMKISCKASGYSFTGYTMNWVKQSHGKNLEWMGLINPYK |
| | Heavy | GVSTYNQKFKDKATLTVDKSSSTAYMELLSLTSEDSAVYYCARSGYYGDSDWYFD |
| | Chain | VWGQGTTLTVFSASTKGPSVFPLAPSSKSTSGGTAALGCLVKDYFPEPVTVSWNSGA |
| | IgG | LTSGVHTFPAVLQSSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVDVSHEDPEVKFNWY |
| | | VDGVEVHNAKTKPREEQYASTYRVVSVLTVLHQDWLNGKEYKCKVSNKALPAPIE |
| | | KTISKAKGQPREPQVYTLPPSRDELTKNQVSLTCLVKGFYPSDIAVEWESNGQPENN |
| | | YKTTPPVLDSDGSFFLYSKLTVDKSRWQQGNVFSCSVMHEALHNHYTQKSLSLSPG |
| | | K. |
| 35. | UCHT1 | EVQLQQSGPELVKPGASMKISCKASGYSFTGYTMNWVKQSHGKNLEWMGLINPYK |
| | Heavy | GVSTYNQKFKDKATLTVDKSSSTAYMELLSLTSEDSAVYYCARSGYYGDSDWYFD |
| | Chain | VWGQGTTLTVFSASTKGPSVFPLAPSSKSTSGGTAALGCLVKDYFPEPVTVSWNSGA |
| | Fab | LTSGVHTFPAVLQSSGLYSLSSVVTVPSSSLGTQTYICNVNHKPSNTKVDKKVEPKSC |
| 36. | Anti- | QVQLQQPGAELVKPGASVKMSCKASGYTFTSYNMHWVKQTPGRGLEWIGAIYPGN |
| | CD20 | GDTSYNQKFKGKATLTADKSSSTAYMQLSSLTSEDSAVYYCARSTYYGGDWYFNV |
| | Heavy | WGAGTTVTVSAASTKGPSVFPLAPSSKSTSGGTAALGCLVKDYFPEPVTVSWNSGAL |
| | Chain | TSGVHTFPAVLQSSGLYSLSSVVTVPSSSLGTQTYICNVNHKPSNTKVDKKVEPKSCD |
| | IgG | KTHTCPPCPAPPVAGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKGLPSSIEKT |
| | | ISKAKGQPREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSLSLSPGK |
| 37. | Anti-CD20 | QVQLQQPGAELVKPGASVKMSCKASGYTFTSYNMHWVKQTPGRGLEWIGAIYPGN |
| | Heavy | GDTSYNQKFKGKATLTADKSSSTAYMQLSSLTSEDSAVYYCARSTYYGGDWYFNV |
| | Chain | WGAGTTVTVSAASTKGPSVFPLAPSSKSTSGGTAALGCLVKDYFPEPVTVSWNSGAL |
| | Fab | TSGVHTFPAVLQSSGLYSLSSVVTVPSSSLGTQTYICNVNHKPSNTKVDKKVEPKSC |
| 38. | Anti- | EVKLQESGPGLVAPSQSLSVTCTVSGVSLPDYGVSWIRQPPRKGLEWLGVIWGSETT |
| | CD19 | YYNSALKSRLTIIKDNSKSQVFLKMNSLQTDDTAIYYCAKHYYYGGSYAMDYWGQ |
| | Heavy | GTSVTVSSASTKGPSVFPLAPSSKSTSGGTAALGCLVKDYFPEPVTVSWNSGALTSGV |
| | Chain | HTFPAVLQSSGLYSLSSVVTVPSSSLGTQTYICNVNHKPSNTKVDKKVEPKSCDKTHT |
| | IgG | CPPCPAPPVAGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKGLPSSIEKTISKA |
| | | KGQPREPQVYTLPPSRDELTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| 39. | Anti-CD19 | EVKLQESGPGLVAPSQSLSVTCTVSGVSLPDYGVSWIRQPPRKGLEWLGVIWGSETT |
| | Heavy | YYNSALKSRLTIIKDNSKSQVFLKMNSLQTDDTAIYYCAKHYYYGGSYAMDYWGQ |
| | Chain | GTSVTVSSASTKGPSVFPLAPSSKSTSGGTAALGCLVKDYFPEPVTVSWNSGALTSGV |
| | Fab | HTFPAVLQSSGLYSLSSVVTVPSSSLGTQTYICNVNHKPSNTKVDKKVEPKSC |
| 40. | Trastuzumab | DIQMTQSPSSLSASVGDRVTITCRASQDVNTAVAWYQQKPGKAPKWYSASFLYSG |
| | Light | VPSRFSGSRSGTDFTLTISSLQPEDFATYYCQQHYTTPPTFGQGTKLEIKRTVAAPSVFI |
| | Chain | FPPSDEQLKSGTASVVCLLNNFYPREAKVQWKVDNALQSGNSQESVTEQDSKDSTY |
| | | SLSSTLTLSKADYEKHKVYACEVTHQGLSSPVTKSFNRGEC |
| 41. | UCHT1 | DIQMTQTTSSLSASLGDRVTISCRASQDIRNYLNWYQQKPDGTVKLLIYYTSRLHSGV |
| | Light | PSKFSGSGSGTDYSLTISNLEQEDIATYFCQQGNTLPWTFAGGTKLEIKRTVAAPSVFI |
| | Chain | FPPSDEQLKSGTASVVCLLNNFYPREAKVQWKVDNALQSGNSQESVTEQDSKDSTY |
| | | SLSSTLTLSKADYEKHKVYACEVTHQGLSSPVTKSFNRGEC |
| 42. | anti- | DIQMTQTTSSLSASLGDRVTISCRASQDISKYLNWYQQKPDGTVKLLIYHTSRLHSGV |
| | CD19 LC | PSRFSGSGSGTDYSLTISNLEQEDIATYFCQQGNTLPYTFGGGTKLEIKRTVAAPSVFIF |
| | | PPSDEQLKSGTASVVCLLNNFYPREAKVQWKVDNALQSGNSQESVTEQDSKDSTYS |
| | | LSSTLTLSKADYEKHKVYACEVTHQGLSSPVTKSFNRGEC |
| 43. | anti- | QIVLSQSPAILSASPGEKVTMTCRASSSVSYIHWFQQKPGSSPKPWIYATSNLASGVPV |
| | CD20 LC | RFSGSGSGTSYSLTISRVEAEDAATYYCQQWTSNPPTFGGGTKLEIKRTVAAPSVFIFP |
| | | PSDEQLKSGTASVVCLLNNFYPREAKVQWKVDNALQSGNSQESVTEQDSKDSTYSL |
| | | SSTLTLSKADYEKHKVYACEVTHQGLSSPVTKSFNRGEC |
| 44. | Palivizumab | QVTLRESGPALVKPTQTLTLTCTFSGFSLSTSGMSVGWIRQPPGKALEWLADIWWDD |
| | Heavy | KKDYNPSLKSRLTISKDTSKNQVVLKVTNMDPADTATYYCARSMITNWYFDVWGA |
| | Chain | GTTVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPKSCDKTHT |
| | | CPPCPAPPVAGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKGLPSSIEKTISKA |
| | | KGQPREPQVYTLPPSRDELTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| 45. | hEPO- | EVQLVESGGGLVQPGGSLRLSCAASGFNIKDTYIHWVRQAPGKGLEWVARTYPTNG |
| | coil- | YTRYADSVKGRFTISADTSKNTAYLQMNSLRAEDTAVYYCSRWGGDGFYAMDYW |
| | Her2- | GQGTLVTVSSASTKGPSVFPLAPSSKSTSGGTAALGCLVKDYFPEPVTVSWNSGALTS |
| | CH1 | GVHTFPAVLQSGGSGAKLAALKAKLAALKGGGGSAPPRLICDSRVLERYLLEAKEAENITT |
| | | GCAEHCSLNENITVPDTKVNFYAWKRMEVGQQAVEVWQGLALLSEAVLRGQALLVNSSQ |
| | | PWEPLQLHVDKAVSGLRSLTTLLRALGAQKEAISPPDAASAAPLRTITADTFRKLFRVYSNF |
| | | LRGKLKLYTGEACRTGDRGGGGSELAALEAELAALEAGGSGLYSLSSVVTVPSSSLGTQT |
| | | YICNVNHKPSNTKVDKKVEPKSCDKTHTCPPCPAPPVAGPSVFLFPPKPKDTLMISRT |
| | | PEVTCVVVDVSHEDPEVKFNWYVDGVEVHNAKTKPREEQYNSTYRVVSVLTVLHQ |
| | | DWLNGKEYKCKVSNKGLPSSIEKTISKAKGQPREPQVYTLPPSRDELTKNQVSLTCL |
| | | VKGFYPSDIAVEWESNGQPENNYKTTPPVLDSDGSFFLYSKLTVDKSRWQQGNVFSC |
| | | SVMHEALHNHYTQKSLSLSPGK. |
| 46. | hEPO- | EVQLVESGGGLVQPGGSLRLSCAASGFNIKDTYIHWVRQAPGKGLEWVARIYPTNG |
| | coil- | YTRYADSVKGRFTISADTSKNTAYLQMNSLRAEDTAVYYCSRWGGDGFYAMDYW |
| | Her2- | GQGTLVTVSSASTKGPSVFPLAPSSKSTSGGTAALGCLVKDYFPEPVTVSWNSGALTS |
| | CH3 | GVHTFPAVLQSSGLYSLSSVVTVPSSSLGTQTYICNVNHKPSNTKVDKKVEPKSCDK |
| | | THTCPPCPAPPVAGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKGLPSSIEKTI |
| | | SKAKGQPREPQVYTLPPSRDELGGSGAKLAALKAKLAALKGGGGSAPPRLICDSRVLERY |
| | | LLEAKEAENITTGCAEHCSLNENITVPDTKVNFYAWKRMEVGQQAVEVWQGLALLSEAVL |
| | | RGQALLVNSSQPWEPLQLHVDKAVSGLRSLTTLLRALGAQKEAISPPDAASAAPLRTITADT |
| | | FRKLFRVYSNFLRGKLKLYTGEACRTGDRGGGGSELAALEAELAALEAGGSGQVSLTCLV |
| | | KGFYPSDIAVEWESNGQPENNYKTTPPVLDSDGSFFLYSKLTVDKSRWQQGNVFSCS |
| | | VMHEALHNHYTQKSLSLSPGK. |
| 47. | CXCR4- | EVQLVESGGGLVQPGGSLRLSCAASGFNIKDTYIHWVRQAPGKGLEWVARIYPTNG |
| | BP-coil- | YTRYADSVKGRFTISADTSKNTAYLQMNSLRAEDTAVYYCSRWGGDGFYAMDYW |
| | Her2- | GQGTLVTVSSASTKGPSVFPLAPSSKSTSGGTAALGCLVKDYFPEPVTVSWNSGALTS |
| | CH1 | GVHTFPAVLQSGGSGAKLAALKAKLAALKAKLYRKCRGGRRWCYQKLEAELAALEAELA |
| | | ALEAGGSGLYSLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK. |
| 48. | hEPO- | DIQMTQSPSSLSASVGDRVTITCRASQDVNTAVAWYQQKPGKAPKWYSASFLYSG |
| | coil- | VPSRFSGSRSGTDFTLTISSLQPEDFATYYCQQHYTTPPTFGQGTKLEIKRTVAAPSVFI |
| | Her2-CL | FPPSDEQLKSGTASVVCLLNNFYPREAKVQWKVDNALQSGNSQESVTEQDSGGSGA |
| | | KLAALKAKLAALKGGGGSAPPRLICDSRVLERYLLEAKEAENITTGCAEHCSLNENITVPDT |
| | | KVNFYAWKRMEVGQQAVEVWQGLALLSEAVLRGQALLVNSSQPWEPLQLHVDKAVSGLR |
| | | SLTTLLRALGAQKEAISPPDAASAAPLRTITADTFRKLFRVYSNFLRGKLKLYTGEACRTGD |
| | | RGGGGSELAALEAELAALEAGGSGDSTYSLSSTLTLSKADYEKHKVYACEVTHQGLSS |
| | | PVTKSFNRGEC |
| 49. | hEPO- | DIQMTQSPSSLSASVGDRVTITCRASQDVNTAVAWYQQKPGKAPKWYSASFLYSG |
| | G4S- | VPSRFSGSRSGTDFTLTISSLQPEDFATYYCQQHYTTPPTFGQGTKLEIKRTVAAPSVFI |
| | Trastuzumab- | FPPSDEQLKSGTASVVCLLNNFYPREAKVQWKVDNALQSGNSQESVTEQDSGGGGS |
| | CL | APPRLICDSRVLERYLLEAKEAENITTGCAEHCSLNENITVPDTKVNFYAWKRMEVGQQAV |
| | | EVWQGLALLSEAVLRGQALLVNSSQPWEPLQLHVDKAVSGLRSLTTLLRALGAQKEAISPP |
| | | DAASAAPLRTITADTFRKLFRVYSNFLRGKLKLYTGEACRTGDRGGGGSDSTYSLSSTLTL |
| | | SKADYEKHKVYACEVTHQGLSSPVTKSFNRGEC |
| 50. | TCP1- | DIQMTQTTSSLSASLGDRVTISCRASQDIRNYLNWYQQKPDGTVKLLIYYTSRLHSGV |
| | coil- | PSKFSGSGSGTDYSLTISNLEQEDIATYFCQQGNTLPWTFAGGTKLEIKRTVAAPSVFI |
| | UCHT1- | FPPSDEQLKSGTASVVCLLNNFYPREAKVQWKVDNALQSGNSQESVTEQDSGGSGA |
| | CL | KLAALKAKLAALKAKLTPSPFSHLEAELAALEAELAALEAGGSGDSTYSLSSTLTLSKADY |
| | | EKHKVYACEVTHQGLSSPVTKSFNRGEC |
| 51. | TCP1- | DIQMTQTTSSLSASLGDRVTISCRASQDIRNYLNWYQQKPDGTVKLLIYYTSRLHSGV |
| | UCHT1- | PSKFSGSGSGTDYSLTISNLEQEDIATYFCQQGNTLPWTFAGGTKLEIKRTVAAPSVFI |
| | CL | FPPSDEQLKSGTASVVCLLNNFYPREAKVQWKVDNALQSGNSQESVTEQDSGGGGS |
| | | CTPSPFSHCGGGGSDSTYSLSSTLTLSKADYEKHKVYACEVTHQGLSSPVTKSFNRGE |
| | | C |
| 52. | NGR- | DIQMTQTTSSLSASLGDRVTISCRASQDIRNYLNWYQQKPDGTVKLLIYYTSRLHSGV |
| | coil- | PSKFSGSGSGTDYSLTISNLEQEDIATYFCQQGNTLPWTFAGGTKLEIKRTVAAPSVFI |
| | UCHT1- | FPPSDEQLKSGTASVVCLLNNFYPREAKVQWKVDNALQSGNSQESVTEQDSGGSGA |
| | CL | KLAALKAKLAALKAKLTYNGRTLEAELAALEAELAALEAGGSGDSTYSLSSTLTLSKADYE |
| | | KHKVYACEVTHQGLSSPVTKSFNRGEC |
| 53. | NGR- | DIQMTQTTSSLSASLGDRVTISCRASQDIRNYLNWYQQKPDGTVKLLIYYTSRLHSGV |
| | UCHT1- | PSKFSGSGSGTDYSLTISNLEQEDIATYFCQQGNTLPWTFAGGTKLEIKRTVAAPSVFI |
| | CL | FPPSDEQLKSGTASVVCLLNNFYPREAKVQWKVDNALQSGNSQESVTEQDSGGGGS |
| | | CNGRCVSGCAGRCGGGGSDSTYSLSSTLTLSKADYEKHKVYACEVTHQGLSSPVTKS |
| | | FNRGEC |
| 54. | Int-coil- | DIQMTQTTSSLSASLGDRVTISCRASQDIRNYLNWYQQKPDGTVKLLIYYTSRLHSGV |
| | UCHT1- | PSKFSGSGSGTDYSLTISNLEQEDIATYFCQQGNTLPWTFAGGTKLEIKRTVAAPSVFI |
| | CL | FPPSDEQLKSGTASVVCLLNNFYPREAKVQWKVDNALQSGNSQESVTEQDSGGSGA |
| | | KLAALKAKLAALKGGGGSGCPQGRGDWAPTSCKQDSDCRAGCVCGPNGFCGGGGGSE |
| | | LAALEAELAALEAGGSGDSTYSLSSTLTLSKADYEKHKVYACEVTHQGLSSPVTKSFN |
| | | RGEC |
| 55. | CXCR4- | QIVLSQSPAILSASPGEKVTMTCRASSSVSYIHWFQQKPGSSPKPWIYATSNLASGVPV |
| | BP-coil- | RFSGSGSGTSYSLTISRVEAEDAATYYCQQWTSNPPTFGGGTKLEIKRTVAAPSVFIFP |
| | CD20-CL | PSDEQLKSGTASVVCLLNNFYPREAKVQWKVDNALQSGNSQESVTEQDSGGSGAKL |
| | | AALKAKLAALKAKLYRKCRGGRRWCYQKLEAELAALEAELAALEAGGSGDSTYSLSSTLT |
| | | LSKADYEKHKVYACEVTHQGLSSPVTKSFNRGEC |
| 56. | CXCR4- | DIQMTQSPSSLSASVGDRVTITCRASQDVNTAVAWYQQKPGKAPKWYSASFLYSG |
| | BP-coil- | VPSRFSGSRSGTDFTLTISSLQPEDFATYYCQQHYTTPPTFGQGTKLEIKRTVAAPSVFI |
| | Her2-CL | FPPSDEQLKSGTASVVCLLNNFYPREAKVQWKVDNALQSGNSQESVTEQDSGGSGA |
| | | KLAALKAKLAALKAKLYRKCRGGRRWCYQKLEAELAALEAELAALEAGGSGDSTYSLSST |
| | | LTLSKADYEKHKVYACEVTHQGLSSPVTKSFNRGEC |
| 57. | GCN4- | DIQMTQTTSSLSASLGDRVTISCRASQDISKYLNWYQQKPDGTVKLLIYHTSRLHSGV |
| | CD19-CL | PSRFSGSGSGTDYSLTISNLEQEDIATYFCQQGNTLPYTFGGGTKLEIKRTVAAPSVFIF |
| | | PPSDEQLKSGTASVVCLLNNFYPREAKVQWKVDNALQSGNSQESVTEQDSGGGGSN |
| | | YHLENEVARLKKLGGGGSDSTYSLSSTLTLSKADYEKHKVYACEVTHQGLSSPVTKSF |
| | | NRGEC |
| 58. | Her2ScFv- | DIQMTQTTSSLSASLGDRVTISCRASQDIRNYLNWYQQKPDGTVKLLIYYTSRLHSGV |
| | UCHT1- | PSKFSGSGSGTDYSLTISNLEQEDIATYFCQQGNTLPWTFAGGTKLEIKRTVAAPSVFI |
| | CL | FPPSDEQLKSGTASVVCLLNNFYPREAKVQWKVDNALQSGNSQESVTEQDSGGGGS |
| | | GGGGSGGGGSDIQMTQSPSSLSASVGDRVTITCRASQDVNTAVAWYQQKPGKAPKWYS |
| | | ASFLYSGVPSRFSGSRSGTDFTLTISSLQPEDFATYYCQQHYTTPPLFGQGTKVEIKRTGSTS |
| | | GSGKPGSGEGSEVQLVESGGGLVQPGGSLRLSCAASGFNIKDLYIHWVRQAPGKGLEWV |
| | | ARIYPTNGYTRYADSVKGRFTISADTSKNTAYLQMNSLRAEDTAVYYCSRWGGDGFYAMDV |
| | | WGQGTLVTVSSGGGGSDSTYSLSSTLTLSKADYEKHKVYACEVTHQGLSSPVTKSFNR |
| | | GEC |
| 59. | anti- | DIQMTQTTSSLSASLGDRVTISCRASQDIRNYLNWYQQKPDGTVKLLIYYTSRLHSGV |
| | CD19ScFv- | PSKFSGSGSGTDYSLTISNLEQEDIATYFCQQGNTLPWTFAGGTKLEIKRTVAAPSVFI |
| | UCHT1- | FPPSDEQLKSGTASVVCLLNNFYPREAKVQWKVDNALQSGNSQESVTEQDSGGGGS |
| | CL | GGGGSGGGGSEVKLQESGPGLVAPSQSLSVTCTVSGVSLPDYGVSWIRQPPRKGLEWLGV |
| | | IWGSETTYYNSALKSRLTIIKDNSKSQVFLKVINSLQTDDTAIYYCAKHYYYGGSYAMDYWG |
| | | QGTSVTVSSGGGGSGGGGSGGGGSDIQMTQLTSSLSASLGDRVTISCRASQDISKYLNWYQ |
| | | QKPDGTVKLLIYHTSRLHSGVPSRFSGSGSGTDYSLTISNLEQEDIATYFCQQGNTLPYLFG |
| | | GGTKLEIGGGGSDSTYSLSSTLTLSKADYEKHKVYACEVTHQGLSSPVTKSFNRGEC |
| 60. | UCHT1 | EVQLVESGGGLVQPGGSLRLSCAASGFNIKDTYIHWVRQAPGKGLEWVARTYPTNG |
| | ScFv- | YTRYADSVKGRFTISADTSKNTAYLQMNSLRAEDTAVYYCSRWGGDGFYAMDYW |
| | Her2- | GQGTLVTVSSASTKGPSVFPLAPSSKSTSGGTAALGCLVKDYFPEPVTVSWNSGALTS |
| | CH1 | GVHTFPAVLQSGGGGSGGGGSGGGGSDIQMTQLTSSLSASLGDRVTISCRASQDIRNYLN |
| | | WYQQKPDGTVKLLIYYTSRLHSGVPSKFSGSGSGTDYSLTISNLEQEDIATYFCQQGNTLP |
| | | WTFAGGTKLEIKGSTSGSGKPGSGEGSTKGEVQLQQSGPELVKPGASMKISCKASGYSFT |
| | | GYTMNWVKQSHGKNLEWMGLINPYKGVSTYNQKFKDKALLTVDKSSSTAYMELLSLTSED |
| | | SAVYYCARSGYYGDSDWYFDVWGQGTTLTVFSGGGGSLYSLSSVVTVPSSSLGTQTYIC |
| | | NVNHKPSNTKVDKKVEPKSCDKTHTCPPCPAPPVAGPSVFLFPPKPKDTLMISRTPEV |
| | | TCVVVDVSHEDPEVKFNWYVDGVEVHNAKTKPREEQYNSTYRVVSVLTVLHQDW |
| | | LNGKEYKCKVSNKGLPSSIEKTISKAKGQPREPQVYTLPPSRDELTKNQVSLTCLVKG |
| | | FYPSDIAVEWESNGQPENNYKTTPPVLDSDGSFFLYSKLTVDKSRWQQGNVFSCSVM |
| | | HEALHNHYTQKSLSLSPGK. |
| 61. | anti- | EVKLQESGPGLVAPSQSLSVTCTVSGVSLPDYGVSWIRQPPRKGLEWLGVIWGSETT |
| | CD19 | YYNSALKSRLTIIKDNSKSQVFLKMNSLQTDDTAIYYCAKHYYYGGSYAMDYWGQ |
| | Fab HC1- | GTSVTVSSASTKGPSVFPLAPSSNYHLENEVARLKKLSGGTAALGCLVKDYFPEPVTVS |
| | GCN4 | WNSGALTSGVHTFPAVLQSSGLYSLSSVVTVPSSSLGTQTYICNVNHKPSNTKVDKK |
| | switch | VEPKSC |
| | Heavy | |
| | Chain | |
| 62. | anti- | EVKLQESGPGLVAPSQSLSVTCTVSGVSLPDYGVSWIRQPPRKGLEWLGVIWGSETT |
| | CD19 | YYNSALKSRLTIIKDNSKSQVFLKMNSLQTDDTAIYYCAKHYYYGGSYAMDYWGQ |
| | IgGHC1- | GTSVTVSSASTKGPSVFPLAPSSNYHLENEVARLKKLSGGTAALGCLVKDYFPEPVTVS |
| | GCN4 | WNSGALTSGVHTFPAVLQSSGLYSLSSVVTVPSSSLGTQTYICNVNHKPSNTKVDKK |
| | switch | VEPKSCDKTHTCPPCPAPPVAGPSVFLFPPKPKDTLMISRTPEVTCVVVDVSHEDPEV |
| | Heavy | KFNWYVDGVEVHNAKTKPREEQYNSTYRVVSVLTVLHQDWLNGKEYKCKVSNKG |
| | Chain | LPSSIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLS |
| | | LSPGK |
| 63. | anti- | EVKLQESGPGLVAPSQSLSVTCTVSGVSLPDYGVSWIRQPPRKGLEWLGVIWGSETT |
| | CD19 | YYNSALKSRLTIIKDNSKSQVFLKMNSLQTDDTAIYYCAKHYYYGGSYAMDYWGQ |
| | Fab C | GTSVTVSSASTKGPSVFPLAPSSKSTSGGTAALGCLVKDYFPEPVTVSWNSGALTSGV |
| | term- | HTFPAVLQSSGLYSLSSVVTVPSSSLGTQTYICNVNHKPSNTKVDKKVEPKSCGGGGS |
| | GCN4 | NYHLENEVARLKKL |
| | switch | |
| | Heavy | |
| | Chain | |
| 64. | anti- | EVKLQESGPGLVAPSQSLSVTCTVSGVSLPDYGVSWIRQPPRKGLEWLGVIWGSETT |
| | CD19 | YYNSALKSRLTIIKDNSKSQVFLKMNSLQTDDTAIYYCAKHYYYGGSYAMDYWGQ |
| | IgG | GTSVTVSSASTKGPSVFPLAPSSKSTSGGTAALGCLVKDYFPEPVTVSWNSGALTSGV |
| | hinge- | HTFPAVLQSSGLYSLSSVVTVPSSSLGTQTYICNVNHKPSNTKVDKKVEPKSCGGGGS |
| | GCN4 | NYHLENEVARLKKLGGSDKTHTCPPCPAPPVAGPSVFLFPPKPKDTLMISRTPEVTCVV |
| | switch | VDV SHEDPEVKFNWYVDGVEVHNAKTKPREEQYNSTYRVVSVLTVLHQDWLNGK |
| | Heavy | EYKCKVSNKGLPSSIEKTISKAKGQPREPQVYTLPPSRDELTKNQVSLTCLVKGFYPS |
| | Chain | DIAVEWESNGQPENNYKTTPPVLDSDGSFFLYSKLTVDKSRWQQGNVFSCSVMHEA |
| | | LHNHYTQKSLSLSPGK |
| 65. | CXCR4- | QIVLSQSPAILSASPGEKVTMTCRASSSVSYIHWFQQKPGSSPKPWIYATSNLASGVPV |
| | BP-coil- | RFSGSGSGTSYSLTISRVEAEDAATYYCQQWTSNPPTFGGGTKLEIKRTVAAPSVFIFP |
| | CD20-CL | PSDEQLKSGTASVVCLLNNFYPREAKVQWKVDNALQSGNSQESVTEQDSGGSGAKL |
| | (IgG) | AALKAKLAALKGGGGSCYRKCRGGRRWCYQKCGGGGSELAALEAELAALEAGGS |
| | | GDSTYSLSSTLTLSKADYEKHKVYACEVTHQGLSSPVTKSFNRGEC |
| 66. | CXCR4- | DIQMTQSPSTLSASVGDRVTITCKCQLSVGYMHWYQQKPGKAPKLLIYDTSKLASGV |
| | BP-coil- | PSRFSGSGSGTEFTLTISSLQPDDFATYYCFQGSGYPFTFGGGTKLEIKRTVAAPSVFIF |
| | Syn-CL | PPSDEQLKSGTASVVCLLNNFYPREAKVQWKVDNALQSGNSQESVTEQDSGGSGAK |
| | | LAALKAKLAALKGGGGSCYRKCRGGRRWCYQKCGGGGSELAALEAELAALEAGG |
| | | SGDSTYSLSSTLTLSKADYEKHKVYACEVTHQGLSSPVTKSFNRGEC |
| 67. | Her2ScFv- | DIQMTQTTSSLSASLGDRVTISCRASQDIRNYLNWYQQKPDGTVKLLIYYTSRLHSGV |
| | UCHT1- | PSKF SGSGSGTDYSLTISNLEQEDIATYFCQQGNTLPWTFAGGTKLEIKRTVAAPSVFI |
| | CL-L2A | FPPSDEQLKSGTASVVCLLNNFYPREAKVQWKVDNALQSGNSQESVTEQDSGGSGA |
| | | KLAALKAKLAALKCGGGGSGGGGSGGGGSDIQMTQSPSSLSASVGDRVTITCRASQ |
| | | DVNTAVAWYQQKPGKAPKWYSASFLYSGVPSRFSGSRSGTDFTLTISSLQPEDFAT |
| | | YYCQQHYTTPPTFGQGTKVEIKRTGSTSGSGKPGSGEGSEVQLVESGGGLVQPGGSL |
| | | RLSCAASGFNIKDTYIHWVRQAPGKGLEWVARTYPTNGYTRYADSVKGRFTISADTS |
| | | KNTAYLQMNSLRAEDTAVYYCSRWGGDGFYAMDVWGQGTLVTVSSGGGGSCELA |
| | | ALEAELAALEAGGSGDSTYSLSSTLTLSKADYEKHKVYACEVTHQGLSSPVTKSFNR |
| | | GEC |
| 68. | Her2ScFv- | DIQMTQTTSSLSASLGDRVTISCRASQDIRNYLNWYQQKPDGTVKLLIYYTSRLHSGV |
| | UCHT1- | PSKFSGSGSGTDYSLTISNLEQEDIATYFCQQGNTLPWTFAGGTKLEIKRTVAAPSVFI |
| | CL-L2B | FPPSDEQLKSGTASVVCLLNNFYPREAKVQWKVDNALQSGNSQESVTEQDSCGGSG |
| | | AKLAALKAKLAALKGGGGSGGGGSGGGGSDIQMTQSPSSLSASVGDRVTITCRASQ |
| | | DVNTAVAWYQQKPGKAPKWYSASFLYSGVPSRFSGSRSGTDFTLTISSLQPEDFAT |
| | | YYCQQHYTTPPTFGQGTKVEIKRTGSTSGSGKPGSGEGSEVQLVESGGGLVQPGGSL |
| | | RLSCAASGFNIKDTYIHWVRQAPGKGLEWVARTYPTNGYTRYADSVKGRFTISADTS |
| | | KNTAYLQMNSLRAEDTAVYYCSRWGGDGFYAMDVWGQGTLVTVSSGGGGSELAA |
| | | LEAELAALEAGGSGCDSTYSLSSTLTLSKADYEKHKVYACEVTHQGLSSPVTKSFNR |
| | | GEC |
| 69. | Her2ScFv- | DIQMTQTTSSLSASLGDRVTISCRASQDIRNYLNWYQQKPDGTVKLLIYYTSRLHSGV |
| | UCHT1- | PSKFSGSGSGTDYSLTISNLEQEDIATYFCQQGNTLPWTFAGGTKLEIKRTVAAPSVFI |
| | CL-L3A | FPPSDEQLKSGTASVVCLLNNFYPREAKVQWKVDNALQSGNSQESVTEQDSKDSTY |
| | | SLSSTLTLSKADYEKHKVYACEVTHQGLGGSGAKLAALKAKLAALKCGGGGSGGG |
| | | GSGGGGSDIQMTQSPSSLSASVGDRVTITCRASQDVNTAVAWYQQKPGKAPKWYS |
| | | ASFLYSGVPSRFSGSRSGTDFTLTISSLQPEDFATYYCQQHYTTPPTFGQGTKVEIKRT |
| | | GSTSGSGKPGSGEGSEVQLVESGGGLVQPGGSLRLSCAASGFNIKDTYIHWVRQAPG |
| | | KGLEWVARTYPTNGYTRYADSVKGRFTISADTSKNTAYLQMNSLRAEDTAVYYCSR |
| | | WGGDGFYAMDVWGQGTLVTVSSGGGGSCELAALEAELAALEAGGSGSPVTKSFNR |
| | | GEC |
| 70. | Her2ScFv- | DIQMTQTTSSLSASLGDRVTISCRASQDIRNYLNWYQQKPDGTVKLLIYYTSRLHSGV |
| | UCHT1- | PSKFSGSGSGTDYSLTISNLEQEDIATYFCQQGNTLPWTFAGGTKLEIKRTVAAPSVFI |
| | CL-L3B | FPPSDEQLKSGTASVVCLLNNFYPREAKVQWKVDNALQSGNSQESVTEQDSKDSTY |
| | | SLSSTLTLSKADYEKHKVYACEVTHQGLCGGSGAKLAALKAKLAALKGGGGSGGG |
| | | GSGGGGSDIQMTQSPSSLSASVGDRVTITCRASQDVNTAVAWYQQKPGKAPKLUYS |
| | | ASFLYSGVPSRFSGSRSGTDFTLTISSLQPEDFATYYCQQHYTTPPTFGQGTKVEIKRT |
| | | GSTSGSGKPGSGEGSEVQLVESGGGLVQPGGSLRLSCAASGFNIKDTYIHWVRQAPG |
| | | KGLEWVARTYPTNGYTRYADSVKGRFTISADTSKNTAYLQMNSLRAEDTAVYYCSR |
| | | WGGDGFYAMDVWGQGTLVTVSSGGGGSELAALEAELAALEAGGSGCSPVTKSFNR |
| | | GEC |

For Table 3: Linker sequences are underlined. Inserted sequences are italicized

TABLE 4

| SEQ | | |
|---|---|---|
| ID NO: | Description | Sequence |
| 71. | Adapter peptide | GGSG |
| | A | |
| 72. | Adapter peptide B | GGGGS |
| 73. | Adapter peptide C | GGGGSGGGGSGGGGS |
| 74. | Adapter peptide | LEAELAALEAELAALEAGGSG |
| | D | |
| 75. | Adapter peptide E | GGSGAKLAALKAKLAALKAKL |
| 76. | Adapter peptide F | GGGGSELAALEAELAALEAGGS |
| | | G |
| 77. | Adapter peptide | GGSGAKLAALKAKLAALKGGG |
| | G | GS |

TABLE 5

| SEQ | | |
|---|---|---|
| ID | | |
| NO: | Description | Sequence |
| 78. | TCP1-short | TPSPFSH |
| 79. | TCP1-long | CTPSPFSHC |
| 80. | NGR-short | TYNGRT |
| 81. | NGR-long | CNGRCVSGCAGRC |
| 82. | Int | GCPQGRGDWAPTSCKQDSDCRAGCVCGPNGFCG |
| 83. | CXCR4-BP | YRKCRGGRRWCYQK |
| 84. | GCN4 | NYHLENEVARLKKL |
| 85. | hEPO | APPRLICDSRVLERYLLEAKEAENITTGCAEHCSLNENITVPDTKVNFYAWKRMEV |
| | | GQQAVEVWQGLALLSEAVLRGQALLVNSSQPWEPLQLHVDKAVSGLRSLTTLLRA |
| | | LGAQKEAISPPDAASAAPLRTITADTFRKLFRVYSNFLRGKLKLYTGEACRTGDR |
| 86. | Her2scFv | DIQMTQSPSSLSASVGDRVTITCRASQDVNTAVAWYQQKPGKAPKWYSASFLYSG |
| | | VPSRFSGSRSGTDFTLTISSLQPEDFATYYCQQHYTTPPTFGQGTKVEIKRTGSTSGS |
| | | GKPGSGEGSEVQLVESGGGLVQPGGSLRLSCAASGFNIKDTYIHWVRQAPGKGLEW |
| | | VARTYPTNGYTRYADSVKGRFTISADTSKNTAYLQMNSLRAEDTAVYYCSRWGGD |
| | | GFYAMDVWGQGTLVTVSS |
| 87. | anti-CD19 | EVKLQESGPGLVAPSQSLSVTCTVSGVSLPDYGVSWIRQPPRKGLEWLGVIWGSETT |
| | scFv | YYNSALKSRLTIIKDNSKSQVFLKMNSLQTDDTAIYYCAKHYYYGGSYAMDYWGQ |
| | | GTSVTVSS |
| 88. | UCHT1scFv | DIQMTQTTSSLSASLGDRVTISCRASQDIRNYLNWYQQKPDGTVKLLIYYTSRLHSG |
| | | VPSKFSGSGSGTDYSLTISNLEQEDIATYFCQQGNTLPWTFAGGTKLEIKGSTSGSGK |
| | | PGSGEGSTKGEVQLQQSGPELVKPGASMKISCKASGYSFTGYTMNWVKQSHGKNL |
| | | EWMGLINPYKGVSTYNQKFKDKATLTVDKSSSTAYMELLSLTSEDSAVYYCARSG |
| | | YYGDSDWYFDVWGQGTTLTVFS |

TABLE 6

| SEQ ID NO: | Description | Sequence |
|---|---|---|
| 89. | IgGl-CH1 consensus insertion sequence A | FPEPVT |
| 90. | IgGl-CH1 consensus insertion sequence B | SSKSTSGGTA |
| 91. | IgGl-CH1 consensus insertion sequence C | FPEPV |
| 92. | IgGl-CH1 consensus insertion sequence D | NSGALTSG |
| 93. | IgGl-CH1 consensus insertion sequence E | QSSGL |
| 94. | IgGl-CH1 consensus insertion sequence F | PSSSLGTQTY |
| 95. | IgGl-CH1 consensus insertion sequence G | KPSN |
| 96. | IgG1-CH2 consensus insertion sequence A | VSHEDPEVK |
| 97. | IgG1-CH2 consensus insertion sequence B | EQYNSTY |
| 98. | IgG1-CH2 consensus insertion sequence C | SNKALPAPI |
| 99. | IgG1-CH3 consensus insertion sequence A | PPSRDELTKN |
| 100. | IgG1-CH3 consensus insertion sequence B | SNGQ |
| 101. | IgG1-CH3 consensus insertion sequence C | KSRWQQGNV |
| 102. | IgG4-CH1 consensus insertion sequence A | PCSRSTSES |
| 103. | IgG4-CH2 consensus insertion sequence A | SQEDPE |
| 104. | IgG4-CH2 consensus insertion sequence B | QFDST |
| 105. | IgG4-CH2 consensus insertion sequence C | NGLPSS |
| 106. | IgG4-CH3 consensus insertion sequence A | PSSQEEMTK |
| 107. | IgG4-CH3 consensus insertion sequence B | NGQPENN |
| 108. | IgG4-CH3 consensus insertion sequence C | EGNV |
| 109. | Kappa-CL consensus insertion sequence A | SDEQLKSGT |
| 110. | Kappa-CL consensus insertion sequence B | FYPREAK |
| 111. | Kappa-CL consensus insertion sequence C | DNA |
| 112. | Kappa-CL consensus insertion sequence D | EQDSKDS |
| 113. | Kappa-CL consensus insertion sequence E | LSKADYEKHK |
| 114. | Kappa-CL consensus insertion sequence F | HQGLSSP |
| 115. | Lambda-CL consensus insertion sequence A | PSSEELET |
| 116. | Lambda-CL consensus insertion sequence B | DFYPGV |
| 117. | Lambda-CL consensus insertion sequence C | GTPVTQ |
| 118. | Lambda-CL consensus insertion sequence D | QPSKQSNNKY |
| 119. | Lambda-CL consensus insertion sequence E | ARAWERHS |
| 120. | Lambda-CL consensus insertion sequence F | HEGH |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1-33. (canceled)

34. A bispecific antibody comprising:
a) a first antibody or antibody fragment comprising a modified constant domain or portion thereof; and
b) a second antibody or antibody fragment,
wherein the second antibody or antibody fragment is located within the modified constant domain or portion thereof.

35. (canceled)

36. (canceled)

37. The bispecific antibody of claim 34, wherein the modified constant domain comprises a CH1 or portion thereof.

38. The bispecific antibody of claim 34, wherein the modified constant domain comprises a CH2 or portion thereof.

39. The bispecific antibody of claim 34, wherein the modified constant domain comprises a CH3 or portion thereof.

40. The bispecific antibody of claim 34, wherein the modified constant domain comprises a hinge region or portion thereof.

41. (canceled)

42. (canceled)

43. The bispecific antibody of claim 34, wherein the second antibody or antibody fragment is a single chain variable fragment.

44. (canceled)

45. (canceled)

46. (canceled)

47. (canceled)

48. (canceled)

49. (canceled)

50. (canceled)

51. (canceled)

52. The bispecific antibody of claim 34, wherein the first antibody or antibody fragment comprises 50 or more consecutive amino acids from any one of SEQ ID NO: 33-44.

53. (canceled)

54. (canceled)

55. (canceled)

56. (canceled)

57. (canceled)

58. (canceled)

59. A method of treating a disease or condition in a subject in need thereof, the method comprising administering to the subject the bispecific antibody of claim 34.

60. (canceled)

61. (canceled)

62. (canceled)

63. (canceled)

64. (canceled)

65. An antibody fusion protein comprising an antibody or antibody fragment comprising a constant domain modified by insertion of a non-antibody polypeptide region comprising 15 or more amino acids within the constant domain, wherein the constant domain is modified from (i) a first constant domain of an antibody heavy chain (CH1) or a portion thereof, or (ii) a constant domain of an antibody light chain (CL) or a portion thereof.

66. The antibody fusion protein of claim 65, wherein the constant domain is modified by replacing 0 to about 20 amino acids of the constant domain with the non-antibody polypeptide

67. The antibody fusion protein of claim 65, wherein the constant domain is modified from the first constant domain of an antibody heavy chain (CH1) or portion thereof.

68. The antibody fusion protein of claim 65, wherein the constant domain is modified from the constant domain of an antibody light chain (CL1) or portion thereof.

69. The antibody fusion protein of claim 65, wherein the antibody fusion protein comprises 50 or more consecutive amino acids from any one of SEQ ID NOs: 33-44.

70. The antibody fusion protein of claim 65, wherein the non-antibody polypeptide region comprises erythropoietin, chemokine receptor-4 binding peptide, a tumor-homing peptide, an integrin αvβ3 binding peptide, or a T-cell epitope peptide.

71. An antibody fusion protein comprising an antibody or antibody fragment comprising a constant domain modified with a non-antibody polypeptide region comprising 15 or more amino acids, wherein the non-antibody polypeptide region is located within the modified constant domain, and the non-antibody region comprises a linker peptide having a coiled-coil secondary structure.

72. The antibody fusion protein of claim 71, wherein the modified constant domain is modified from a heavy chain constant domain or a portion thereof.

73. The antibody fusion protein of claim 71, wherein the modified constant domain is modified from a light chain constant domain or a portion thereof.

74. The antibody fusion protein of claim 71, wherein the modified constant domain comprises an antibody hinge region or a portion thereof.

75. The antibody fusion protein of claim 71, comprising 50 or more consecutive amino acids from any one of SEQ ID NOs: 33-44.

76. The antibody fusion protein of claim 71, wherein the linker peptide is not an amino acid sequence from the antibody or antibody fragment.


SEQUENCE LISTING (120 sequences)

SEQ ID NO: 1 - DNA, 1353 bp, Artificial Sequence
  Description of Artificial Sequence Synthetic polynucleotide
gaagtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 2 - DNA, 1362 bp, Artificial Sequence
  Description of Artificial Sequence Synthetic polynucleotide
gaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 3 - DNA, 675 bp, Artificial Sequence
  Description of Artificial Sequence Synthetic polynucleotide
gaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgt

SEQ ID NO: 4 - DNA, 1356 bp, Artificial Sequence
  Description of Artificial Sequence Synthetic polynucleotide
caggtgcagctccagcagccgggagcagaattggttaagcctggggcctcagtgaaaatgagctgtaaggccagcggctacaccttcacctcctataacatgcattgggtaaaacagacccccggcagaggtctcgagtggatcggagcgatttatccgggcaatggagacacttcctataatcagaaatttaagggcaaggccactctcacagccgacaagtcttcatccaccgcttatatgcagctgagctccttgacctctgaggacagcgccgtttactattgcgcacgaagcacgtactacgggggagattggtactttaacgtgtggggggccggaaccaccgtgactgtgtctgctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 5 - DNA, 672 bp, Artificial Sequence
  Description of Artificial Sequence Synthetic polynucleotide
caggtgcagctccagcagccgggagcagaattggttaagcctggggcctcagtgaaaatgagctgtaaggccagcggctacaccttcacctcctataacatgcattgggtaaaacagacccccggcagaggtctcgagtggatcggagcgatttatccgggcaatggagacacttcctataatcagaaatttaagggcaaggccactctcacagccgacaagtcttcatccaccgcttatatgcagctgagctccttgacctctgaggacagcgccgtttactattgcgcacgaagcacgtactacgggggagattggtactttaacgtgtggggggccggaaccaccgtgactgtgtctgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgt

SEQ ID NO: 6 - DNA, 1347 bp, Artificial Sequence
  Description of Artificial Sequence Synthetic polynucleotide
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - DNA, 669 bp, Artificial Sequence
  Description of Artificial Sequence Synthetic polynucleotide
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgt

SEQ ID NO: 8 - DNA, 642 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 9 - DNA, 642 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaagacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - DNA, 1347 bp, Artificial Sequence
  Description of Artificial Sequence Synthetic polynucleotide
caggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 11 - DNA, 1983 bp, Artificial Sequence
  Description of Artificial Sequence Synthetic polynucleotide
gaagtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtccggcggaagcggagcaaagctcgccgcactgaaagccaagctggccgctctgaaggggggtggcggaagcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggcggaggtgggagtgaactggccgcactggaagctgagctggctgccctcgaagctggaggctctggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 12 - DNA, 1980 bp, Artificial Sequence
  Description of Artificial Sequence Synthetic polynucleotide
gaagtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgggcggaagcggagcaaagctcgccgcactgaaagccaagctggccgctctgaaggggggtggcggaagcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggcggaggtgggagtgaactggccgcactggaagctgagctggctgccctcgaagctggaggctctggacaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 13 - DNA, 1518 bp, Artificial Sequence
  Description of Artificial Sequence Synthetic polynucleotide
gaagtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtccggcggaagcggagcaaagctcgccgcactgaaagccaagctggccgctctgaaggctaagttgtatcgcaaatgtagaggaggccgaaggtggtgctaccaaaagcttgaggctgaactggccgcactggaagctgagctggctgccctcgaagctggaggctctggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 14 - DNA, 1275 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggggggtggcggaagcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggcggaggtgggagtgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 15 - DNA, 1167 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcgggggtggcggaagcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggcggaggtgggagtgacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 16 - DNA, 786 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggccaagctgactcccagccctttctcacacctggaagctgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 17 - DNA, 696 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcggcggaggcgggagctgtactcccagccctttctcacactgtggtggcggaggcagcgacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 18 - DNA, 783 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggctaagttgacatataatgggaggacacttgaggctgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 19 - DNA, 708 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcggcggaggcgggagctgtaacggaagatgtgtgtccggttgcgctggccgctgtggtggcggaggcagcgacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 20 - DNA, 876 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggggggtggcggaagcggttgccctcaagggcgcggggattgggcacccacctcctgtaagcaagactctgactgccgcgctggctgcgtgtgcggtcccaatggtttttgcgggggaggcggtgggagcgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 21 - DNA, 804 bp, Artificial Sequence
  Description of Artificial Sequence Synthetic polynucleotide
cagattgtgttgtctcagtcccccgcaattctcagtgcgtcccccggcgaaaaggtgaccatgacctgccgcgcttcctcctcagtgagttatatccactggttccagcagaagccaggatcaagcccgaagccgtggatctacgccaccagcaacctggccagcggagtgcctgtgaggttctctggttctggcagcgggaccagttactcactcaccatttcccgggttgaggccgaagatgccgctacttattattgccaacagtggacctccaatccgccaacatttgggggagggactaaactggagattaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggctaagttgtatcgcaaatgtagaggaggccgaaggtggtgctaccaaaagcttgaggctgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 22 - DNA, 807 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggctaagttgtatcgcaaatgtagaggaggccgaaggtggtgctaccaaaagcttgaggctgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 23 - DNA, 711 bp, Artificial Sequence
  Description of Artificial Sequence Synthetic polynucleotide
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaggcgggagcaattatcatcttgaaaatgaggtcgctcgtctcaagaaactcggtggcggaggcagcgacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 24 - DNA, 1431 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcgggggcggcggatctggcggaggcgggtctggcgggggtggatctgatattcagatgacccagagccctagctctcttagcgcatccgttggtgaccgcgtaactattacttgcagagccagtcaggatgtgaatacggctgtggcctggtatcagcagaaacctgggaaagcccccaagctgctgatctactccgccagcttcctgtattctggtgtgccgagcagatttagcgggtccagaagcggcaccgactttacccttactatttcatccctgcagccggaggatttcgccacatattattgtcagcagcactacaccacacctcccacattcggccagggcactaaggtggagatcaaacgcacagggtcaacttcaggttccggcaagcccggttctggagaggggagcgaagtgcagctcgtcgagtccggcggtggtctggtccagccgggaggaagcctgcgactgagctgtgcagcgtctggattcaacatcaaggacacctacatccactgggtgcgccaggcacccggcaaaggccttgagtgggtggcacggatctacccaactaacgggtataccagatacgccgatagcgtgaagggacggttcacaataagcgcagatacttctaagaacactgcctatctgcagatgaactcactgcgggctgaggacactgccgtgtattattgtagcagatggggtggcgatgggttctacgccatggatgtctggggtcagggtactttggtgaccgtgtcttcagggggcggcggcagtgacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 25 - DNA, 1422 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcgggggtggcggaagtgggggcggaggcagtgggggaggcggtagtgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggaggcggaggatccggaggcggtggcagcggcggcggaggttctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagcttgagatcggtggcggtgggtctgacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 26 - DNA, 2148 bp, Artificial Sequence
  Description of Artificial Sequence Synthetic polynucleotide
gaagtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtccggagggggaggaagtggtggcggggggagcggcggaggaggctccgacattcagatgacccagaccaccagctctctgagtgccagccttggggatcgggtgacaatttcctgccgggcctctcaggatatacgcaactacctgaactggtaccagcagaagcctgatggcacagtgaaactgctgatttactatacgtccagactgcactcaggggttcccagtaaattcagcggctccggctccggaacggactactcactgaccatctcaaacttggagcaggaggacattgccacttatttctgccaacaggggaacaccctcccctggactttcgctggaggaactaagctcgaaataaagggatcaacttcagggtcagggaagcctggtagcggtgaggggtccacgaagggtgaagtgcagctgcagcagtctggacccgagctggtgaagccgggtgcatctatgaaaatttcctgcaaagcaagcgggtattcctttaccgggtacactatgaattgggtgaagcagagccacgggaagaatctggaatggatgggactgataaatccttacaagggcgtcagcacatacaatcagaaattcaaggataaggctacacttacagtagacaaaagttcctccactgcatatatggagctgctttcactcacctcagaagactccgccgtgtattattgtgctagatcagggtactatggcgactcagactggtacttcgatgtatggggacagggtaccacactgaccgtgttcagcggaggaggcggcagcctctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 27 - DNA, 822 bp, Artificial Sequence
  Description of Artificial Sequence Synthetic polynucleotide
cagattgtgttgtctcagtcccccgcaattctcagtgcgtcccccggcgaaaaggtgaccatgacctgccgcgcttcctcctcagtgagttatatccactggttccagcagaagccaggatcaagcccgaagccgtggatctacgccaccagcaacctggccagcggagtgcctgtgaggttctctggttctggcagcgggaccagttactcactcaccatttcccgggttgaggccgaagatgccgctacttattattgccaacagtggacctccaatccgccaacatttgggggagggactaaactggagattaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggggggtggcggaagctgctatcgcaaatgtagaggaggccgaaggtggtgctaccaaaagtgtggcggaggtgggagtgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 28 - DNA, 822 bp, Artificial Sequence
  Description of Artificial Sequence Synthetic polynucleotide
gacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaaggggggtggcggaagctgctatcgcaaatgtagaggaggccgaaggtggtgctaccaaaagtgtggcggaggtgggagtgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 29 - DNA, 1545 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcggcggaagcggagcaaagctcgccgcactgaaagccaagctggccgctctgaagtgtggaggaggaggaagtgggggaggcggcagcgggggaggtggatccgacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctccggaggcggtgggagctgtgaactggccgcactggaagctgagctggctgccctcgaagctggaggctctggagacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 30 - DNA, 1545 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagctgtggcggaagcggagcaaagctcgccgcactgaaagccaagctggccgctctgaagggaggaggaggaagtgggggaggcggcagcgggggaggtggatccgacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctccggaggcggtgggagcgaactggccgcactggaagctgagctggctgccctcgaagctggaggctctggatgtgacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 31 - DNA, 1545 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgggcggaagcggagcaaagctcgccgcactgaaagccaagctggccgctctgaagtgtggaggaggaggaagtgggggaggcggcagcgggggaggtggatccgacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctccggaggcggtgggagctgtgaactggccgcactggaagctgagctggctgccctcgaagctggaggctctggatcgcccgtcacaaagagcttcaacaggggagagtgt

SEQ ID NO: 32 - DNA, 1545 bp, Artificial Sequence
  Description of Artificial Sequence Synthetic polynucleotide
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtgtggcggaagcggagcaaagctcgccgcactgaaagccaagctggccgctctgaagggaggaggaggaagtgggggaggcggcagcgggggaggtggatccgacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctccggaggcggtgggagcgaactggccgcactggaagctgagctggctgccctcgaagctggaggctctggatgttcgcccgtcacaaagagcttcaacaggggagagtgt

SEQ ID NO: 33 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 452 aa, Artificial Sequence
  Description of Artificial Sequence Synthetic polypeptide
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 225 aa, Artificial Sequence
  Description of Artificial Sequence Synthetic polypeptide
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 450 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 224 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 38 - protein, 449 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 223 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 40 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 214 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 214 aa, Artificial Sequence
  Description of Artificial Sequence Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 449 aa, Artificial Sequence
  Description of Artificial Sequence Synthetic polypeptide
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 659 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSGGSGAKLAALKAKLAALKGGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSELAALEAELAALEAGGSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 658 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELGGSGAKLAALKAKLAALKGGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSELAALEAELAALEAGGSGQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 504 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSGGSGAKLAALKAKLAALKAKLYRKCRGGRRWCYQKLEAELAALEAELAALEAGGSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 425 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSGGSGAKLAALKAKLAALKGGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSELAALEAELAALEAGGSGDSTYSLSSTLTLSKADYEKHKVYACEVTHQGLSSPVTKSFNRGEC

SEQ ID NO: 49 - protein, 389 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSGGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGGSDSTYSLSSTLTLSKADYEKHKVYACEVTHQGLSSPVTKSFNRGEC

SEQ ID NO: 50 - protein, 262 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSGGSGAKLAALKAKLAALKAKLTPSPFSHLEAELAALEAELAALEAGGSGDSTYSLSSTLTLSKADYEKHKVYACEVTHQGLSSPVTKSFNRGEC

SEQ ID NO: 51 - protein, 232 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSGGGGSCTPSPFSHCGGGGSDSTYSLSSTLTLSKADYEKHKVYACEVTHQGLSSPVTKSFNRGEC

SEQ ID NO: 52 - protein, 261 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSGGSGAKLAALKAKLAALKAKLTYNGRTLEAELAALEAELAALEAGGSGDSTYSLSSTLTLSKADYEKHKVYACEVTHQGLSSPVTKSFNRGEC

SEQ ID NO: 53 - protein, 236 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSGGGGSCNGRCVSGCAGRCGGGGSDSTYSLSSTLTLSKADYEKHKVYACEVTHQGLSSPVTKSFNRGEC

SEQ ID NO: 54 - protein, 292 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSGGSGAKLAALKAKLAALKGGGGSGCPQGRGDWAPTSCKQDSDCRAGCVCGPNGFCGGGGGSELAALEAELAALEAGGSGDSTYSLSSTLTLSKADYEKHKVYACEVTHQGLSSPVTKSFNRGEC

SEQ ID NO: 55 - protein, 268 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSGGSGAKLAALKAKLAALKAKLYRKCRGGRRWCYQKLEAELAALEAELAALEAGGSGDSTYSLSSTLTLSKADYEKHKVYACEVTHQGLSSPVTKSFNRGEC

SEQ ID NO: 56 - protein, 269 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSGGSGAKLAALKAKLAALKAKLYRKCRGGRRWCYQKLEAELAALEAELAALEAGGSGDSTYSLSSTLTLSKADYEKHKVYACEVTHQGLSSPVTKSFNRGEC

SEQ ID NO: 57 - protein, 237 aa, Artificial Sequence
  Description of Artificial Sequence Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKRTVAAPSVFIFPPSDEQLKSGTASVVCLLNNFYPREAKVQWKVDNALQSGNSQESVTEQDSGGGGSNYHLENEVARLKKLGGGGSDSTYSLSSTLTLSKADYEKHKVYACEVTHQGLSSPVTKSFNRGEC

SEQ ID NO: 58 - protein, 477 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSDSTYSLSSTLTLSKADYEKHKVYACEVTHQGLSSPVTKSFNRGEC

SEQ ID NO: 59 - protein, 474 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIGGGGSDSTYSLSSTLTLSKADYEKHKVYACEVTHQGLSSPVTKSFNRGEC

SEQ ID NO: 60 - protein, 714 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGSTSGSGKPGSGEGSTKGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 234 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSNYHLENEVARLKKLSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 62 - protein, 460 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSNYHLENEVARLKKLSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 242 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSNYHLENEVARLKKL

SEQ ID NO: 64 - protein, 471 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSNYHLENEVARLKKLGGSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 274 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSGGSGAKLAALKAKLAALKGGGGSCYRKCRGGRRWCYQKCGGGGSELAALEAELAALEAGGSGDSTYSLSSTLTLSKADYEKHKVYACEVTHQGLSSPVTKSFNRGEC

SEQ ID NO: 66 - protein, 274 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSGGSGAKLAALKAKLAALKGGGGSCYRKCRGGRRWCYQKCGGGGSELAALEAELAALEAGGSGDSTYSLSSTLTLSKADYEKHKVYACEVTHQGLSSPVTKSFNRGEC

SEQ ID NO: 67 - protein, 515 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSGGSGAKLAALKAKLAALKCGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSCELAALEAELAALEAGGSGDSTYSLSSTLTLSKADYEKHKVYACEVTHQGLSSPVTKSFNRGEC

SEQ ID NO: 68 - protein, 515 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSCGGSGAKLAALKAKLAALKGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSELAALEAELAALEAGGSGCDSTYSLSSTLTLSKADYEKHKVYACEVTHQGLSSPVTKSFNRGEC

SEQ ID NO: 69 - protein, 515 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLGGSGAKLAALKAKLAALKCGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSCELAALEAELAALEAGGSGSPVTKSFNRGEC

SEQ ID NO: 70 - protein, 515 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLCGGSGAKLAALKAKLAALKGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSELAALEAELAALEAGGSGCSPVTKSFNRGEC

SEQ ID NO: 71 - protein, 4 aa, Artificial Sequence
  Description of Artificial Sequence Synthetic peptide
GGSG

SEQ ID NO: 72 - protein, 5 aa, Artificial Sequence
  Description of Artificial Sequence Synthetic peptide
GGGGS

SEQ ID NO: 73 - protein, 15 aa, Artificial Sequence
  Description of Artificial Sequence Synthetic peptide
GGGGSGGGGSGGGGS

SEQ ID NO: 74 - protein, 21 aa, Artificial Sequence
  Description of Artificial Sequence Synthetic peptide
LEAELAALEAELAALEAGGSG

SEQ ID NO: 75 - protein, 21 aa, Artificial Sequence
  Description of Artificial Sequence Synthetic peptide
GGSGAKLAALKAKLAALKAKL

SEQ ID NO: 76 - protein, 23 aa, Artificial Sequence
  Description of Artificial Sequence Synthetic peptide
GGGGSELAALEAELAALEAGGSG

SEQ ID NO: 77 - protein, 23 aa, Artificial Sequence
  Description of Artificial Sequence Synthetic peptide
GGSGAKLAALKAKLAALKGGGGS

SEQ ID NO: 78 - protein, 7 aa, Artificial Sequence
  Description of Artificial Sequence Synthetic peptide
TPSPFSH

SEQ ID NO: 79 - protein, 9 aa, Artificial Sequence
  Description of Artificial Sequence Synthetic peptide
CTPSPFSHC

SEQ ID NO: 80 - protein, 6 aa, Artificial Sequence
  Description of Artificial Sequence Synthetic peptide
TYNGRT

SEQ ID NO: 81 - protein, 13 aa, Artificial Sequence
  Description of Artificial Sequence Synthetic peptide
CNGRCVSGCAGRC

SEQ ID NO: 82 - protein, 33 aa, Artificial Sequence
  Description of Artificial Sequence Synthetic polypeptide
GCPQGRGDWAPTSCKQDSDCRAGCVCGPNGFCG

SEQ ID NO: 83 - protein, 14 aa, Artificial Sequence
  Description of Artificial Sequence Synthetic peptide
YRKCRGGRRWCYQK

SEQ ID NO: 84 - protein, 14 aa, Artificial Sequence
  Description of Artificial Sequence Synthetic peptide
NYHLENEVARLKKL

SEQ ID NO: 85 - protein, 166 aa, Artificial Sequence
  Description of Artificial Sequence Synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 86 - protein, 244 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 87 - protein, 120 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 88 - protein, 247 aa, Artificial Sequence
  Description of Artificial Sequence Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGSTSGSGKPGSGEGSTKGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 89 - protein, 6 aa, Artificial Sequence
  Description of Artificial Sequence Synthetic peptide
FPEPVT

SEQ ID NO: 90 - protein, 10 aa, Artificial Sequence
  Description of Artificial Sequence Synthetic peptide
SSKSTSGGTA

SEQ ID NO: 91 - protein, 5 aa, Artificial Sequence
  Description of Artificial Sequence Synthetic peptide
FPEPV

SEQ ID NO: 92 - protein, 8 aa, Artificial Sequence
  Description of Artificial Sequence Synthetic peptide
NSGALTSG

SEQ ID NO: 93 - protein, 5 aa, Artificial Sequence
  Description of Artificial Sequence Synthetic peptide
QSSGL

SEQ ID NO: 94 - protein, 10 aa, Artificial Sequence
  Description of Artificial Sequence Synthetic peptide
PSSSLGTQTY

SEQ ID NO: 95 - protein, 4 aa, Artificial Sequence
  Description of Artificial Sequence Synthetic peptide
KPSN

SEQ ID NO: 96 - protein, 9 aa, Artificial Sequence
  Description of Artificial Sequence Synthetic peptide
VSHEDPEVK

SEQ ID NO: 97 - protein, 7 aa, Artificial Sequence
  Description of Artificial Sequence Synthetic peptide
EQYNSTY

SEQ ID NO: 98 - protein, 9 aa, Artificial Sequence
  Description of Artificial Sequence Synthetic peptide
SNKALPAPI

SEQ ID NO: 99 - protein, 10 aa, Artificial Sequence
  Description of Artificial Sequence Synthetic peptide
PPSRDELTKN

SEQ ID NO: 100 - protein, 4 aa, Artificial Sequence
  Description of Artificial Sequence Synthetic peptide
SNGQ

SEQ ID NO: 101 - protein, 9 aa, Artificial Sequence
  Description of Artificial Sequence Synthetic peptide
KSRWQQGNV

SEQ ID NO: 102 - protein, 9 aa, Artificial Sequence
  Description of Artificial Sequence Synthetic peptide
PCSRSTSES

SEQ ID NO: 103 - protein, 6 aa, Artificial Sequence
  Description of Artificial Sequence Synthetic peptide
SQEDPE

SEQ ID NO: 104 - protein, 5 aa, Artificial Sequence
  Description of Artificial Sequence Synthetic peptide
QFDST

SEQ ID NO: 105 - protein, 6 aa, Artificial Sequence
  Description of Artificial Sequence Synthetic peptide
NGLPSS

SEQ ID NO: 106 - protein, 9 aa, Artificial Sequence
  Description of Artificial Sequence Synthetic peptide
PSSQEEMTK

SEQ ID NO: 107 - protein, 7 aa, Artificial Sequence
  Description of Artificial Sequence Synthetic peptide
NGQPENN

SEQ ID NO: 108 - protein, 4 aa, Artificial Sequence
  Description of Artificial Sequence Synthetic peptide
EGNV

SEQ ID NO: 109 - protein, 9 aa, Artificial Sequence
  Description of Artificial Sequence Synthetic peptide
SDEQLKSGT

SEQ ID NO: 110 - protein, 7 aa, Artificial Sequence
  Description of Artificial Sequence Synthetic peptide
FYPREAK

SEQ ID NO: 111 - protein, 3 aa, Artificial Sequence
  Description of Artificial Sequence Synthetic peptide
DNA

SEQ ID NO: 112 - protein, 7 aa, Artificial Sequence
  Description of Artificial Sequence Synthetic peptide
EQDSKDS

SEQ ID NO: 113 - protein, 10 aa, Artificial Sequence
  Description of Artificial Sequence Synthetic peptide
LSKADYEKHK

SEQ ID NO: 114 - protein, 7 aa, Artificial Sequence
  Description of Artificial Sequence Synthetic peptide
HQGLSSP

SEQ ID NO: 115 - protein, 8 aa, Artificial Sequence
  Description of Artificial Sequence Synthetic peptide
PSSEELET

SEQ ID NO: 116 - protein, 6 aa, Artificial Sequence
  Description of Artificial Sequence Synthetic peptide
DFYPGV

SEQ ID NO: 117 - protein, 6 aa, Artificial Sequence
  Description of Artificial Sequence Synthetic peptide
GTPVTQ

SEQ ID NO: 118 - protein, 10 aa, Artificial Sequence
  Description of Artificial Sequence Synthetic peptide
QPSKQSNNKY

SEQ ID NO: 119 - protein, 8 aa, Artificial Sequence
  Description of Artificial Sequence Synthetic peptide
ARAWERHS

SEQ ID NO: 120 - protein, 4 aa, Artificial Sequence
  Description of Artificial Sequence Synthetic peptide
HEGH